(19) United States
(12) Patent Application Publication
(10) Pub. No.: US 2023/0200229 A1
(43) Pub. Date: Jun. 22, 2023

(54) ORGANIC LIGHT EMITTING DEVICE

(71) Applicant: LG CHEM, LTD., Seoul (KR)

(72) Inventors: Sang Duk SUH, Daejeon (KR); Min Woo JUNG, Daejeon (KR); Jungha LEE, Daejeon (KR); Su Jin HAN, Daejeon (KR); Seulchan PARK, Daejeon (KR); Sunghyun HWANG, Daejeon (KR); Dong Hoon LEE, Daejeon (KR)

(21) Appl. No.: 17/925,084

(22) PCT Filed: Aug. 4, 2021
(86) PCT No.: PCT/KR2021/010271

§ 371 Date: Nov. 14, 2022

(30) Foreign Application Priority Data
Aug. 6, 2020 (KR) 10-2020-0098672
Aug. 3, 2021 (KR) 10-2021-0101881

(51) Int. Cl. H10K 85/60; C07D 487/04; C09K 11/06; C07D 209/86

(52) U.S. Cl. CPC H10K 85/6572; C07D 487/04; C09K 11/06; H10K 85/6574; C07D 209/86; H10K 85/615; H10K 85/654; H10K 85/6576; H10K 50/11

(57) ABSTRACT

Provided is an organic light-emitting device with improved driving voltage, efficiency, and lifetime, comprising: an anode; a cathode; a light emitting layer between the anode and the cathode; an electron blocking layer between the anode and the light-emitting layer; and a hole transport layer between the electron blocking layer and the anode, wherein the light-emitting layer includes a compound of Formula 1, a compound of Formula 2, and a compound of Formula 3:

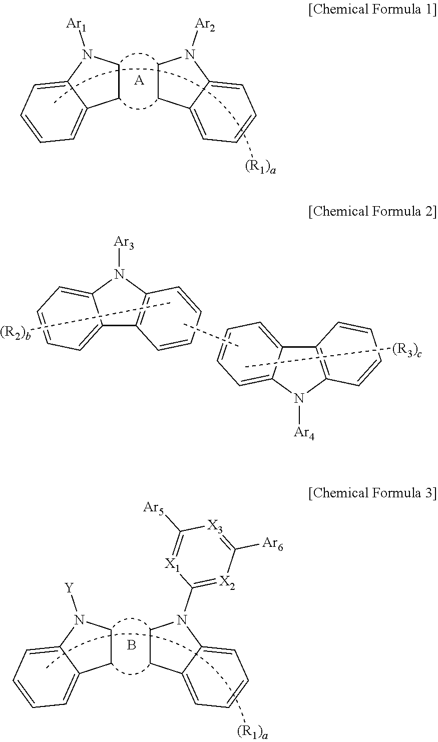

where A is a benzene ring condensed with two adjacent pentagonal rings, B is a benzene ring condensed with two adjacent pentagonal rings, and the other substituents are as defined in the specification.

### CROSS-REFERENCE TO RELATED APPLICATION(S)

This application is a National Stage Application of International Application No. PCT/KR2021/010271 filed on Aug. 4, 2021, which claims the benefit of Korean Patent Applications No. 10-2020-0098672 filed on Aug. 6, 2020 and No. 10-2021-0101881 filed on Aug. 3, 2021 in the Korean Intellectual Property Office, the disclosures of which are incorporated herein by reference in their entirety.

### TECHNICAL FIELD

The present disclosure relates to an organic light emitting device having improved driving voltage, efficiency, and lifetime.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of an organic light emitting device having improved driving voltage, efficiency, and lifetime.

### PRIOR ART

### Patent Documents

(Patent Literature 1) Korean Unexamined Patent Publication No. 10-2000-0051826

### BRIEF DESCRIPTION

### Technical Problem

The present disclosure relates to an organic light emitting device having improved driving voltage, efficiency, and lifetime.

### Technical Solution

In the present disclosure, there is provided an organic light emitting device, including

an anode;

a cathode;

a light emitting layer between the anode and the cathode;

an electron blocking layer between the anode and the light emitting layer; and

a hole transport layer between the electron blocking layer and the anode,

wherein the light emitting layer includes a compound of the following Chemical Formula 1, a compound of the following Chemical Formula 2, and a compound of the following Chemical Formula 3:

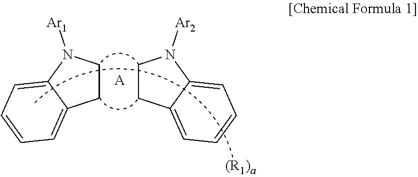

wherein in Chemical Formula 1:

A is a benzene ring condensed with two adjacent pentagonal rings;

Ar₁ is a substituted or unsubstituted C₆₋₆₀ aryl;

Ar₂ is a substituted or unsubstituted C₆₋₆₀ aryl, or a substituted or unsubstituted C₂₋₆₀ heteroaryl containing at least one heteroatom selected from the group consisting of O and S;

R₁ is hydrogen, deuterium, a substituted or unsubstituted C₆₋₆₀ aryl, or a substituted or unsubstituted C₂₋₆₀ heteroaryl containing at least one heteroatom selected from the group consisting of N, O and S; and

a is an integer of 1 to 10;

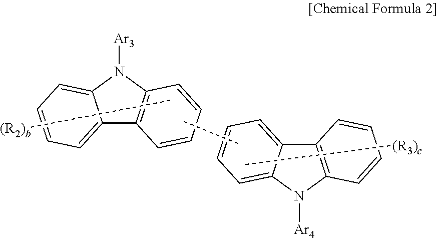

wherein in Chemical Formula 2:

Ar₃ and Ar₄ are each independently a substituted or unsubstituted C₆₋₆₀ aryl, or a substituted or unsubstituted C₂₋₆₀ heteroaryl containing at least one heteroatom selected from the group consisting of N, O and S;

R₂ and R₃ are each independently hydrogen, deuterium, a substituted or unsubstituted C₆₋₆₀ aryl, or a substituted or unsubstituted C₂₋₆₀ heteroaryl containing at least one heteroatom selected from the group consisting of N, O and S; and

b and c are each independently an integer of 1 to 7;

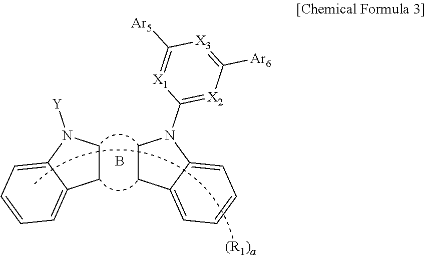

wherein in Chemical Formula 3:

B is a benzene ring condensed with two adjacent pentagonal rings;

X₁ to X₃ are each independently CH, or N, provided that at least one of X₁ to X₃ is N;

Ar₅ and Ar₆ are each independently a substituted or unsubstituted C₆₋₆₀ aryl, or a substituted or unsubstituted C₂₋₆₀ heteroaryl containing at least one heteroatom selected from the group consisting of N, O and S;

Y is NAr₇, wherein Ar₇ is a substituted or unsubstituted C₆₋₆₀ aryl, or a substituted or unsubstituted C₂₋₆₀ heteroaryl containing at least one heteroatom selected from the group consisting of N, O and S;

R₄ is hydrogen, deuterium, a substituted or unsubstituted C₆₋₆₀ aryl, or a substituted or unsubstituted C₂₋₆₀ heteroaryl containing at least one heteroatom selected from the group consisting of N, O and S; and

d is an integer of 1 to 10.

### Advantageous Effects

The above-described organic light emitting device has improved driving voltage, efficiency, and lifetime.

### BRIEF DESCRIPTION OF THE DRAWINGS

FIG. 1 shows an example of an organic light emitting device including a substrate 1, an anode 2, a hole transport layer 6, an electron blocking layer 3, a light emitting layer 4, an electron transport layer 7, and a cathode 5.

### DETAILED DESCRIPTION

Hereinafter, embodiments of the present disclosure will be described in more detail to facilitate understanding of the invention.

As used herein, the notation

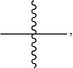

or [symbol] means a bond linked to another substituent

As used herein, the term “a substituted or unsubstituted” means being unsubstituted or substituted with one or more substituents selected from the group consisting of deuterium, a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heterocyclic group containing at least one of N, O and S atoms, or being unsubstituted or substituted with a substituent in which two or more substituents of the above-exemplified substituents are connected. For example, “a substituent in which two or more substituents are connected” can be a biphenyl group. Namely, a biphenyl group can be an aryl group, or it can also be interpreted as a substituent in which two phenyl groups are connected.

In the present disclosure, the carbon number of a carbonyl group is not particularly limited, but is preferably 1 to 40. Specifically, the carbonyl group can be a compound having the following structural formulae, but is not limited thereto:

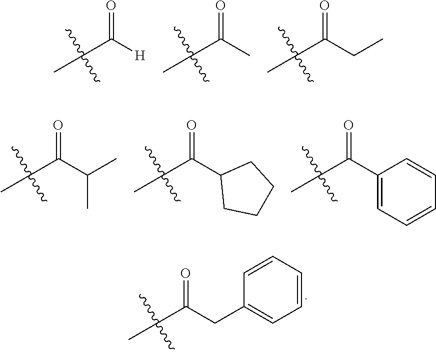

In the present disclosure, an ester group can have a structure in which oxygen of the ester group is substituted by a straight-chain, branched-chain, or cyclic alkyl group having 1 to 25 carbon atoms, or an aryl group having 6 to 25 carbon atoms. Specifically, the ester group can be a compound having the following structural formulae, but is not limited thereto:

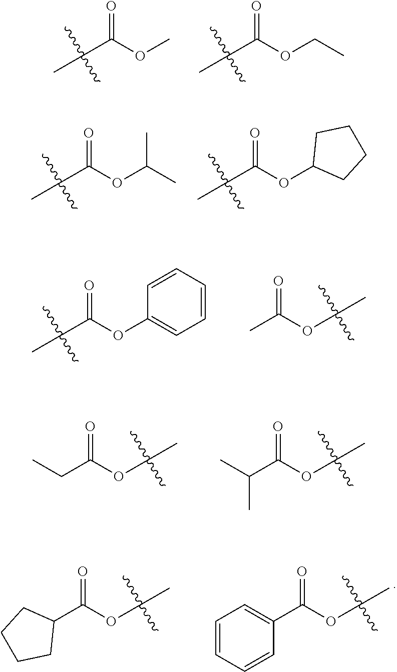

In the present disclosure, the carbon number of an imide group is not particularly limited, but is preferably 1 to 25. Specifically, the imide group can be a compound having the following structural formulae, but is not limited thereto:

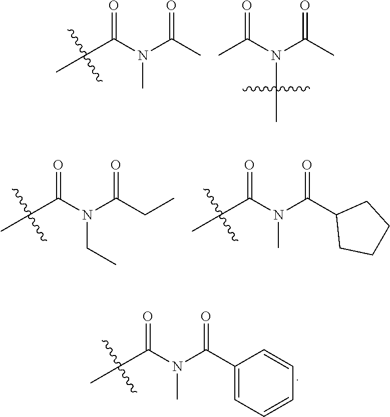

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, a phenylboron group and the like, but is not limited thereto.

In the present disclosure, examples of a halogen group include fluorine, chlorine, bromine, or iodine.

In the present disclosure, the alkyl group can be straight-chain, or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, aryl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The monocyclic aryl group includes a phenyl group, a biphenyl group, a terphenyl group and the like, but is not limited thereto. The polycyclic aryl group includes a naphthyl group, an anthracenyl group, a phenanthryl group, a pyrenyl group, a perylenyl group, a chrysenyl group, a fluorenyl group or the like, but is not limited thereto.

In the present disclosure, a fluorenyl group can be substituted, and two substituents can be bonded to each other to form a spiro structure. In the case where the fluorenyl group is substituted,

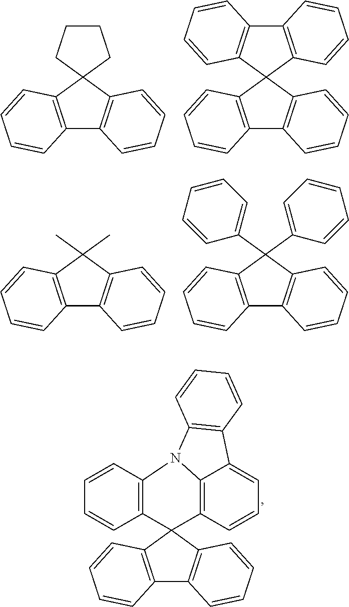

and the like can be formed. However, the structure is not limited thereto.

In the present disclosure, a heterocyclic group is a heterocyclic group containing at least one heteroatom of O, N, Si and S as a heterogeneous element, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, a triazole group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, a thiazolyl group, an isoxazolyl group, an oxadiazolyl group, a thiadiazolyl group, a benzothiazolyl group, a phenothiazinyl group, a dibenzofuranyl group, and the like, but are not limited thereto.

In the present disclosure, the aryl group in the aralkyl group, the aralkenyl group, the alkylaryl group, and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can apply the aforementioned description of the heterocyclic group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

Hereinafter, the present invention will be described in detail for each configuration.

Anode and Cathode

The anode and cathode used in the present disclosure refer to electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Light Emitting Layer

The light emitting layer used in the present disclosure refers to a layer that emits light in the visible light region by combining holes and electrons transported from the anode and the cathode. Generally, the light emitting layer includes a host material and a dopant material.

The host material can further include a condensed aromatic ring derivative, a hetero ring-containing compound, or the like. In the present disclosure, a compound of Chemical Formula 1, a compound of Chemical Formula 2, and a compound of Chemical Formula 3 are mixed and used as the host material.

Specifically, the indolocarbazole-based compound (Chemical Formula 1) and the biscarbazole-based compound (Chemical Formula 2) have excellent hole transport ability, thereby serving as a P-type host; and the compound in which pyridine, pyrimidine, or triazine is bonded to N of indolocarbazole (Chemical Formula 3) serves as an N-type host.

Furthermore, the organic light emitting device in which the three types of compounds are applied as the host material of the light emitting layer can exhibit improved driving voltage (low voltage), high efficiency and long lifetime, compared to the case in which three types of compounds completely different from the above three types are mixed and used as the host material (applying three types of hosts), any one or two of the above three types of compounds are changed to another compound (applying three types of hosts), or only one or two of the above three types of compounds are used as the host material of the light emitting layer (applying one or two types of hosts), etc.

In general, since an exciplex is formed when a P-type host and an N-type host are mixed and applied as the host of the light emitting layer, the characteristics of the device can be further improved compared to the case in which only one of the P-type host and the N-type host is applied.

In particular, when using a mixture of the two P-type hosts which are Chemical Formulae 1 and 2 and the N-type host of the Chemical Formula 3 (Chemical Formula 1+Chemical Formula 2+Chemical Formula 3), the characteristics of the device can be further improved compared to the case in which only one P-type host of Chemical Formulae 1 and 2 is mixed with the N-type host of the Chemical Formula 3 (Chemical Formula 1+Chemical Formula 3; or Chemical Formula 2+Chemical Formula 3).

The P-type host of the Chemical Formula 1 exhibits low voltage due to its structure containing indolocarbazole, and the P-type host of the Chemical Formula 2 exhibits high efficiency and long lifetime due to its structure containing biscarbazole. Therefore, using a mixture thereof is advantageous for uniformly improving the voltage, efficiency, and lifetime of the device.

Meanwhile, when several types of hosts are mixed and used, intermediate characteristics of the mixed hosts appear.

The P-type host of the Chemical Formula 3 contributes to overall improvement in voltage, efficiency, and lifetime of the device due to its structure in which pyridine, pyrimidine, or triazine is bonded to N of indolocarbazole.

When mixing the two P-type hosts of the Chemical Formulae 1 and 2 and the N-type host of the Chemical Formula 3, the voltage, efficiency, and lifetime of the device can be overall improved compared to the case in which a compound having a structure completely different from that of the Chemical Formula 3 is replaced with the N-type host.

Preferably, the compound of Chemical Formula 1, the compound of Chemical Formula 2, and the compound of Chemical Formula 3 can be included in the light emitting layer in a weight ratio of 0.5-1.5:0.5-1.5:0.5-1.5. More preferably, the weight ratio is 0.8-1.2:0.8-1.2:0.8-1.2.

Hereinafter, the three types of compounds will be described in detail.

In Chemical Formula 1, A is a benzene ring condensed with two adjacent pentagonal rings.

Specifically, depending on the condensed form of A, the Chemical Formula 1 can have a structure of any one of the following Chemical Formulae 1-1 to 1-4:

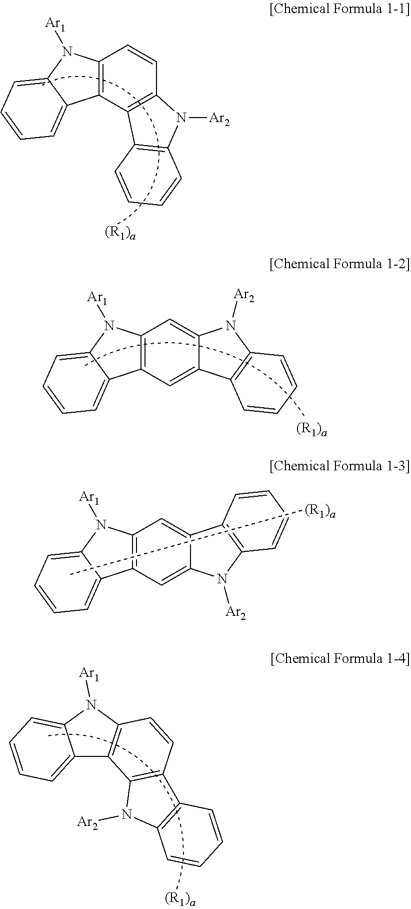

wherein in Chemical Formulae 1-1 to 1-4, Ar₁, Ar₂, R₁, and a have the same definitions as described above.

Specifically, Ar₁ and Ar₂ can each independently be phenyl, biphenylyl, terphenylyl, (phenyl)biphenylyl, dimethylfluorenyl, (dimethylfluorenyl)phenyl, dibenzofuranyl, (dibenzofuranyl)phenyl, dibenzothiophenyl, or (dibenzothiophenyl)phenyl.

Ar₁ can be a substituted or unsubstituted C₆₋₃₀ aryl. Specifically, Ar₁ can be phenyl, biphenylyl, or terphenylyl.

Ar₂ can be a substituted or unsubstituted C₆₋₃₀ aryl, or C₂₋₃₀ heteroaryl containing at least one heteroatom selected from the group consisting of O and S. Specifically, Ar₂ can be biphenylyl, terphenylyl, (phenyl)biphenylyl, dimethylfluorenyl, (dimethylfluorenyl)phenyl, dibenzofuranyl, (dibenzofuranyl)phenyl, dibenzothiophenyl, or (dibenzothiophenyl)phenyl.

R₁ can be hydrogen, deuterium, a substituted or unsubstituted C₆₋₃₀ aryl, or a substituted or unsubstituted C₂₋₃₀ heteroaryl containing at least one heteroatom selected from the group consisting of N, O and S.

Specifically, R₁ can be hydrogen.

a is an integer of 1 to 10.

Representative examples of the compound of Chemical Formula 1 are as follows:

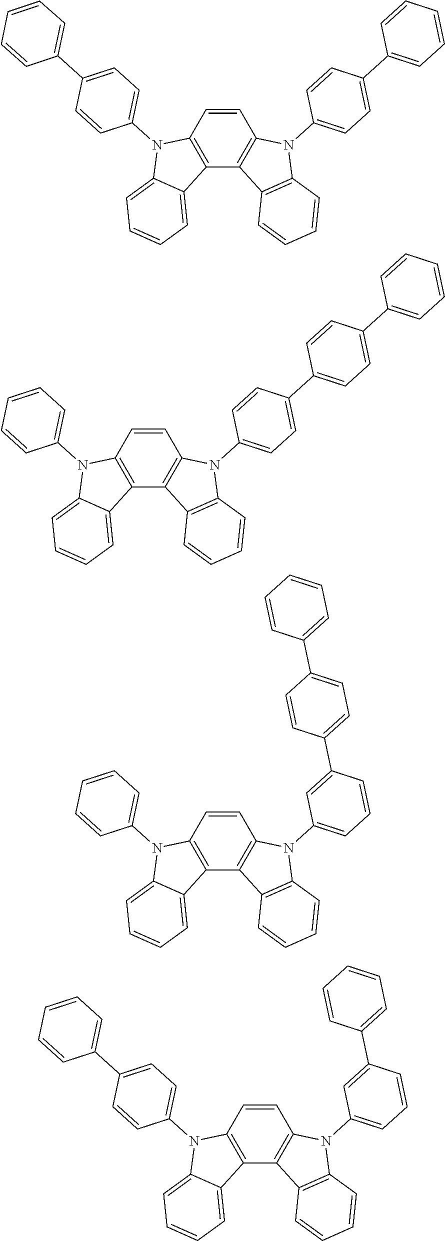

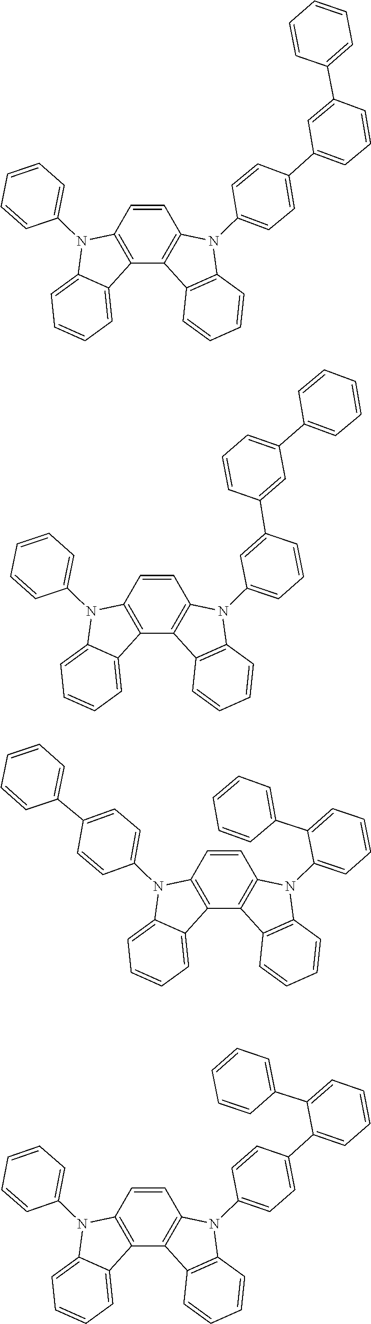 [SMILES: c1ccc(-c2cccc(-c3ccc(-n4c5ccccc5c5c6c7ccccc7n(-c7ccccc7)c6ccc54)cc3)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6ccccc6-c6ccccc6)c5ccc43)cc2)cc1 ; c1ccc(-c2ccccc2-c2ccc(-n3c4ccccc4c4c5c6ccccc6n(-c6ccccc6)c5ccc43)cc2)cc1]

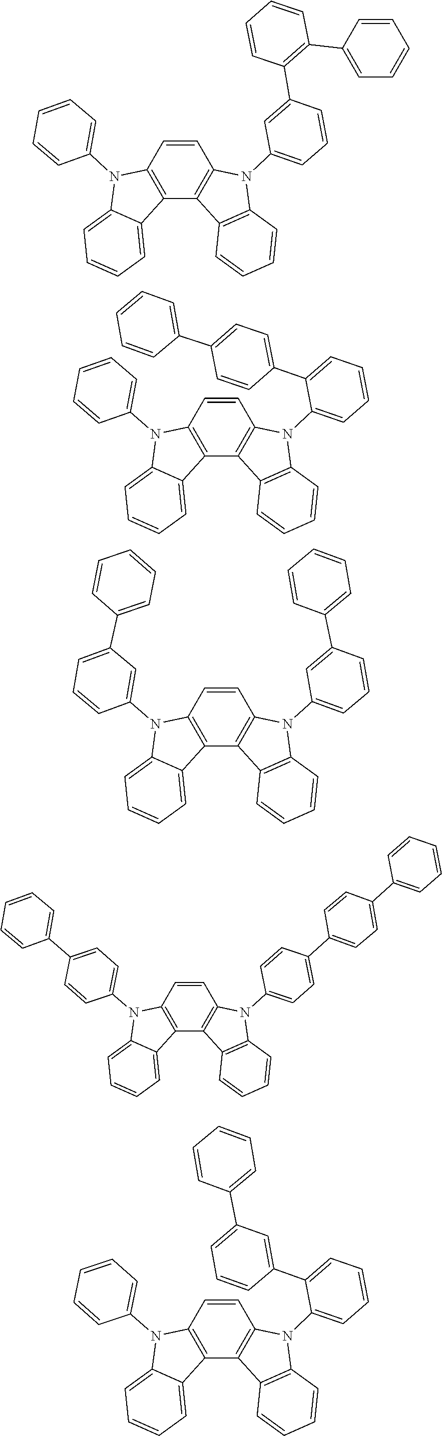

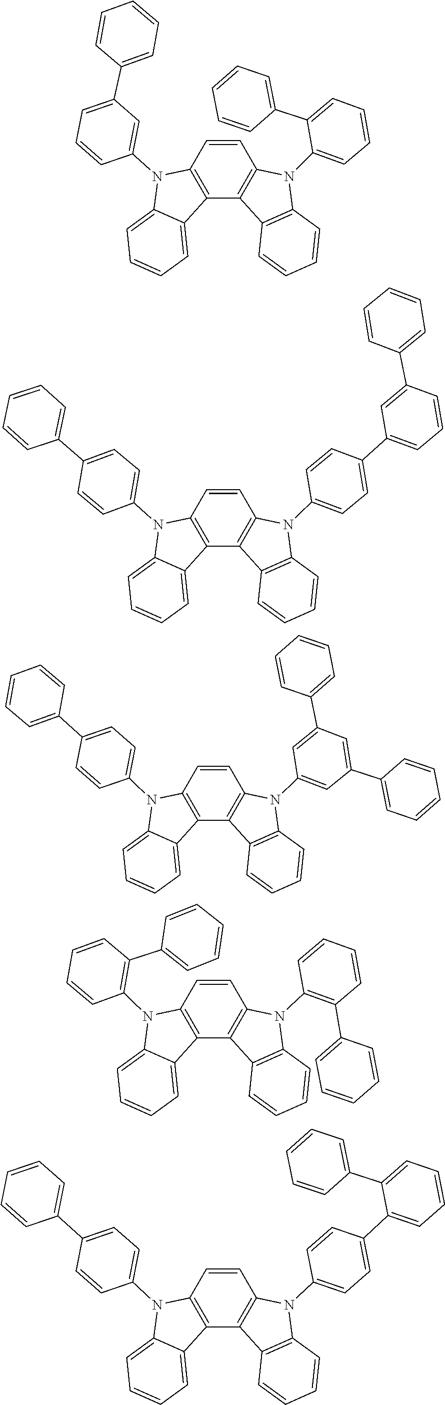

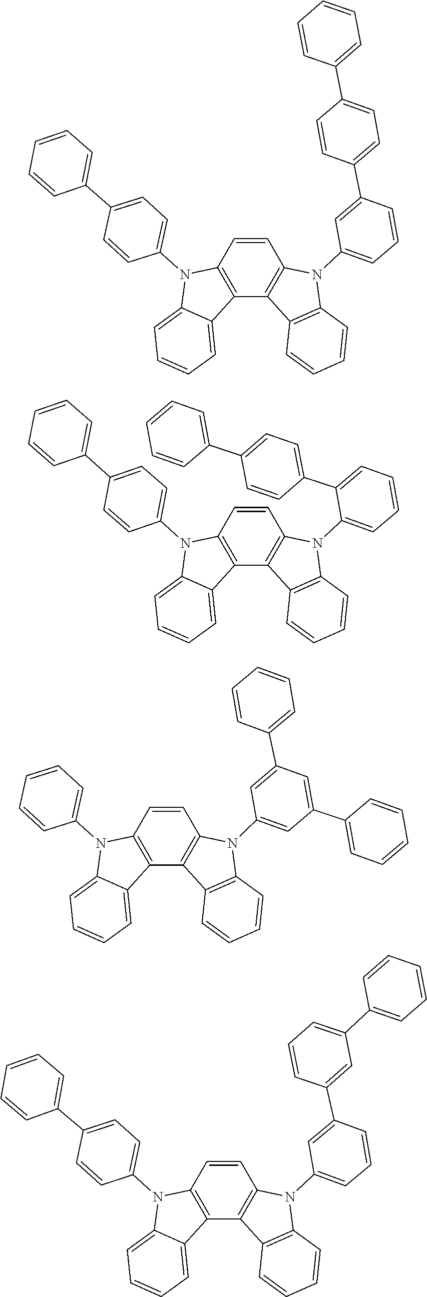

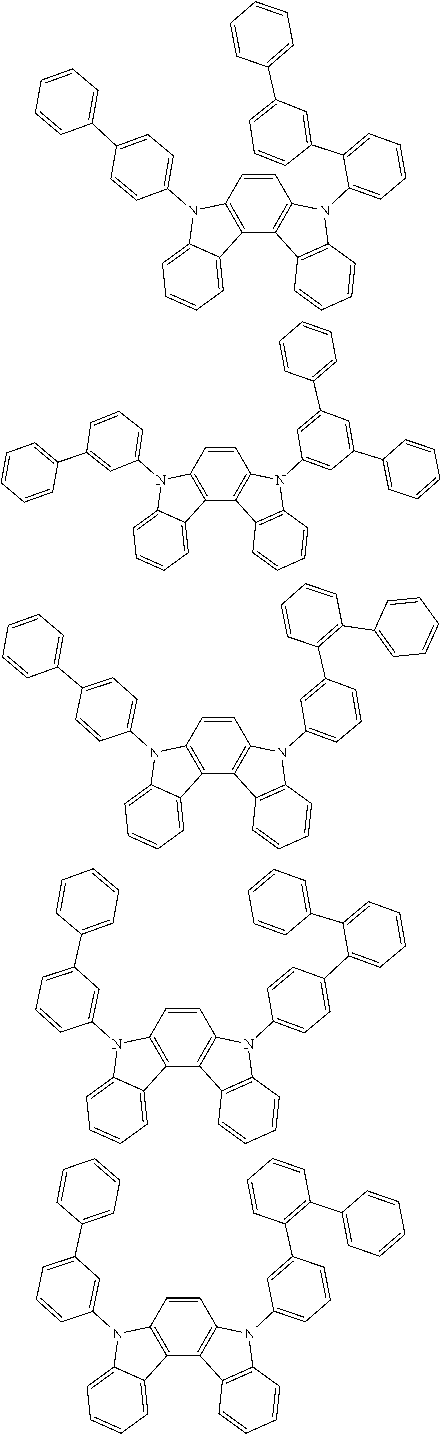

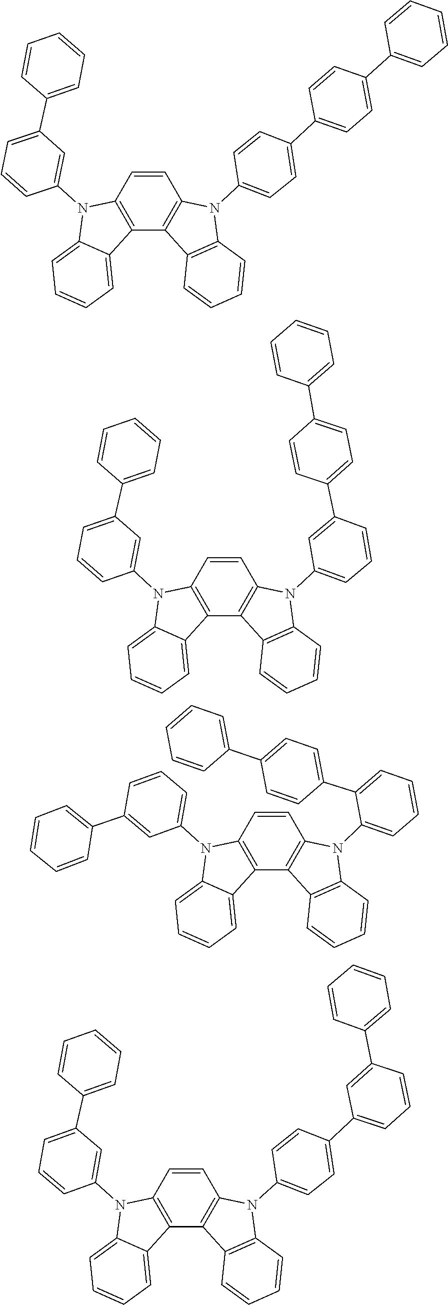

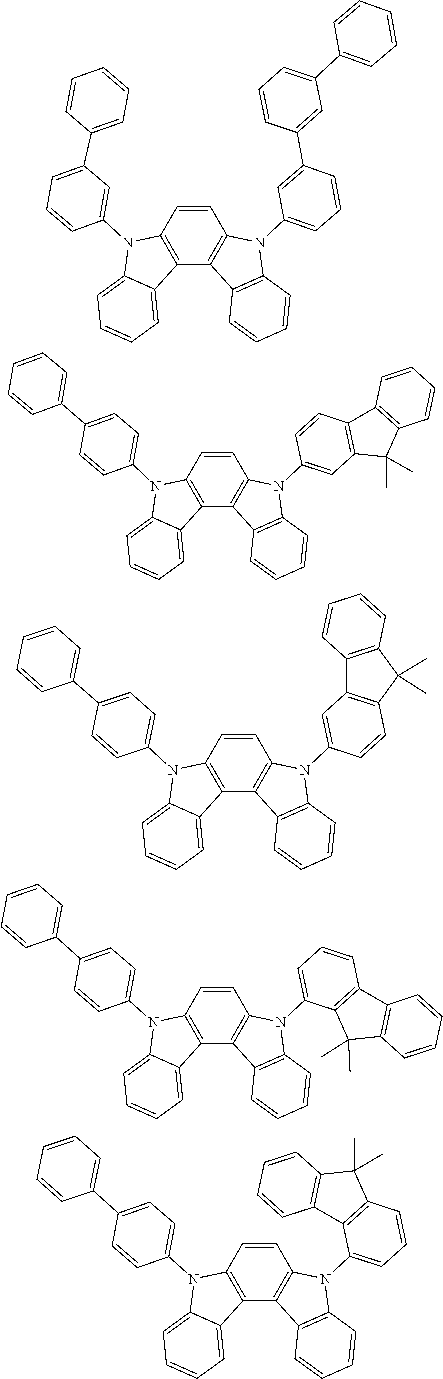

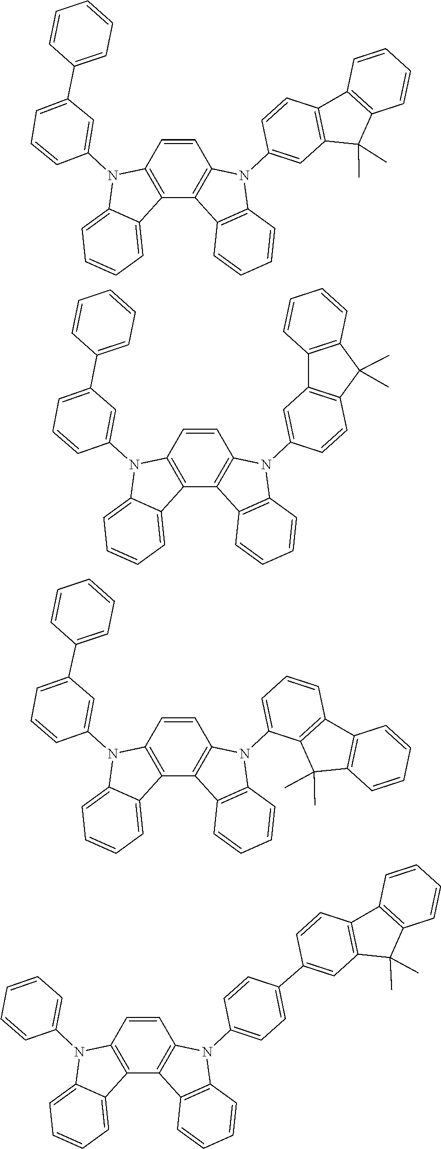

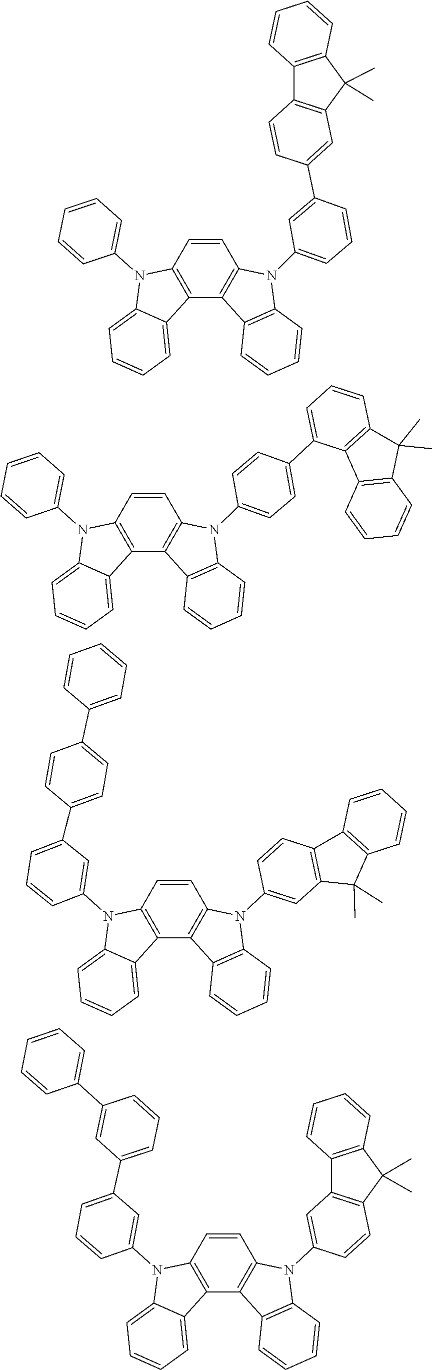

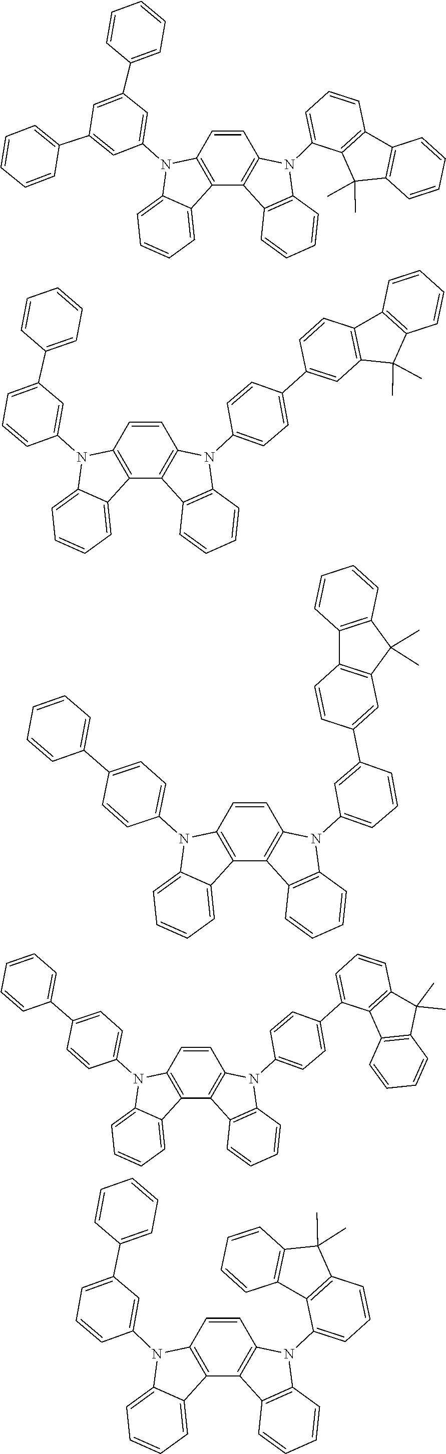

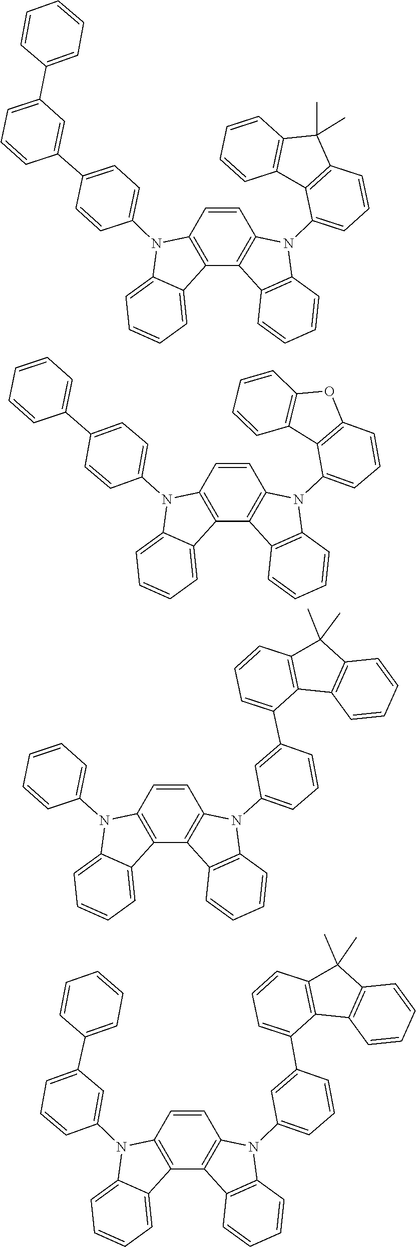

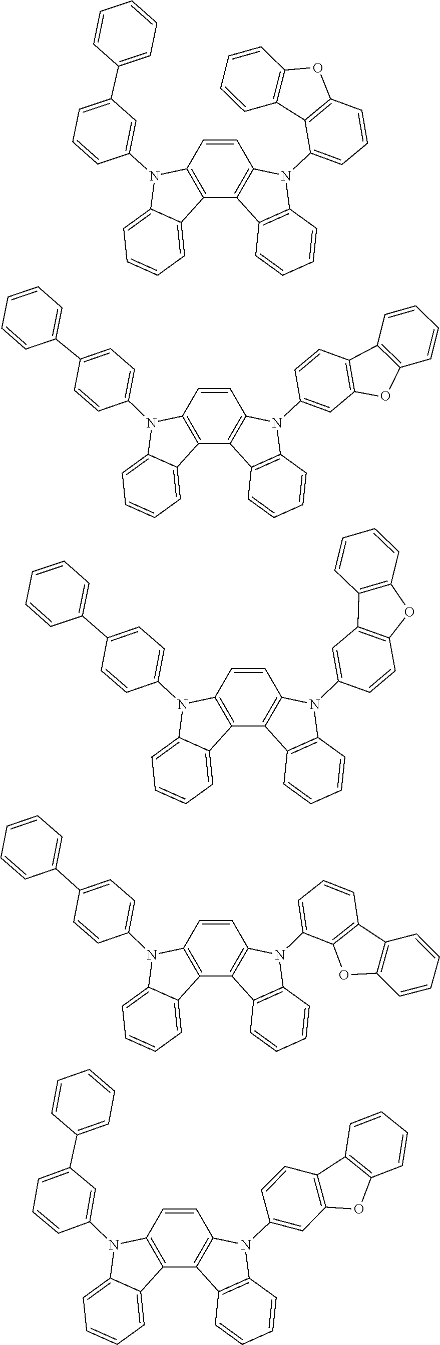 [SMILES: c1ccc(-c2cccc(-n3c4ccccc4c4c5c6ccccc6n(-c6cccc7oc8ccccc8c67)c5ccc43)c2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2ccc(-n3c4ccccc4c4c5c6ccccc6n(-c6ccc7oc8ccccc8c7c6)c5ccc43)cc2)cc1 ; c1ccc(-c2ccc(-n3c4ccccc4c4c5c6ccccc6n(-c6cccc7c6oc6ccccc67)c5ccc43)cc2)cc1 ; c1ccc(-c2cccc(-n3c4ccccc4c4c5c6ccccc6n(-c6ccc7c(c6)oc6ccccc67)c5ccc43)c2)cc1]

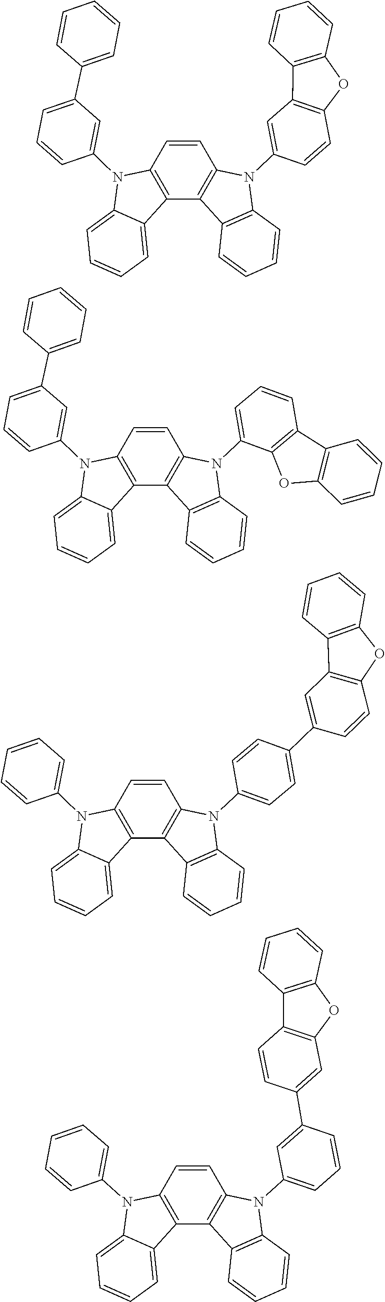

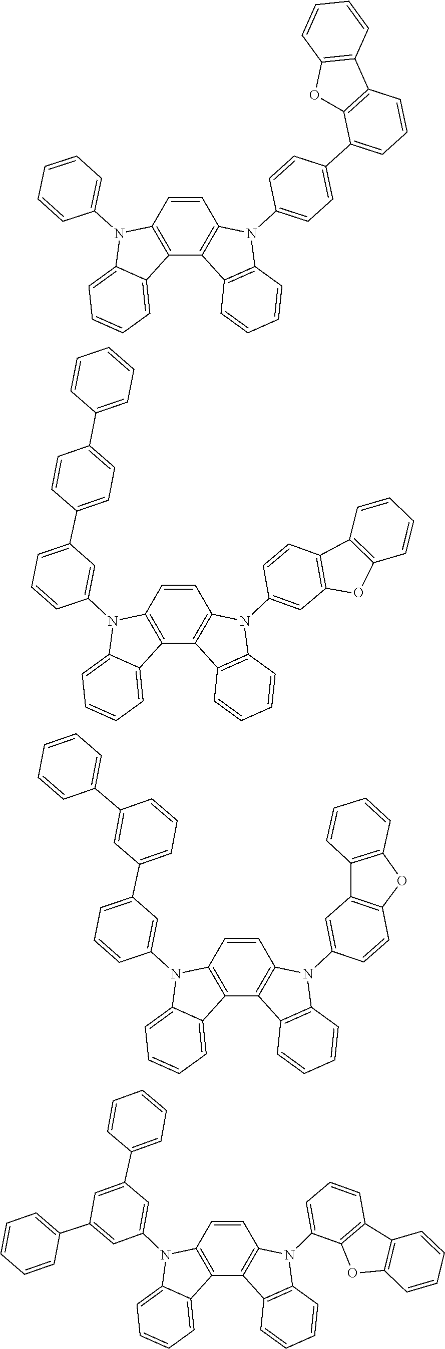

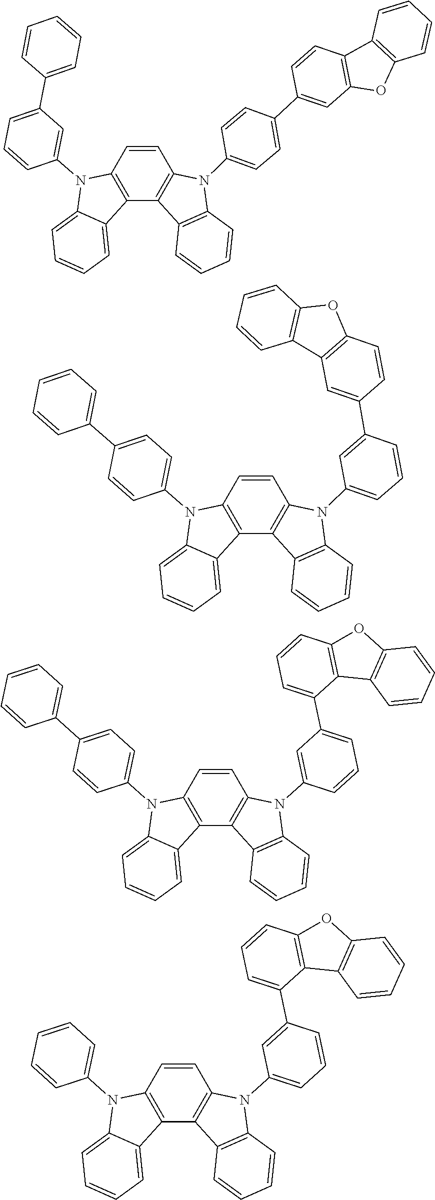

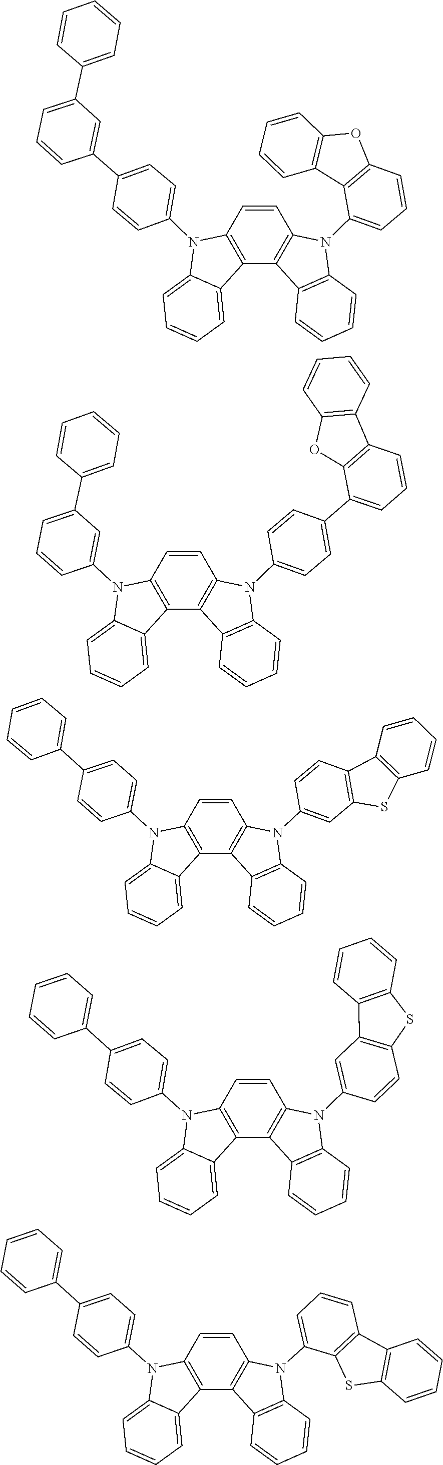

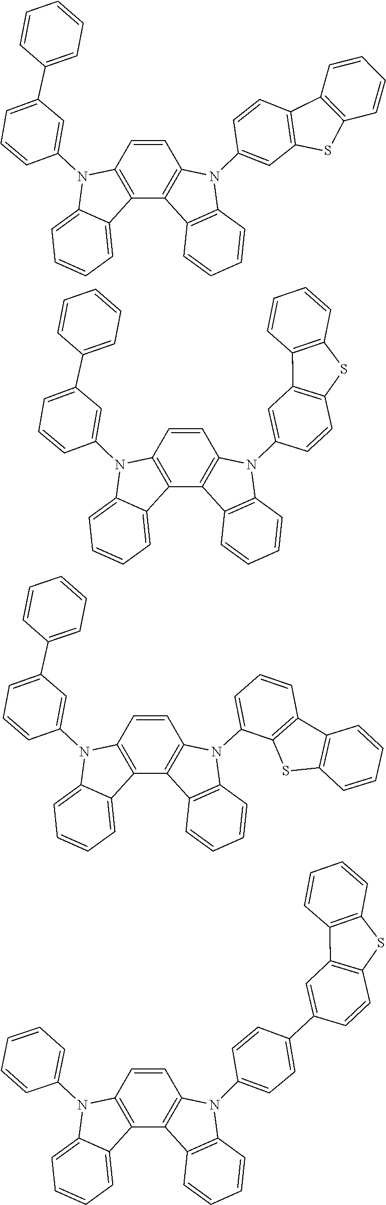

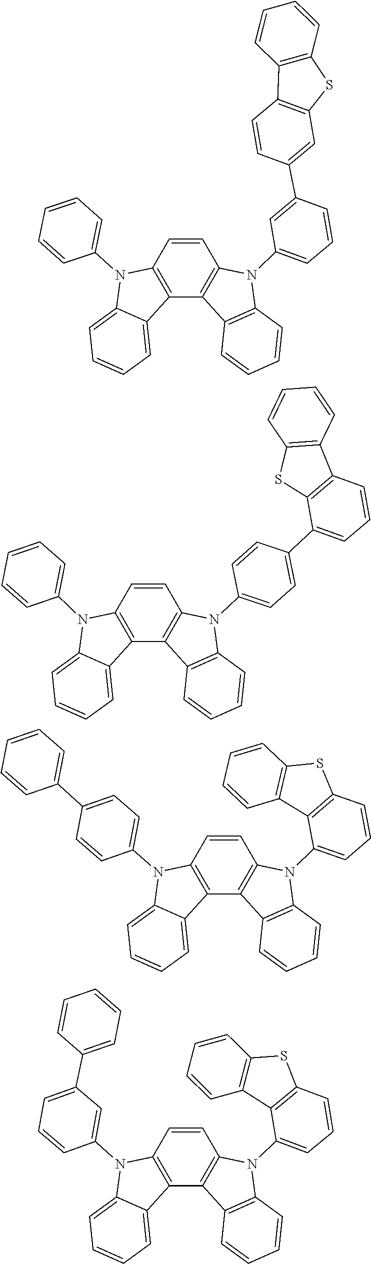

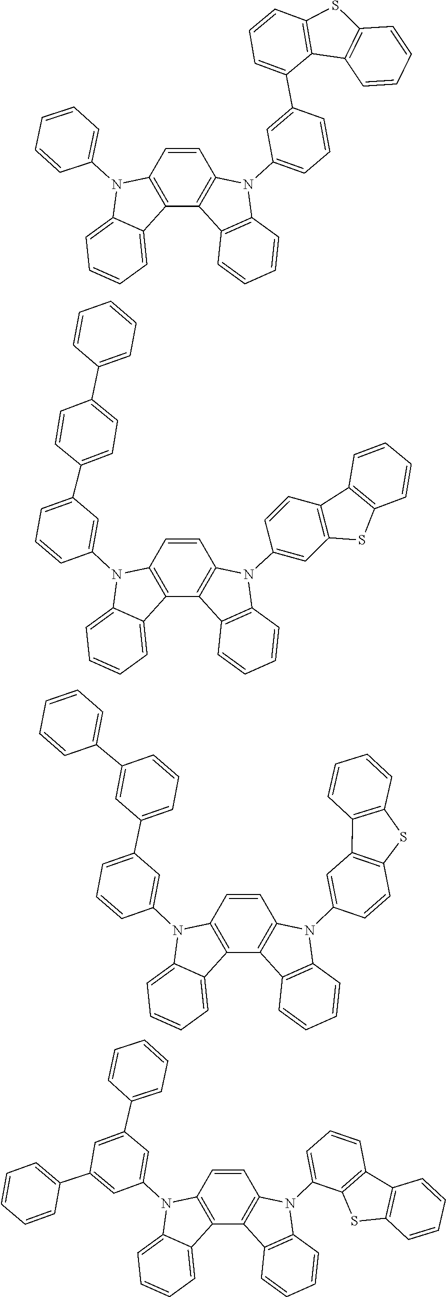

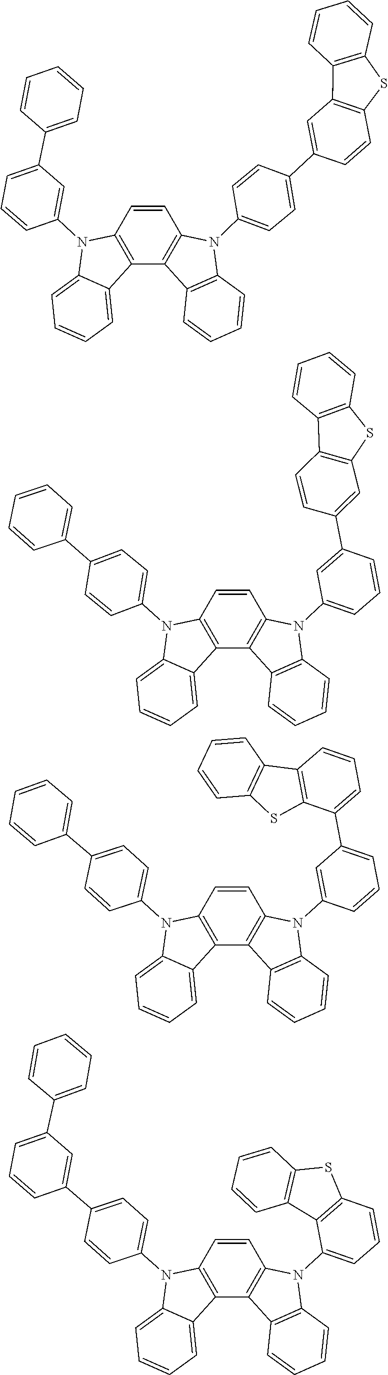

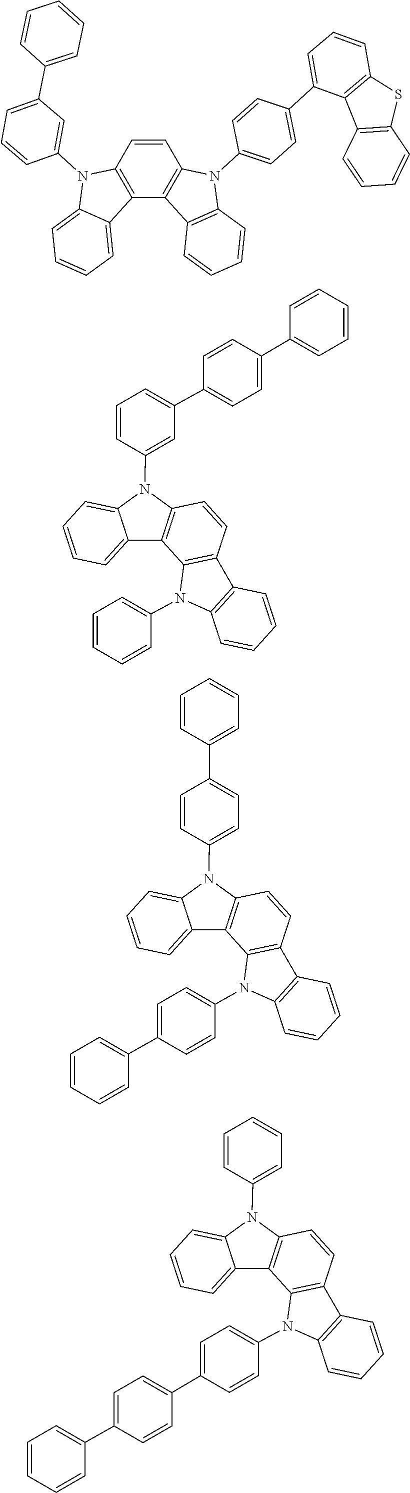

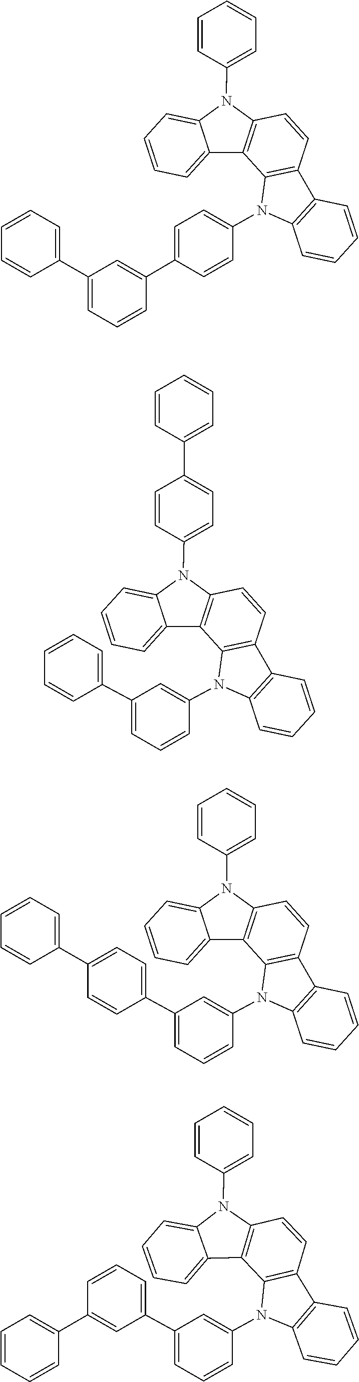 [SMILES: c1ccc(-c2cccc(-c3ccc(-n4c5ccccc5c5ccc6c(c7ccccc7n6-c6ccccc6)c54)cc3)c2)cc1 ; c1ccc(-c2ccc(-n3c4ccccc4c4c3ccc3c5ccccc5n(-c5cccc(-c6ccccc6)c5)c34)cc2)cc1 ; c1ccc(-c2ccc(-c3cccc(-n4c5ccccc5c5ccc6c(c7ccccc7n6-c6ccccc6)c54)c3)cc2)cc1 ; c1ccc(-c2cccc(-c3cccc(-n4c5ccccc5c5ccc6c(c7ccccc7n6-c6ccccc6)c54)c3)c2)cc1]

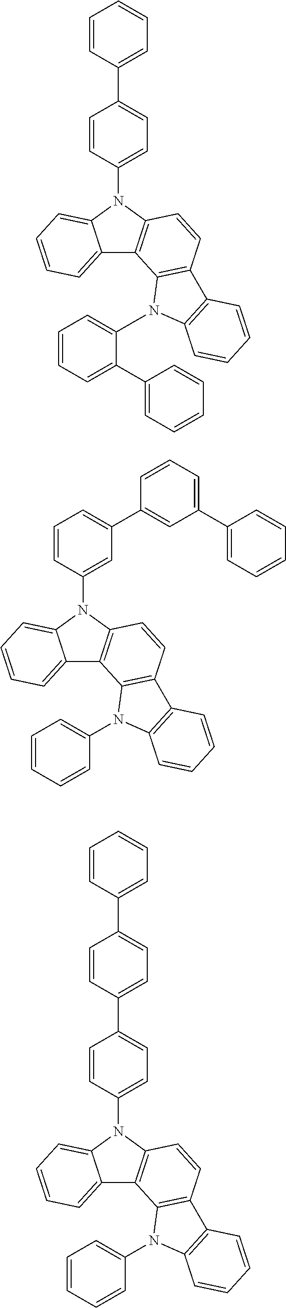

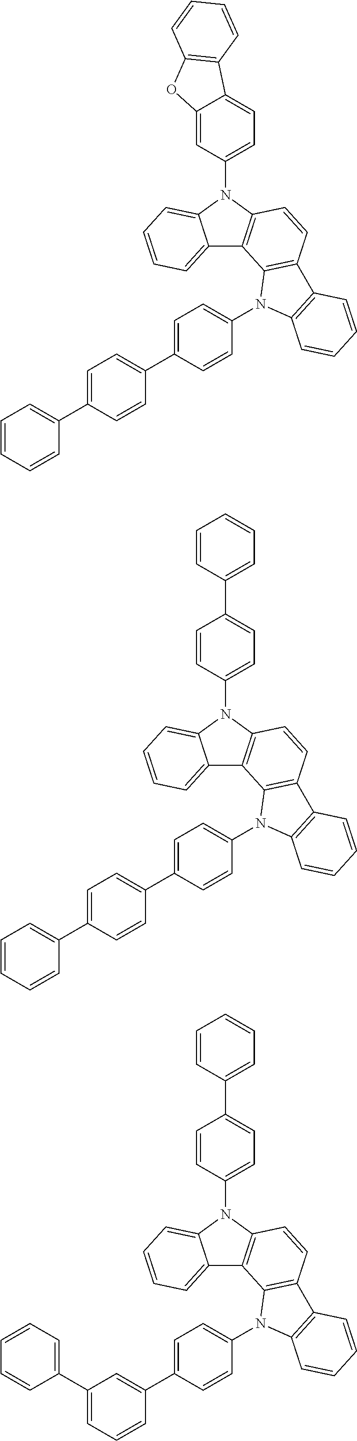 [SMILES: c1ccc(-c2ccc(-c3ccc(-n4c5ccccc5c5ccc6c(c7ccccc7n6-c6ccc7c(c6)oc6ccccc67)c54)cc3)cc2)cc1 ; c1ccc(-c2ccc(-c3ccc(-n4c5ccccc5c5ccc6c(c7ccccc7n6-c6ccc(-c7ccccc7)cc6)c54)cc3)cc2)cc1 ; c1ccc(-c2ccc(-n3c4ccccc4c4c3ccc3c5ccccc5n(-c5ccc(-c6cccc(-c7ccccc7)c6)cc5)c34)cc2)cc1]

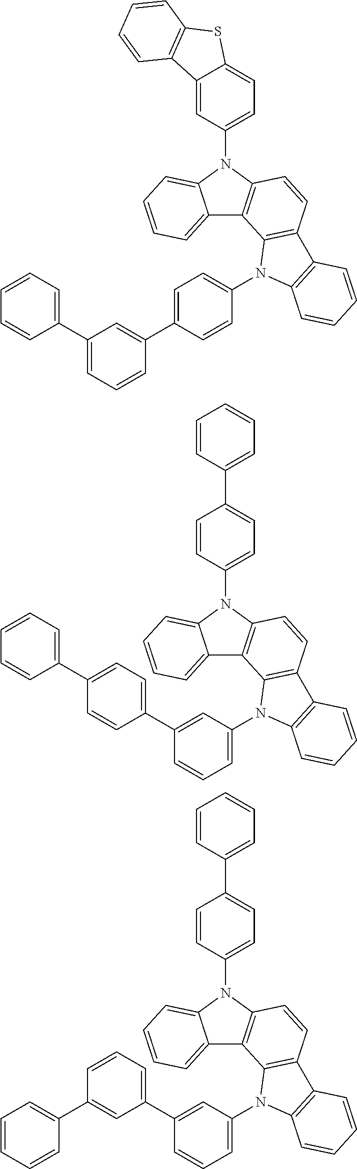

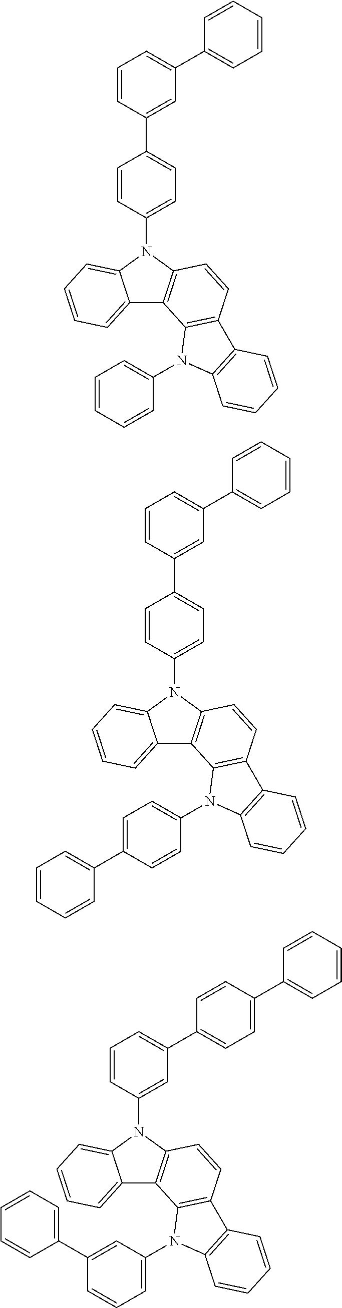

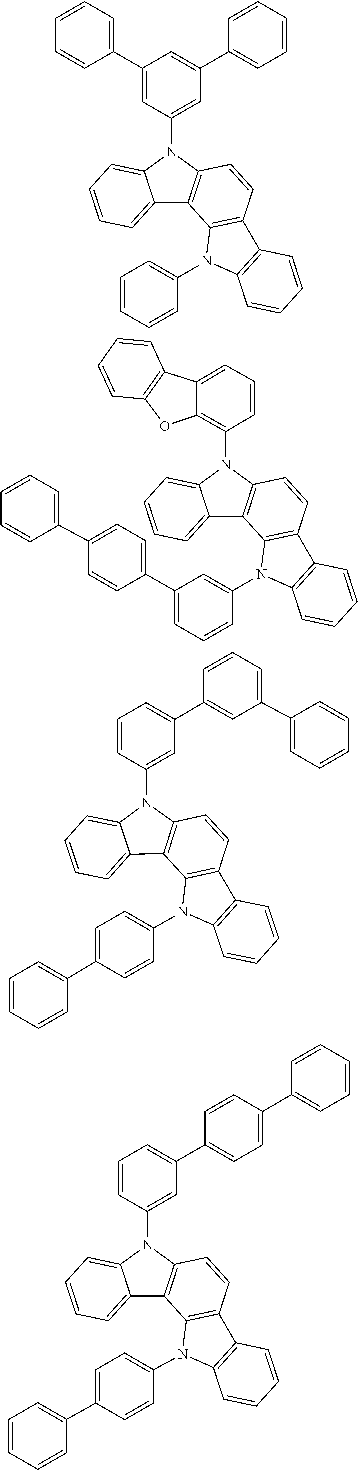

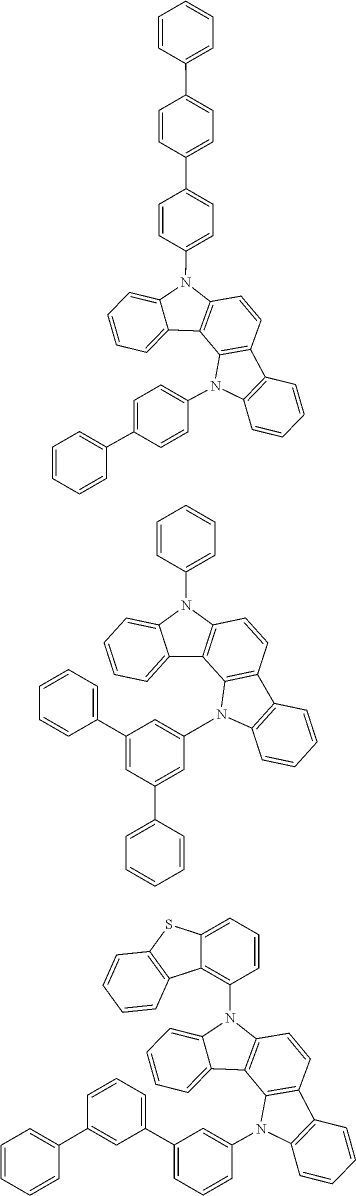

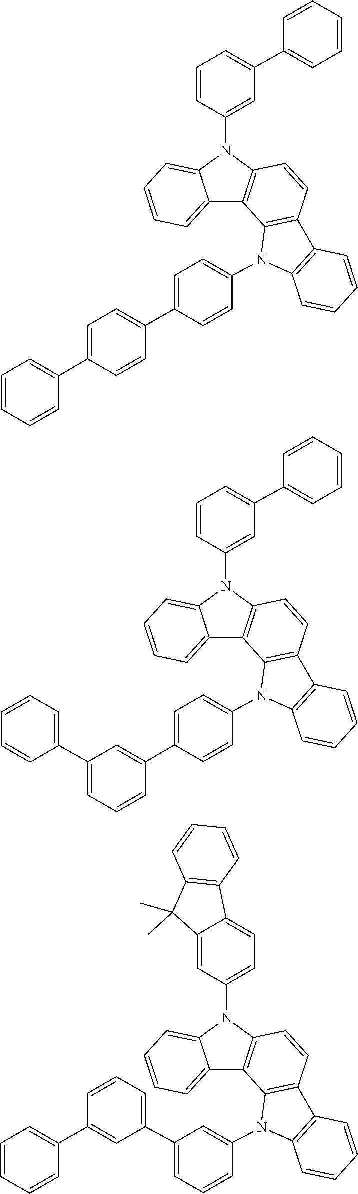 [SMILES: c1ccc(-c2ccc(-c3ccc(-n4c5ccccc5c5ccc6c(c7ccccc7n6-c6cccc(-c7ccccc7)c6)c54)cc3)cc2)cc1 ; c1ccc(-c2cccc(-c3ccc(-n4c5ccccc5c5ccc6c(c7ccccc7n6-c6cccc(-c7ccccc7)c6)c54)cc3)c2)cc1 ; CC1(C)c2ccccc2-c2ccc(-n3c4ccccc4c4c3ccc3c5ccccc5n(-c5cccc(-c6cccc(-c7ccccc7)c6)c5)c34)cc21]

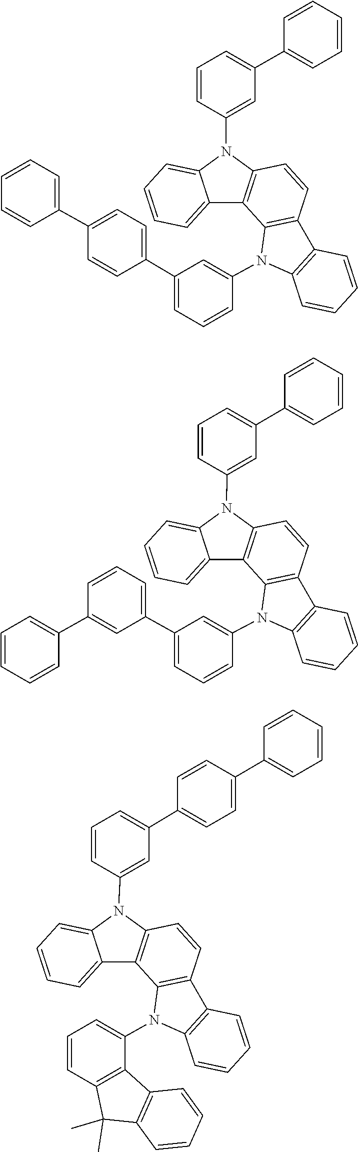 [SMILES: c1ccc(-c2ccc(-c3cccc(-n4c5ccccc5c5ccc6c(c7ccccc7n6-c6cccc(-c7ccccc7)c6)c54)c3)cc2)cc1 ; c1ccc(-c2cccc(-c3cccc(-n4c5ccccc5c5ccc6c(c7ccccc7n6-c6cccc(-c7ccccc7)c6)c54)c3)c2)cc1 ; CC1(C)c2ccccc2-c2c(-n3c4ccccc4c4ccc5c(c6ccccc6n5-c5cccc(-c6ccc(-c7ccccc7)cc6)c5)c43)cccc21]

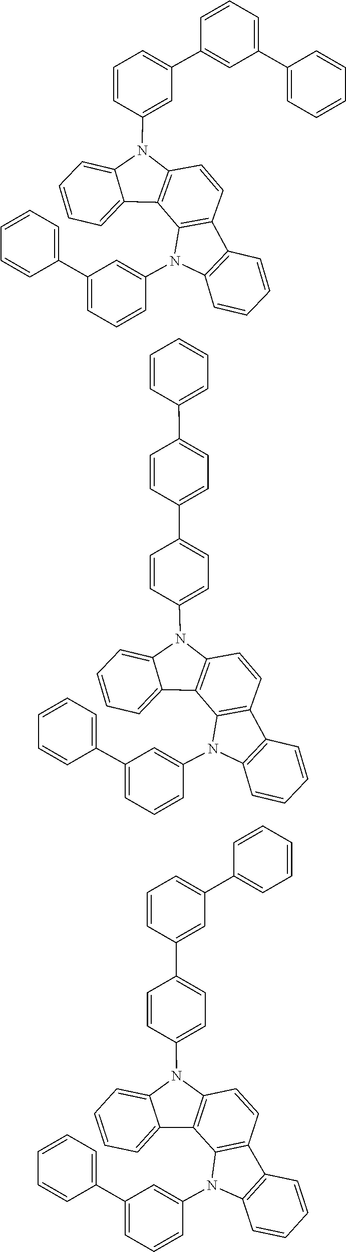

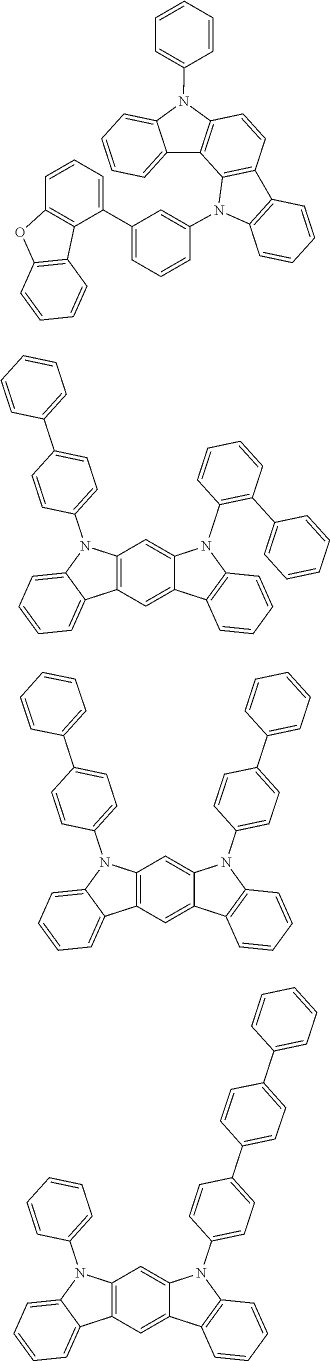

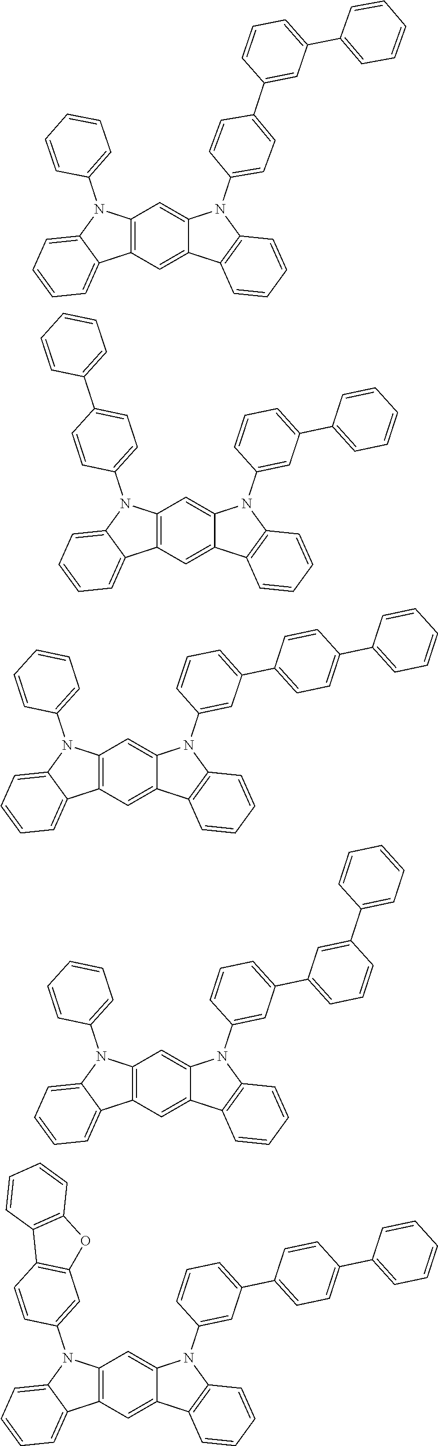

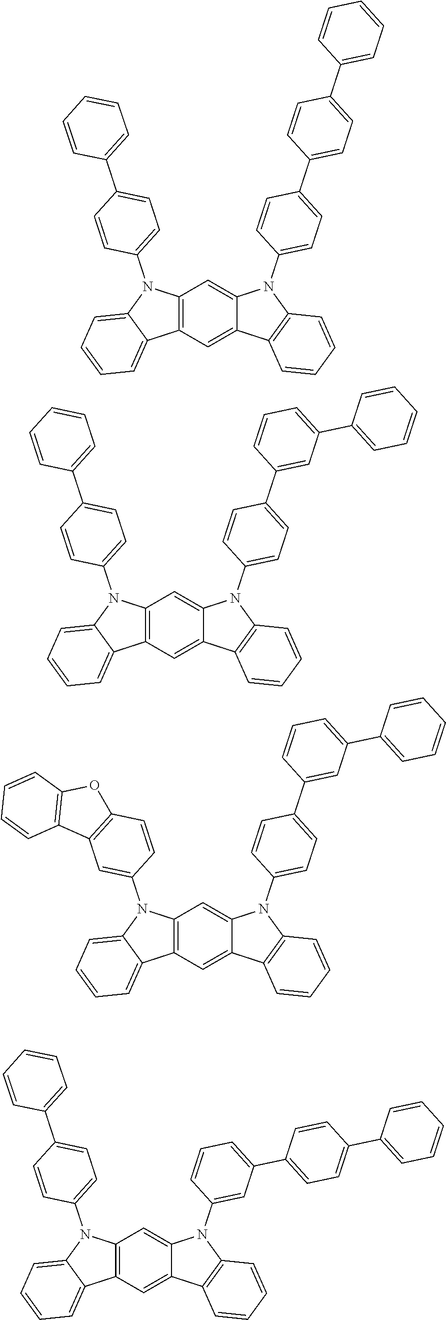

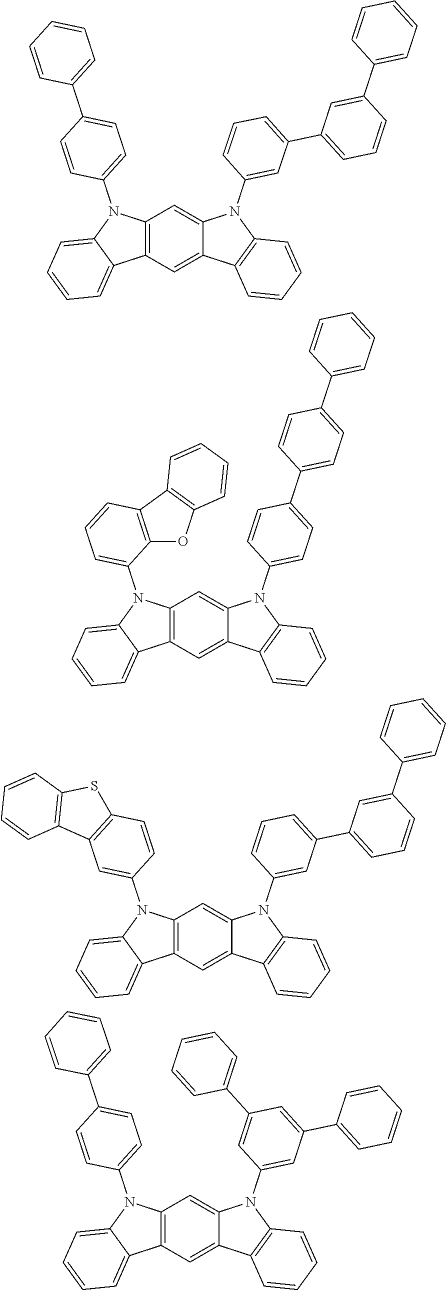

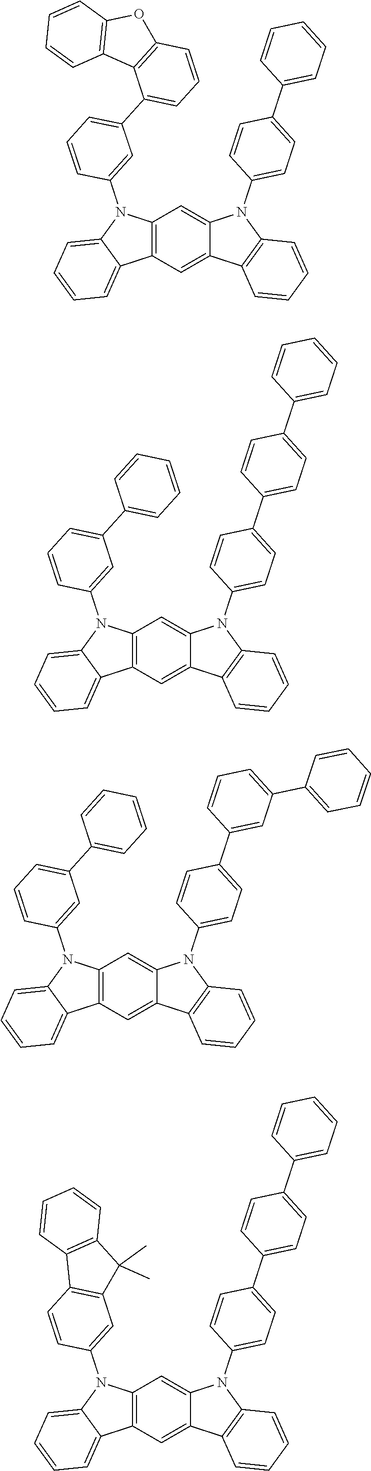

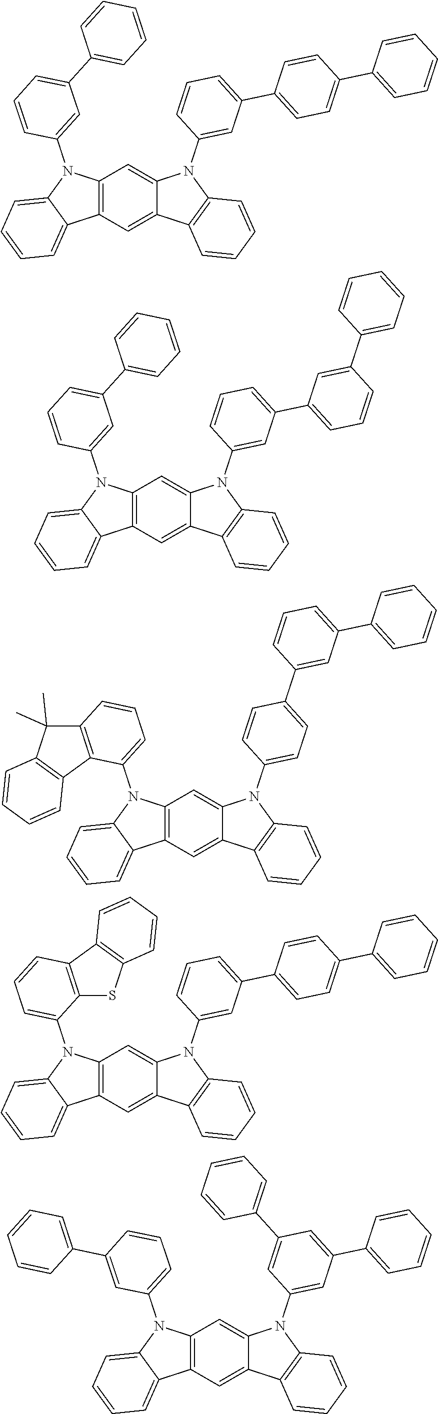

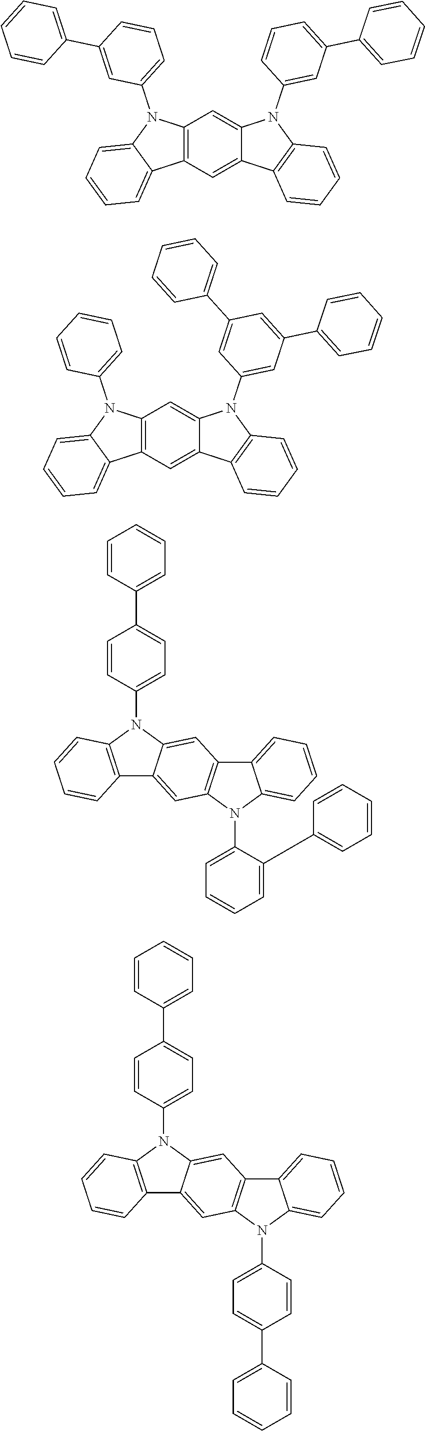 [SMILES: c1ccc(-c2cccc(-n3c4ccccc4c4cc5c6ccccc6n(-c6cccc(-c7ccccc7)c6)c5cc43)c2)cc1 ; c1ccc(-c2cc(-c3ccccc3)cc(-n3c4ccccc4c4cc5c6ccccc6n(-c6ccccc6)c5cc43)c2)cc1 ; c1ccc(-c2ccc(-n3c4ccccc4c4cc5c(cc43)c3ccccc3n5-c3ccccc3-c3ccccc3)cc2)cc1 ; c1ccc(-c2ccc(-n3c4ccccc4c4cc5c(cc43)c3ccccc3n5-c3ccc(-c4ccccc4)cc3)cc2)cc1]

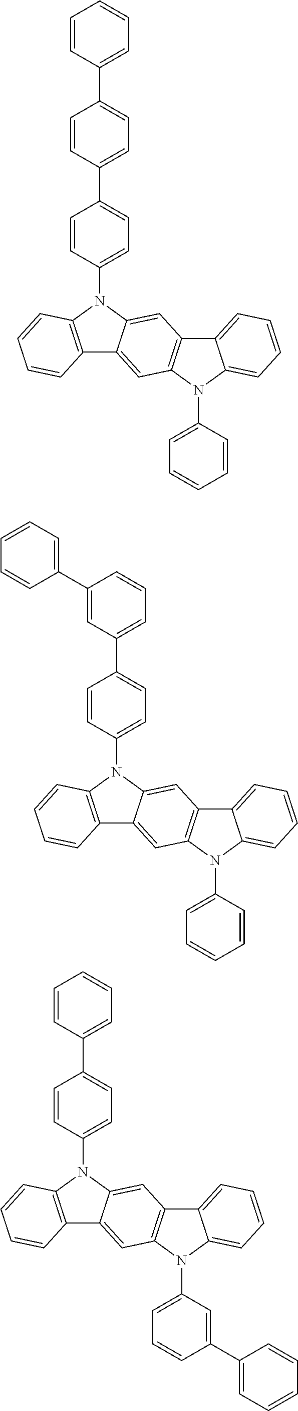 [SMILES: c1ccc(-c2ccc(-c3ccc(-n4c5ccccc5c5cc6c(cc54)c4ccccc4n6-c4ccccc4)cc3)cc2)cc1 ; c1ccc(-c2cccc(-c3ccc(-n4c5ccccc5c5cc6c(cc54)c4ccccc4n6-c4ccccc4)cc3)c2)cc1 ; c1ccc(-c2ccc(-n3c4ccccc4c4cc5c(cc43)c3ccccc3n5-c3cccc(-c4ccccc4)c3)cc2)cc1]

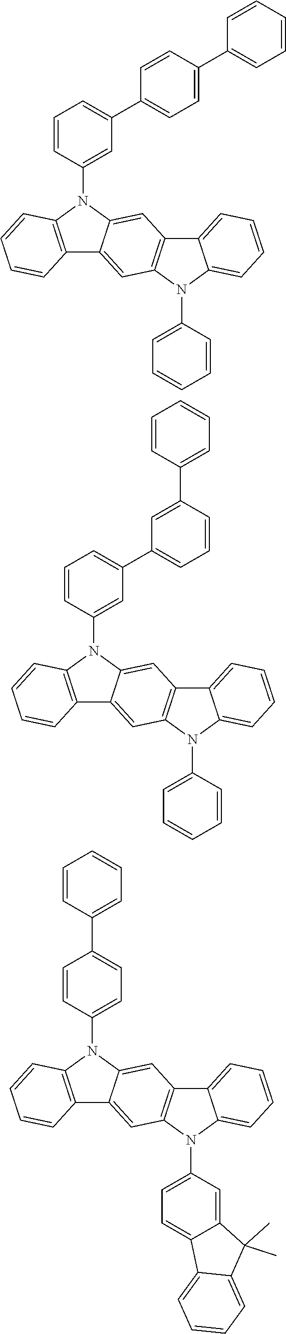

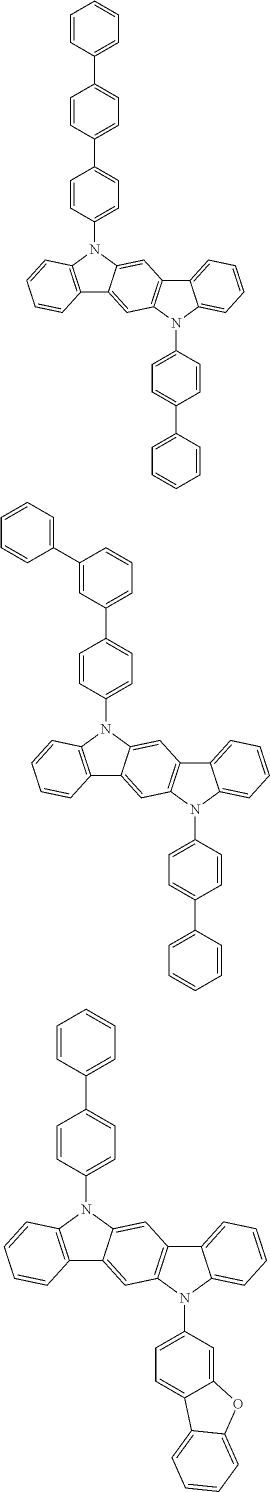 [SMILES: c1ccc(-c2ccc(-c3ccc(-n4c5ccccc5c5cc6c(cc54)c4ccccc4n6-c4ccc(-c5ccccc5)cc4)cc3)cc2)cc1 ; c1ccc(-c2ccc(-n3c4ccccc4c4cc5c(cc43)c3ccccc3n5-c3ccc(-c4cccc(-c5ccccc5)c4)cc3)cc2)cc1 ; c1ccc(-c2ccc(-n3c4ccccc4c4cc5c(cc43)c3ccccc3n5-c3ccc4c(c3)oc3ccccc34)cc2)cc1]

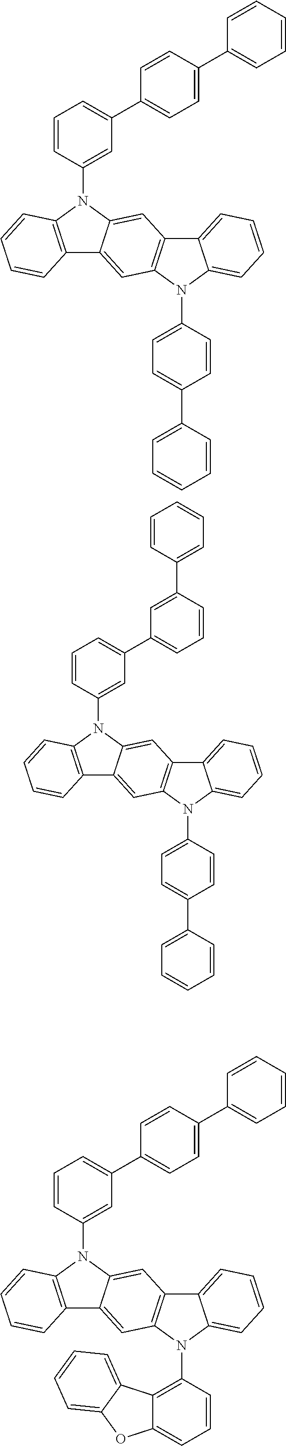

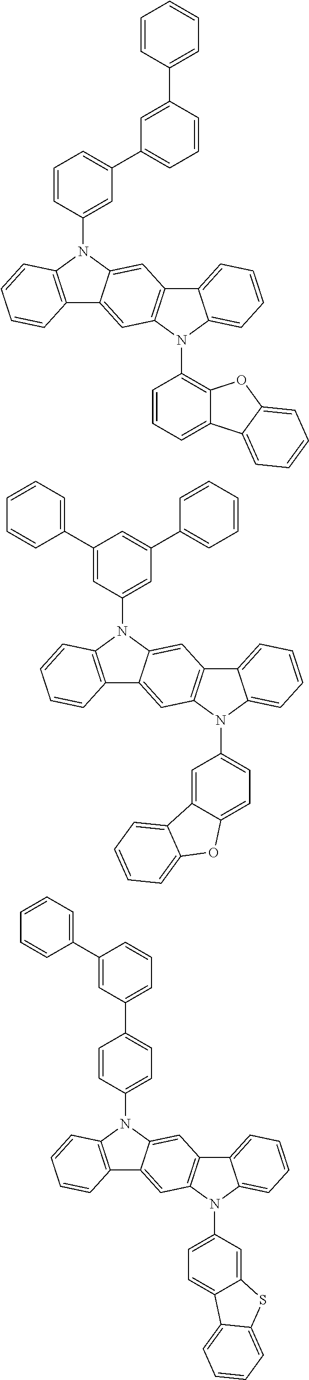

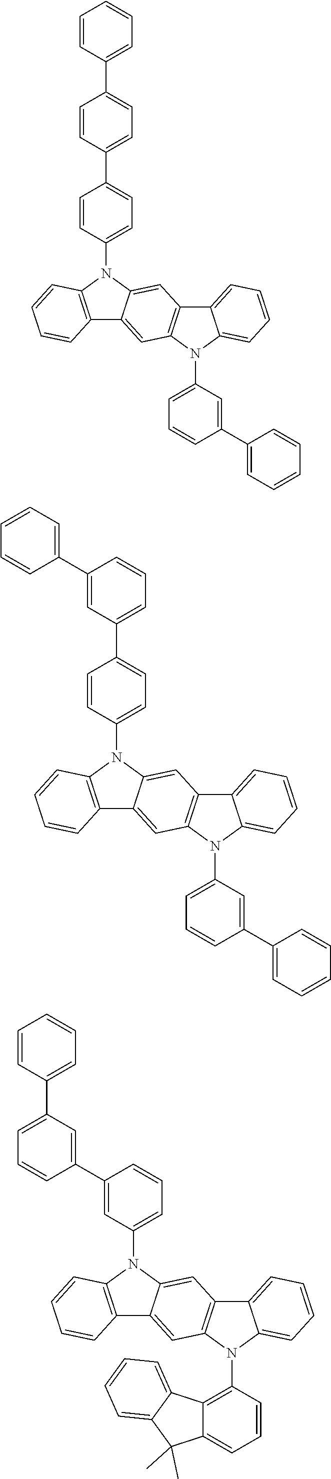 [SMILES: c1ccc(-c2ccc(-c3ccc(-n4c5ccccc5c5cc6c(cc54)c4ccccc4n6-c4cccc(-c5ccccc5)c4)cc3)cc2)cc1 ; c1ccc(-c2cccc(-c3ccc(-n4c5ccccc5c5cc6c(cc54)c4ccccc4n6-c4cccc(-c5ccccc5)c4)cc3)c2)cc1 ; CC1(C)c2ccccc2-c2c(-n3c4ccccc4c4cc5c(cc43)c3ccccc3n5-c3cccc(-c4cccc(-c5ccccc5)c4)c3)cccc21]

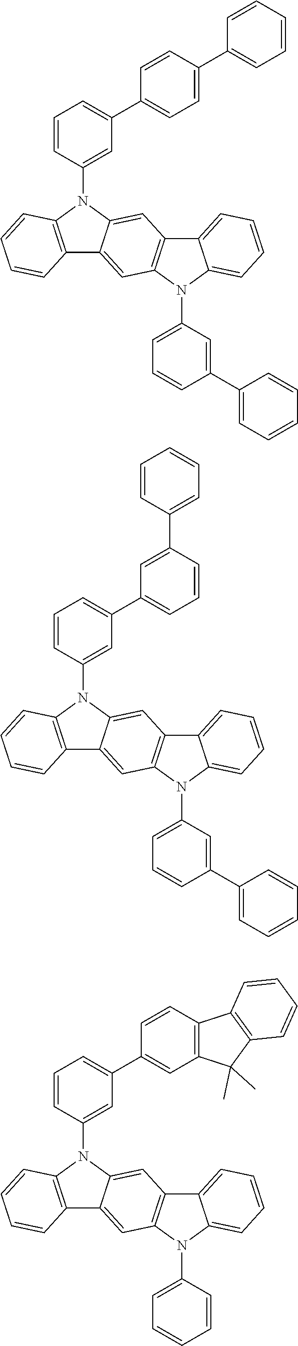

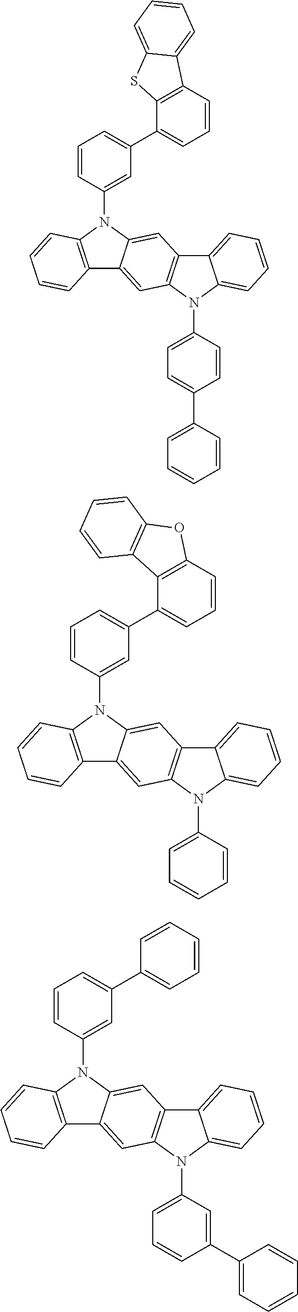

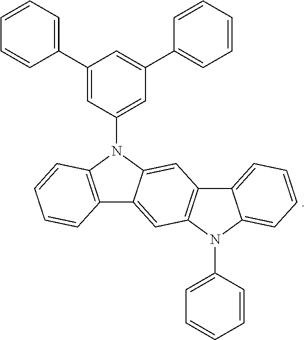 [SMILES: c1ccc(-c2cc(-c3ccccc3)cc(-n3c4ccccc4c4cc5c(cc43)c3ccccc3n5-c3ccccc3)c2)cc1]

The compound of Chemical Formula 1 can be prepared by a series of processes as shown in Reaction Schemes 1-1 and 1-2 below. (The definition of each substituent in Reaction Schemes 1-1 and 1-2 is the same as described above.)

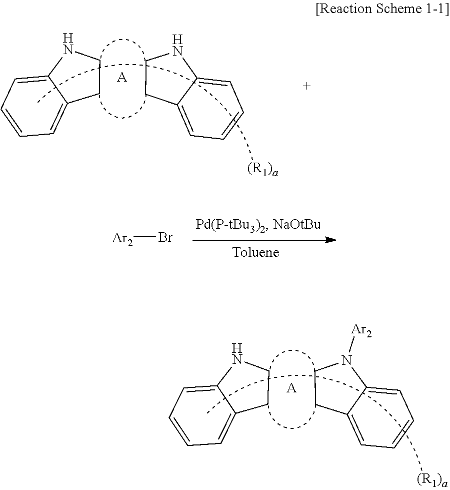

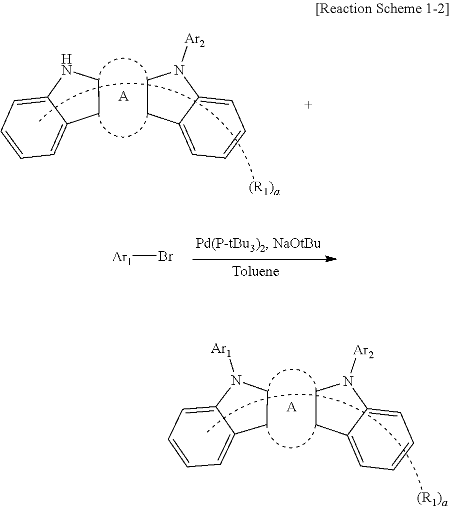

However, the series of processes of Reaction Schemes 1-1 and 1-2 are only examples, and the method for preparing the compound of Chemical Formula 1 can be more specifically described in Synthesis Examples to be described later.

Meanwhile, the Chemical Formula 2 can be the following Chemical Formula 2-1.

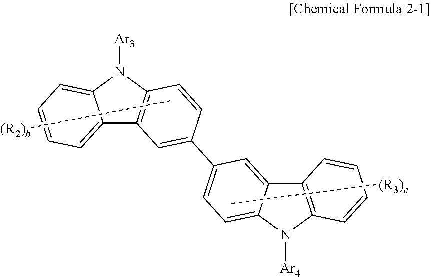

wherein in Chemical Formula 2-1, Ar₃, Ar₄, R₂, R₃, b and c have the same definitions as in claim 1.

Ar₃ and Ar₄ can each independently be a substituted or unsubstituted C₆₋₃₀ aryl, or a substituted or unsubstituted C₂₋₃₀ heteroaryl containing at least one heteroatom selected from the group consisting of N, O and S.

Specifically, Ar₃ and Ar₄ can each independently be phenyl, biphenylyl, (phenyl)biphenylyl, naphthyl, dimethylfluorenyl, dibenzofuranyl, or dibenzothiophenyl.

R₂ and R₃ can each independently be hydrogen, deuterium, a substituted or unsubstituted C₆₋₃₀ aryl, or a substituted or unsubstituted C₂₋₃₀ heteroaryl containing at least one heteroatom selected from the group consisting of N, O and S.

Specifically, R₂ and R₃ can each independently be hydrogen or phenyl.

More specifically, any one of R₂ and R₃ can be phenyl, and the other can be hydrogen.

b and c are each independently an integer of 1 to 7.

Representative examples of the compound of Chemical Formula 2 are as follows:

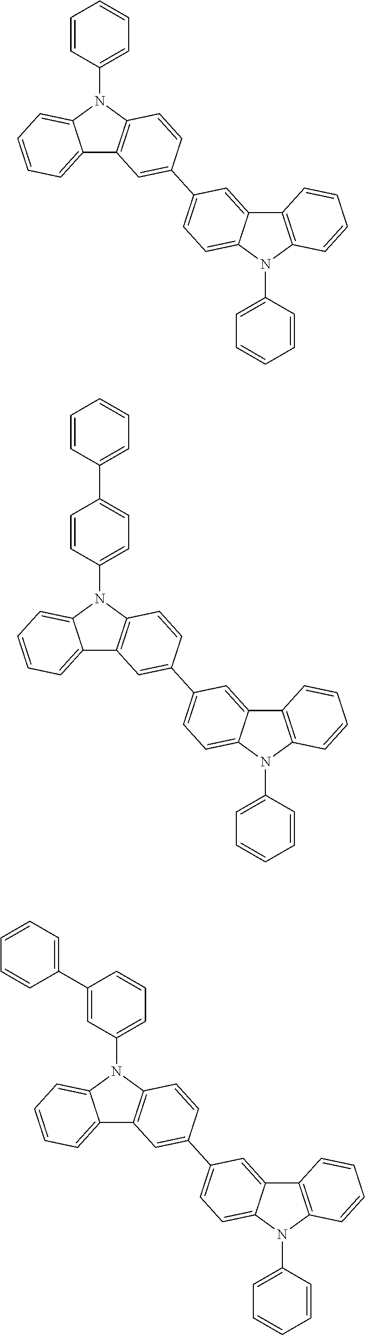 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

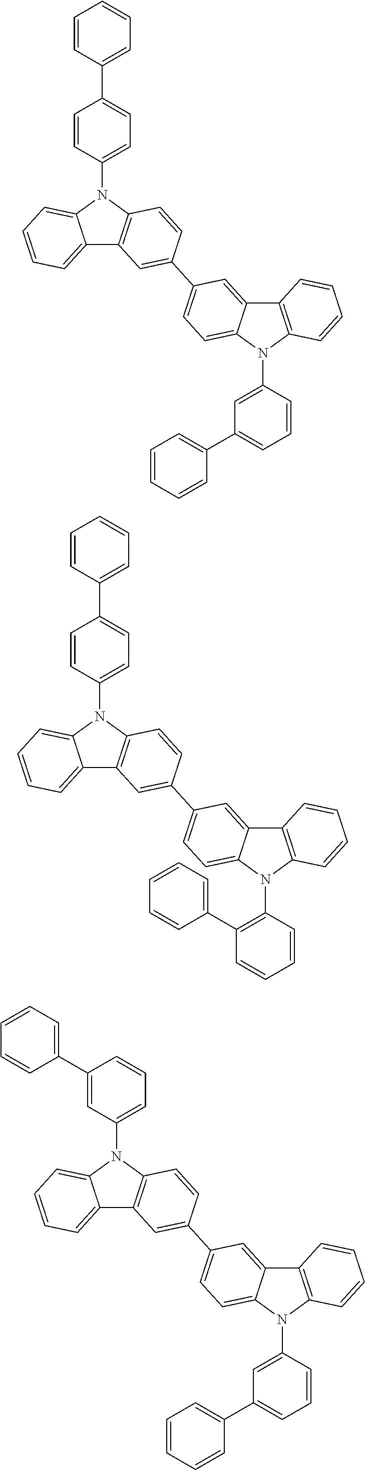 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

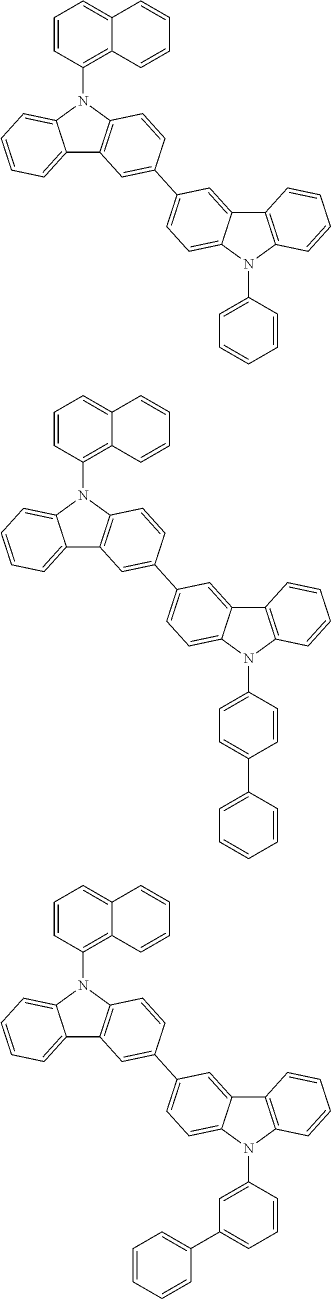 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1]

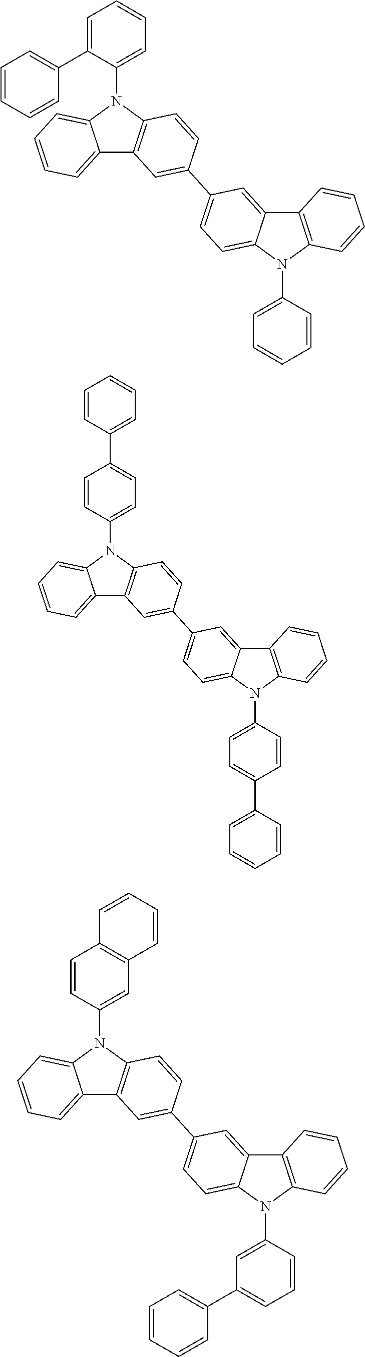 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccc6ccccc6c5)ccc43)c2)cc1]

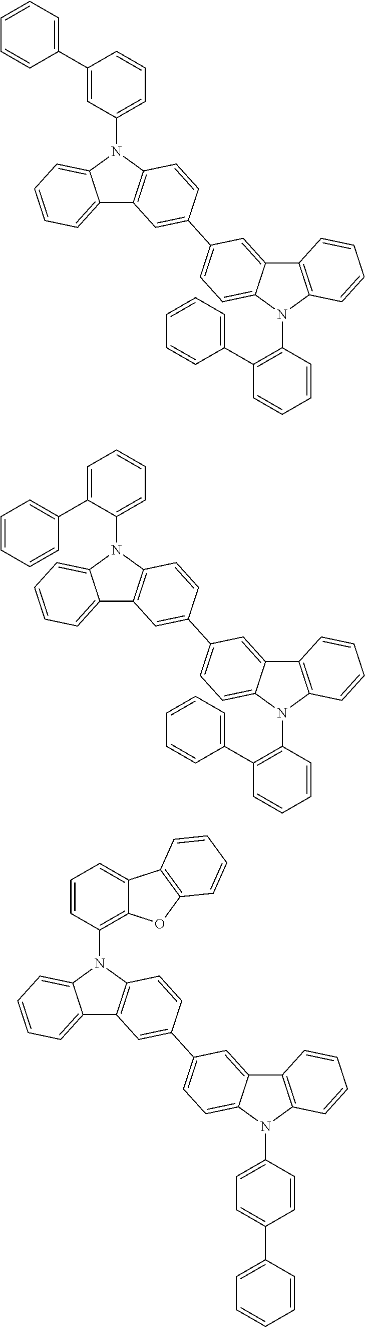 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5cccc6c5oc5ccccc56)ccc43)cc2)cc1]

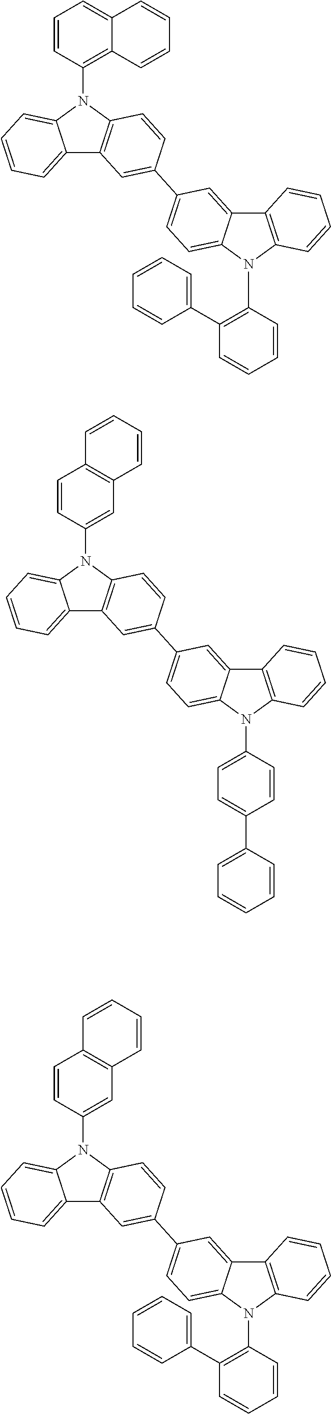 [SMILES: c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccccc2-n2c3ccccc3c3cc(-c4ccc5c(c4)c4ccccc4n5-c4ccc5ccccc5c4)ccc32)cc1]

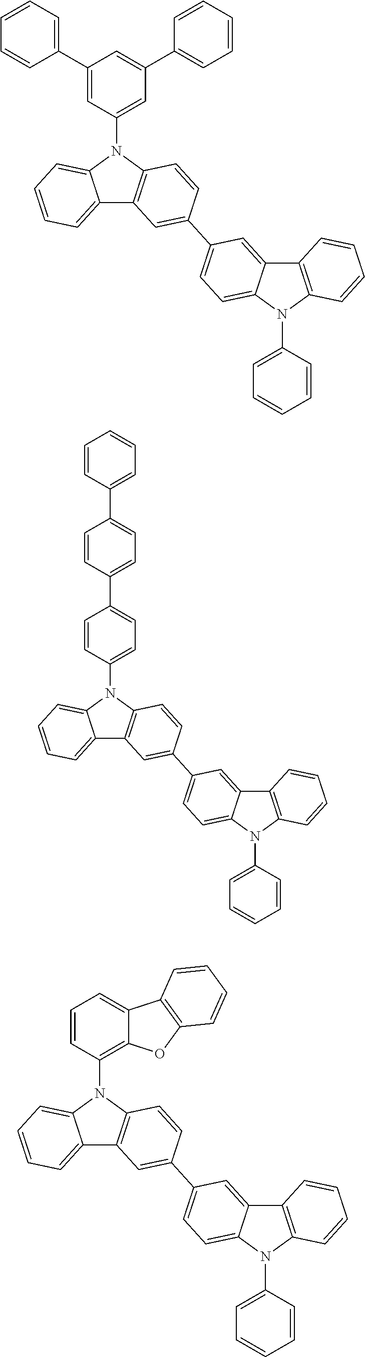 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-n2c3ccccc3c3cc(-c4ccc5c(c4)c4ccccc4n5-c4cccc5c4oc4ccccc45)ccc32)cc1]

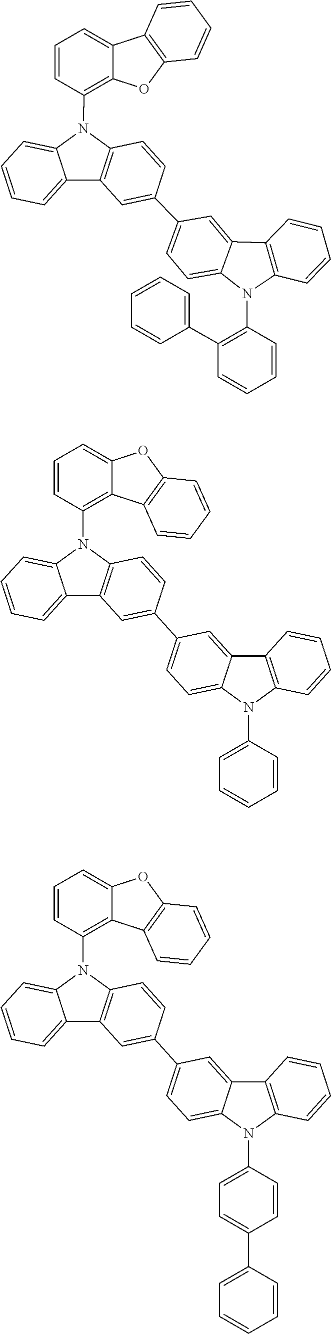 [SMILES: c1ccc(-c2ccccc2-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc5oc6ccccc6c45)ccc32)cc1 ; c1ccc(-c2ccc(-n3c4ccccc4c4cc(-c5ccc6c(c5)c5ccccc5n6-c5cccc6oc7ccccc7c56)ccc43)cc2)cc1]

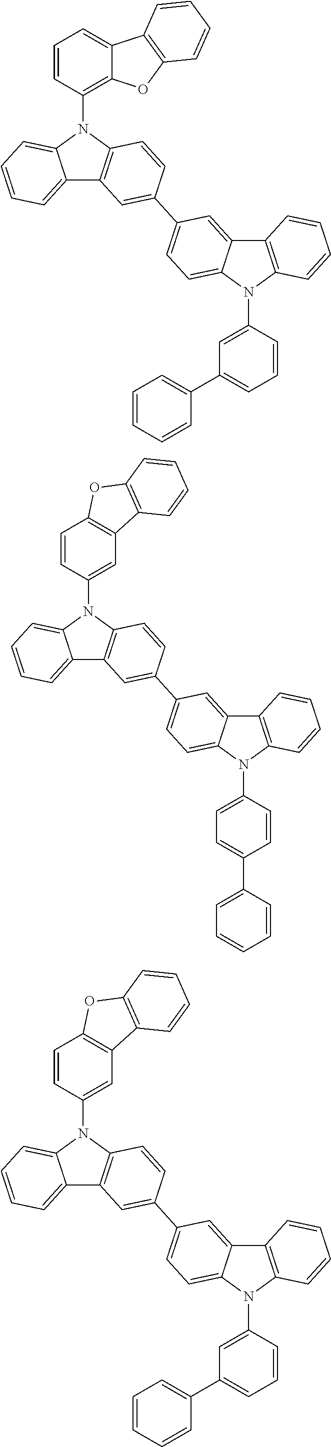

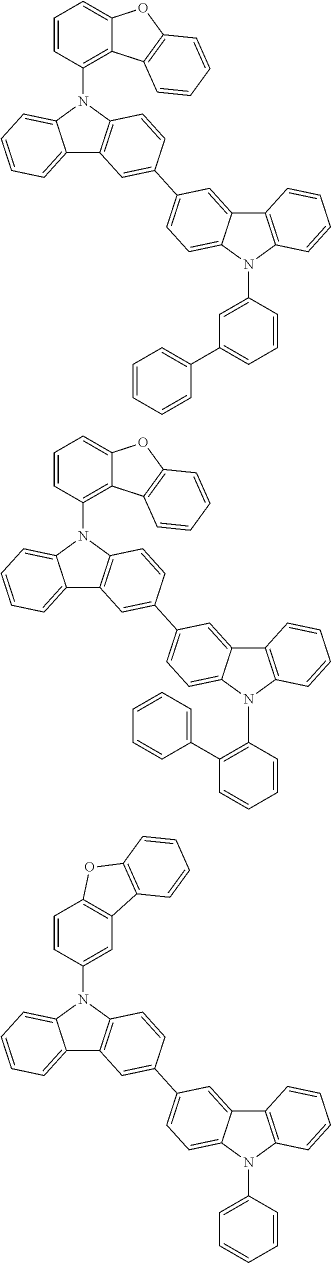

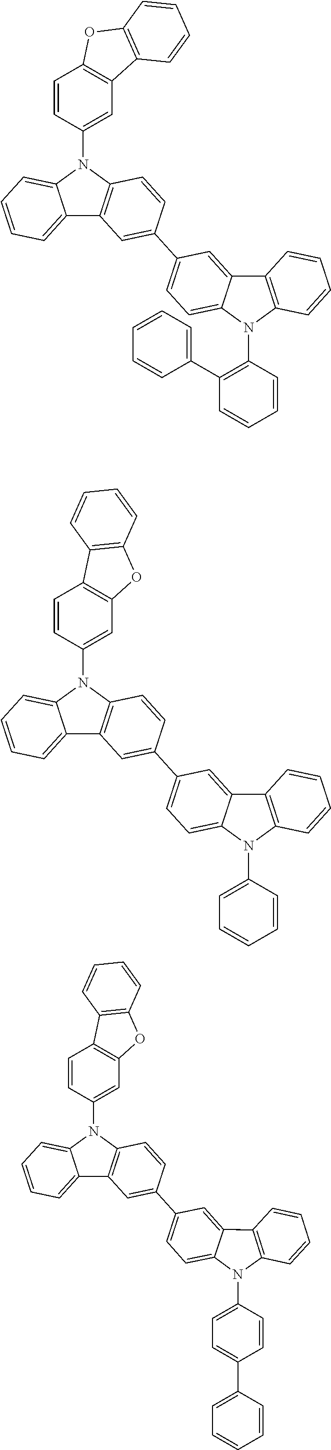 [SMILES: c1ccc(-c2ccccc2-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)oc4ccccc45)ccc32)cc1 ; c1ccc(-c2ccc(-n3c4ccccc4c4cc(-c5ccc6c(c5)c5ccccc5n6-c5ccc6c(c5)oc5ccccc56)ccc43)cc2)cc1]

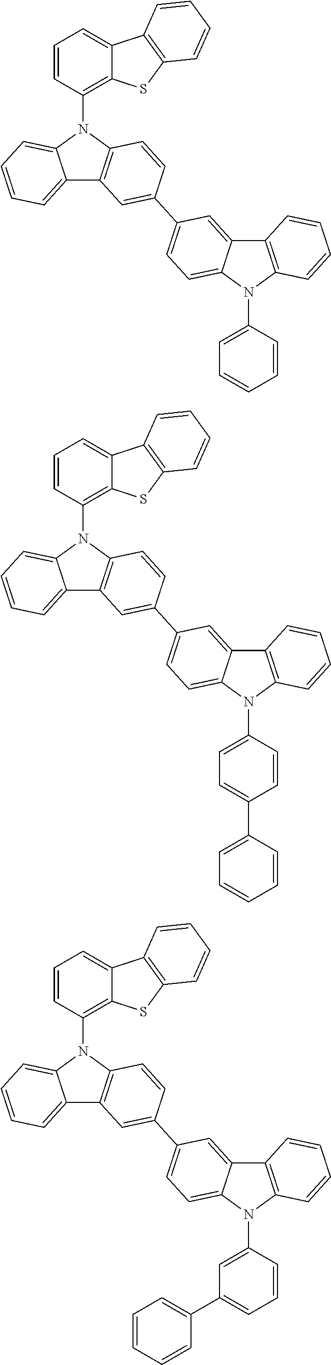

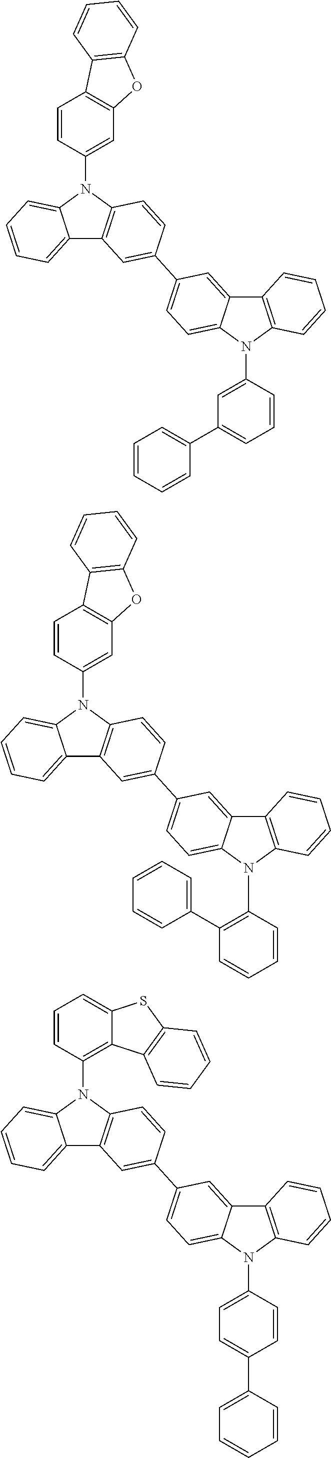

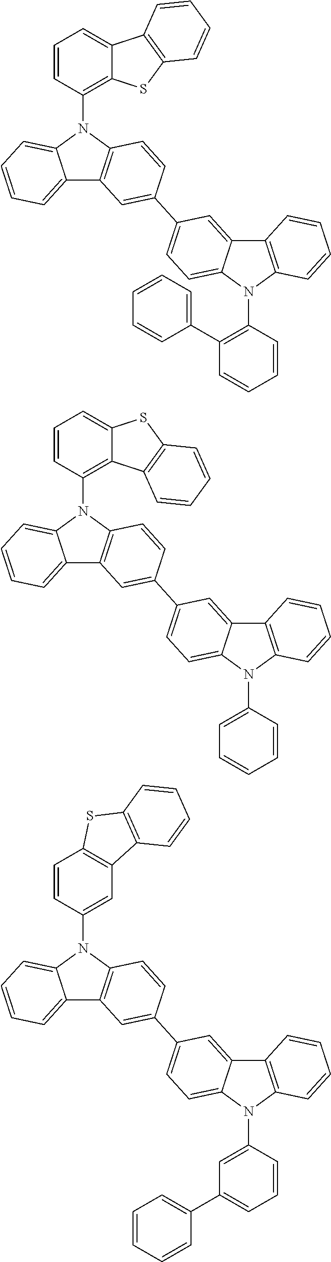 [SMILES: c1ccc(-c2ccccc2-n2c3ccccc3c3cc(-c4ccc5c(c4)c4ccccc4n5-c4cccc5c4sc4ccccc45)ccc32)cc1 ; c1ccc(-n2c3ccccc3c3cc(-c4ccc5c(c4)c4ccccc4n5-c4cccc5sc6ccccc6c45)ccc32)cc1 ; c1ccc(-c2cccc(-n3c4ccccc4c4cc(-c5ccc6c(c5)c5ccccc5n6-c5ccc6sc7ccccc7c6c5)ccc43)c2)cc1]

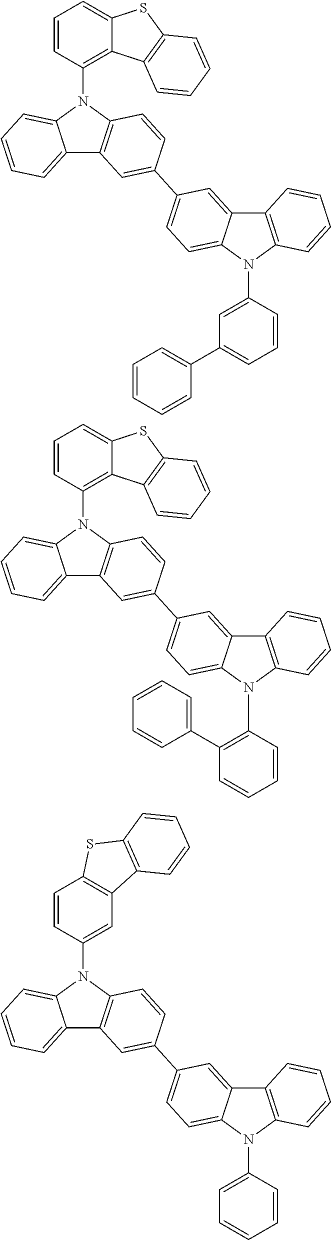

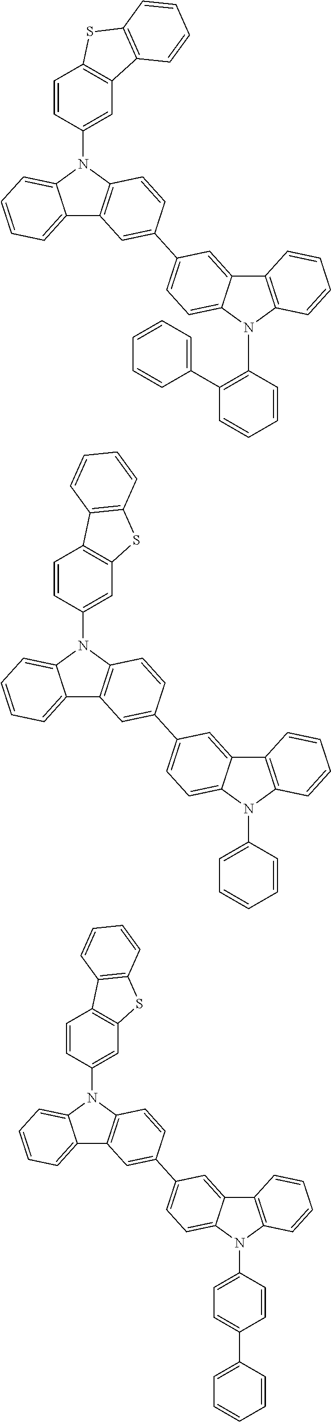 [SMILES: c1ccc(-c2ccccc2-n2c3ccccc3c3cc(-c4ccc5c(c4)c4ccccc4n5-c4ccc5sc6ccccc6c5c4)ccc32)cc1 ; c1ccc(-n2c3ccccc3c3cc(-c4ccc5c(c4)c4ccccc4n5-c4ccc5c(c4)sc4ccccc45)ccc32)cc1 ; c1ccc(-c2ccc(-n3c4ccccc4c4cc(-c5ccc6c(c5)c5ccccc5n6-c5ccc6c(c5)sc5ccccc56)ccc43)cc2)cc1]

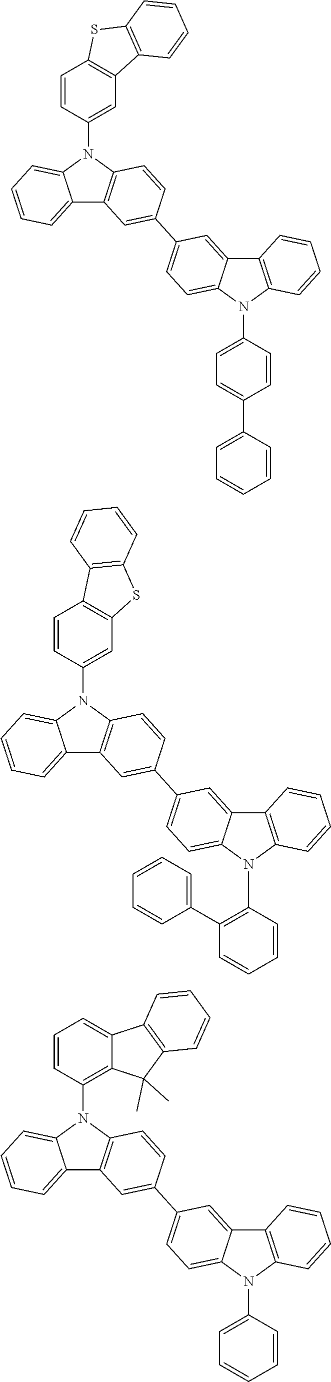

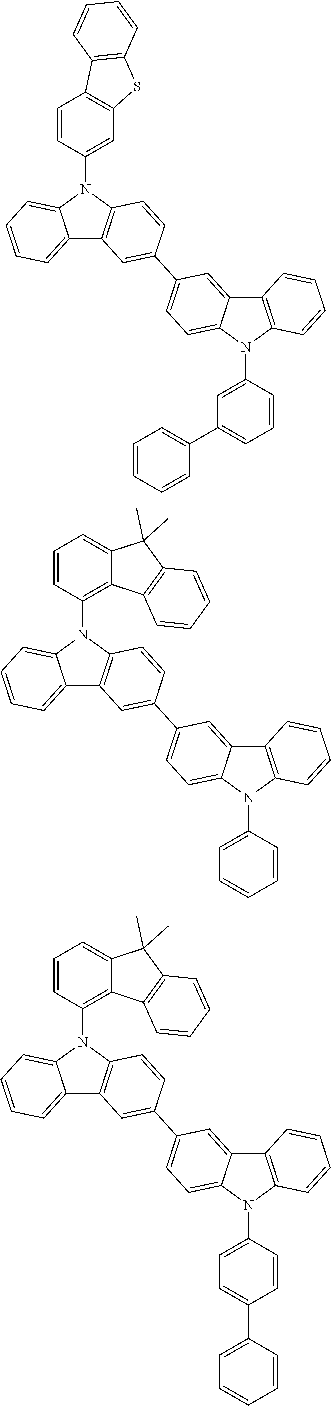

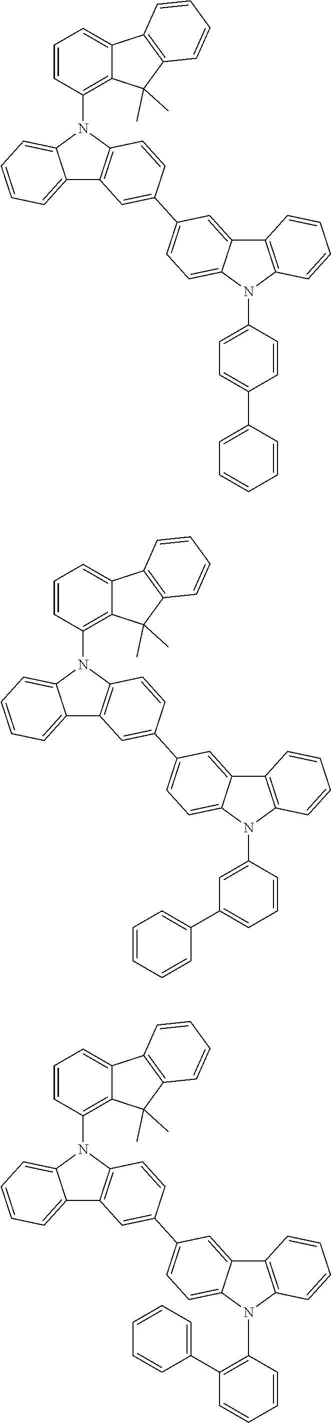 [SMILES: CC1(C)c2ccccc2-c2cccc(-n3c4ccccc4c4cc(-c5ccc6c(c5)c5ccccc5n6-c5ccc(-c6ccccc6)cc5)ccc43)c21 ; CC1(C)c2ccccc2-c2cccc(-n3c4ccccc4c4cc(-c5ccc6c(c5)c5ccccc5n6-c5cccc(-c6ccccc6)c5)ccc43)c21 ; CC1(C)c2ccccc2-c2cccc(-n3c4ccccc4c4cc(-c5ccc6c(c5)c5ccccc5n6-c5ccccc5-c5ccccc5)ccc43)c21]

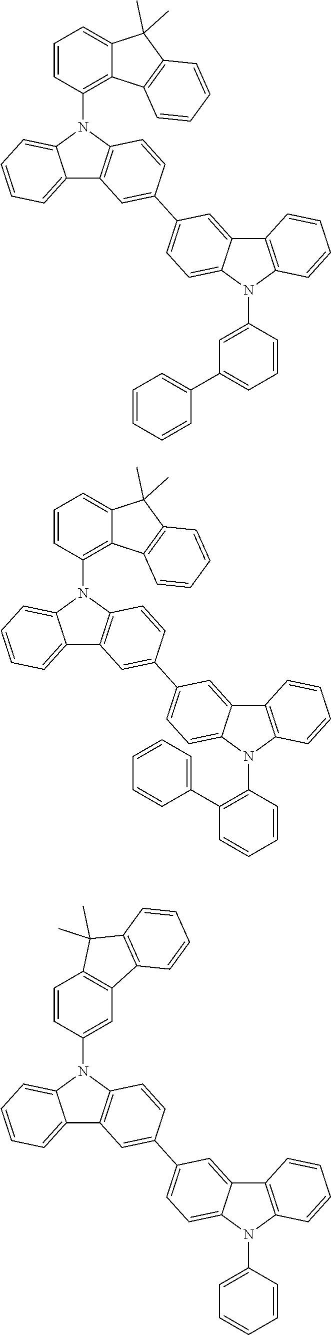

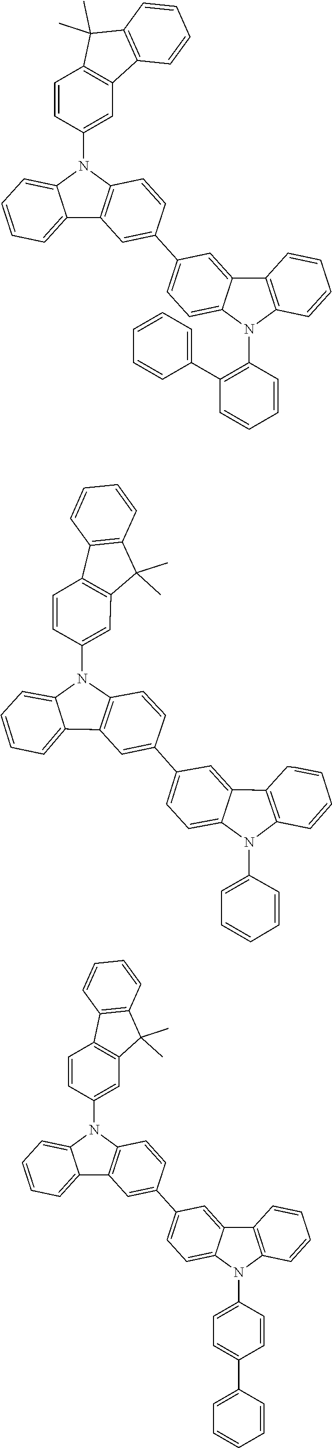 [SMILES: CC1(C)c2ccccc2-c2cc(-n3c4ccccc4c4cc(-c5ccc6c(c5)c5ccccc5n6-c5ccccc5-c5ccccc5)ccc43)ccc21 ; CC1(C)c2ccccc2-c2ccc(-n3c4ccccc4c4cc(-c5ccc6c(c5)c5c(n6-c6ccccc6)C=CC=CC5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21]

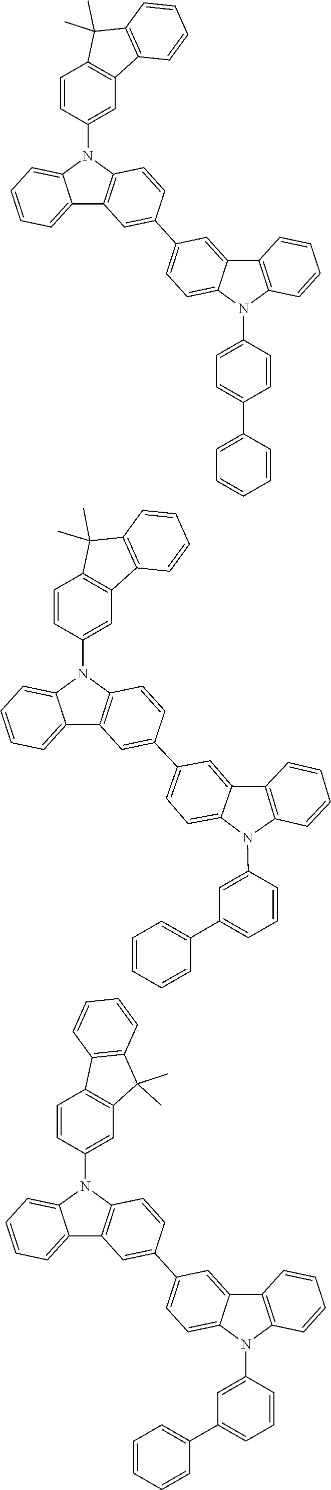

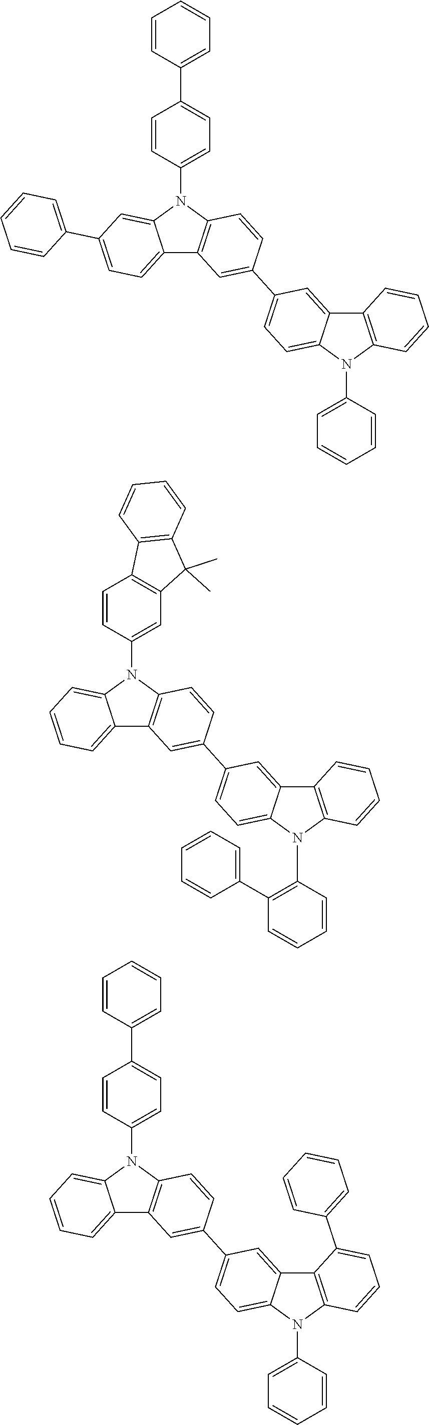 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccc(-n3c4ccccc4c4cc(-c5ccc6c(c5)c5c(-c7ccccc7)cccc5n6-c5ccccc5)ccc43)cc2)cc1]

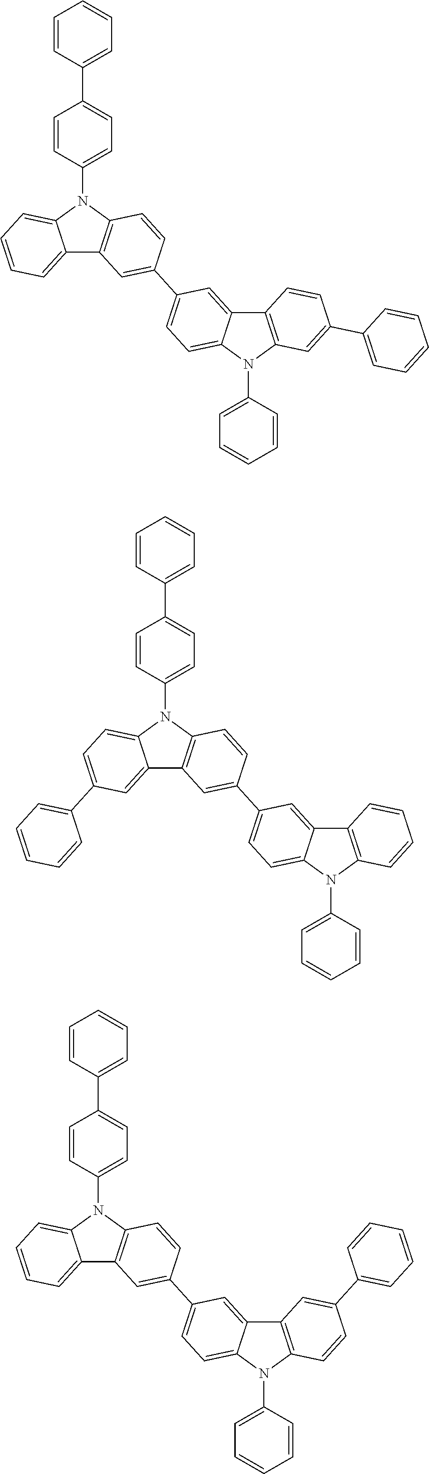 [SMILES: c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

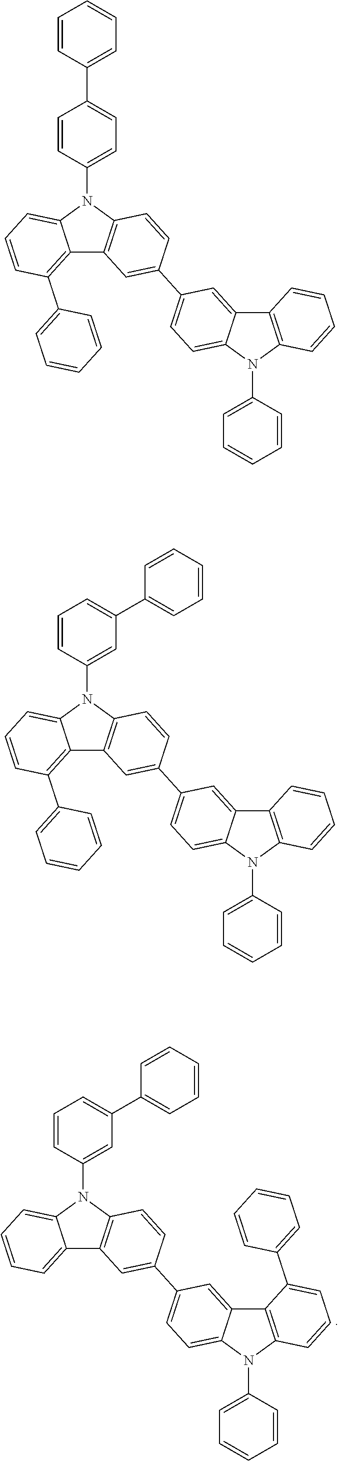 [SMILES: c1ccc(-c2ccc(-n3c4ccc(-c5ccc6c(c5)c5ccccc5n6-c5ccccc5)cc4c4c(-c5ccccc5)cccc43)cc2)cc1 ; c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2cccc(-n3c4ccccc4c4cc(-c5ccc6c(c5)c5c(-c7ccccc7)cccc5n6-c5ccccc5)ccc43)c2)cc1]

The compound of Chemical Formula 2 can be prepared by a series of processes as shown in Reaction Schemes 2-1 and 2-2 below. (The definition of each substituent in Reaction Schemes 2-1 and 2-2 is the same as described above.)

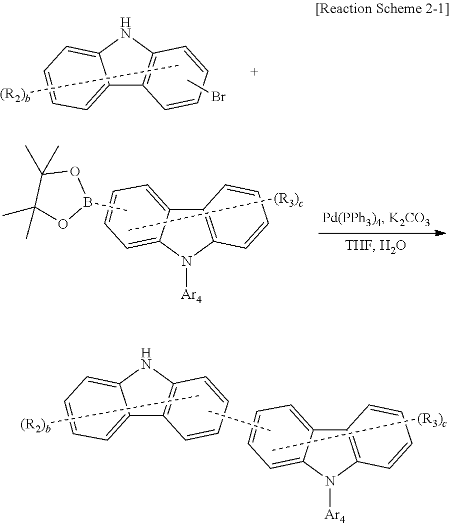

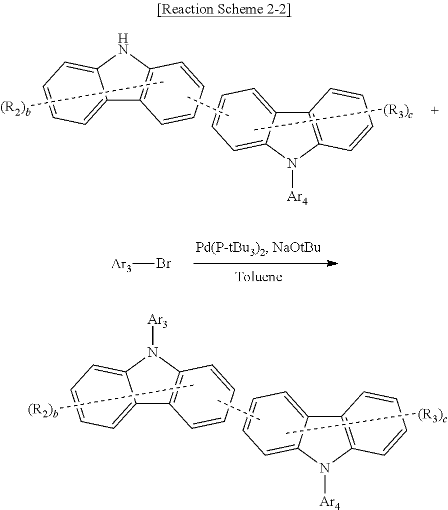

However, the series of processes of Reaction Schemes 2-1 and 2-2 are only examples, and the method for preparing the compound of Chemical Formula 2 can be more specifically described in Synthesis Examples to be described later.

In Chemical Formula 3, B is a benzene ring condensed with two adjacent pentagonal rings, and

the Chemical Formula 3 can have a structure of any one of the following Chemical Formulae 3-1 to 3-6 depending on the condensed form of B:

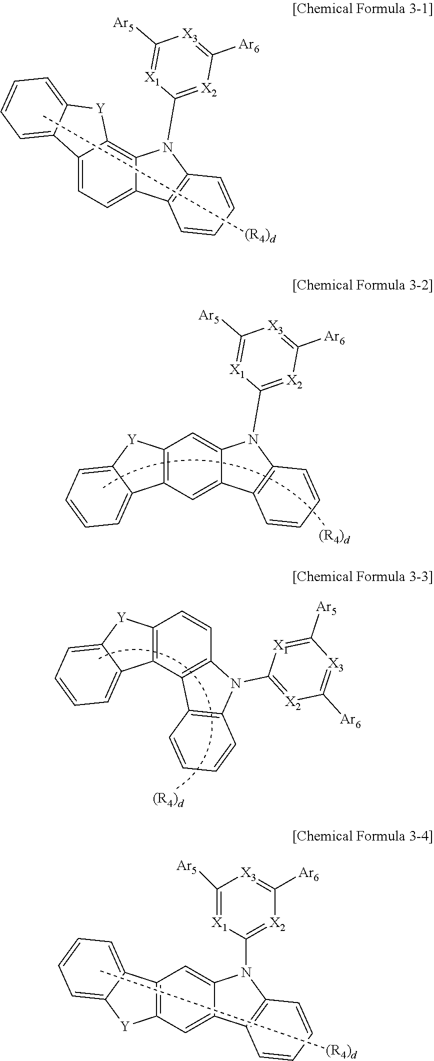

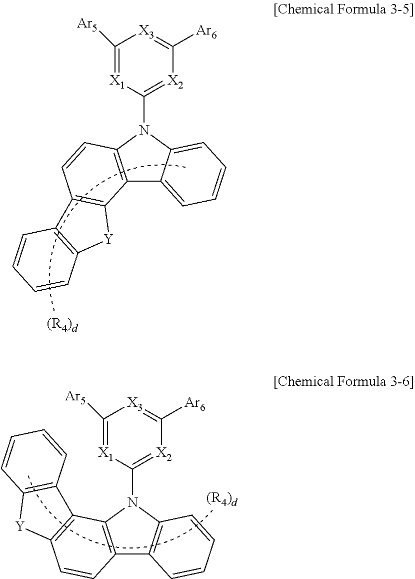

wherein in Chemical Formulae 3-1 to 3-6, X₁, X₂, X₃, Ar₅, Ar₆, R₄, Y, and d have the same definitions as described above.

X₁ to X₃ are each independently CH, or N, provided that at least one of X₁ to X₃ is N.

Specifically, one, two, or all of X₁ to X₃ can be N.

Ar₅ and Ar₆ can each independently be a substituted or unsubstituted C₆₋₃₀ aryl, or a substituted or unsubstituted C₂₋₃₀ heteroaryl containing at least one heteroatom selected from the group consisting of N, O and S.

Specifically, Ar₅ and Ar₆ can each independently be phenyl, biphenylyl, (phenyl)biphenylyl, dimethylfluorenyl, dibenzofuranyl, or dibenzothiophenyl, wherein Ar₅ and Ar₆ can each independently be unsubstituted or substituted with at least one deuterium.

Y is NAr₇, wherein Ar₇ is a substituted or unsubstituted C₆₋₃₀ aryl, or a substituted or unsubstituted C₂₋₃₀ heteroaryl containing at least one heteroatom selected from the group consisting of N, O and S.

Specifically, Ar₇ is phenyl, biphenylyl, or terphenylyl; and Ar₇ can be unsubstituted or substituted with at least one deuterium, for example, 5 deuteriums.

R₄ can be hydrogen, deuterium, a substituted or unsubstituted C₆₋₃₀ aryl, or a substituted or unsubstituted C₂₋₃₀ heteroaryl containing at least one heteroatom selected from the group consisting of N, O and S.

Specifically, R₄ can be hydrogen or deuterium.

d is an integer of 1 to 10.

Representative examples of the compound of Chemical Formula 3 are as follows:

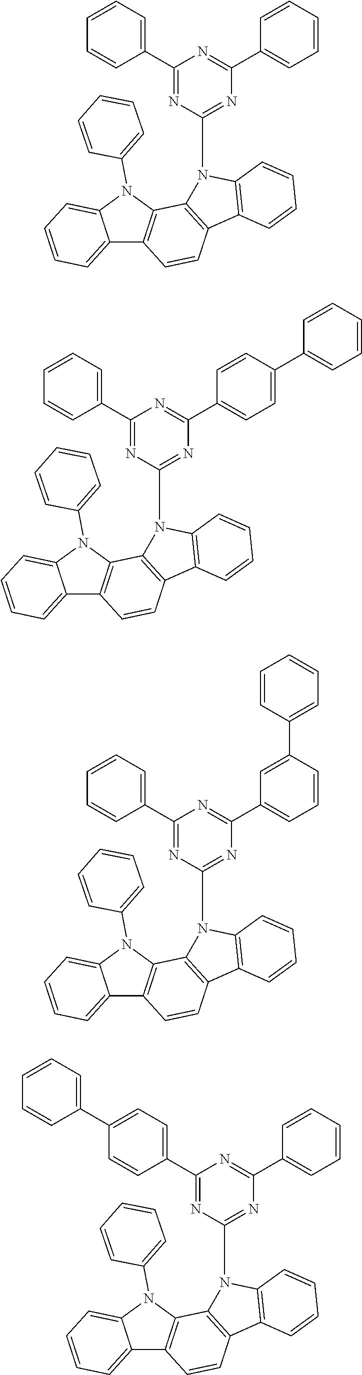

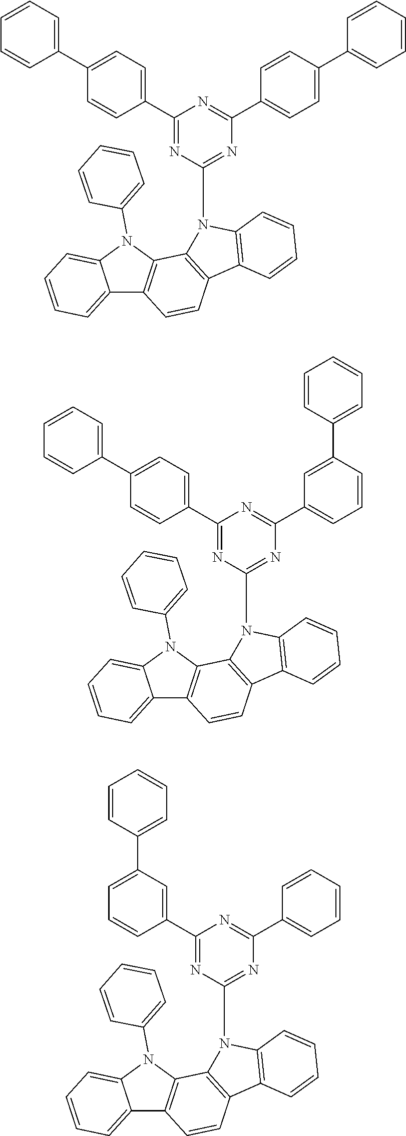 [SMILES: c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1 ; c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1 ; c1ccc(-c2cccc(-c3nc(-c4ccccc4)nc(-n4c5ccccc5c5ccc6c7ccccc7n(-c7ccccc7)c6c54)n3)c2)cc1]

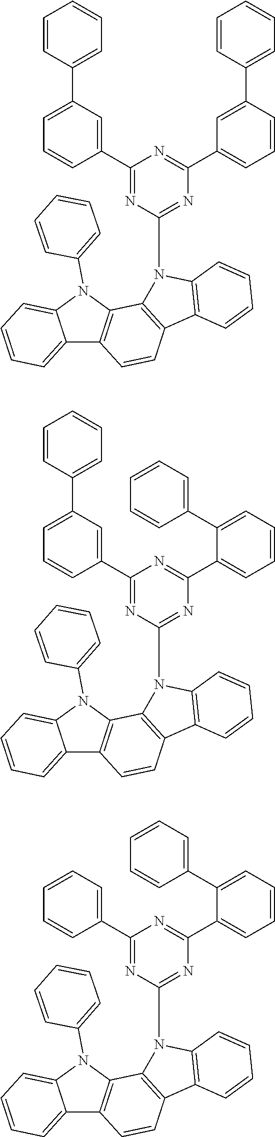 [SMILES: c1ccc(-c2cccc(-c3nc(-c4cccc(-c5ccccc5)c4)nc(-n4c5ccccc5c5ccc6c7ccccc7n(-c7ccccc7)c6c54)n3)c2)cc1 ; c1ccc(-c2cccc(-c3nc(-c4ccccc4-c4ccccc4)nc(-n4c5ccccc5c5ccc6c7ccccc7n(-c7ccccc7)c6c54)n3)c2)cc1 ; c1ccc(-c2nc(-c3ccccc3-c3ccccc3)nc(-n3c4ccccc4c4ccc5c6ccccc6n(-c6ccccc6)c5c43)n2)cc1]

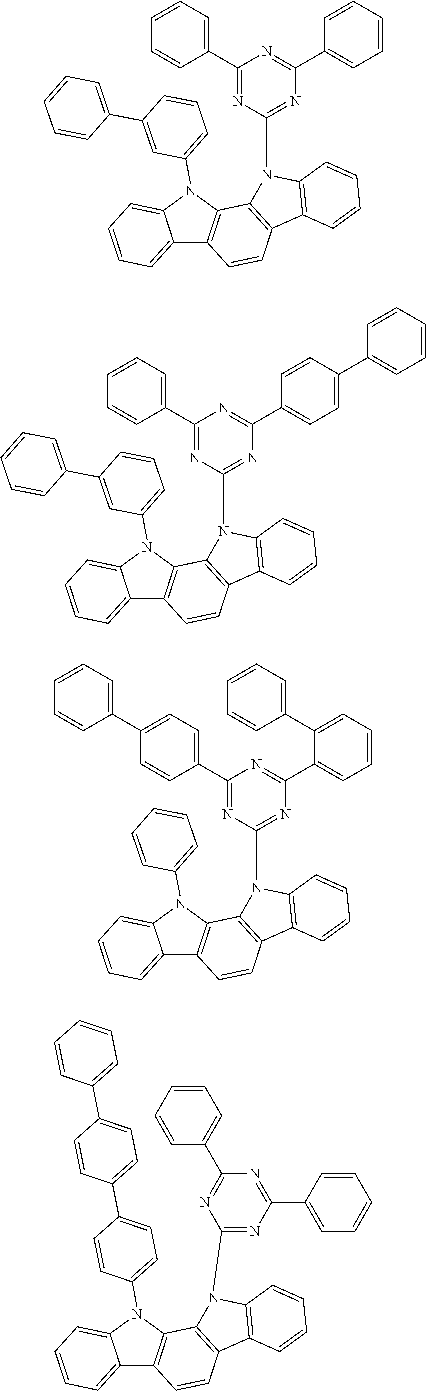 [SMILES: c1ccc(-c2cccc(-n3c4ccccc4c4ccc5c6ccccc6n(-c6nc(-c7ccccc7)nc(-c7ccccc7)n6)c5c43)c2)cc1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2ccc(-c3nc(-c4ccccc4-c4ccccc4)nc(-n4c5ccccc5c5ccc6c7ccccc7n(-c7ccccc7)c6c54)n3)cc2)cc1 ; c1ccc(-c2ccc(-c3ccc(-n4c5ccccc5c5ccc6c7ccccc7n(-c7nc(-c8ccccc8)nc(-c8ccccc8)n7)c6c54)cc3)cc2)cc1]

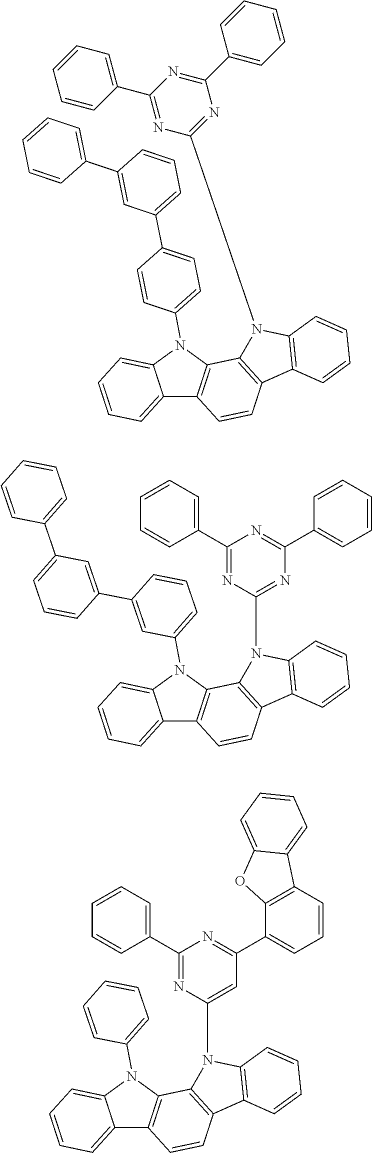 [SMILES: c1ccc(-c2cccc(-c3ccc(-n4c5ccccc5c5ccc6c7ccccc7n(-c7nc(-c8ccccc8)nc(-c8ccccc8)n7)c6c54)cc3)c2)cc1 ; c1ccc(-c2cccc(-c3cccc(-n4c5ccccc5c5ccc6c7ccccc7n(-c7nc(-c8ccccc8)nc(-c8ccccc8)n7)c6c54)c3)c2)cc1 ; c1ccc(-c2nc(-c3cccc4c3oc3ccccc34)cc(-n3c4ccccc4c4ccc5c6ccccc6n(-c6ccccc6)c5c43)n2)cc1]

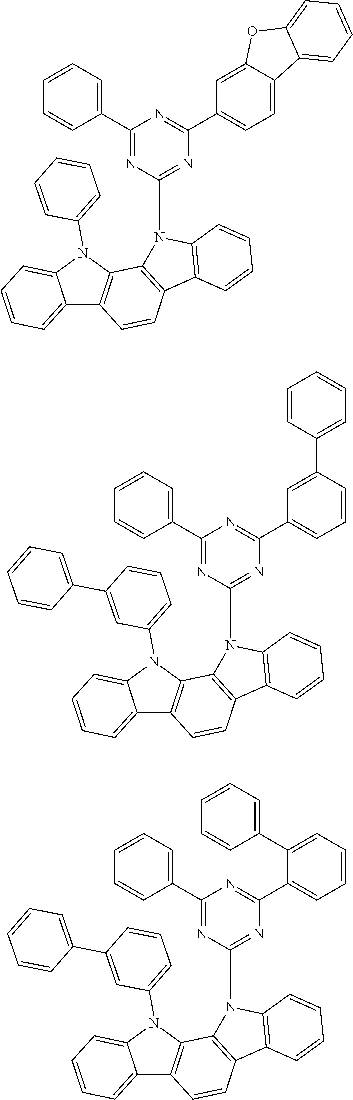 [SMILES: c1ccc(-c2nc(-c3ccc4c(c3)oc3ccccc34)nc(-n3c4ccccc4c4ccc5c6ccccc6n(-c6ccccc6)c5c43)n2)cc1 ; c1ccc(-c2cccc(-c3nc(-c4ccccc4)nc(-n4c5ccccc5c5ccc6c7ccccc7n(-c7cccc(-c8ccccc8)c7)c6c54)n3)c2)cc1 ; c1ccc(-c2cccc(-n3c4ccccc4c4ccc5c6ccccc6n(-c6nc(-c7ccccc7)nc(-c7ccccc7-c7ccccc7)n6)c5c43)c2)cc1]

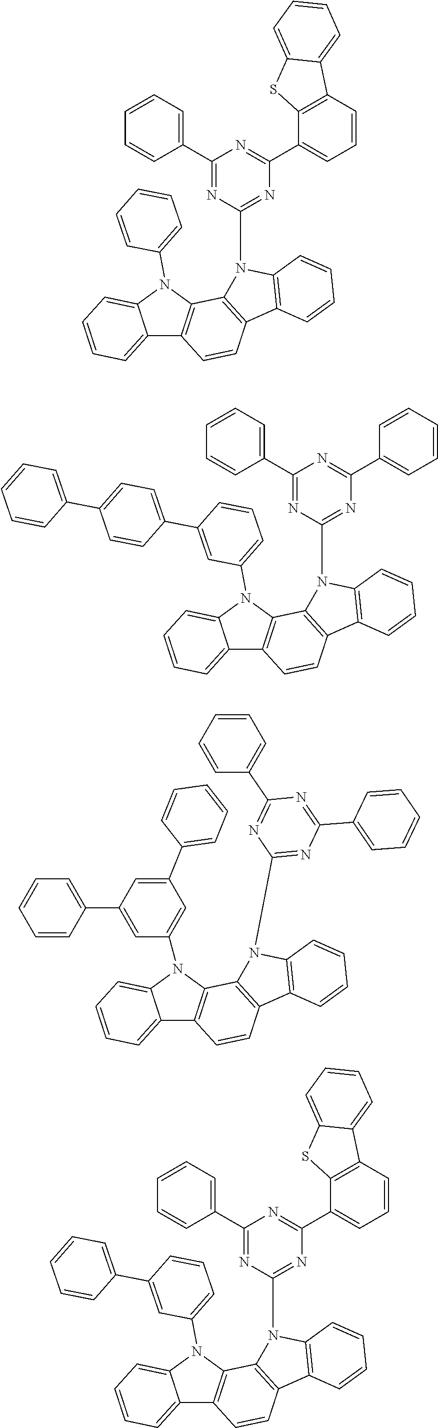

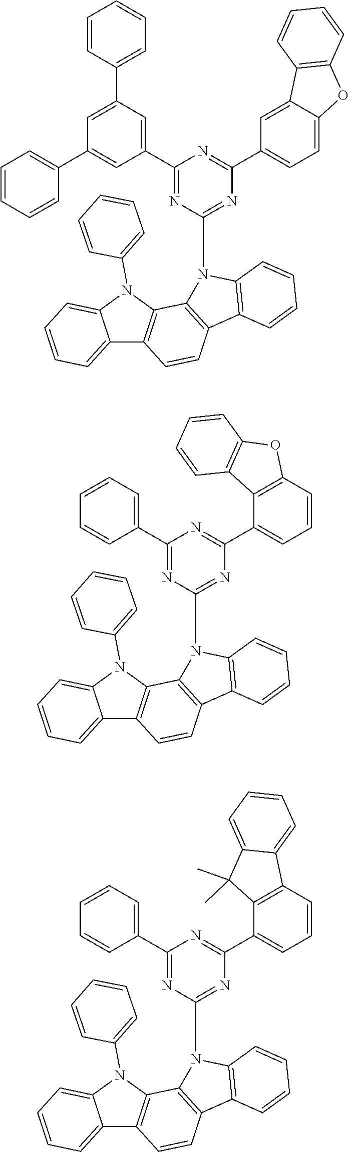 [SMILES: c1ccc(-c2cc(-c3ccccc3)cc(-c3nc(-c4ccc5oc6ccccc6c5c4)nc(-n4c5ccccc5c5ccc6c7ccccc7n(-c7ccccc7)c6c54)n3)c2)cc1 ; c1ccc(-c2nc(-c3cccc4oc5ccccc5c34)nc(-n3c4ccccc4c4ccc5c6ccccc6n(-c6ccccc6)c5c43)n2)cc1 ; CC1(C)c2ccccc2-c2cccc(-c3nc(-c4ccccc4)nc(-n4c5ccccc5c5ccc6c7ccccc7n(-c7ccccc7)c6c54)n3)c21]

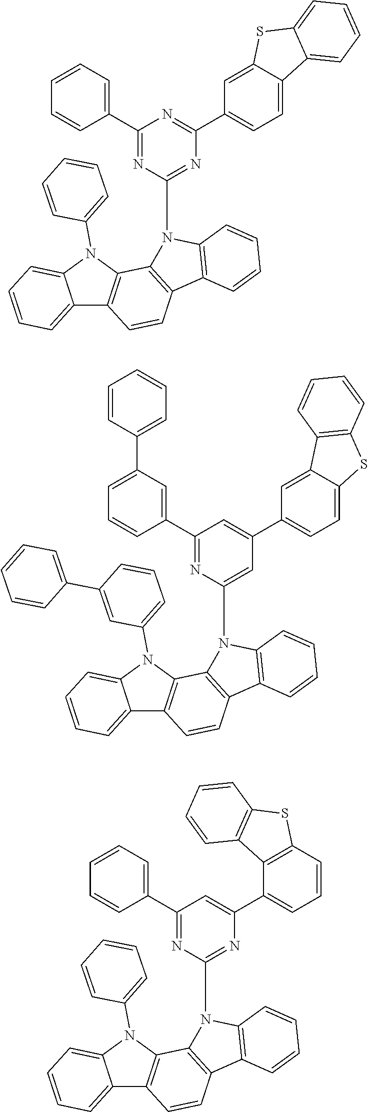

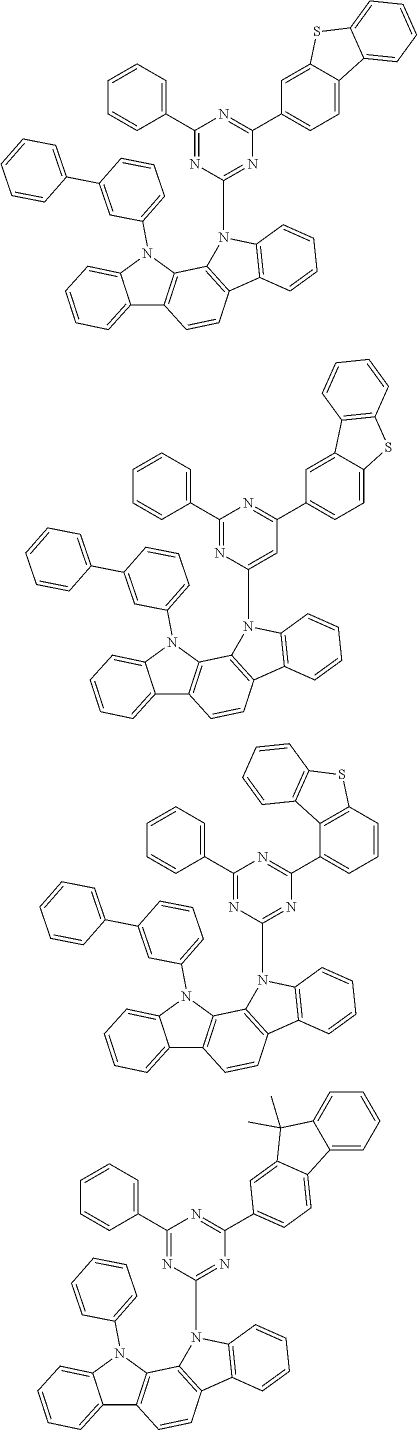

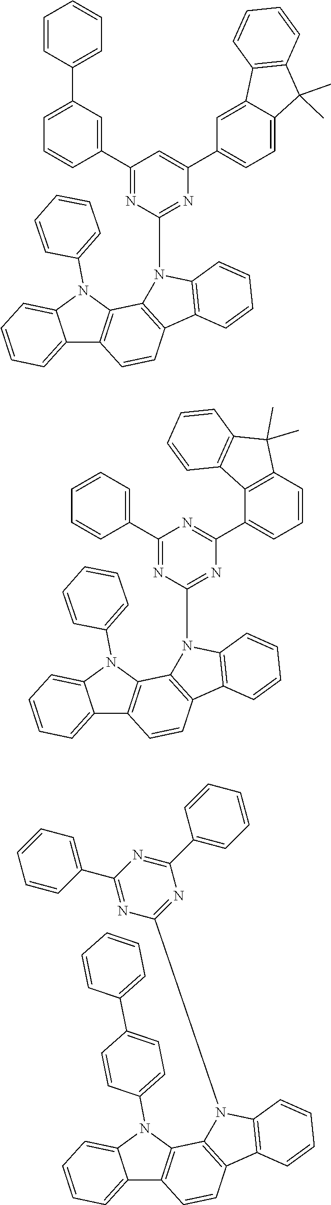 [SMILES: CC1(C)c2ccccc2-c2cc(-c3cc(-c4cccc(-c5ccccc5)c4)nc(-n4c5ccccc5c5ccc6c7ccccc7n(-c7ccccc7)c6c54)n3)ccc21 ; CC1(C)c2ccccc2-c2c(-c3nc(-c4ccccc4)nc(-n4c5ccccc5c5ccc6c7ccccc7n(-c7ccccc7)c6c54)n3)cccc21 ; c1ccc(-c2ccc(-n3c4ccccc4c4ccc5c6ccccc6n(-c6nc(-c7ccccc7)nc(-c7ccccc7)n6)c5c43)cc2)cc1]

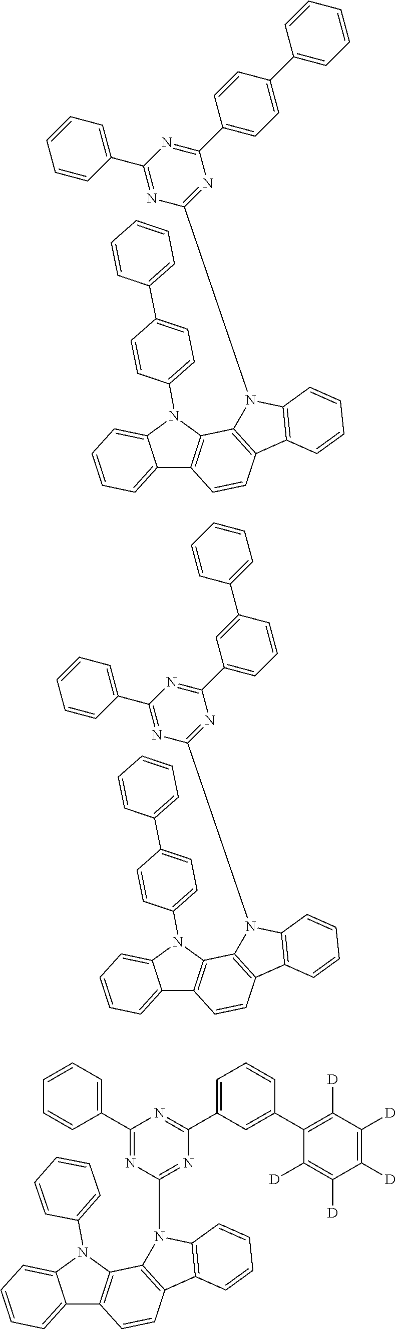

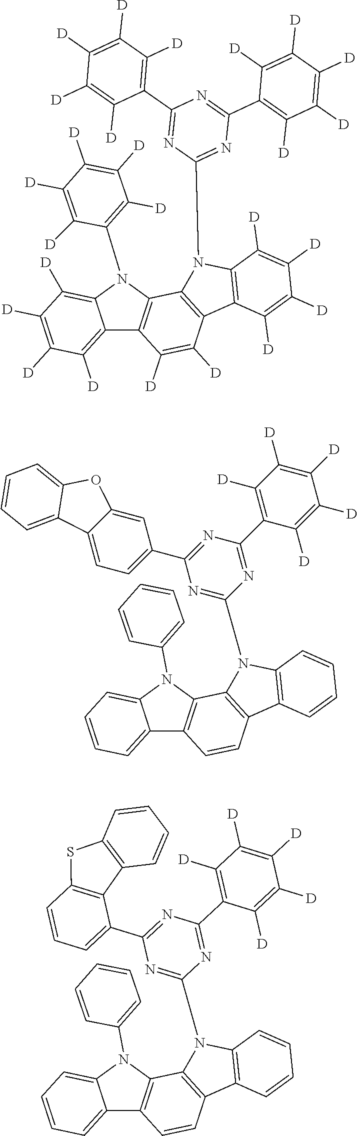 [SMILES: [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c([2H])c5c6c([2H])c([2H])c([2H])c([2H])c6n(-c6c([2H])c([2H])c([2H])c([2H])c6[2H])c5c43)n2)c([2H])c1[2H] ; [2H]c1c([2H])c([2H])c(-c2nc(-c3ccc4c(c3)oc3ccccc34)nc(-n3c4ccccc4c4ccc5c6ccccc6n(-c6ccccc6)c5c43)n2)c([2H])c1[2H] ; [2H]c1c([2H])c([2H])c(-c2nc(-c3cccc4sc5ccccc5c34)nc(-n3c4ccccc4c4ccc5c6ccccc6n(-c6ccccc6)c5c43)n2)c([2H])c1[2H]]

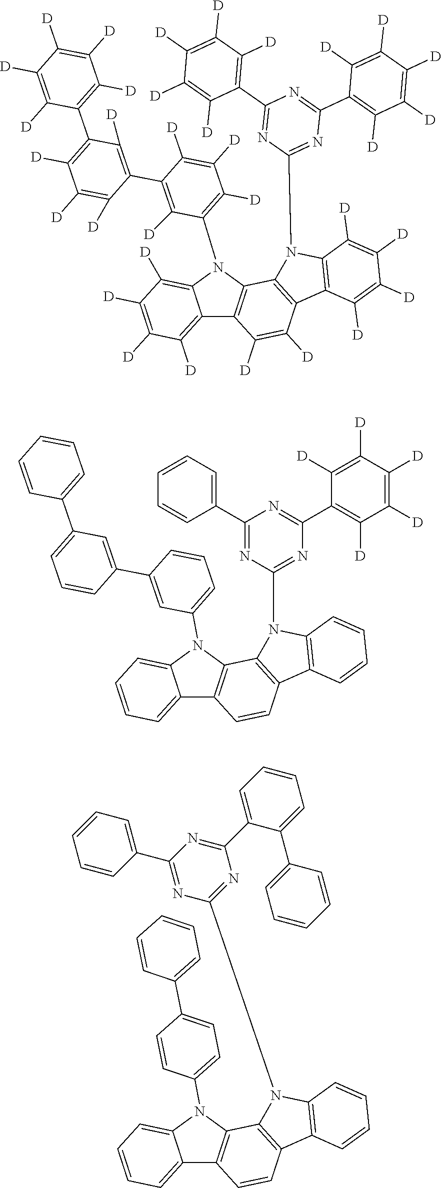

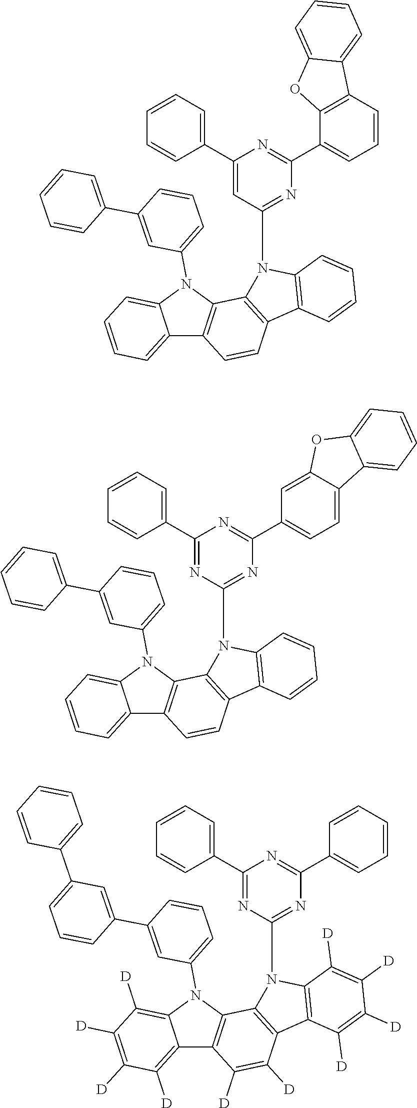 [SMILES: c1ccc(-c2cccc(-n3c4ccccc4c4ccc5c6ccccc6n(-c6cc(-c7ccccc7)nc(-c7cccc8c7oc7ccccc78)n6)c5c43)c2)cc1 ; c1ccc(-c2cccc(-n3c4ccccc4c4ccc5c6ccccc6n(-c6nc(-c7ccccc7)nc(-c7ccc8c(c7)oc7ccccc78)n6)c5c43)c2)cc1 ; [2H]c1c([2H])c([2H])c2c(c1[2H])c1c([2H])c([2H])c3c4c([2H])c([2H])c([2H])c([2H])c4n(-c4nc(-c5ccccc5)nc(-c5ccccc5)n4)c3c1n2-c1cccc(-c2cccc(-c3ccccc3)c2)c1]

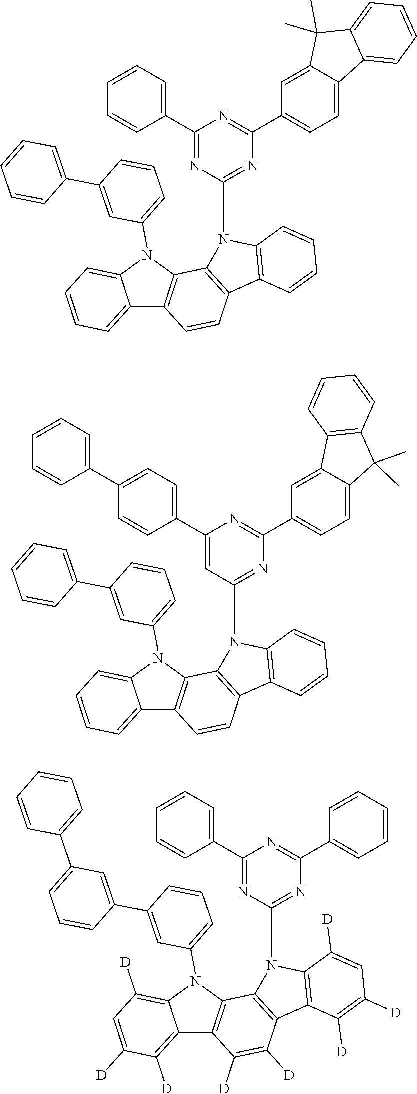 [SMILES: CC1(C)c2ccccc2-c2ccc(-c3nc(-c4ccccc4)nc(-n4c5ccccc5c5ccc6c7ccccc7n(-c7cccc(-c8ccccc8)c7)c6c54)n3)cc21 ; CC1(C)c2ccccc2-c2cc(-c3nc(-c4ccc(-c5ccccc5)cc4)cc(-n4c5ccccc5c5ccc6c7ccccc7n(-c7cccc(-c8ccccc8)c7)c6c54)n3)ccc21 ; [2H]c1cc([2H])c2c(c1[2H])c1c([2H])c([2H])c3c4c([2H])c([2H])cc([2H])c4n(-c4nc(-c5ccccc5)nc(-c5ccccc5)n4)c3c1n2-c1cccc(-c2cccc(-c3ccccc3)c2)c1]

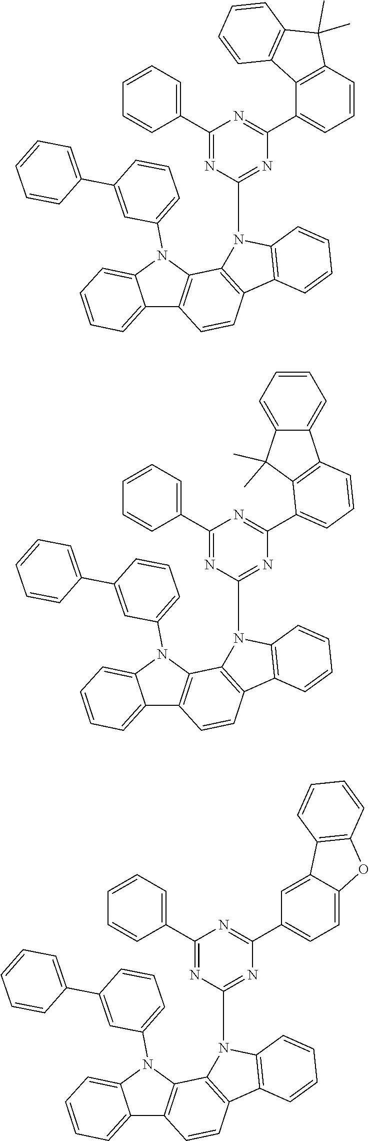 [SMILES: CC1(C)c2ccccc2-c2c(-c3nc(-c4ccccc4)nc(-n4c5ccccc5c5ccc6c7ccccc7n(-c7cccc(-c8ccccc8)c7)c6c54)n3)cccc21 ; CC1(C)c2ccccc2-c2cccc(-c3nc(-c4ccccc4)nc(-n4c5ccccc5c5ccc6c7ccccc7n(-c7cccc(-c8ccccc8)c7)c6c54)n3)c21 ; c1ccc(-c2cccc(-n3c4ccccc4c4ccc5c6ccccc6n(-c6nc(-c7ccccc7)nc(-c7ccc8oc9ccccc9c8c7)n6)c5c43)c2)cc1]

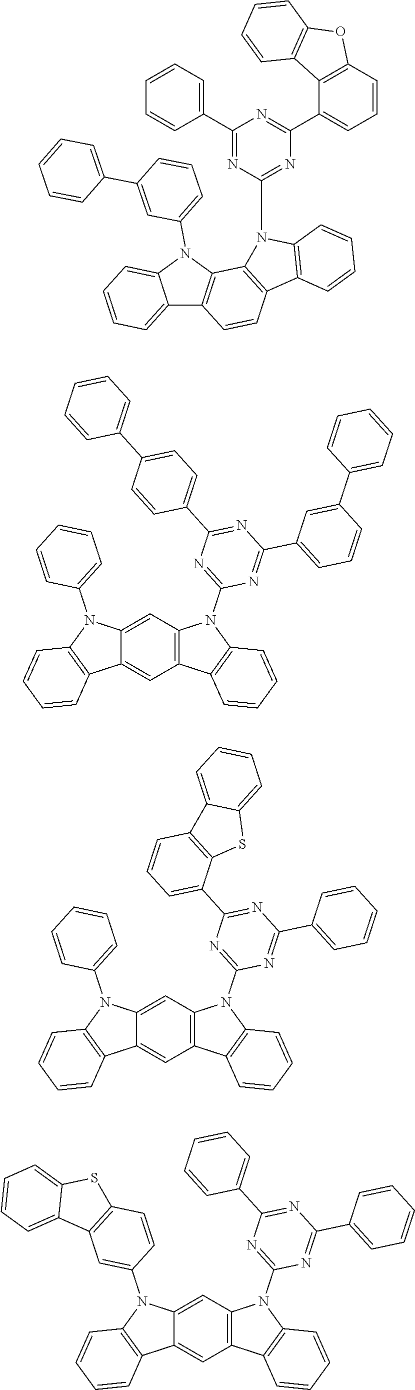

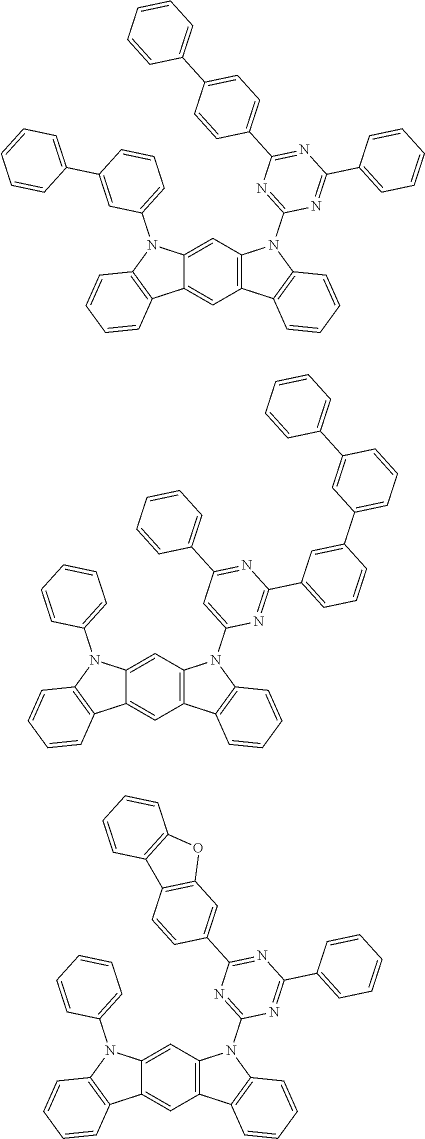

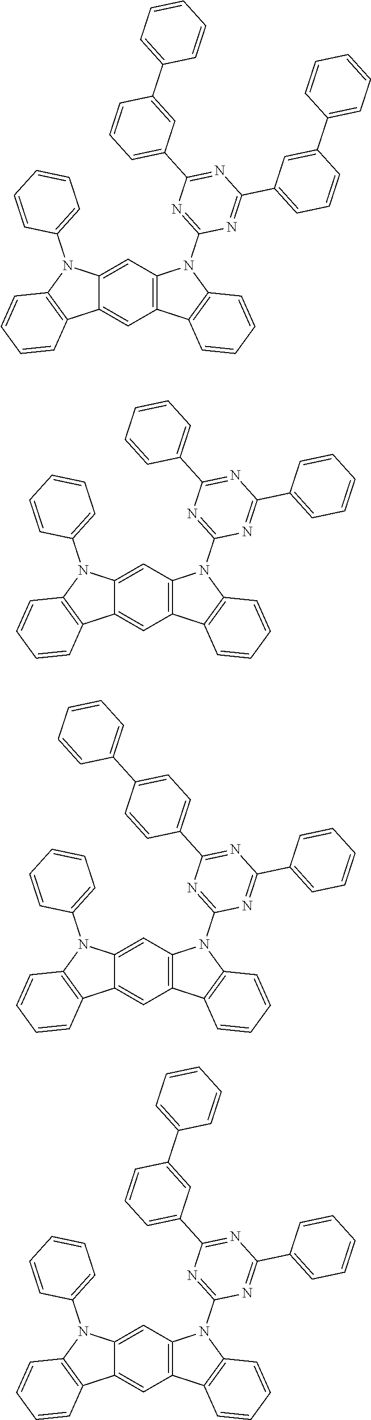

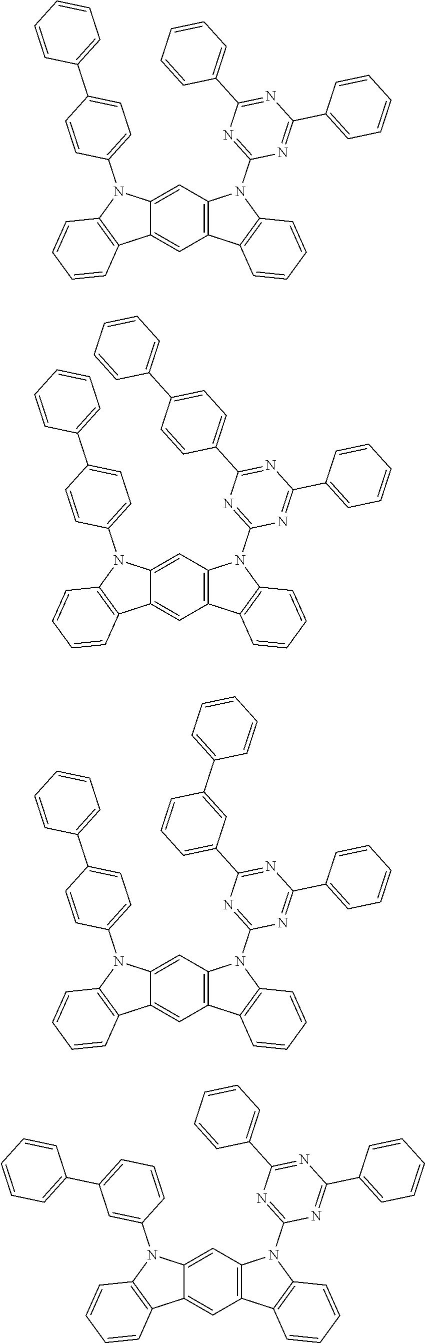

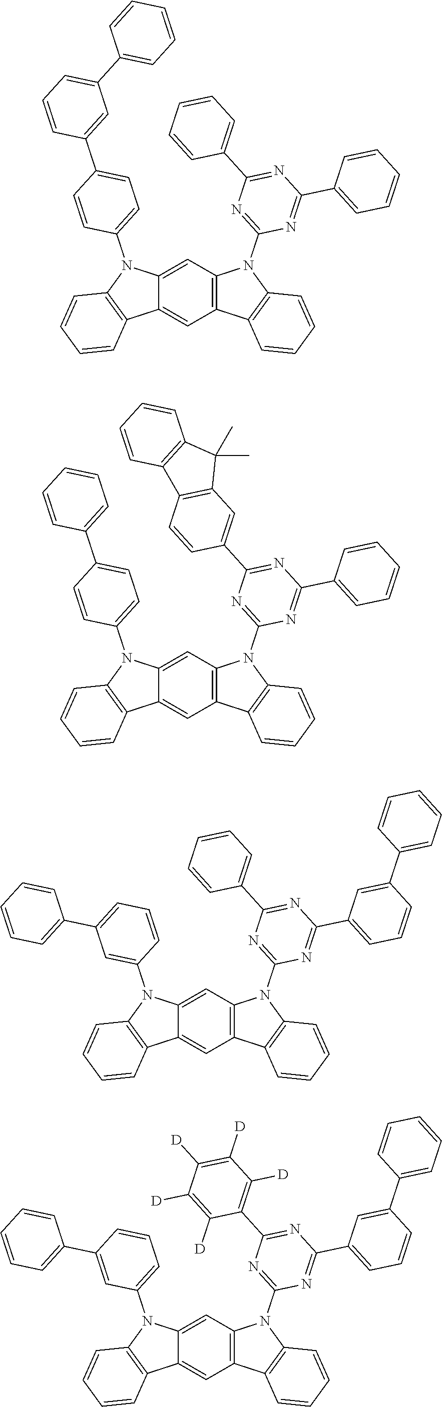 [SMILES: c1ccc(-c2cccc(-c3ccc(-n4c5ccccc5c5cc6c7ccccc7n(-c7nc(-c8ccccc8)nc(-c8ccccc8)n7)c6cc54)cc3)c2)cc1 ; CC1(C)c2ccccc2-c2ccc(-c3nc(-c4ccccc4)nc(-n4c5ccccc5c5cc6c7ccccc7n(-c7ccc(-c8ccccc8)cc7)c6cc54)n3)cc21 ; c1ccc(-c2cccc(-c3nc(-c4ccccc4)nc(-n4c5ccccc5c5cc6c7ccccc7n(-c7cccc(-c8ccccc8)c7)c6cc54)n3)c2)cc1 ; [2H]c1c([2H])c([2H])c(-c2nc(-c3cccc(-c4ccccc4)c3)nc(-n3c4ccccc4c4cc5c6ccccc6n(-c6cccc(-c7ccccc7)c6)c5cc43)n2)c([2H])c1[2H]]

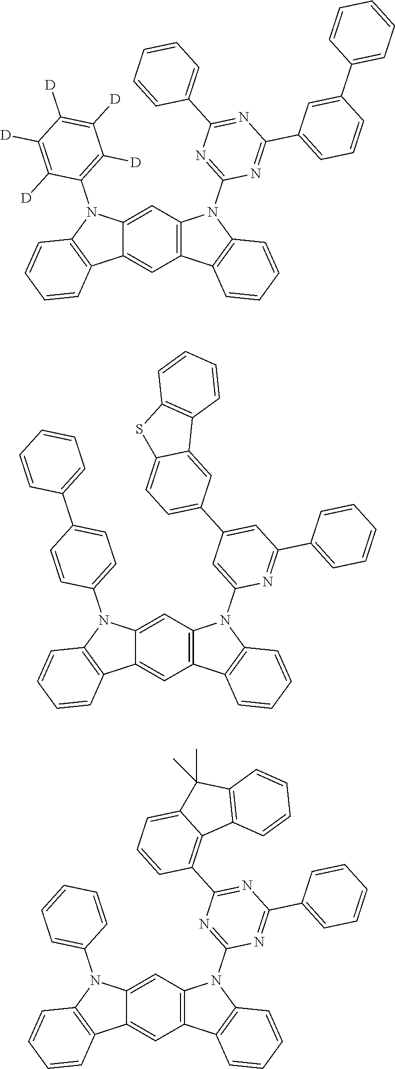 [SMILES: [2H]c1c([2H])c([2H])c(-n2c3ccccc3c3cc4c5ccccc5n(-c5nc(-c6ccccc6)nc(-c6cccc(-c7ccccc7)c6)n5)c4cc32)c([2H])c1[2H] ; c1ccc(-c2ccc(-n3c4ccccc4c4cc5c6ccccc6n(-c6cc(-c7ccc8sc9ccccc9c8c7)cc(-c7ccccc7)n6)c5cc43)cc2)cc1 ; CC1(C)c2ccccc2-c2c(-c3nc(-c4ccccc4)nc(-n4c5ccccc5c5cc6c7ccccc7n(-c7ccccc7)c6cc54)n3)cccc21]

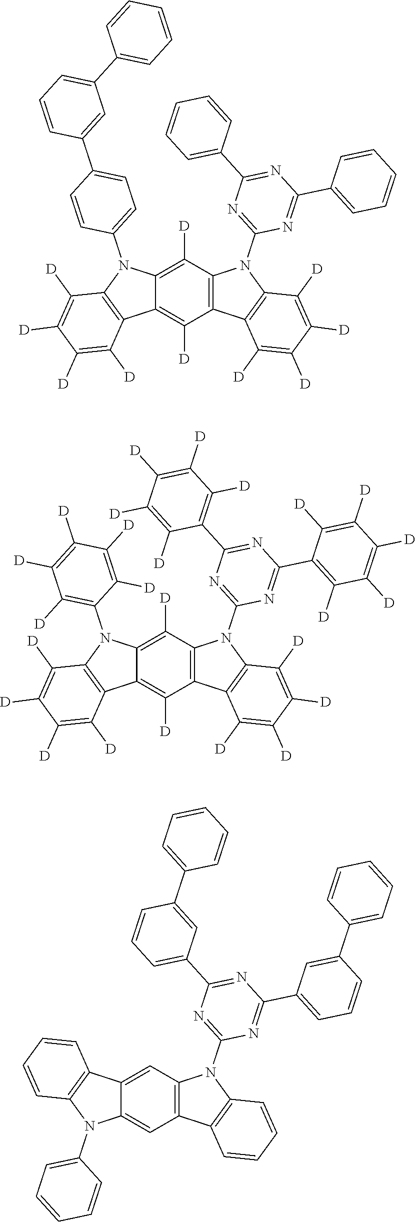 [SMILES: [2H]c1c([2H])c([2H])c2c(c1[2H])c1c([2H])c3c4c([2H])c([2H])c([2H])c([2H])c4n(-c4nc(-c5ccccc5)nc(-c5ccccc5)n4)c3c([2H])c1n2-c1ccc(-c2cccc(-c3ccccc3)c2)cc1 ; [2H]c1c([2H])c([2H])c(-c2nc(-c3c([2H])c([2H])c([2H])c([2H])c3[2H])nc(-n3c4c([2H])c([2H])c([2H])c([2H])c4c4c([2H])c5c6c([2H])c([2H])c([2H])c([2H])c6n(-c6c([2H])c([2H])c([2H])c([2H])c6[2H])c5c([2H])c43)n2)c([2H])c1[2H] ; c1ccc(-c2cccc(-c3nc(-c4cccc(-c5ccccc5)c4)nc(-n4c5ccccc5c5cc6c(cc54)c4ccccc4n6-c4ccccc4)n3)c2)cc1]

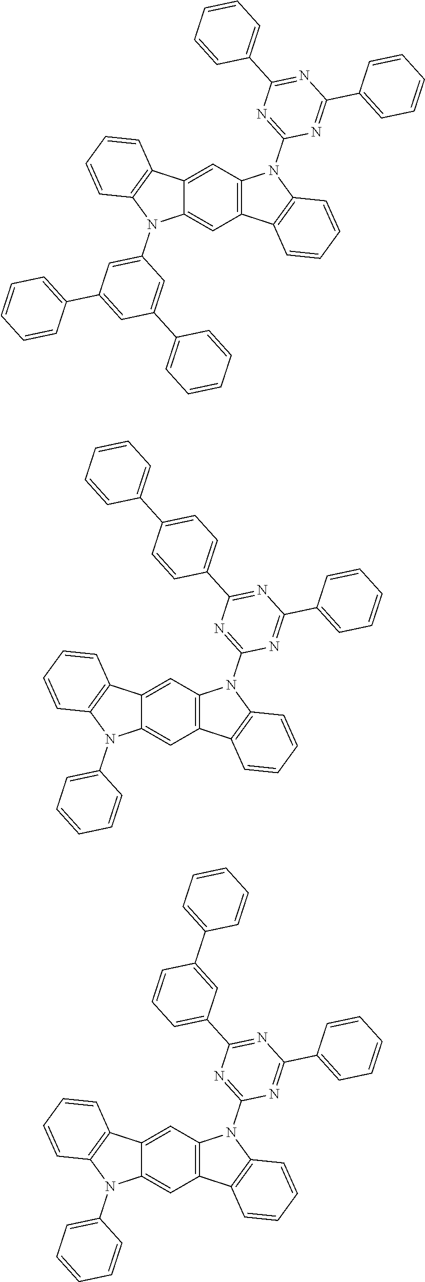 [SMILES: c1ccc(-c2cc(-c3ccccc3)cc(-n3c4ccccc4c4cc5c(cc43)c3ccccc3n5-c3nc(-c4ccccc4)nc(-c4ccccc4)n3)c2)cc1 ; c1ccc(-c2ccc(-c3nc(-c4ccccc4)nc(-n4c5ccccc5c5cc6c(cc54)c4ccccc4n6-c4ccccc4)n3)cc2)cc1 ; c1ccc(-c2cccc(-c3nc(-c4ccccc4)nc(-n4c5ccccc5c5cc6c(cc54)c4ccccc4n6-c4ccccc4)n3)c2)cc1]

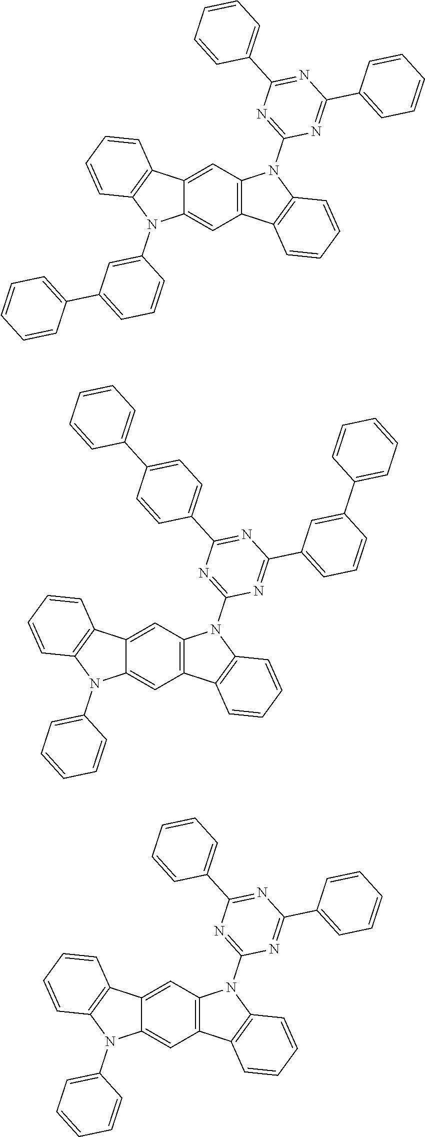 [SMILES: c1ccc(-c2cccc(-n3c4ccccc4c4cc5c(cc43)c3ccccc3n5-c3nc(-c4ccccc4)nc(-c4ccccc4)n3)c2)cc1 ; c1ccc(-c2ccc(-c3nc(-c4cccc(-c5ccccc5)c4)nc(-n4c5ccccc5c5cc6c(cc54)c4ccccc4n6-c4ccccc4)n3)cc2)cc1 ; c1ccc(-c2nc(-c3ccccc3)nc(-n3c4ccccc4c4cc5c(cc43)c3ccccc3n5-c3ccccc3)n2)cc1]

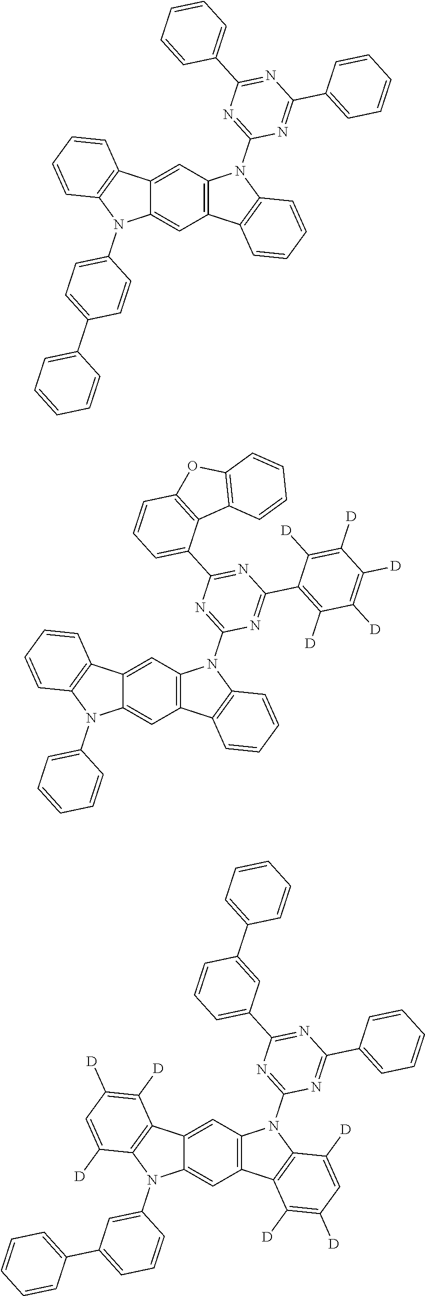 [SMILES: c1ccc(-c2ccc(-n3c4ccccc4c4cc5c(cc43)c3ccccc3n5-c3nc(-c4ccccc4)nc(-c4ccccc4)n3)cc2)cc1 ; [2H]c1c([2H])c([2H])c(-c2nc(-c3cccc4oc5ccccc5c34)nc(-n3c4ccccc4c4cc5c(cc43)c3ccccc3n5-c3ccccc3)n2)c([2H])c1[2H] ; [2H]c1cc([2H])c2c(c1[2H])c1cc3c(cc1n2-c1cccc(-c2ccccc2)c1)c1c([2H])c([2H])cc([2H])c1n3-c1nc(-c2ccccc2)nc(-c2cccc(-c3ccccc3)c2)n1]

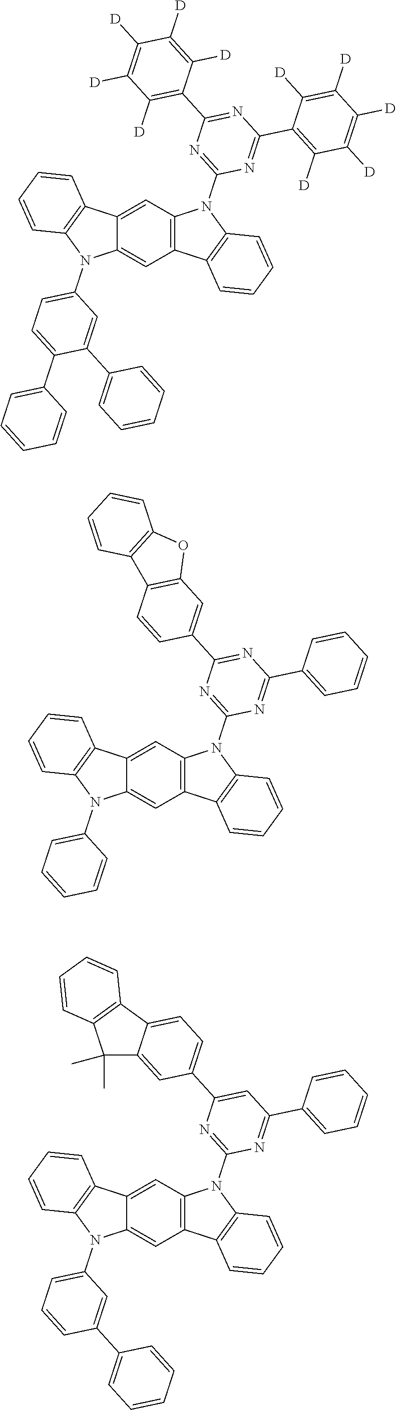 [SMILES: [2H]c1c([2H])c([2H])c(-c2nc(-c3c([2H])c([2H])c([2H])c([2H])c3[2H])nc(-n3c4ccccc4c4cc5c(cc43)c3ccccc3n5-c3ccc(-c4ccccc4)c(-c4ccccc4)c3)n2)c([2H])c1[2H] ; c1ccc(-c2nc(-c3ccc4c(c3)oc3ccccc34)nc(-n3c4ccccc4c4cc5c(cc43)c3ccccc3n5-c3ccccc3)n2)cc1 ; CC1(C)c2ccccc2-c2ccc(-c3cc(-c4ccccc4)nc(-n4c5ccccc5c5cc6c(cc54)c4ccccc4n6-c4cccc(-c5ccccc5)c4)n3)cc21]

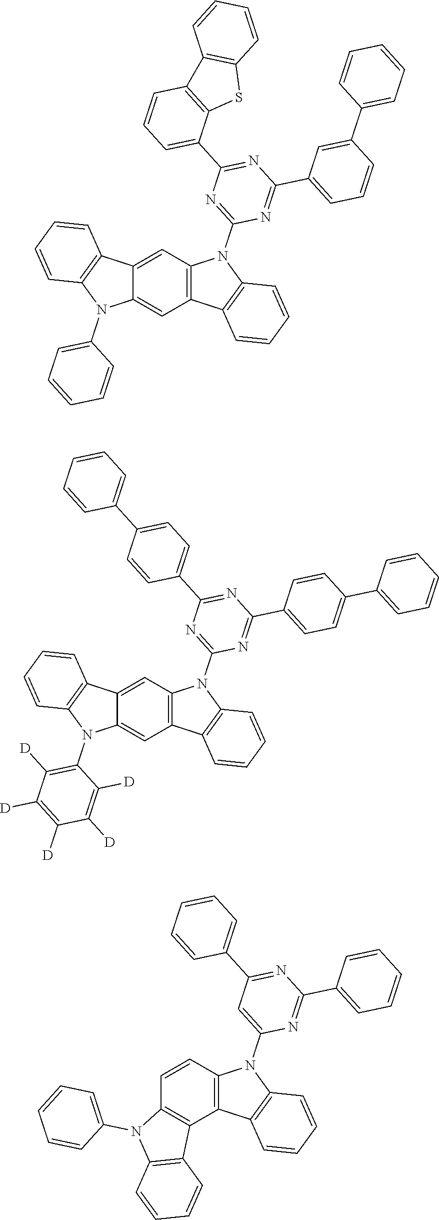 [SMILES: c1ccc(-c2cccc(-c3nc(-c4cccc5c4sc4ccccc45)nc(-n4c5ccccc5c5cc6c(cc54)c4ccccc4n6-c4ccccc4)n3)c2)cc1 ; [2H]c1c([2H])c([2H])c(-n2c3ccccc3c3cc4c(cc32)c2ccccc2n4-c2nc(-c3ccc(-c4ccccc4)cc3)nc(-c3ccc(-c4ccccc4)cc3)n2)c([2H])c1[2H] ; c1ccc(-c2cc(-n3c4ccccc4c4c5c6ccccc6n(-c6ccccc6)c5ccc43)nc(-c3ccccc3)n2)cc1]

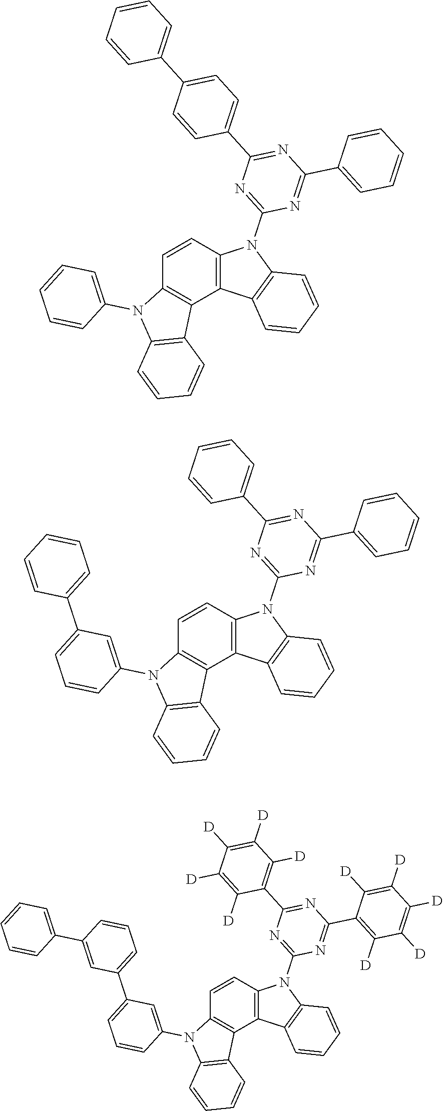 [SMILES: c1ccc(-c2ccc(-c3nc(-c4ccccc4)nc(-n4c5ccccc5c5c6c7ccccc7n(-c7ccccc7)c6ccc54)n3)cc2)cc1 ; c1ccc(-c2cccc(-n3c4ccccc4c4c5c6ccccc6n(-c6nc(-c7ccccc7)nc(-c7ccccc7)n6)c5ccc43)c2)cc1 ; [2H]c1c([2H])c([2H])c(-c2nc(-c3c([2H])c([2H])c([2H])c([2H])c3[2H])nc(-n3c4ccccc4c4c5c6ccccc6n(-c6cccc(-c7cccc(-c8ccccc8)c7)c6)c5ccc43)n2)c([2H])c1[2H]]

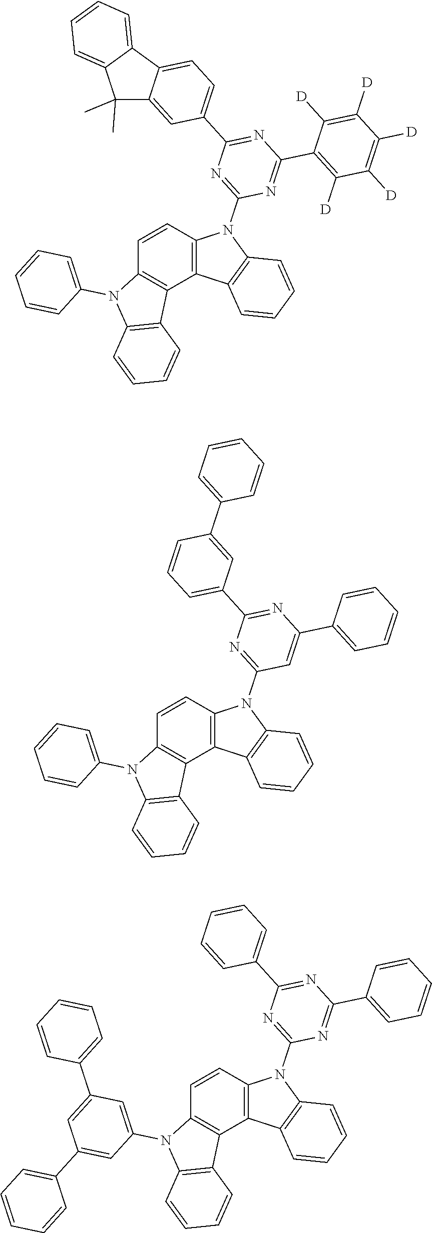 [SMILES: [2H]c1c([2H])c([2H])c(-c2nc(-c3ccc4c(c3)C(C)(C)c3ccccc3-4)nc(-n3c4ccccc4c4c5c6ccccc6n(-c6ccccc6)c5ccc43)n2)c([2H])c1[2H] ; c1ccc(-c2cccc(-c3nc(-c4ccccc4)cc(-n4c5ccccc5c5c6c7ccccc7n(-c7ccccc7)c6ccc54)n3)c2)cc1 ; c1ccc(-c2cc(-c3ccccc3)cc(-n3c4ccccc4c4c5c6ccccc6n(-c6nc(-c7ccccc7)nc(-c7ccccc7)n6)c5ccc43)c2)cc1]

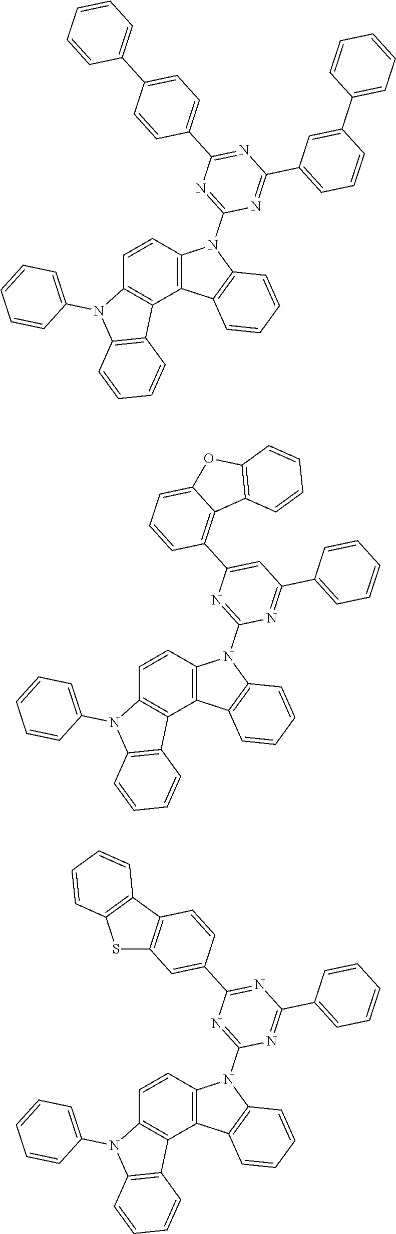 [SMILES: c1ccc(-c2ccc(-c3nc(-c4cccc(-c5ccccc5)c4)nc(-n4c5ccccc5c5c6c7ccccc7n(-c7ccccc7)c6ccc54)n3)cc2)cc1 ; c1ccc(-c2cc(-c3cccc4oc5ccccc5c34)nc(-n3c4ccccc4c4c5c6ccccc6n(-c6ccccc6)c5ccc43)n2)cc1 ; c1ccc(-c2nc(-c3ccc4c(c3)sc3ccccc34)nc(-n3c4ccccc4c4c5c6ccccc6n(-c6ccccc6)c5ccc43)n2)cc1]

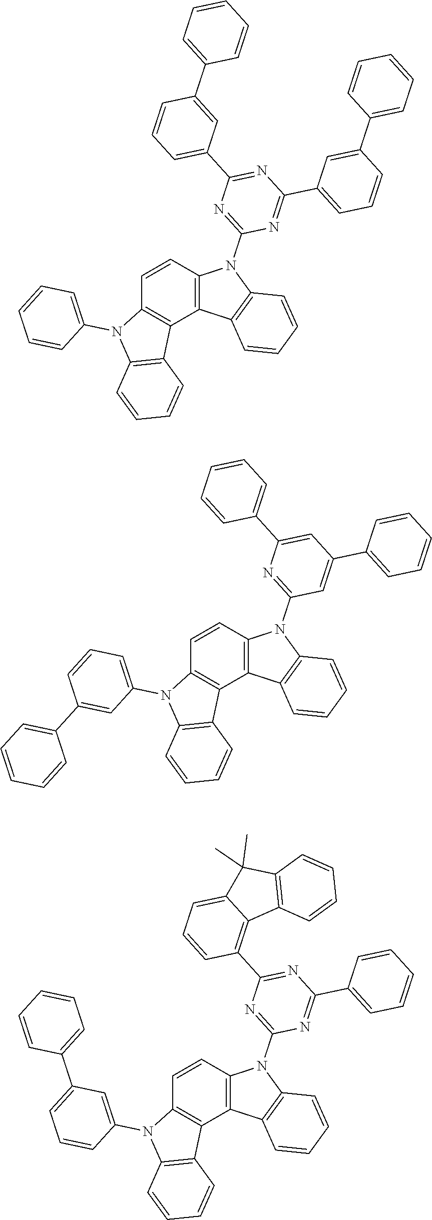 [SMILES: c1ccc(-c2cccc(-c3nc(-c4cccc(-c5ccccc5)c4)nc(-n4c5ccccc5c5c6c7ccccc7n(-c7ccccc7)c6ccc54)n3)c2)cc1 ; c1ccc(-c2cccc(-n3c4ccccc4c4c5c6ccccc6n(-c6cc(-c7ccccc7)cc(-c7ccccc7)n6)c5ccc43)c2)cc1 ; CC1(C)c2ccccc2-c2c(-c3nc(-c4ccccc4)nc(-n4c5ccccc5c5c6c7ccccc7n(-c7cccc(-c8ccccc8)c7)c6ccc54)n3)cccc21]

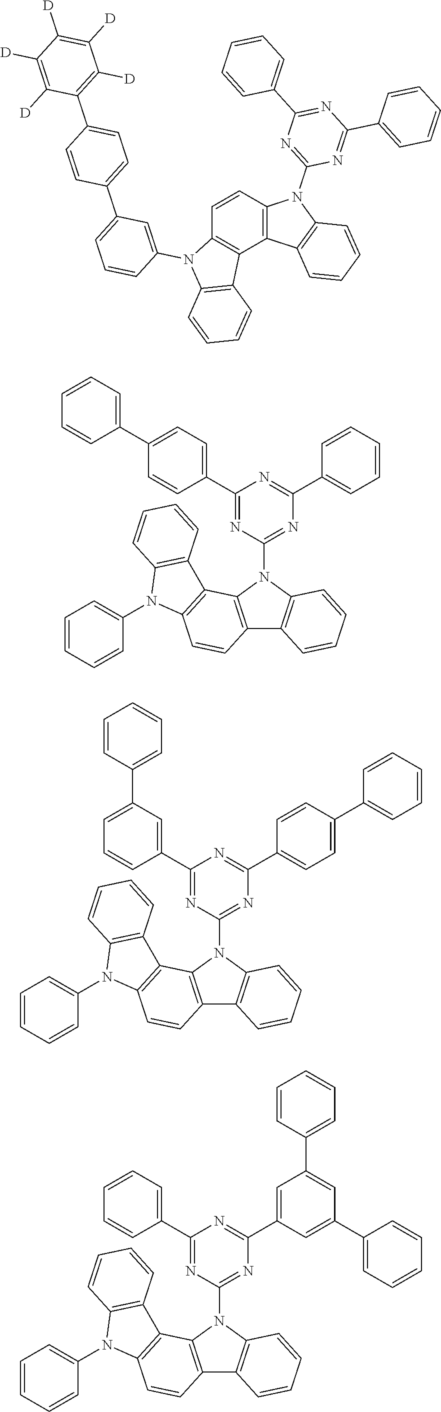 [SMILES: [2H]c1c([2H])c([2H])c(-c2ccc(-c3cccc(-n4c5ccccc5c5c6c7ccccc7n(-c7nc(-c8ccccc8)nc(-c8ccccc8)n7)c6ccc54)c3)cc2)c([2H])c1[2H] ; c1ccc(-c2ccc(-c3nc(-c4ccccc4)nc(-n4c5ccccc5c5ccc6c(c7ccccc7n6-c6ccccc6)c54)n3)cc2)cc1 ; c1ccc(-c2ccc(-c3nc(-c4cccc(-c5ccccc5)c4)nc(-n4c5ccccc5c5ccc6c(c7ccccc7n6-c6ccccc6)c54)n3)cc2)cc1 ; c1ccc(-c2cc(-c3ccccc3)cc(-c3nc(-c4ccccc4)nc(-n4c5ccccc5c5ccc6c(c7ccccc7n6-c6ccccc6)c54)n3)c2)cc1]

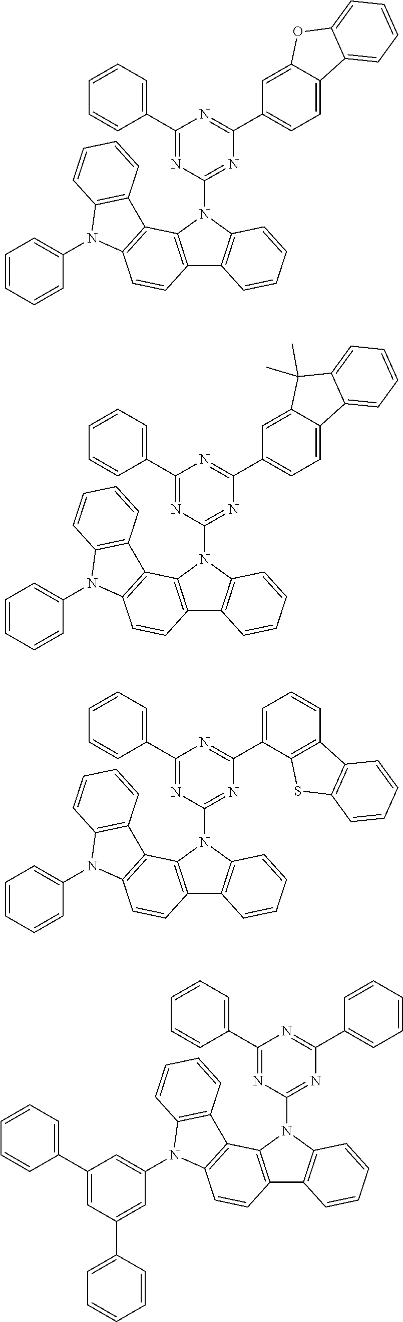 [SMILES: c1ccc(-c2nc(-c3ccc4c(c3)oc3ccccc34)nc(-n3c4ccccc4c4ccc5c(c6ccccc6n5-c5ccccc5)c43)n2)cc1 ; CC1(C)c2ccccc2-c2ccc(-c3nc(-c4ccccc4)nc(-n4c5ccccc5c5ccc6c(c7ccccc7n6-c6ccccc6)c54)n3)cc21 ; c1ccc(-c2nc(-c3cccc4c3sc3ccccc34)nc(-n3c4ccccc4c4ccc5c(c6ccccc6n5-c5ccccc5)c43)n2)cc1 ; c1ccc(-c2cc(-c3ccccc3)cc(-n3c4ccccc4c4c3ccc3c5ccccc5n(-c5nc(-c6ccccc6)nc(-c6ccccc6)n5)c34)c2)cc1]

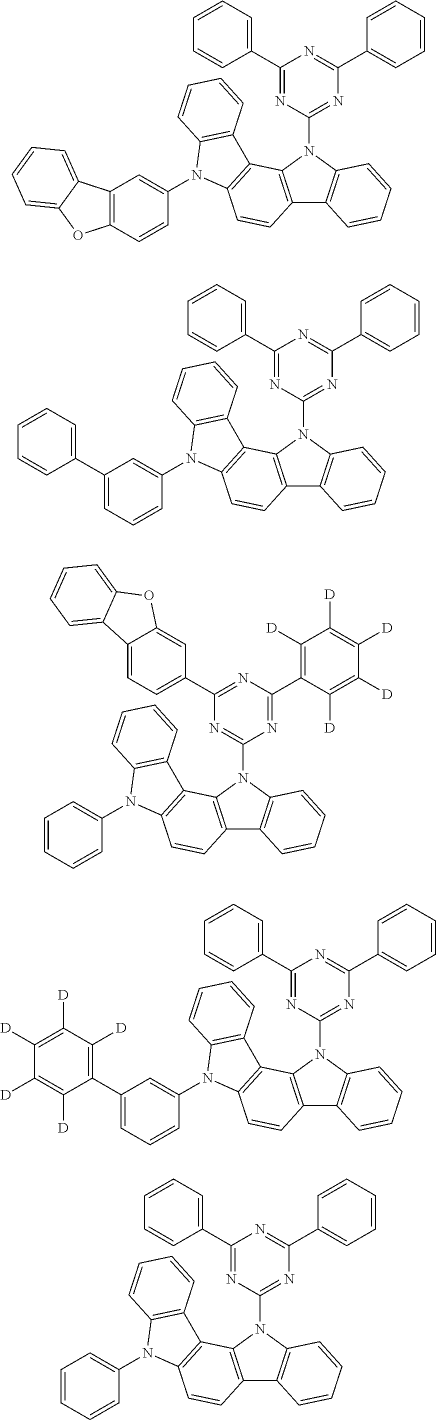

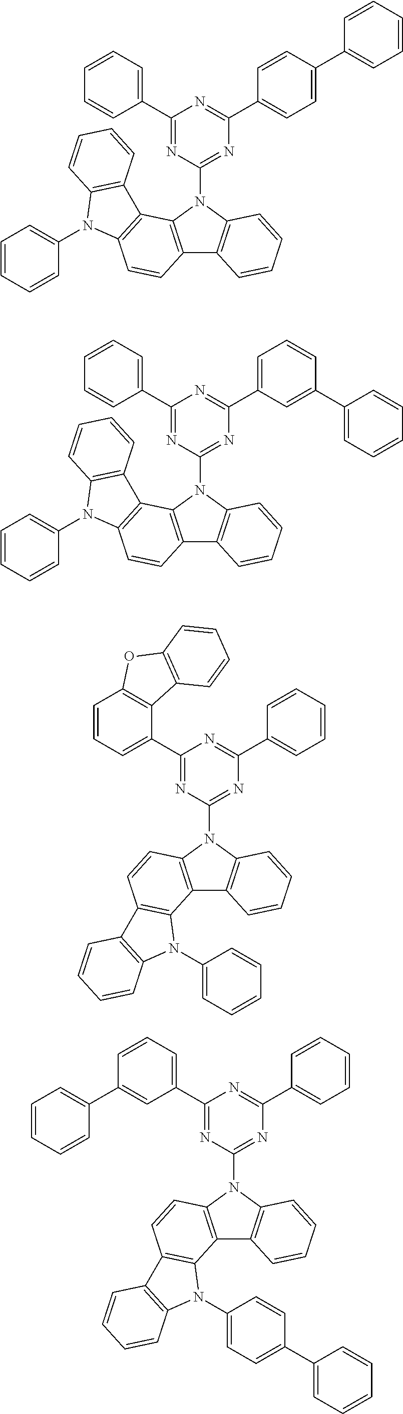

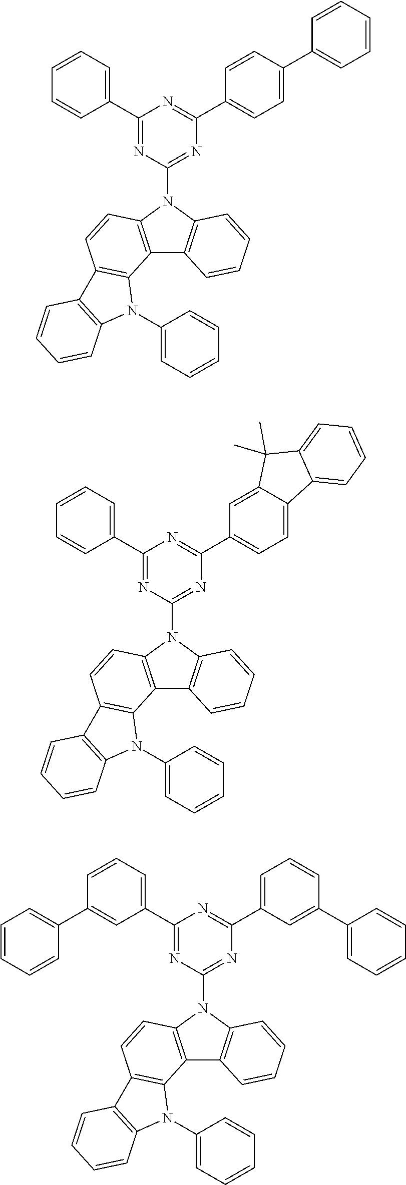 [SMILES: c1ccc(-c2ccc(-c3nc(-c4ccccc4)nc(-n4c5ccccc5c5c4ccc4c6ccccc6n(-c6ccccc6)c45)n3)cc2)cc1 ; CC1(C)c2ccccc2-c2ccc(-c3nc(-c4ccccc4)nc(-n4c5ccccc5c5c4ccc4c6ccccc6n(-c6ccccc6)c45)n3)cc21 ; c1ccc(-c2cccc(-c3nc(-c4cccc(-c5ccccc5)c4)nc(-n4c5ccccc5c5c4ccc4c6ccccc6n(-c6ccccc6)c45)n3)c2)cc1]

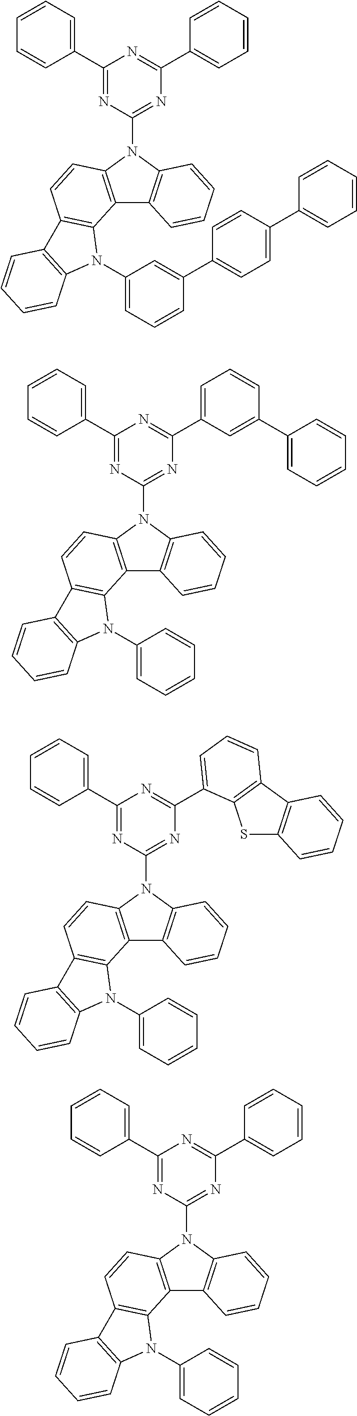 [SMILES: c1ccc(-c2ccc(-c3cccc(-n4c5ccccc5c5ccc6c(c7ccccc7n6-c6nc(-c7ccccc7)nc(-c7ccccc7)n6)c54)c3)cc2)cc1 ; c1ccc(-c2cccc(-c3nc(-c4ccccc4)nc(-n4c5ccccc5c5c4ccc4c6ccccc6n(-c6ccccc6)c45)n3)c2)cc1 ; c1ccc(-c2nc(-c3cccc4c3sc3ccccc34)nc(-n3c4ccccc4c4c3ccc3c5ccccc5n(-c5ccccc5)c34)n2)cc1 ; c1ccc(-c2nc(-c3ccccc3)nc(-n3c4ccccc4c4c3ccc3c5ccccc5n(-c5ccccc5)c34)n2)cc1]

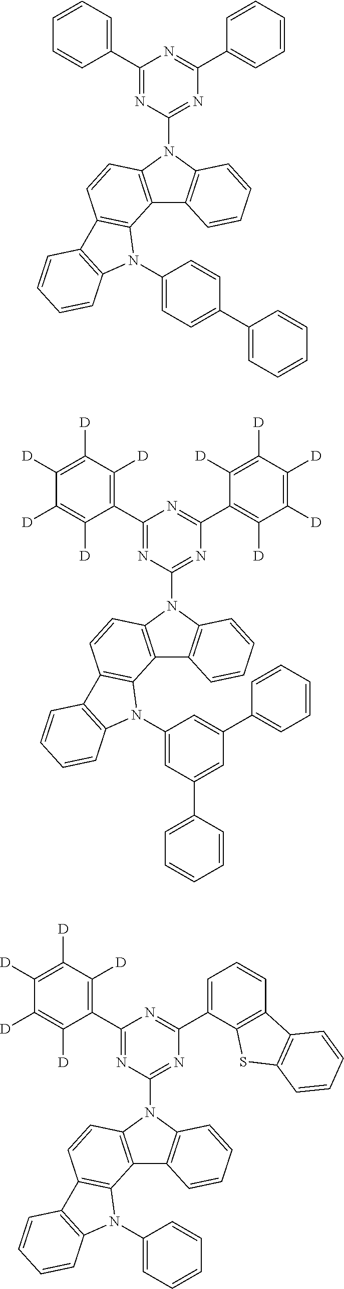 [SMILES: c1ccc(-c2ccc(-n3c4ccccc4c4ccc5c(c6ccccc6n5-c5nc(-c6ccccc6)nc(-c6ccccc6)n5)c43)cc2)cc1 ; [2H]c1c([2H])c([2H])c(-c2nc(-c3c([2H])c([2H])c([2H])c([2H])c3[2H])nc(-n3c4ccccc4c4c3ccc3c5ccccc5n(-c5cc(-c6ccccc6)cc(-c6ccccc6)c5)c34)n2)c([2H])c1[2H] ; [2H]c1c([2H])c([2H])c(-c2nc(-c3cccc4c3sc3ccccc34)nc(-n3c4ccccc4c4c3ccc3c5ccccc5n(-c5ccccc5)c34)n2)c([2H])c1[2H]]

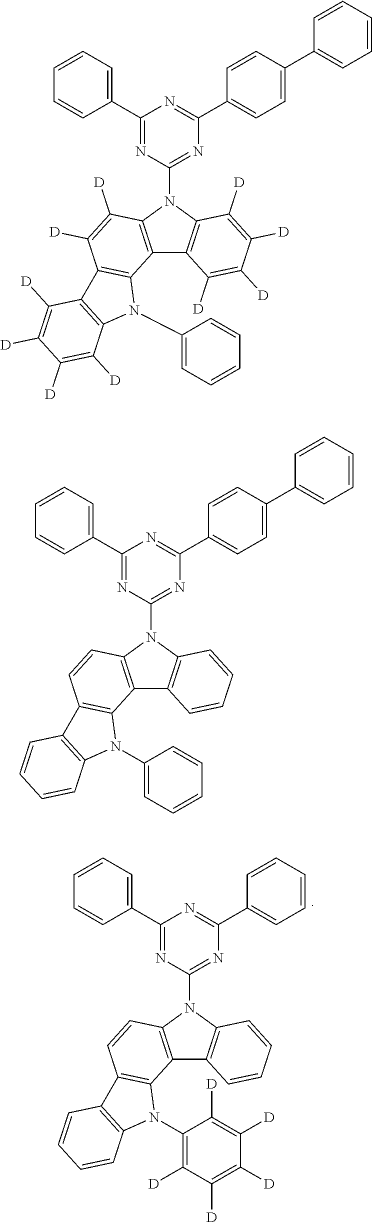 [SMILES: [2H]c1c([2H])c([2H])c2c(c1[2H])c1c(c([2H])c([2H])c3c4c([2H])c([2H])c([2H])c([2H])c4n(-c4ccccc4)c31)n2-c1nc(-c2ccccc2)nc(-c2ccc(-c3ccccc3)cc2)n1 ; c1ccc(-c2ccc(-c3nc(-c4ccccc4)nc(-n4c5ccccc5c5c4ccc4c6ccccc6n(-c6ccccc6)c45)n3)cc2)cc1 ; [2H]c1c([2H])c([2H])c(-n2c3ccccc3c3ccc4c(c5ccccc5n4-c4nc(-c5ccccc5)nc(-c5ccccc5)n4)c32)c([2H])c1[2H]]

The compound of Chemical Formula 3 can be prepared by a series of processes as shown in Reaction Schemes 3-1 and 3-2 below. (The definition of each substituent in Reaction Schemes 3-1 and 3-2 is the same as described above.)

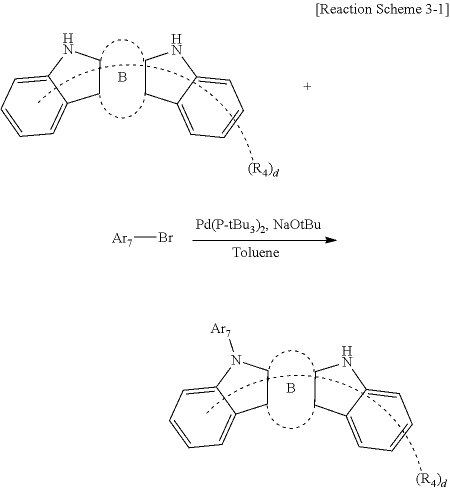

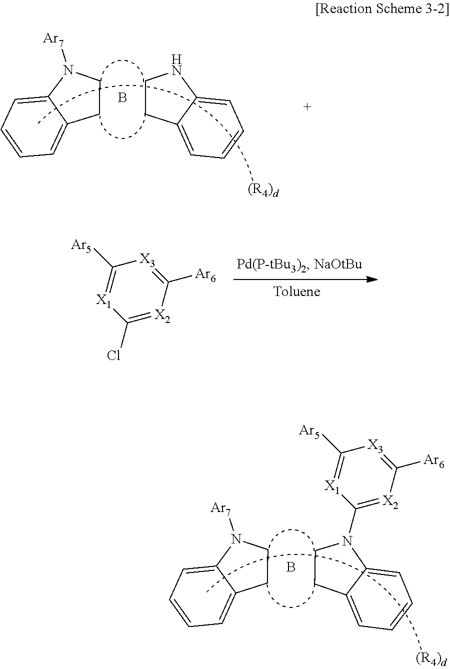

However, the series of processes of Reaction Schemes 3-1 and 3-2 are only examples, and the method for preparing the compound of Chemical Formula 3 can be more specifically described in Synthesis Examples to be described later.

Meanwhile, the dopant material is not particularly limited as long as it is a material used in an organic light emitting device. For example, the dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in a substituted or unsubstituted arylamine, in which one or two or more substituent groups selected from the group consisting of an aryl group, a silyl group, an alkyl group, a cycloalkyl group, and an arylamino group are a substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

Hole Transport Layer

The organic light emitting device according to the present disclosure can include a hole transport layer between the electron blocking layer and the anode.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer.

Specific examples of the hole transport material include an arylamine-based organic material, a conductive polymer, a block copolymer in which a conjugate portion and a non-conjugate portion are present together, and the like, but are not limited thereto.

Electron Blocking Layer

The organic light emitting device according to the present disclosure includes an electron blocking layer between the anode and the light emitting layer. Preferably, the electron blocking layer is included in contact with the anode side of the light emitting layer.

The electron blocking layer serves to improve the efficiency of an organic light emitting device by suppressing electrons injected from the cathode from being transferred to the anode without recombination in the light emitting layer.

The electron blocking layer includes an electron blocking material, and examples thereof include an arylamine-based organic material and the like, but are not limited thereto.

Hole Injection Layer

The organic light emitting device according to the present disclosure can further include a hole injection layer between the anode and the hole transport layer, if necessary.

The hole injection layer is a layer for injecting holes from the electrode, and the hole injection material is preferably a compound which can transport the holes, thus has a hole-injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

Electron Transport Layer

The organic light emitting device according to the present disclosure can include an electron transport layer between the light emitting layer and the cathode.

The electron transport layer is a layer which receives electrons from a cathode or an electron injection layer formed on the cathode and transports the electrons to a light emitting layer, and can suppress the transfer of holes in the light emitting layer. An electron transport material is suitably a material which can receive electrons well from a cathode and transport the electrons to a light emitting layer, and has large mobility for electrons.

Specific examples of the electron transport material include an Al complex of 8-hydroxyquinoline; a complex including Alq₃; an organic radical compound; a hydroxyflavone-metal complex, and the like, but are not limited thereto. The electron transport layer can be used with any desired cathode material, as used in the related art. In particular, appropriate examples of the cathode material are typical materials having a low work function, followed by an aluminum layer or a silver layer. Specific examples thereof include cesium, barium, calcium, ytterbium, and samarium, in each case followed by an aluminum layer or a silver layer.

Electron Injection Layer

The organic light emitting device according to the present disclosure can further include an electron injection layer between the electron transport layer and the cathode, if necessary.

The electron injection layer is a layer which injects electrons from an electrode, and the electron injection material is preferably a compound which can transport electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the material that can be used as the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Organic Light Emitting Device

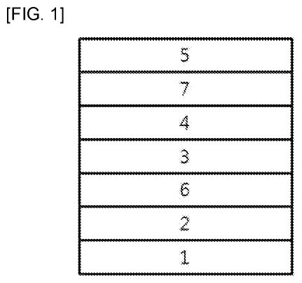

A structure of the organic light emitting device according to the present disclosure is illustrated in FIG. 1. FIG. 1 shows an example of an organic light emitting device including a substrate 1, an anode 2, a hole transport layer 6, an electron blocking layer 3, a light emitting layer 4, an electron transport layer 7, and a cathode 5.

The organic light emitting device according to the present disclosure can be manufactured by sequentially laminating the above-described components. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming the above-mentioned respective layers thereon, and then depositing a material that can be used as the cathode thereon. In addition to such a method, the organic light emitting device can be manufactured by sequentially depositing the above-described components from a cathode material to an anode material in the reverse order on a substrate (WO 2003/012890). Further, the light emitting layer can be formed using the host and the dopant by a solution coating method as well as a vacuum deposition method. Herein, the solution coating method means a spin coating, a dip coating, a doctor blading, an inkjet printing, a screen printing, a spray method, a roll coating, or the like, but is not limited thereto.

Meanwhile, the organic light emitting device according to the present disclosure can be a front side emission type, a backside emission type, or a double-sided emission type according to the used material.

Hereinafter, preferred examples are presented to help the understanding of the present invention. However, these examples are presented for illustrative purposes only, and are not intended to limit the scope of the present disclosure.

### SYNTHESIS EXAMPLES

### Synthesis Example 1-1: Synthesis of Compound 1-1

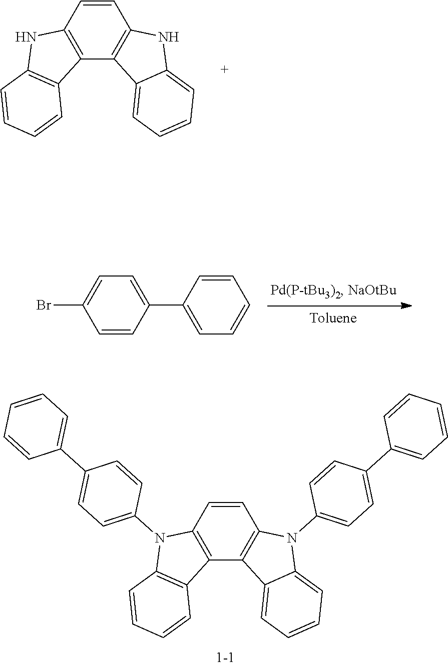 [SMILES: c1ccc2c(c1)[nH]c1ccc3[nH]c4ccccc4c3c12 ; Brc1ccc(-c2ccccc2)cc1 ; c1ccc(-c2ccc(-n3c4ccccc4c4c5c6ccccc6n(-c6ccc(-c7ccccc7)cc6)c5ccc43)cc2)cc1]

11,12-dihydroindolo[2,3-a]carbazole (15.0 g, 58.5 mmol) and 4-bromo-1,1′-biphenyl (30.0 g, 128.8 mmol) were added to toluene (300 ml) under a nitrogen atmosphere, and the mixture was stirred and refluxed. Then, sodium tert-butoxide (16.9 g, 175.6 mmol) and bis(tri-tert-butylphosphine)palladium(0) (0.9 g, 1.8 mmol) were added thereto. After 12 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography, and then 9.8 g of Compound 1-1 was prepared through sublimation purification (yield 30%, MS: [M+H]⁺=562).

### Synthesis Example 1-2: Synthesis of Compound 1-2

### Step 1) Synthesis of Intermediate 1-2-1

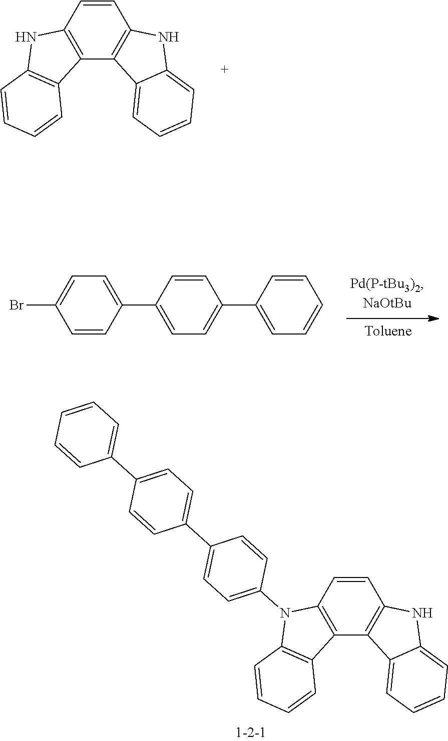 [SMILES: c1ccc2c(c1)[nH]c1ccc3[nH]c4ccccc4c3c12 ; Brc1ccc(-c2ccc(-c3ccccc3)cc2)cc1 ; c1ccc(-c2ccc(-c3ccc(-n4c5ccccc5c5c6c(ccc54)[nH]c4ccccc46)cc3)cc2)cc1]

11,12-dihydroindolo[2,3-a]carbazole (15.0 g, 58.5 mmol) and 4-bromo-1,1′:4′,1″-terphenyl (19.9 g, 64.4 mmol) were added to toluene (300 ml) under a nitrogen atmosphere, and the mixture was stirred and refluxed. Then, sodium tert-butoxide (8.4 g, 87.8 mmol), and bis(tri-tert-butylphosphine)palladium(0) (0.9 g, 1.8 mmol) were added thereto. After 11 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.3 g of Intermediate 1-2-1 (yield 68%, MS: [M+H]⁺=486).

### Step 2) Synthesis of Compound 1-2

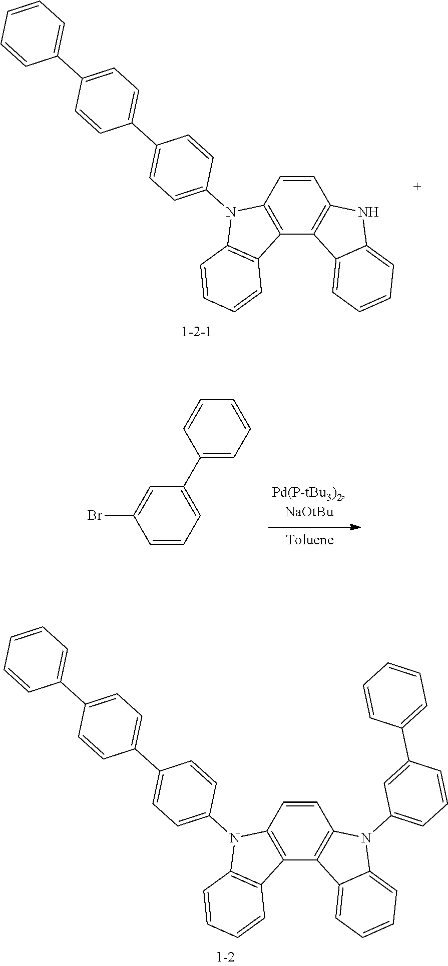 [SMILES: c1ccc(-c2ccc(-c3ccc(-n4c5ccccc5c5c6c(ccc54)[nH]c4ccccc46)cc3)cc2)cc1 ; Brc1cccc(-c2ccccc2)c1 ; c1ccc(-c2ccc(-c3ccc(-n4c5ccccc5c5c6c7ccccc7n(-c7cccc(-c8ccccc8)c7)c6ccc54)cc3)cc2)cc1]

Intermediate 1-2-1 (15.0 g, 31.0 mmol) and 3-bromo-1,1′-biphenyl (7.9 g, 34.0 mmol) were added to toluene (300 ml) under a nitrogen atmosphere, and the mixture was stirred and refluxed. Then, sodium tert-butoxide (4.5 g, 46.4 mmol), and bis(tri-tert-butylphosphine)palladium(0) (0.5 g, 0.9 mmol) were added thereto. After 7 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography, and then 9.5 g of Compound 1-2 was prepared through sublimation purification (yield 48%, MS: [M+H]⁺=638).

### Synthesis Example 1-3: Synthesis of Compound 1-3

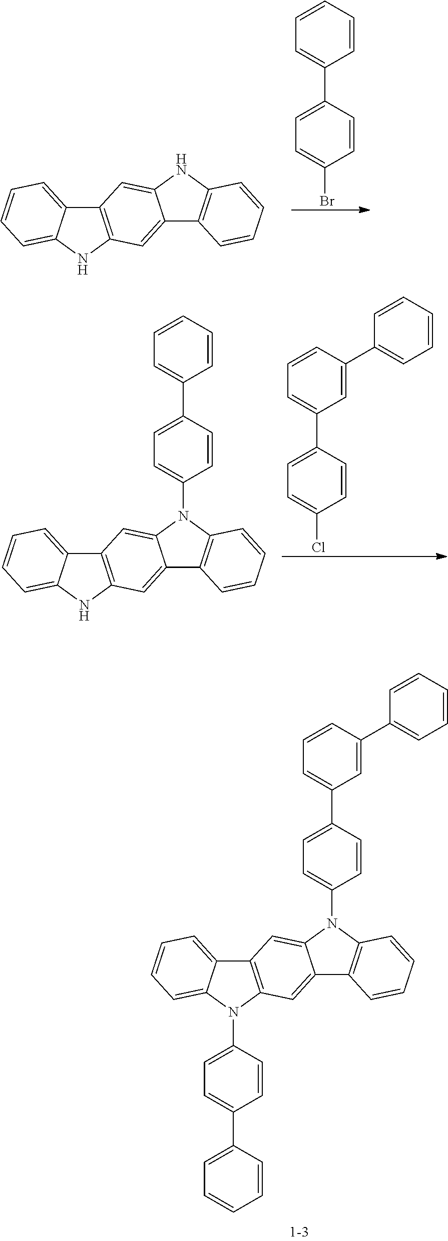 [SMILES: c1ccc2c(c1)[nH]c1cc3c(cc12)[nH]c1ccccc13 ; Brc1ccc(-c2ccccc2)cc1 ; c1ccc(-c2ccc(-n3c4ccccc4c4cc5[nH]c6ccccc6c5cc43)cc2)cc1 ; Clc1ccc(-c2cccc(-c3ccccc3)c2)cc1 ; c1ccc(-c2ccc(-n3c4ccccc4c4cc5c(cc43)c3ccccc3n5-c3ccc(-c4cccc(-c5ccccc5)c4)cc3)cc2)cc1]

Compound 1-3 was prepared in the same manner as in the preparation method of Compound 1-2, except that 5,8-dihydroindolo[2,3-c]carbazole was changed to 5,11-dihydroindolo[3,2-b]carbazole, 4-bromo-1,1′:4′,1″-terphenyl was changed to 4-bromo-1,1′-biphenyl, and 3-bromo-1,1′-biphenyl was changed to 4-chloro-1,1′:3′,1″-terphenyl in Synthesis Example 1-2 (MS: [M+H]⁺=638).

### Synthesis Example 1-4: Synthesis of Compound 1-4

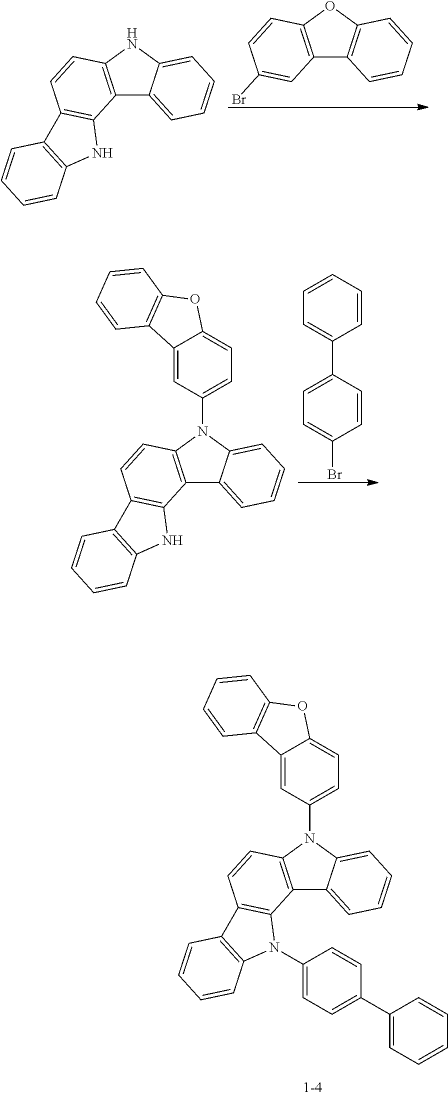

Compound 1-4 was prepared in the same manner as in the preparation method of Compound 1-2, except that 5,8-dihydroindolo[2,3-c]carbazole was changed to 5,12-dihydroindolo[3,2-a]carbazole, 4-bromo-1,1′:4′,1″-terphenyl was changed to 2-bromodibenzo[b,d]furan, and 3-bromo-1,1′-biphenyl was changed to 4-bromo-1,1′-biphenyl in Synthesis Example 1-2 (MS: [M+H]⁺=576).

### Synthesis Example 2-1: Synthesis of Compound 2-1

Step 1) Synthesis of Compound 2-1-1

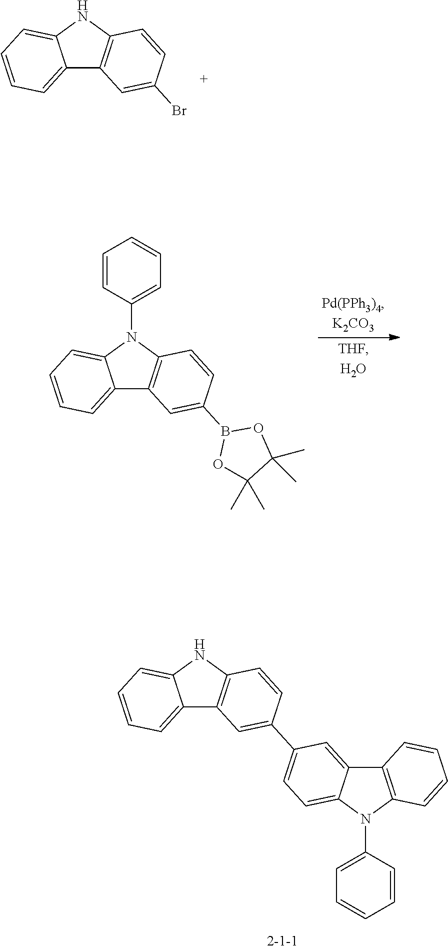 [SMILES: Brc1ccc2[nH]c3ccccc3c2c1 ; CC1(C)OB(c2ccc3c(c2)c2ccccc2n3-c2ccccc2)OC1(C)C ; c1ccc(-n2c3ccccc3c3cc(-c4ccc5[nH]c6ccccc6c5c4)ccc32)cc1]

3-bromo-9H-carbazole (15.0 g, 60.9 mmol) and 9-phenyl-3-(4,4,5,5-tetramethyl-1,3,2-dioxaborolan-2-yl)-9H-carbazole (24.8 g, 67.0 mmol) were added to 300 ml of THF under a nitrogen atmosphere, and the mixture was stirred and refluxed. Then, potassium carbonate (33.7 g, 243.8 mmol) was dissolved in 101 ml of water, and then added thereto. Thereafter, it was stirred sufficiently, followed by adding tetrakis(triphenylphosphine)-palladium(0) (2.1 g, 1.8 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.2 g of Compound 2-1-a (yield 61%, MS: [M+H]⁺=410).

### Step 2) Synthesis of Compound 2-1

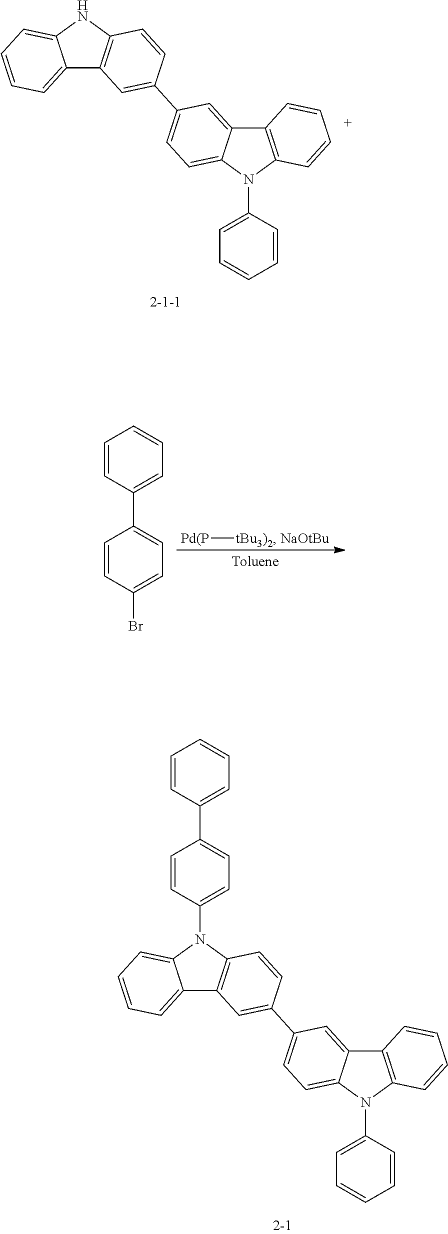

Compound 2-1-1 (15.0 g, 36.7 mmol) and 4-bromo-1,1′-biphenyl (9.4 g, 40.4 mmol) were added to toluene (300 ml) under a nitrogen atmosphere, and the mixture was stirred and refluxed. Then, sodium tert-butoxide (5.3 g, 55.1 mmol) and bis(tri-tert-butylphosphine)palladium(0) (0.6 g, 1.1 mmol) were added thereto. After 10 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography, and then 9.7 g of Compound 2-1 was prepared through sublimation purification (yield 47%, MS: [M+H]⁺=562).

### Synthesis Example 2-2: Synthesis of Compound 2-2

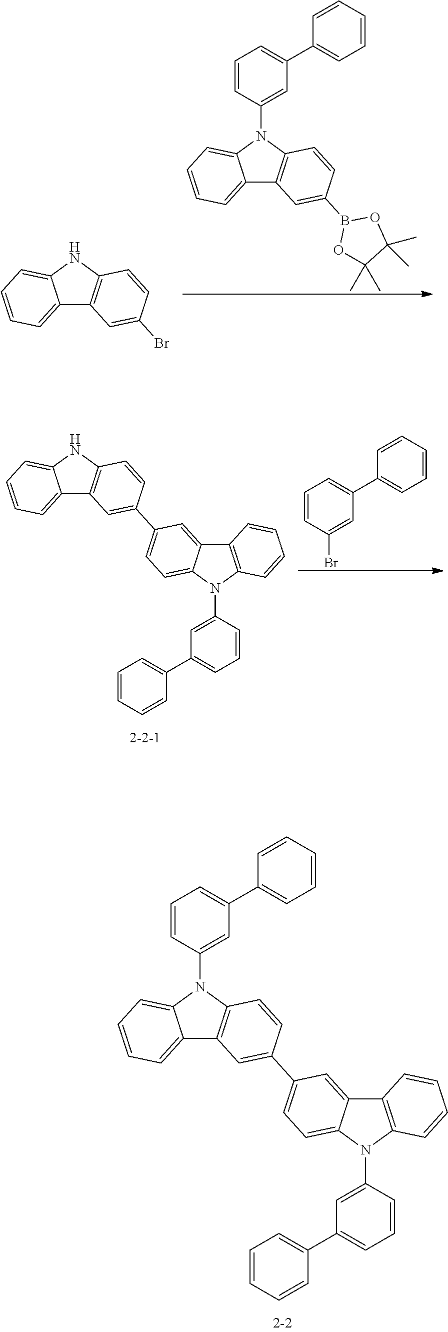

Compound 2-2 was prepared in the same manner as in the preparation method of Compound 2-1, except that 9-phenyl-3-(4,4,5,5-tetramethyl-1,3,2-dioxaborolan-2-yl)-9H-carbazole was changed to 9-([1,1′-biphenyl]-3-yl)-3-(4,4,5,5-tetramethyl-1,3,2-dioxaborolan-2-yl)-9H-carbazole, and 4-bromo-1,1′-biphenyl was changed to 3-bromo-1,1′-biphenyl in Synthesis Example 2-1 (MS: [M+H]⁺=638).

### Synthesis Example 2-3: Synthesis of Compound 2-3

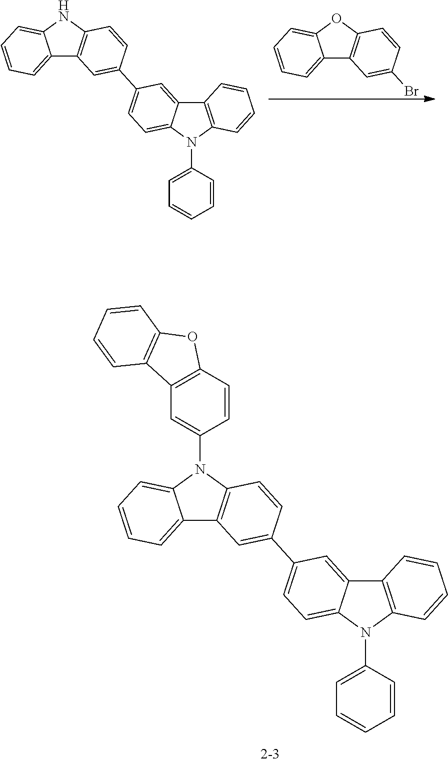

Compound 2-3 was prepared in the same manner as in the preparation method of Compound 2-1, except that 4-bromo-1,1′-biphenyl was changed to 2-bromodibenzo[b,d]furan in Step 2 of Synthesis Example 2-1 (MS: [M+H]⁺=576).

### Synthesis Example 2-4: Synthesis of Compound 2-4

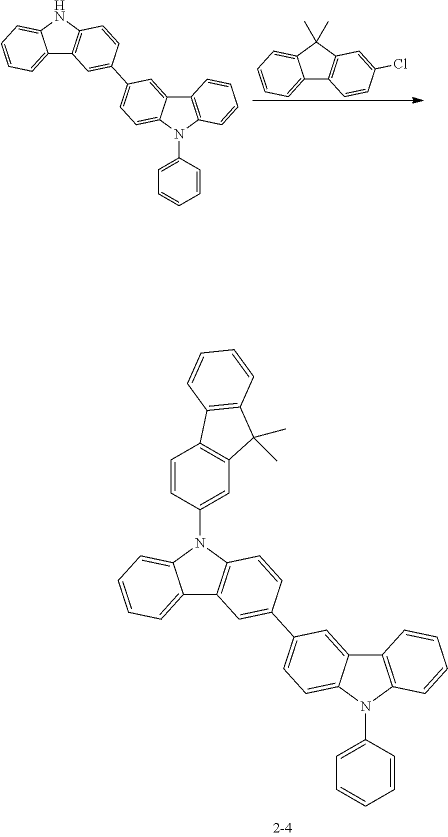

Compound 2-4 was prepared in the same manner as in the preparation method of Compound 2-1, except that 4-bromo-1,1′-biphenyl was changed to 2-chloro-9,9-dimethyl-9H-fluorene in Synthesis Example 2-1 (MS: [M+H]⁺=602).

### Synthesis Example 3-1: Synthesis of Compound 3-1

Step 1) Synthesis of Compound 3-1-1

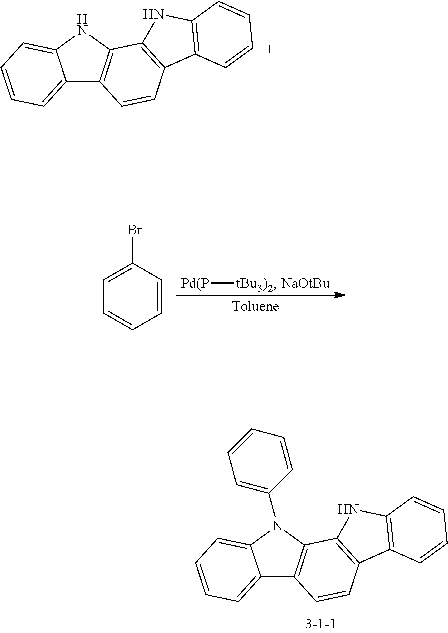

11,12-dihydroindolo[2,3-a]carbazole (15 g, 58.5 mmol) and bromobenzene (10.1 g, 64.4 mmol) were added to toluene (300 ml) under a nitrogen atmosphere, and the mixture was stirred and refluxed. Then, sodium tert-butoxide (8.4 g, 87.8 mmol), and bis(tri-tert-butylphosphine)palladium(0) (0.9 g, 1.8 mmol) were added thereto. After 9 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15 g of Compound 3-1-1 (yield 77%, MS: [M+H]⁺=333).

Step 2) Synthesis of Compound 3-1

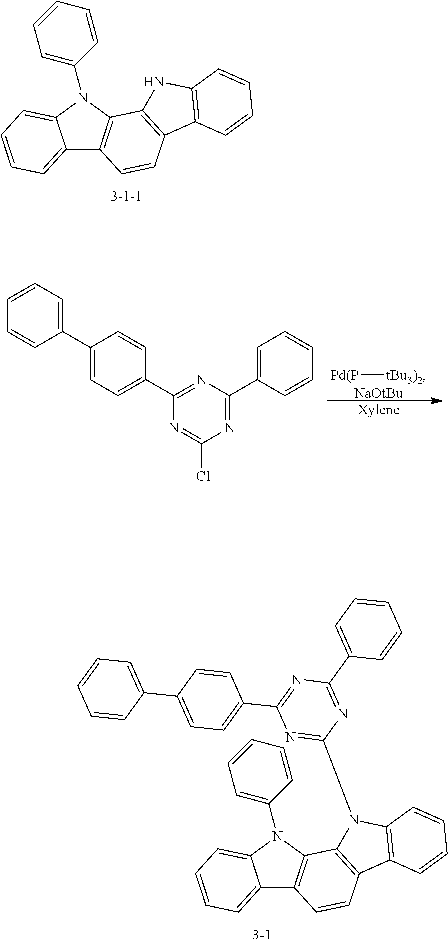

Compound 3-1-1 (15.0 g, 45.1 mmol) and 2-([1,1′-biphenyl]-4-yl)-4-chloro-6-phenyl-1,3,5-triazine (17.1 g, 49.6 mmol) were added to xylene (300 ml) under a nitrogen atmosphere, and the mixture was stirred and refluxed. Then, sodium tert-butoxide (6.5 g, 67.7 mmol) and bis(tri-tert-butylphosphine)palladium(0) (0.7 g, 1.4 mmol) were added thereto. After 6 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography, and then 10.4 g of Compound 3-1 was prepared through sublimation purification (yield 36%, MS: [M+H]⁺=641).

### Synthesis Example 3-2: Synthesis of Compound 3-2

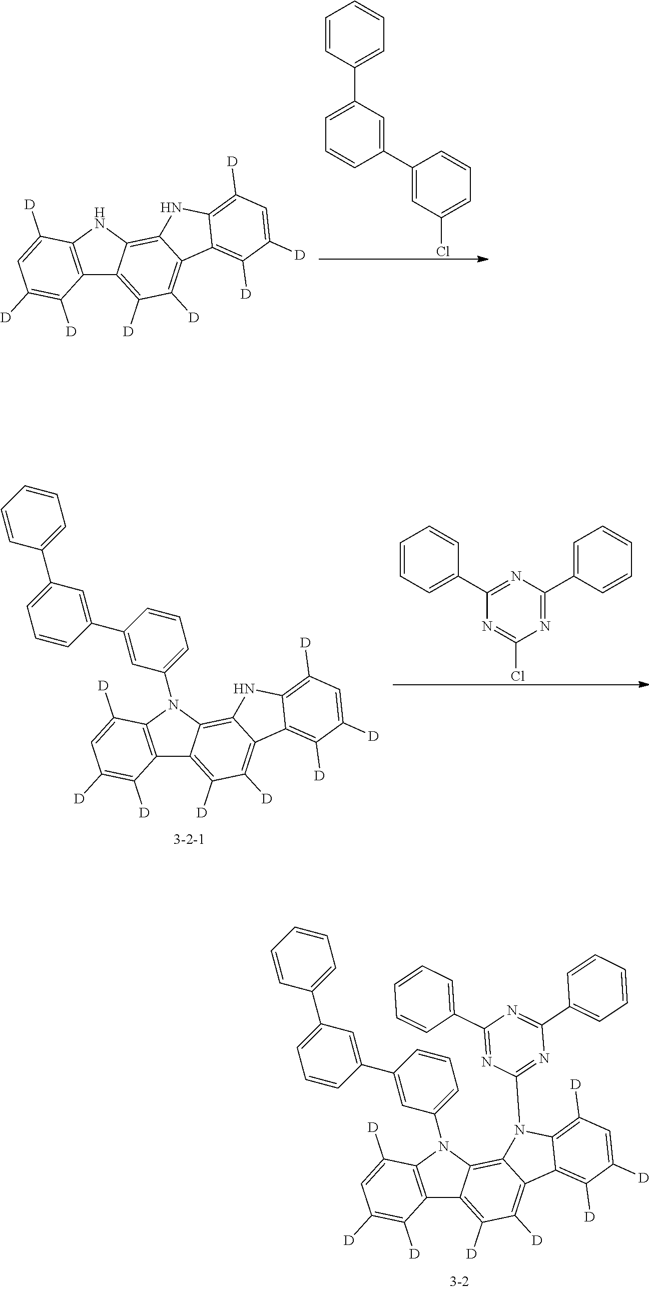 [SMILES: [2H]c1cc([2H])c2[nH]c3c4[nH]c5c([2H])cc([2H])c([2H])c5c4c([2H])c([2H])c3c2c1[2H] ; Clc1cccc(-c2cccc(-c3ccccc3)c2)c1 ; [2H]c1cc([2H])c2[nH]c3c(c([2H])c([2H])c4c5c([2H])c([2H])cc([2H])c5n(-c5cccc(-c6cccc(-c7ccccc7)c6)c5)c34)c2c1[2H] ; Clc1nc(-c2ccccc2)nc(-c2ccccc2)n1 ; [2H]c1cc([2H])c2c(c1[2H])c1c([2H])c([2H])c3c4c([2H])c([2H])cc([2H])c4n(-c4nc(-c5ccccc5)nc(-c5ccccc5)n4)c3c1n2-c1cccc(-c2cccc(-c3ccccc3)c2)c1]

Compound 3-2 was prepared in the same manner as in the preparation method of Compound 3-1, except that 11,12-dihydroindolo[2,3-a]carbazole was changed to 11,12-dihydroindolo[2,3-a]carbazole-1,3,4,5,6,7,8,10-d8, bromobenzene was changed to 3-chloro-1,1′:3′,1″-terphenyl, and 2-([1,1′-biphenyl]-4-yl)-4-chloro-6-phenyl-1,3,5-triazine was changed to 2-chloro-4,6-diphenyl-1,3,5-triazine in Synthesis Example 3-1 (MS: [M+H]⁺=725).

### Synthesis Example 3-3: Synthesis of Compound 3-3

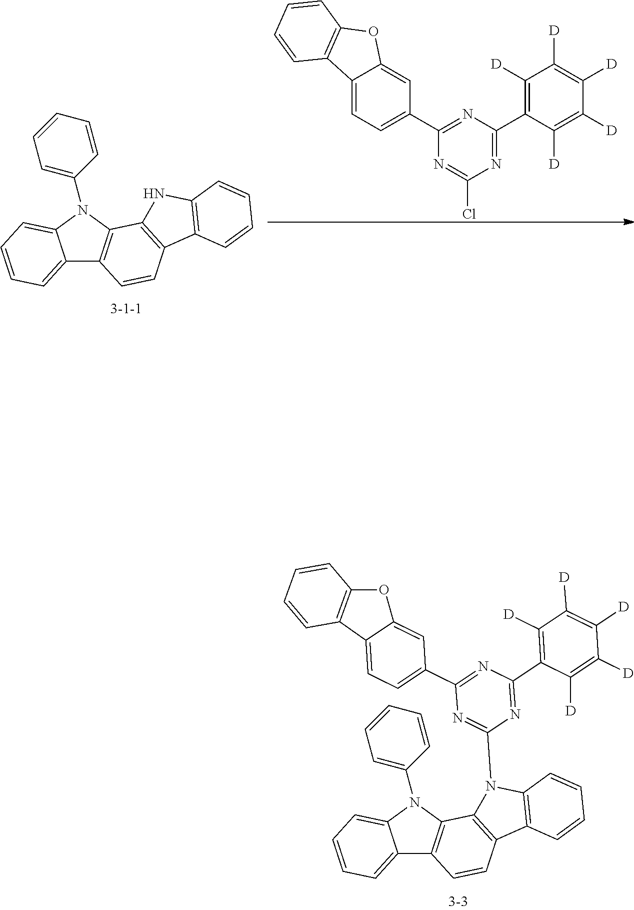 [SMILES: c1ccc(-n2c3ccccc3c3ccc4c5ccccc5[nH]c4c32)cc1 ; [2H]c1c([2H])c([2H])c(-c2nc(Cl)nc(-c3ccc4c(c3)oc3ccccc34)n2)c([2H])c1[2H] ; [2H]c1c([2H])c([2H])c(-c2nc(-c3ccc4c(c3)oc3ccccc34)nc(-n3c4ccccc4c4ccc5c6ccccc6n(-c6ccccc6)c5c43)n2)c([2H])c1[2H]]

Compound 3-3 was prepared in the same manner as in the preparation method of Compound 3-1, except that 2-([1,1′-biphenyl]-4-yl)-4-chloro-6-phenyl-1,3,5-triazine was changed to 2-chloro-4-(dibenzo[b,d]furan-3-yl)-6-(phenyl-d5)-1,3,5-triazine in Step 2 of Synthesis Example 3-1 (MS: [M+H]⁺=660).

### Synthesis Example 3-4: Synthesis of Compound 3-4

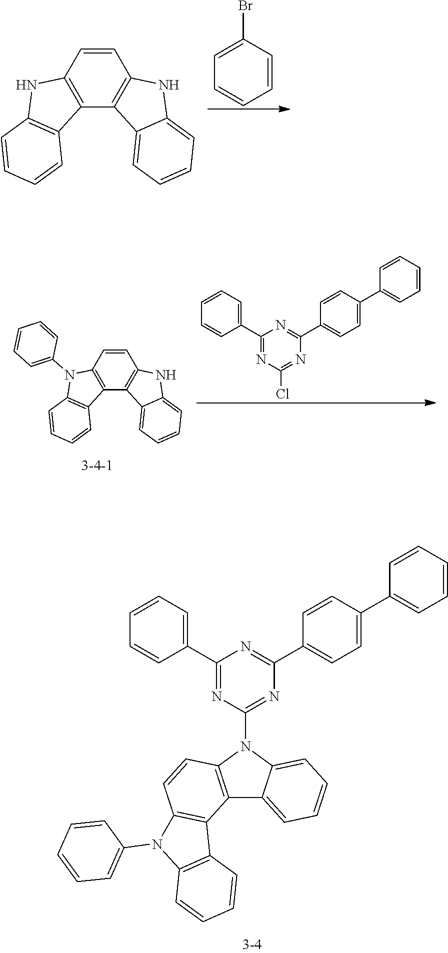 [SMILES: c1ccc2c(c1)[nH]c1ccc3[nH]c4ccccc4c3c12 ; Brc1ccccc1 ; c1ccc(-n2c3ccccc3c3c4c(ccc32)[nH]c2ccccc24)cc1 ; Clc1nc(-c2ccccc2)nc(-c2ccc(-c3ccccc3)cc2)n1 ; c1ccc(-c2ccc(-c3nc(-c4ccccc4)nc(-n4c5ccccc5c5c6c7ccccc7n(-c7ccccc7)c6ccc54)n3)cc2)cc1]

Compound 3-4 was prepared in the same manner as in the preparation method of Compound 3-1, except that 11,12-dihydroindolo[2,3-a]carbazole was changed to 5,8-dihydroindolo[2,3-c]carbazole in Synthesis Example 3-1 (MS: [M+H]⁺=641).

### Synthesis Example 3-5: Synthesis of Compound 3-5

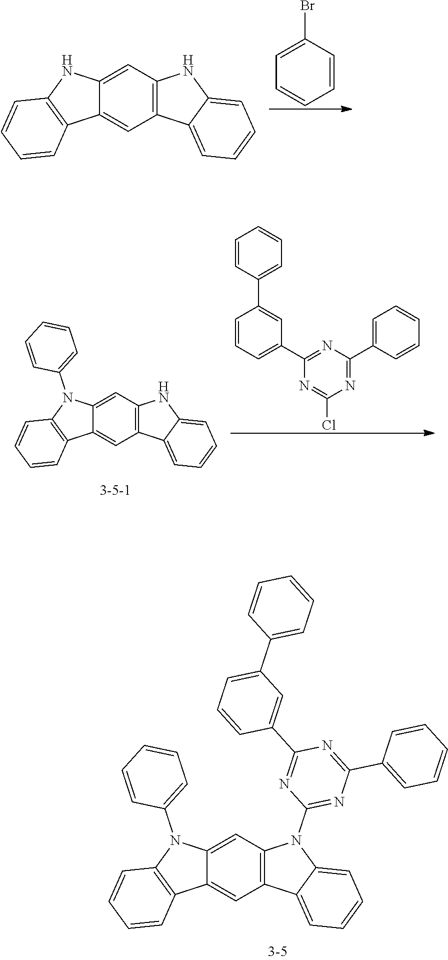

Compound 3-5 was prepared in the same manner as in the preparation method of Compound 3-1, except that 11,12-dihydroindolo[2,3-a]carbazole was changed to 5,7-dihydroindolo[2,3-b]carbazole, and 2-([1,1′-biphenyl]-4-yl)-4-chloro-6-phenyl-1,3,5-triazine was changed to 2-([1,1′-biphenyl]-3-yl)-4-chloro-6-phenyl-1,3,5-triazine in Synthesis Example 3-1 (MS: [M+H]⁺=641).

### Synthesis Example 3-6: Synthesis of Compound 3-6

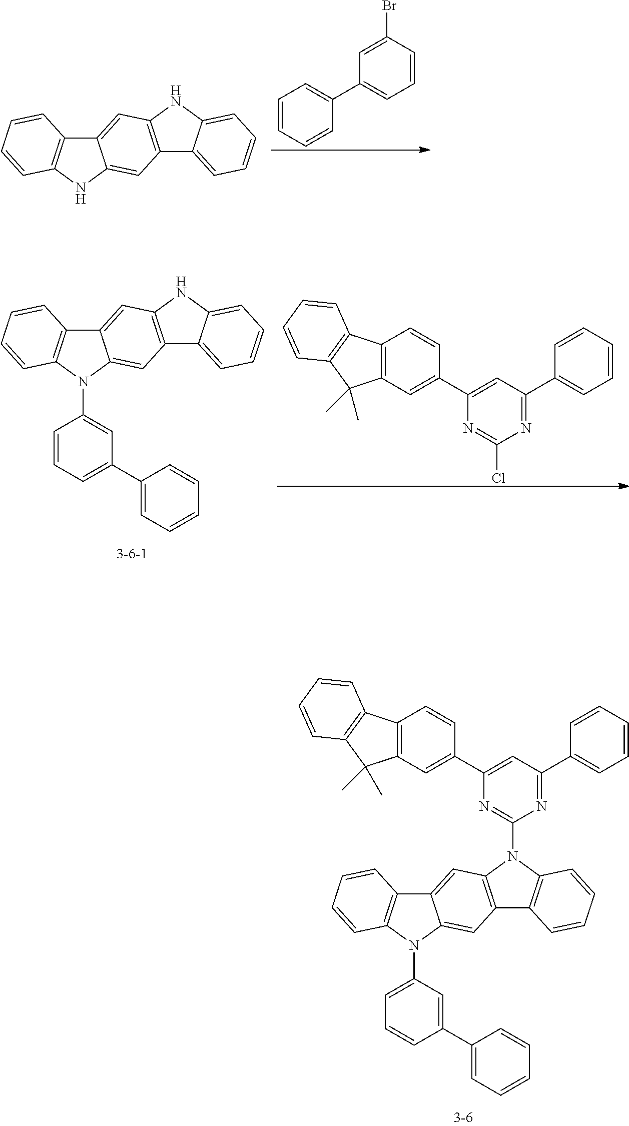

Compound 3-6 was prepared in the same manner as in the preparation method of Compound 3-1, except that 11,12-dihydroindolo[2,3-a]carbazole was changed to 5,11-dihydroindolo[2,3-b]carbazole, bromobenzene was changed to 3-bromo-1,1′-biphenyl, and 2-([1,1′-biphenyl]-4-yl)-4-chloro-6-phenyl-1,3,5-triazine was changed to 2-chloro-4-(9,9-dimethyl-9H-fluoren-2-yl)-6-phenylpyrimidine in Synthesis Example 3-1 (MS: [M+H]⁺=756).

### Synthesis Example 3-7: Synthesis of Compound 3-7

Step 1) Synthesis of Compound 3-7-1

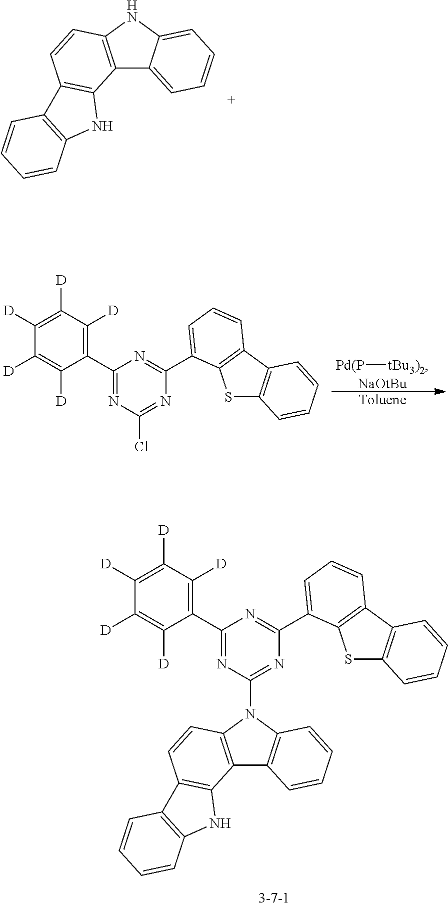

5,12-dihydroindolo[3,2-a]carbazole (15.0 g, 58.5 mmol) and 2-chloro-4-(dibenzo[b,d]thiophen-4-yl)-6-phenyl-1,3,5-triazine (24.4 g, 64.4 mmol) were added to toluene (300 ml) under a nitrogen atmosphere, and the mixture was stirred and refluxed. Then, sodium tert-butoxide (8.4 g, 87.8 mmol), and bis(tri-tert-butylphosphine)palladium(0) (0.9 g, 1.8 mmol) were added thereto. After 6 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.4 g of Compound 3-7-1 (yield 61%, MS: [M+H]⁺=600).

Step 2) Synthesis of Compound 3-7

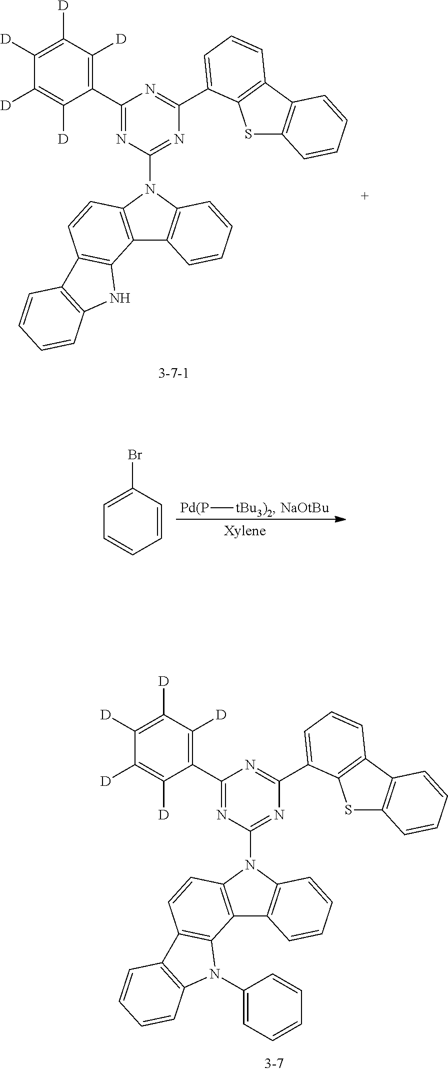

Compound 3-7-1 (15.0 g, 25.1 mmol) and bromobenzene (4.3 g, 27.6 mmol) were added to xylene (300 ml) under a nitrogen atmosphere, and the mixture was stirred and refluxed. Then, sodium tert-butoxide (3.6 g, 37.6 mmol) and bis(tri-tert-butylphosphine)palladium(0) (0.4 g, 0.8 mmol) were added thereto. After 8 hours of reaction, it was cooled to room temperature and the organic layer was separated using chloroform and water, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography, and then 7.3 g of Compound 3-7 was prepared through sublimation purification (yield 43%, MS: [M+H]⁺=676).

### Examples

### Example 1: Preparation of Organic Light Emitting Device

A glass substrate on which ITO (Indium Tin Oxide) was coated as a thin film to a thickness of 1400 A was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. The substrate was cleaned for 5 minutes using oxygen plasma and then transferred to a vacuum depositor.

95 wt % of the following compound HT-A and 5 wt % of the following compound PH2D were thermally vacuum-deposited on the prepared ITO transparent electrode to a thickness of 100 A to form a hole injection layer. Then, only the HT-A material was deposited to a thickness of 1150 A to form a hole transport layer. The following compound HT-BH3 was thermally vacuum-deposited thereon to a thickness of 450 A as an electron blocking layer.

Thereafter, 92 wt % of the host in which Compound 1-1 as a first host, Compound 2-1 as a second host, and Compound 3-1 as a third host were mixed at a weight ratio of 35:35:30 and 8 wt % of the following compound GD were vacuum-deposited on the electron blocking layer to a thickness of 350 A to form a light emitting layer.

Then, the following compound ET-A was vacuum-deposited to a thickness of 50 A as a hole blocking layer. Subsequently, the following compound ET-B and Liq were thermally vacuum-deposited at a ratio 1:1 to a thickness of 300 A as an electron transport layer, and then Yb was vacuum-deposited to a thickness of 10 A as an electron injection layer.

Magnesium and silver were deposited on the electron injection layer at a ratio of 1:4 to a thickness of 150 A to form a cathode, thereby manufacturing an organic light emitting device.

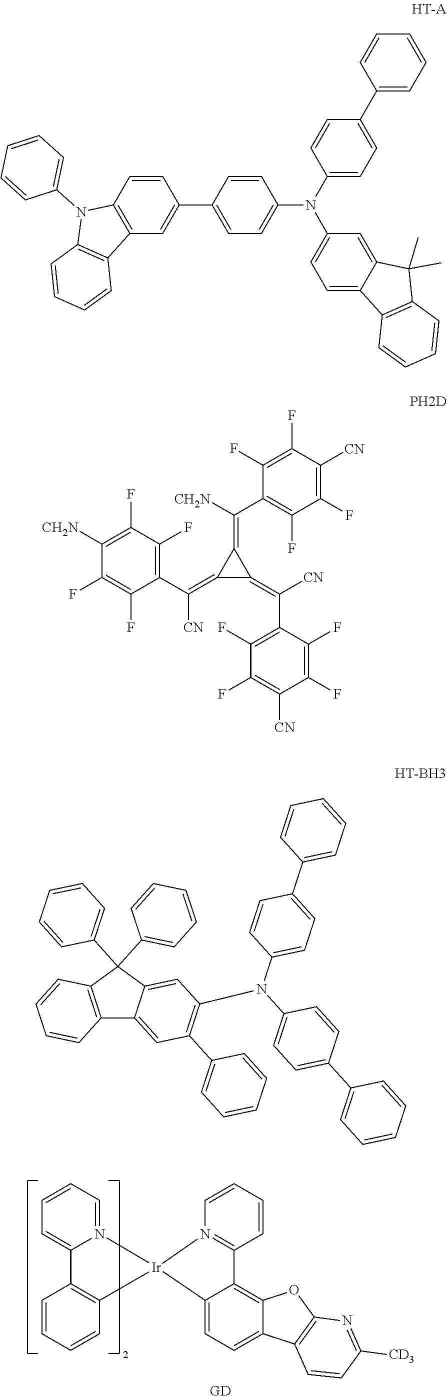

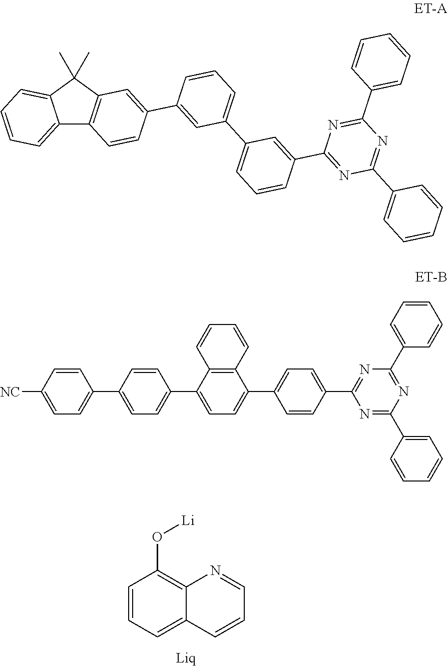

In the above process, the deposition rate of the organic material was maintained at 0.4 to 0.7 A/sec, the deposition rate of magnesium and silver was maintained at 2 A/sec, and the degree of vacuum during the deposition was maintained at 2×10⁻⁷ to 5×10⁻⁶ torr, thereby manufacturing an organic light emitting device.

### Example 2 to Example 21 and Comparative Example 1 to Comparative Example 16

Organic light emitting devices of Examples 2 to 21 and Comparative Examples 1 to 13 were respectively manufactured in the same manner as in the Example 1, except that the host materials were changed as shown in Table 1 and Table 2 below. Herein, the ratio means a weight ratio of the first host, the second host, and the third host. In addition, Compounds GH-A, GH-B, GH-C and GH-D described in Table 2 are as follows.

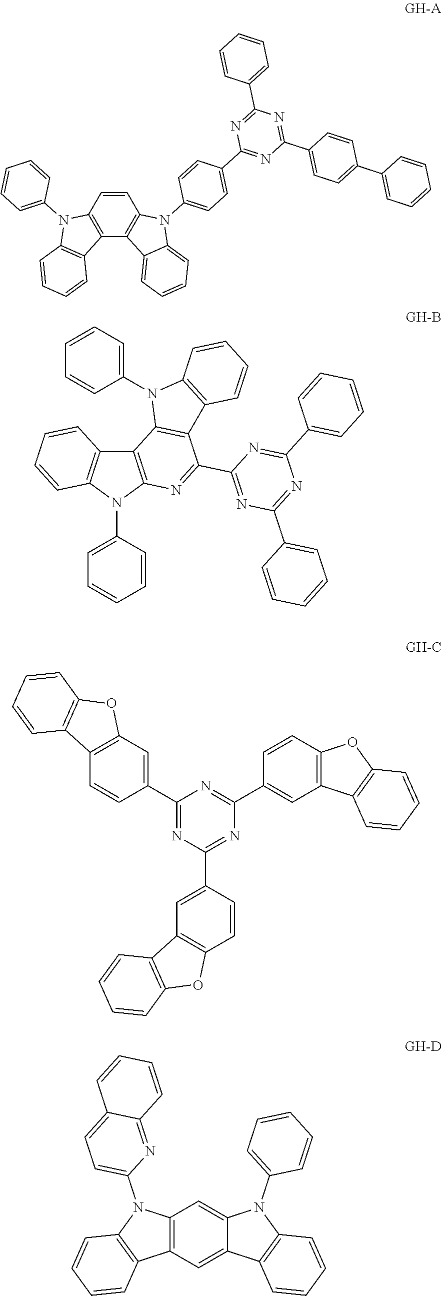

Evaluation of Device Characteristics

The organic light emitting devices prepared in the above Examples 1 to 21 and Comparative Examples 1 to 16 were heat-treated in an oven at 120° C. for 30 minutes, and then taken out. Then, the voltage, efficiency, and lifetime (T95) were measured by applying a current, and the results are shown in Table 1 and Table 2 below. Herein, the voltage and efficiency were measured by applying a current density of 10 mA/cm², and T95 means the time taken (hr) until the initial luminance decreases to 95% at a current density of 20 mA/cm².

TABLE 1

| | | | | | | | @20 |
|---|---|---|---|---|---|---|---|
| | | | | | @10 mA/cm² | | mA/cm² |
| | | | | | Voltage | Efficiency | Lifetime |
| | 1st host | 2nd host | 3rd host | Ratio | (V) | (cd/A) | (T95, hr) |
| Ex. 1 | Com. 1-1 | Com. 2-1 | Com. 3-1 | 35:35:30 | 3.85 | 78.4 | 212 |
| Ex. 2 | | Com. 2-1 | Com. 3-2 | 35:35:30 | 3.81 | 78.1 | 238 |
| Ex. 3 | | Com. 2-1 | Com. 3-3 | 35:35:30 | 3.82 | 78.2 | 241 |
| Ex. 4 | | Com. 2-2 | Com. 3-4 | 35:35:30 | 3.93 | 77.5 | 213 |
| Ex. 5 | | Com. 2-2 | Com. 3-5 | 35:35:30 | 3.94 | 76.1 | 206 |
| Ex. 6 | | Com. 2-2 | Com. 3-6 | 35:35:30 | 3.96 | 75.2 | 213 |
| Ex. 7 | | Com. 2-2 | Com. 3-7 | 35:35:30 | 3.91 | 77.3 | 227 |
| Ex. 8 | Com. 1-2 | Com. 2-1 | Com. 3-1 | 35:35:30 | 3.84 | 78.1 | 219 |
| Ex. 9 | | Com. 2-1 | Com. 3-2 | 35:35:30 | 3.83 | 78.6 | 231 |
| Ex. 10 | | Com. 2-2 | Com. 3-3 | 35:35:30 | 3.86 | 77.9 | 242 |
| Ex. 11 | | Com. 2-2 | Com. 3-4 | 35:35:30 | 3.97 | 77.0 | 210 |
| Ex. 12 | | Com. 2-3 | Com. 3-5 | 35:35:30 | 3.91 | 75.2 | 198 |
| Ex. 13 | | Com. 2-3 | Com. 3-6 | 35:35:30 | 3.96 | 74.3 | 196 |
| Ex. 14 | | Com. 2-4 | Com. 3-7 | 35:35:30 | 4.02 | 76.7 | 215 |
| Ex. 15 | Com. 1-3 | Com. 2-1 | Com. 3-1 | 35:35:30 | 3.85 | 76.4 | 206 |
| Ex. 16 | | Com. 2-1 | Com. 3-3 | 35:35:30 | 3.90 | 76.1 | 213 |
| Ex. 17 | | Com. 2-2 | Com. 3-5 | 35:35:30 | 3.92 | 76.0 | 203 |
| Ex. 18 | | Com. 2-2 | Com. 3-7 | 35:35:30 | 3.94 | 75.3 | 219 |
| Ex. 19 | Com. 1-4 | Com. 2-3 | Com. 3-2 | 35:35:30 | 4.01 | 74.1 | 209 |
| Ex. 20 | | Com. 2-3 | Com. 3-4 | 35:35:30 | 4.06 | 73.9 | 196 |
| Ex. 21 | | Com. 2-4 | Com. 3-6 | 35:35:30 | 4.03 | 73.3 | 197 |

TABLE 2

| | | | | | | | @20 |
|---|---|---|---|---|---|---|---|
| | | | | | @10 mA/cm² | | mA/cm² |
| | | | | | Voltage | Efficiency | Lifetime |
| | 1st host | 2nd host | 3rd host | Ratio | (V) | (cd/A) | (T95, hr) |
| Comparative Ex. 1 | Com. 1-1 | — | — | 100:0:0 | 6.93 | 13.9 | 16 |
| Comparative Ex. 2 | — | Com. 2-1 | — | 0:100:0 | 7.83 | 8.5 | 23 |
| Comparative Ex. 3 | — | — | Com. 3-1 | 0:0:100 | 5.38 | 53.6 | 59 |
| Comparative Ex. 4 | Com. 1-1 | Com. 2-1 | — | 50:50:0 | 7.26 | 15.3 | 41 |
| Comparative Ex. 5 | Com. 1-1 | — | Com. 3-1 | 70:0:30 | 4.23 | 67.2 | 126 |
| Comparative Ex. 6 | — | Com. 2-1 | Com. 3-1 | 0:70:30 | 4.46 | 71.5 | 183 |
| Comparative Ex. 7 | Com. 1-2 | Com. 2-2 | — | 50:50:0 | 7.31 | 16.2 | 32 |
| Comparative Ex. 8 | Com. 1-3 | — | Com. 3-5 | 70:0:30 | 4.21 | 64.9 | 143 |
| Comparative Ex. 9 | — | Com. 2-3 | Com. 3-4 | 0:70:30 | 4.49 | 70.8 | 176 |
| Comparative Ex. 10 | Com. 1-1 | — | GH-A | 70:0:30 | 4.56 | 66.5 | 103 |
| Comparative Ex. 11 | Com. 1-1 | Com. 2-2 | GH-A | 35:35:30 | 4.43 | 65.3 | 143 |
| Comparative Ex. 12 | Com. 1-2 | — | GH-B | 70:0:30 | 5.56 | 54.5 | 59 |
| Comparative Ex. 13 | Com. 1-3 | — | GH-C | 70:0:30 | 5.33 | 60.1 | 89 |
| Comparative Ex. 14 | Com. 1-1 | Com. 2-1 | GH-B | 35:35:30 | 5.98 | 48.7 | 42 |
| Comparative Ex. 15 | Com. 1-1 | Com. 2-1 | GH-C | 35:35:30 | 5.73 | 49.2 | 31 |
| Comparative Ex. 16 | GH-D | Com. 2-1 | Com. 3-1 | 35:35:30 | 6.46 | 24.6 | 26 |

In Tables 1 and 2, it was confirmed that the devices of Examples 1 to 21 had significantly lower driving voltage and significantly improved efficiency and lifetime compared to the devices of Comparative Examples 1 to 16.

The indolocarbazole-based compound (Chemical Formula 1) and the biscarbazole-based compound (Chemical Formula 2) have excellent hole transport ability, thereby serving as a P-type host; and the compound in which pyridine, pyrimidine, or triazine is bonded to N of indolocarbazole (Chemical Formula 3) serves as an N-type host.

In general, since an exciplex is formed when a P-type host and an N-type host are mixed and applied as the host of the light emitting layer, the characteristics of the device can be further improved compared to the case in which only one of the P-type host and the N-type host is applied.

In the present disclosure, in Examples 1 to 21 in which a P-type host and an N-type host are mixed and applied as the host of the light emitting layer, the driving voltage of the device was significantly lowered, and the efficiency and lifetime were significantly improved compared to Comparative Examples 1 to 4 and 7 in which only one of the P-type host and the N-type host is applied.

In particular, when using a mixture of the two P-type hosts which are Chemical Formulae 1 and 2 and the N-type host of the Chemical Formula 3 (Chemical Formula 1+Chemical Formula 2+Chemical Formula 3), the characteristics of the device can be further improved compared to the case in which only one P-type host of Chemical Formulae 1 and 2 is mixed with the N-type host of the Chemical Formula 3 (Chemical Formula 1+Chemical Formula 3; or Chemical Formula 2+Chemical Formula 3).

The P-type host of the Chemical Formula 1 exhibits low voltage due to its structure containing indolocarbazole, and the P-type host of the Chemical Formula 2 exhibits high efficiency and long lifetime due to its structure containing biscarbazole. Therefore, using a mixture thereof is advantageous for uniformly improving the voltage, efficiency, and lifetime of the device.

In the present disclosure, in Examples 1 to 21 in which the two P-type hosts of the Chemical Formulae 1 and 2 and the N-type host of the Chemical Formula 3 are mixed, the voltage, efficiency, and lifetime of the device were overall improved compared to Comparative Examples 5, 6, 8, 9, 10, 12, and 13 in which only one P-type host in Chemical Formulae 1 and 2 is mixed with the N-type host.

Meanwhile, when several types of hosts are mixed and used, intermediate characteristics of the mixed hosts appear.

In Examples 1 to 21 (particularly, Examples 4 to 7) in which the two P-type hosts of the Chemical Formulae 1 and 2 and the N-type host of the Chemical Formula 3 are mixed, it can be seen that the overall improvement in voltage, efficiency, and lifetime of the device compared to Comparative Example 11 in which a compound having a structure completely different from that of the Chemical Formula 3 is replaced with the N-type host was influenced by the P-type host of the Chemical Formula 3.

The N-type host of the Chemical Formula 3 contributes to overall improvement in voltage, efficiency, and lifetime of the device due to its structure in which pyridine, pyrimidine, or triazine is bonded to N of indolocarbazole, and shows a greater synergistic effect when mixed with two P-type hosts of the Chemical Formulae 1 and 2.

DESCRIPTION OF SYMBOLS

| | |
|---|---|
| 1: Substrate | 2: Anode |
| 3: Electron blocking layer | 4: Light emitting layer |
| 5: Cathode | 6: Hole transport layer |
| 7: Electron transport layer | |

1. An organic light emitting device, comprising
an anode,
a cathode,
a light emitting layer between the anode and the cathode;
an electron blocking layer between the anode and the light emitting layer, and
a hole transport layer between the electron blocking layer and the anode,
wherein the light emitting layer comprises a compound of the following Chemical Formula 1, a compound of the following Chemical Formula 2, and a compound of the following Chemical Formula 3: 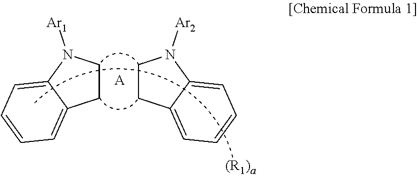
wherein in Chemical Formula 1;
A is a benzene ring condensed with two adjacent pentagonal rings;
Ar₁ is a substituted or unsubstituted C₆₋₆₀ aryl;
Ar₂ is a substituted or unsubstituted C₆₋₆₀ aryl, or a substituted or unsubstituted C₂₋₆₀ heteroaryl containing at least one heteroatom selected from the group consisting of O and S;
R₁ is hydrogen, deuterium, a substituted or unsubstituted C₆₋₆₀ aryl, or a substituted or unsubstituted C₂₋₆₀ heteroaryl containing at least one heteroatom selected from the group consisting of N, O and S; and
a is an integer of 1 to 10; 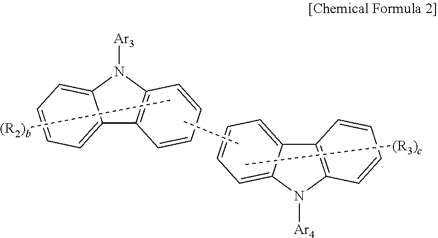
wherein in Chemical Formula 2-2:
Ar₃ and Ar₄ are each independently a substituted or unsubstituted C₆₋₆₀ aryl, or a substituted or unsubstituted C₂₋₆₀ heteroaryl containing at least one heteroatom selected from the group consisting of N, O and S;
R₂ and R₃ are each independently hydrogen, deuterium, a substituted or unsubstituted C₆₋₆₀ aryl, or a substituted or unsubstituted C₂₋₆₀ heteroaryl containing at least one heteroatom selected from the group consisting of N, O and S; and
b and c are each independently an integer of 1 to 7; 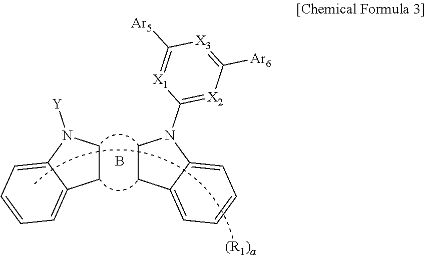
wherein in Chemical Formula 3;
B is a benzene ring condensed with two adjacent pentagonal rings;
X₁ to X₃ are each independently CH, or N, provided that at least one of X₁ to X₃ is N;
Ar₅ and Ar₆ are each independently a substituted or unsubstituted C₆₋₆₀ aryl, or a substituted or unsubstituted C₂₋₆₀ heteroaryl containing at least one heteroatom selected from the group consisting of N, O and S;
Y is NAr₇, wherein Ar₇ is a substituted or unsubstituted C₆₋₆₀ aryl, or a substituted or unsubstituted C₂₋₆₀ heteroaryl containing at least one heteroatom selected from the group consisting of N, O and S;
R₄ is hydrogen, deuterium, a substituted or unsubstituted C₆₋₆₀ aryl, or a substituted or unsubstituted C₂₋₆₀ heteroaryl containing at least one heteroatom selected from the group consisting of N, O and S; and
d is an integer of 1 to 10.

2. The organic light emitting device of claim 1,
wherein Chemical Formula 1 is any one of the following Chemical Formulae 1-1 to 1-4: 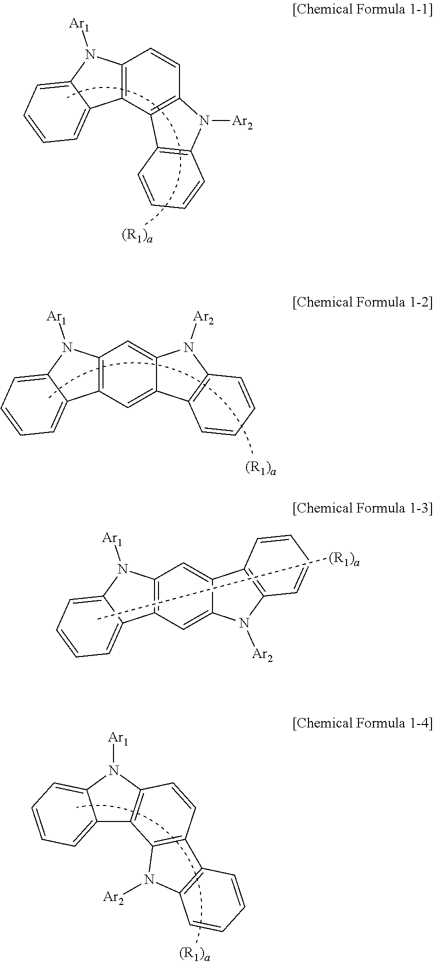
wherein in Chemical Formulae 1-1 to 1-4, Ar₁, Ar₂, R₁, and a have the same definitions as in claim 1.

3. The organic light emitting device of claim 1, wherein Ari is phenyl, biphenylyl, or terphenylyl.

4. The organic light emitting device of claim 1, wherein Ar₂ is biphenylyl, terphenylyl, (phenyl)biphenylyl, dimethylfluorenyl, (dimethylfluorenyl)-phenyl, dibenzofuranyl, (dibenzofuranyl)phenyl, dibenzothiophenyl, or (dibenzothiophenyl)phenyl.

5. The organic light emitting device of claim 1, wherein R₁ is hydrogen, or deuterium.

6. The organic light emitting device of claim 1,
wherein the compound of Chemical Formula 1 is any one compound selected from the group consisting of the following compounds:

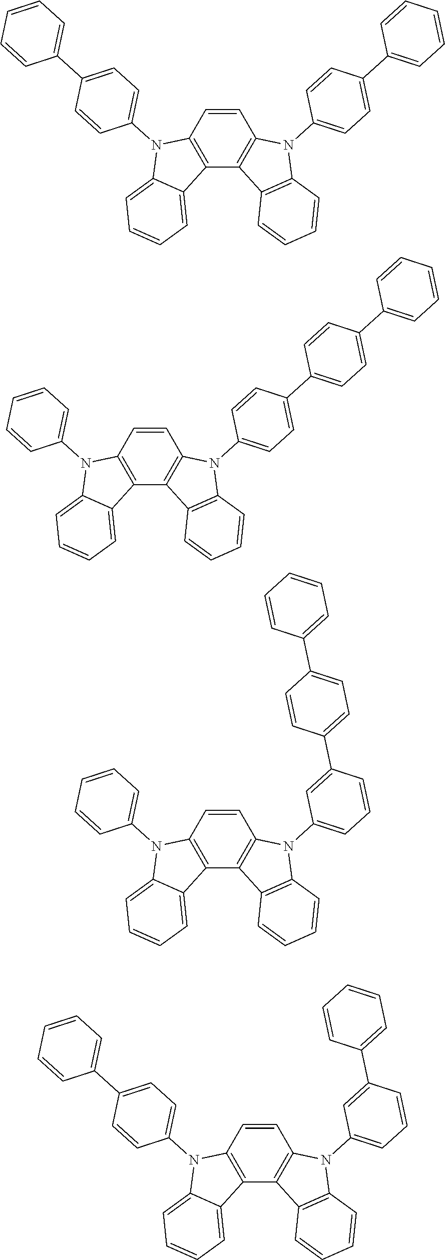 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2ccc(-c3cccc(-n4c5ccccc5c5c6c7ccccc7n(-c7ccccc7)c6ccc54)c3)cc2)cc1 ; c1ccc(-c2ccc(-n3c4ccccc4c4c5c6ccccc6n(-c6cccc(-c7ccccc7)c6)c5ccc43)cc2)cc1]

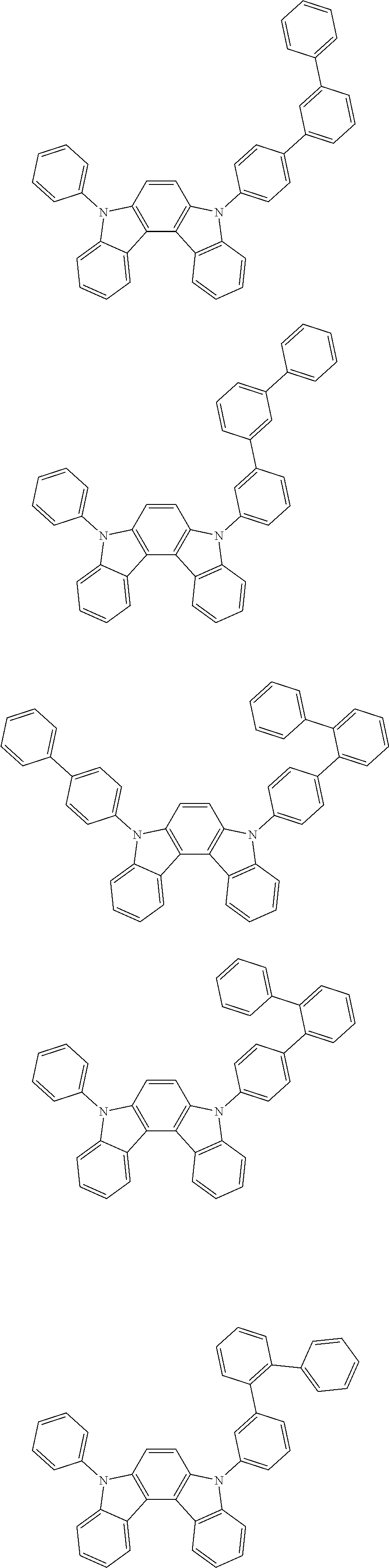

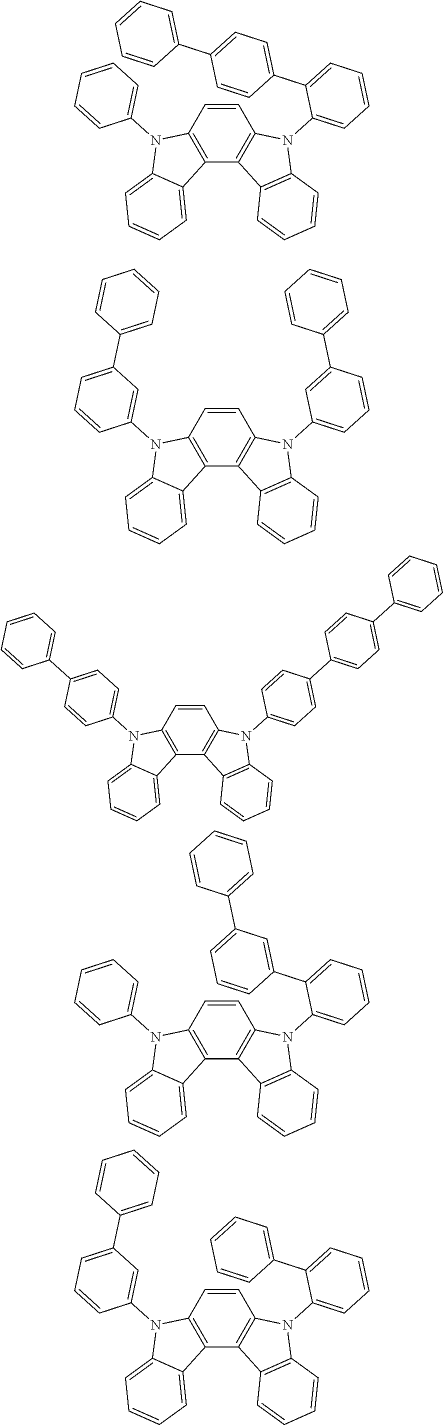

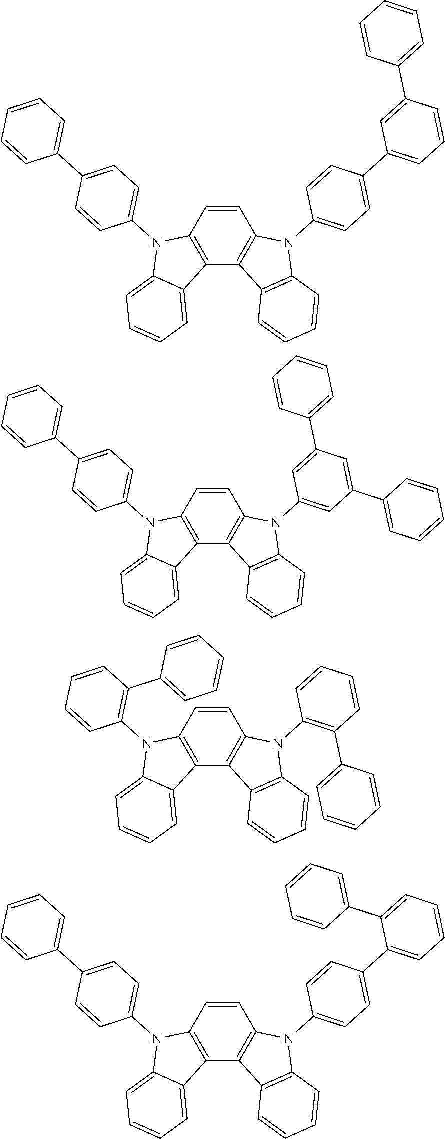

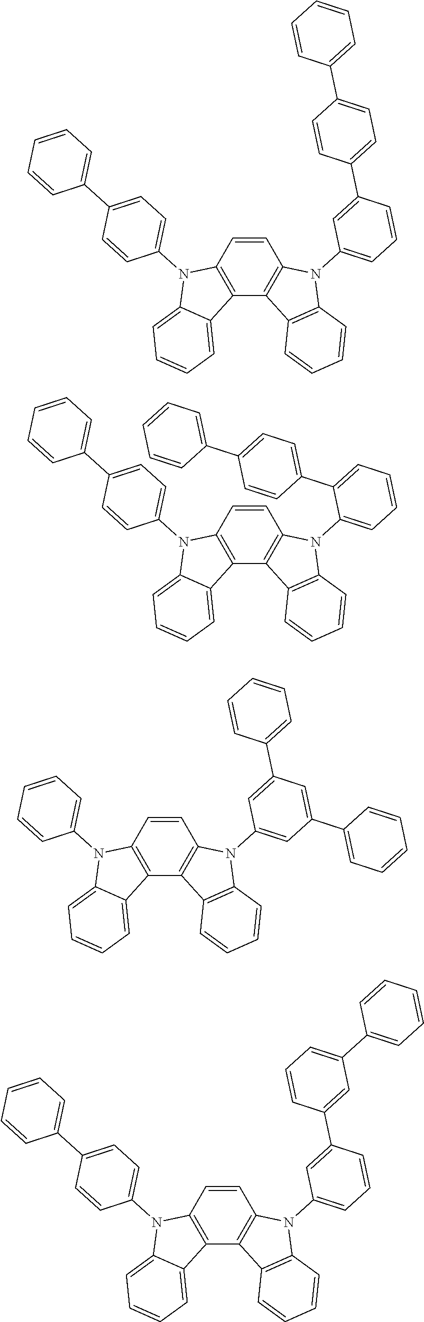

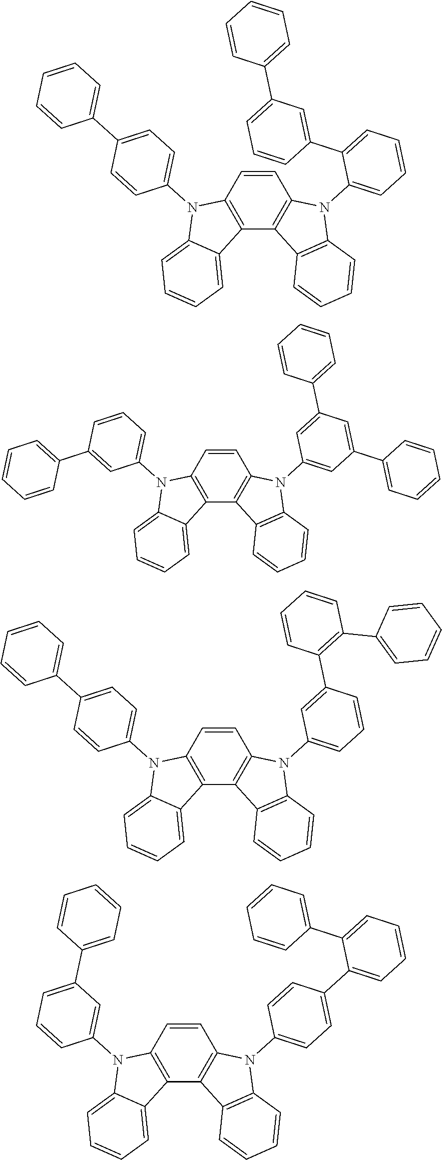

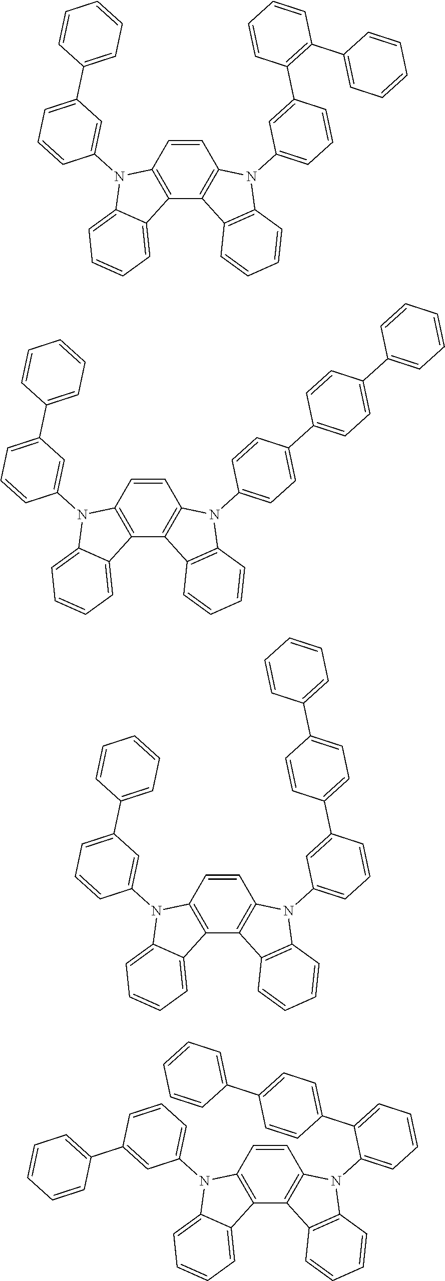

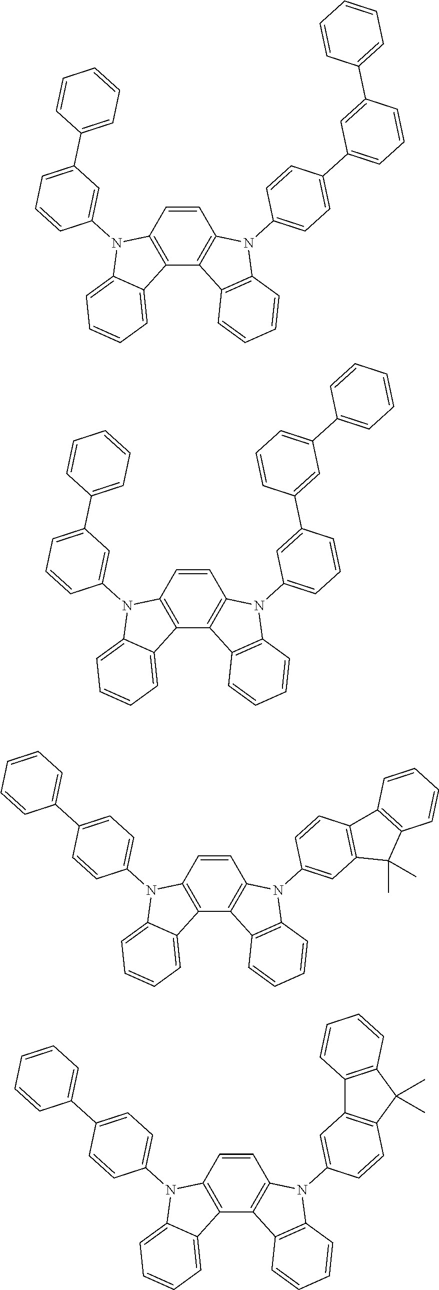 [SMILES: c1ccc(-c2cccc(-c3ccc(-n4c5ccccc5c5c6c7ccccc7n(-c7cccc(-c8ccccc8)c7)c6ccc54)cc3)c2)cc1 ; c1ccc(-c2cccc(-c3cccc(-n4c5ccccc5c5c6c7ccccc7n(-c7cccc(-c8ccccc8)c7)c6ccc54)c3)c2)cc1 ; CC1(C)c2ccccc2-c2ccc(-n3c4ccccc4c4c5c6ccccc6n(-c6ccc(-c7ccccc7)cc6)c5ccc43)cc21 ; CC1(C)c2ccccc2-c2cc(-n3c4ccccc4c4c5c6ccccc6n(-c6ccc(-c7ccccc7)cc6)c5ccc43)ccc21]

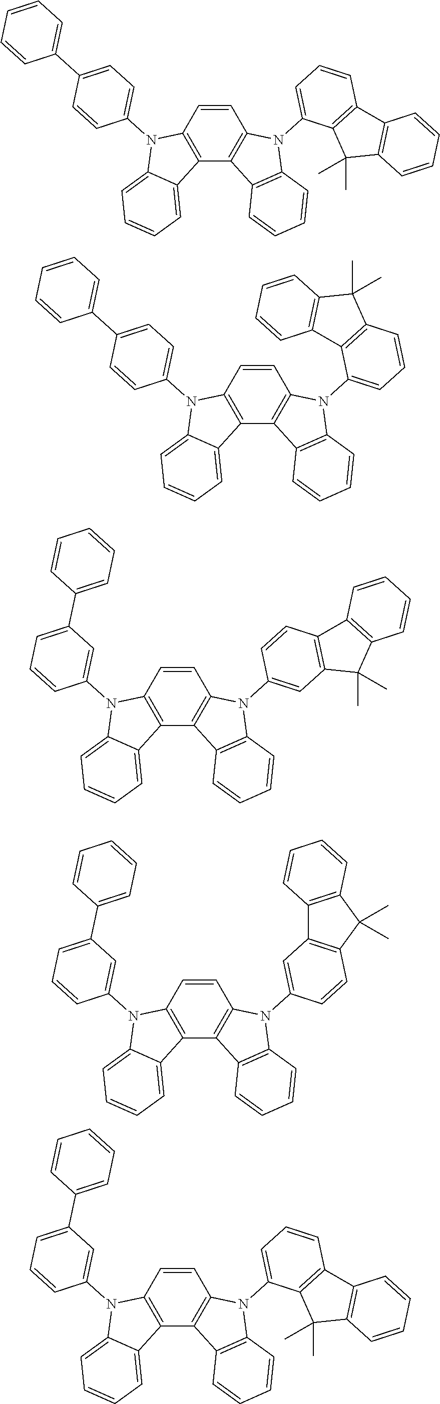

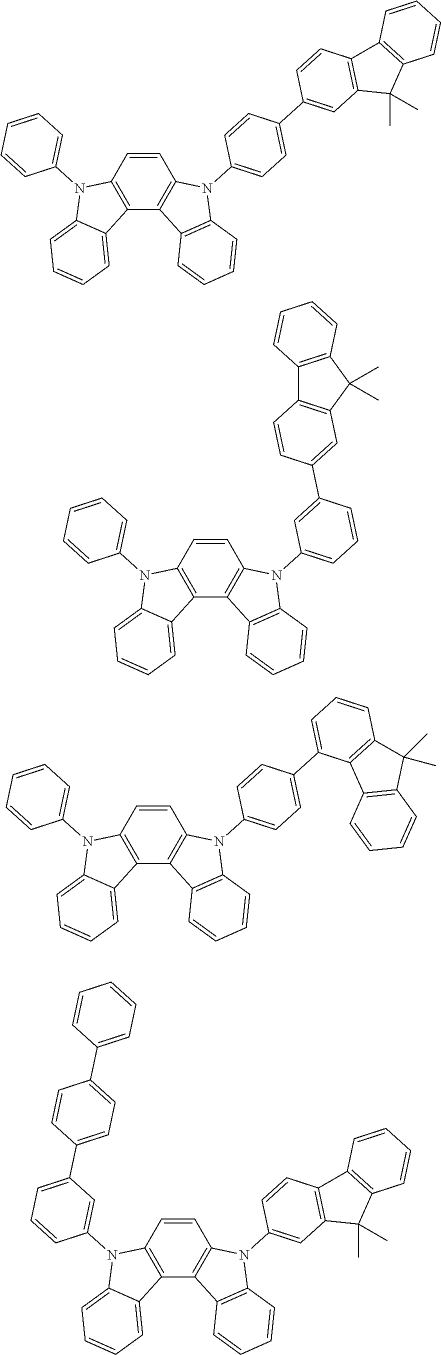

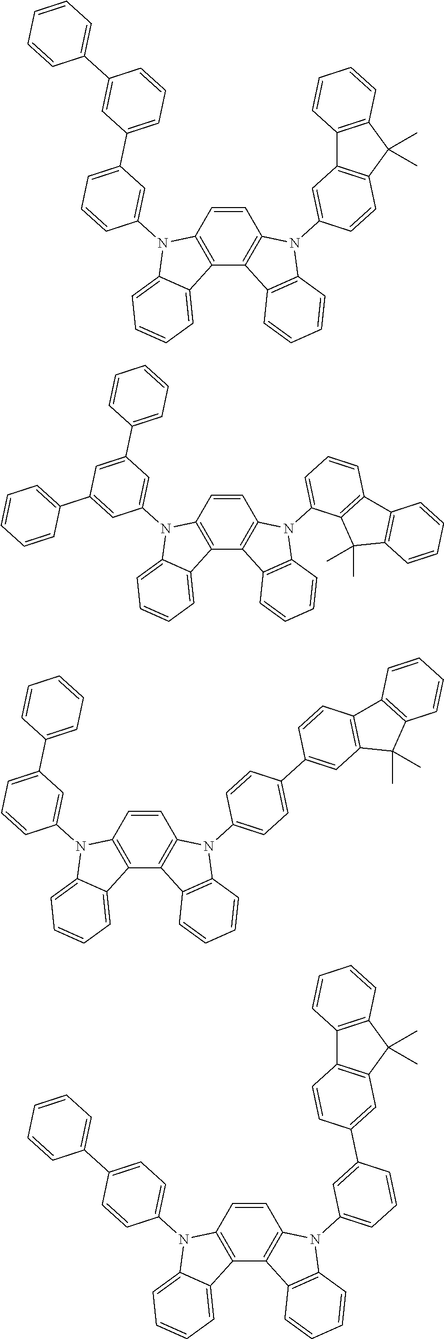

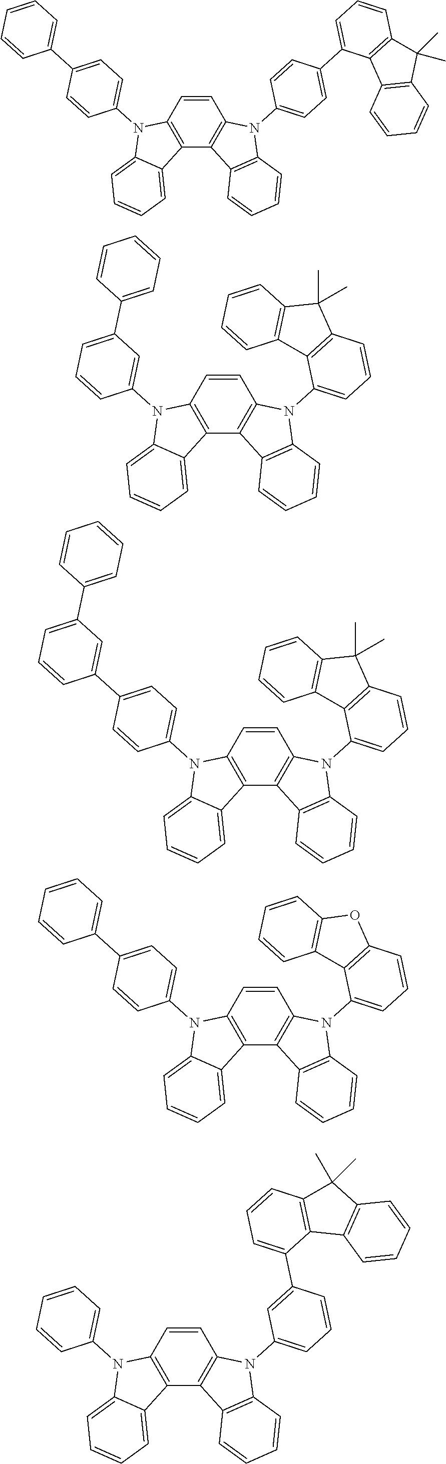

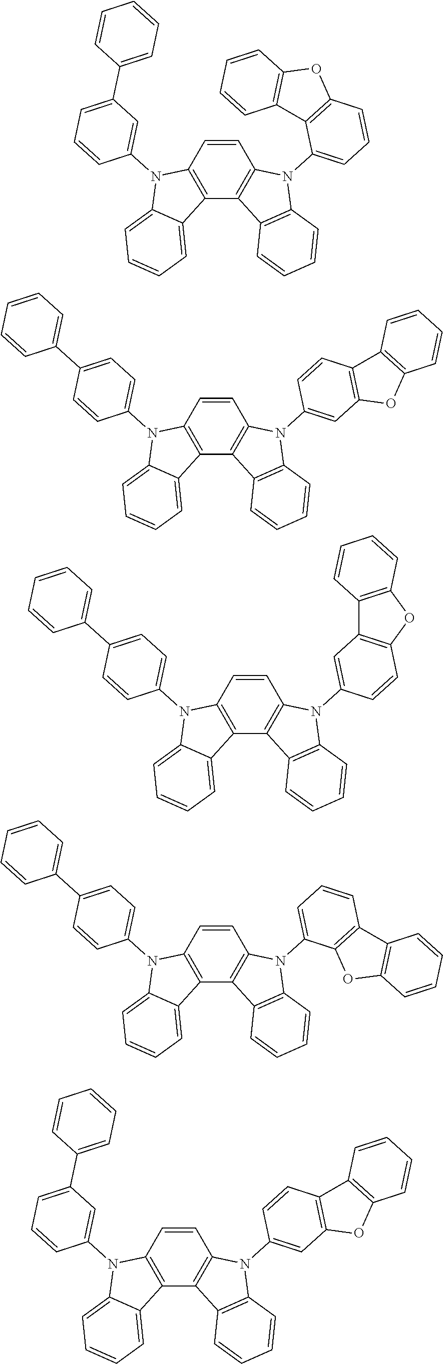

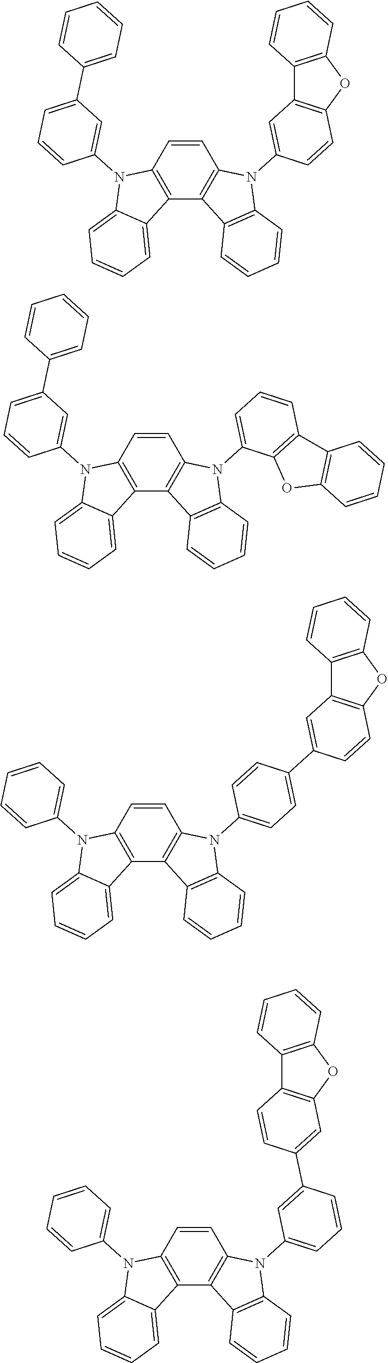

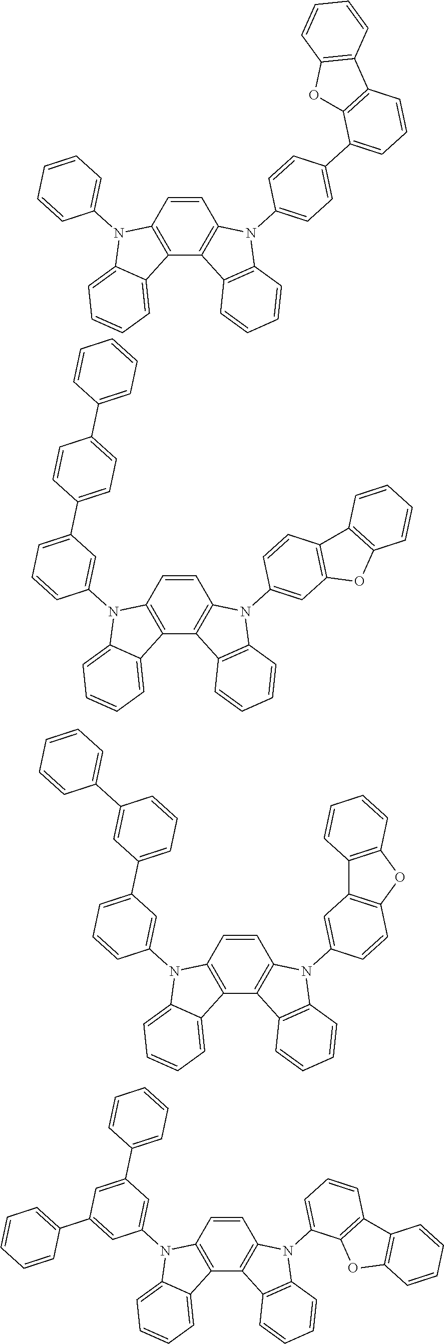

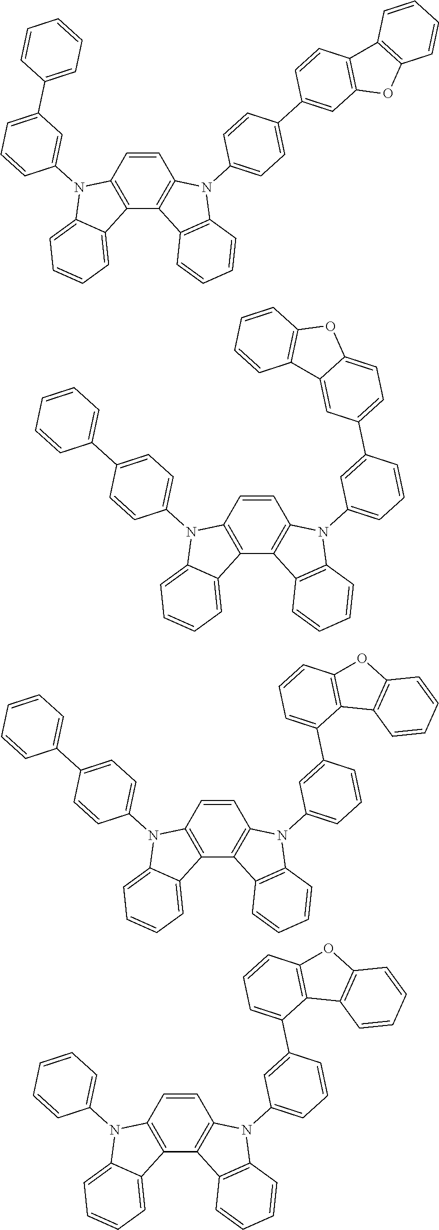

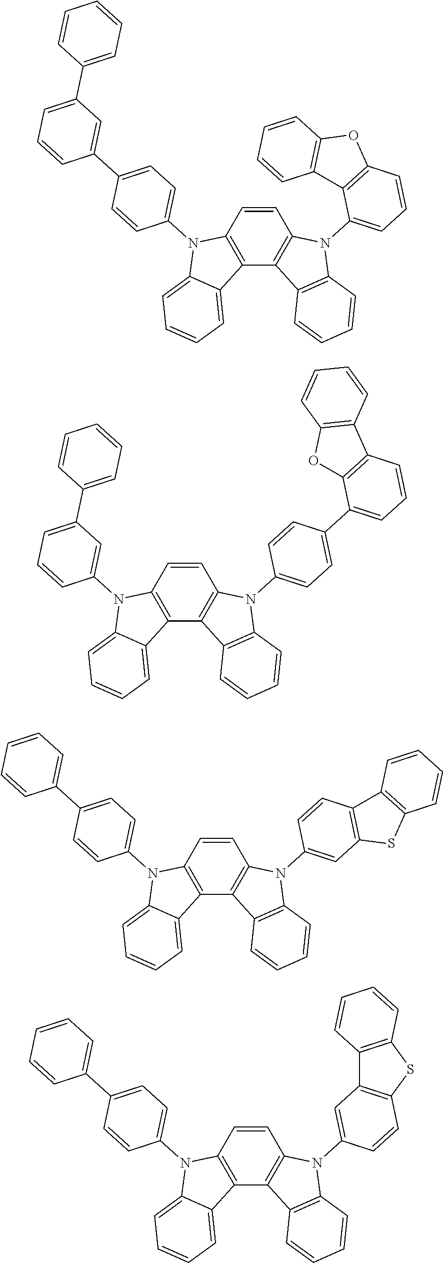

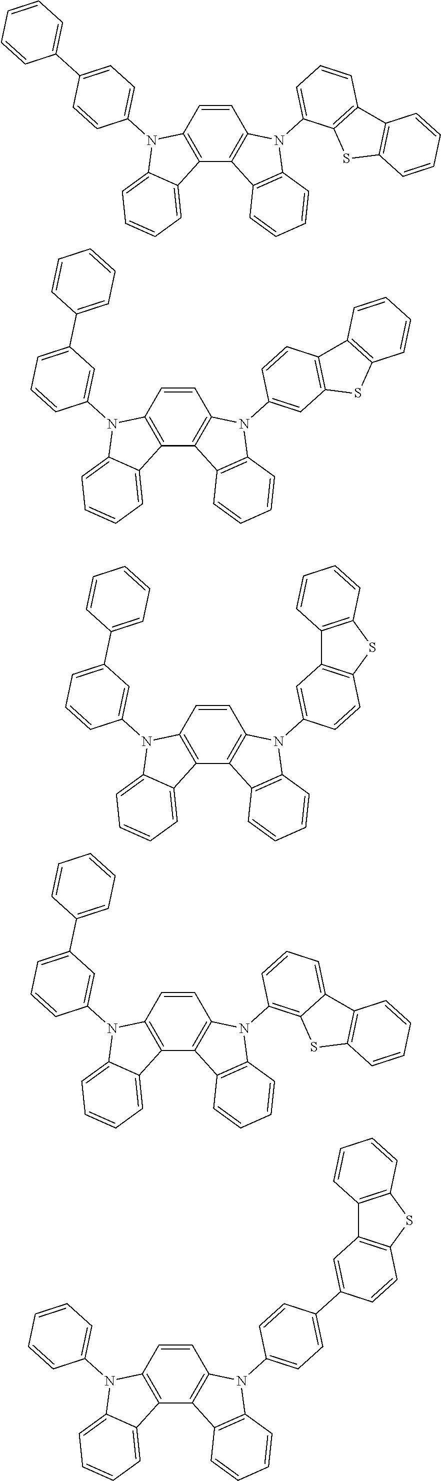

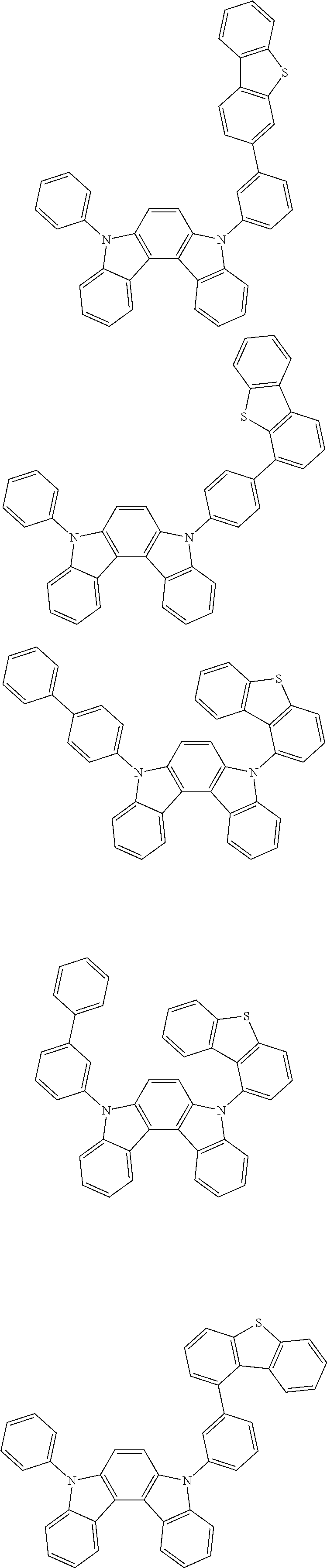

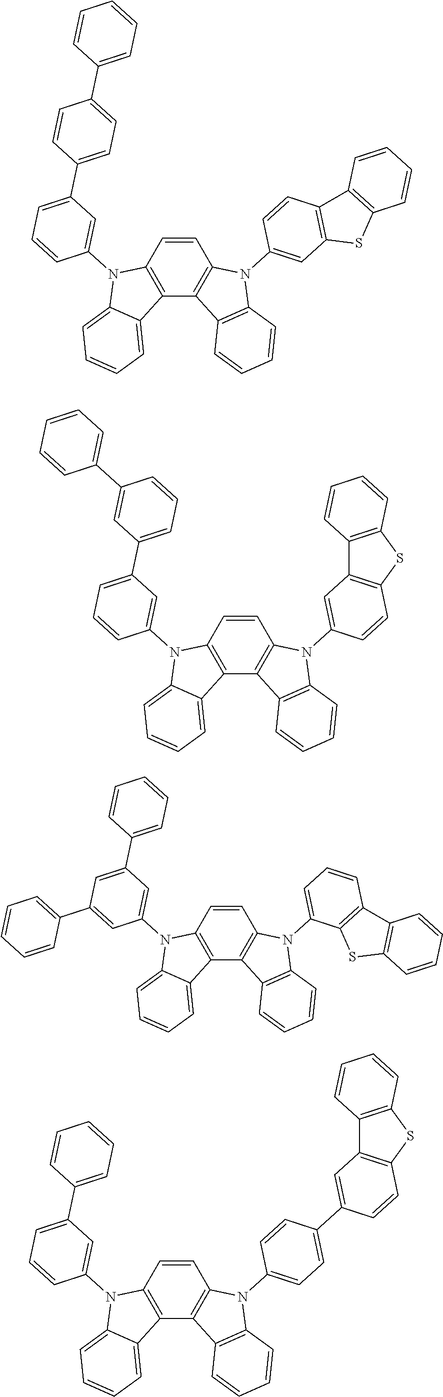

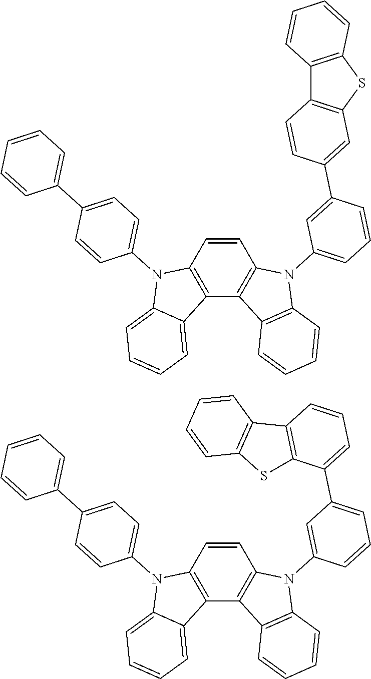

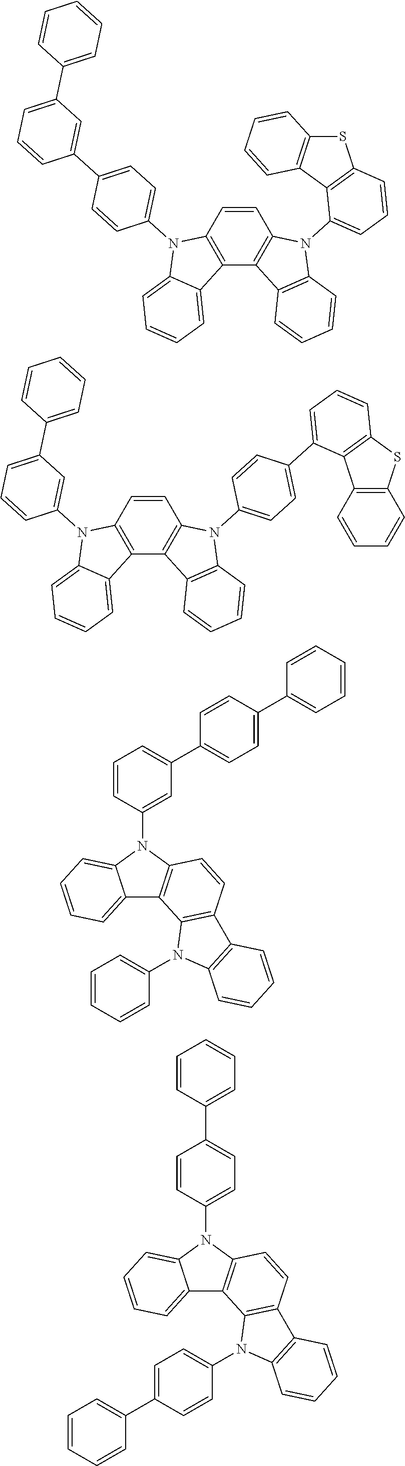

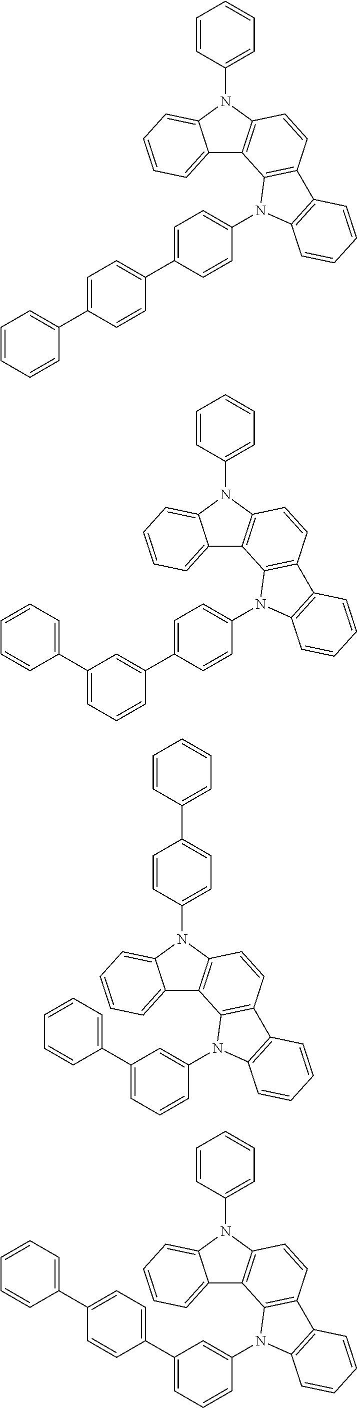 [SMILES: c1ccc(-c2ccc(-c3ccc(-n4c5ccccc5c5ccc6c(c7ccccc7n6-c6ccccc6)c54)cc3)cc2)cc1 ; c1ccc(-c2cccc(-c3ccc(-n4c5ccccc5c5ccc6c(c7ccccc7n6-c6ccccc6)c54)cc3)c2)cc1 ; c1ccc(-c2ccc(-n3c4ccccc4c4c3ccc3c5ccccc5n(-c5cccc(-c6ccccc6)c5)c34)cc2)cc1 ; c1ccc(-c2ccc(-c3cccc(-n4c5ccccc5c5ccc6c(c7ccccc7n6-c6ccccc6)c54)c3)cc2)cc1]

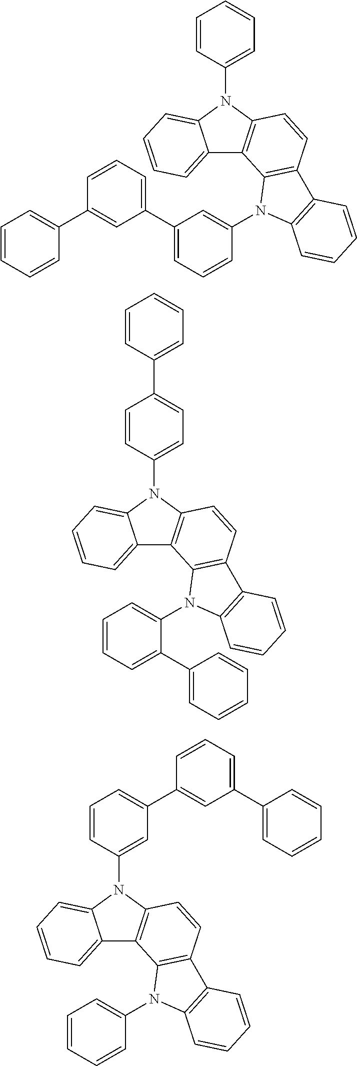

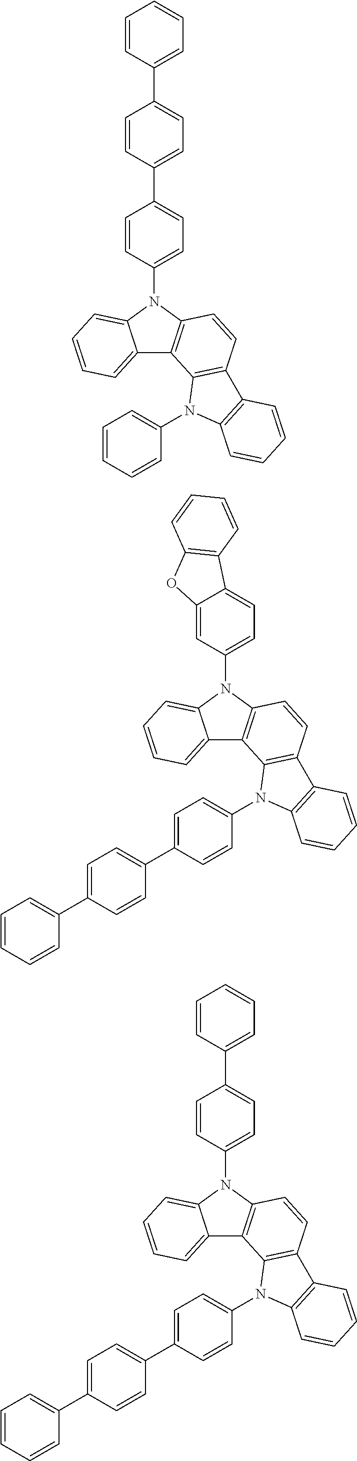

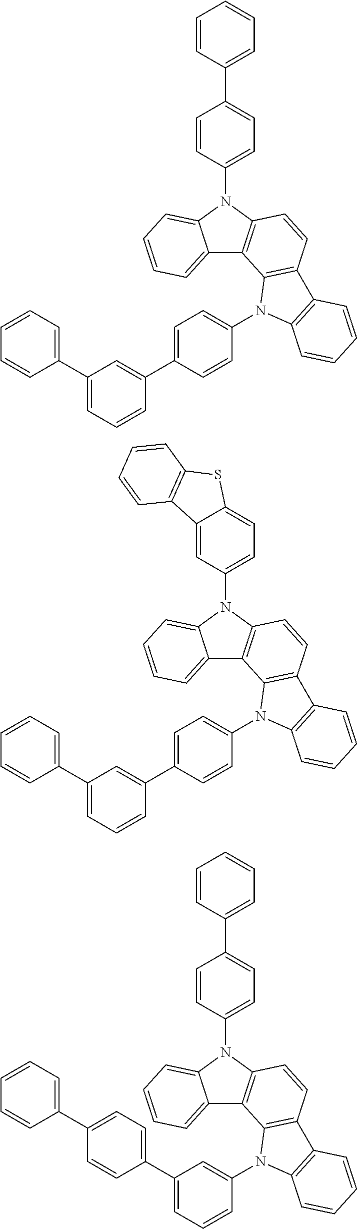

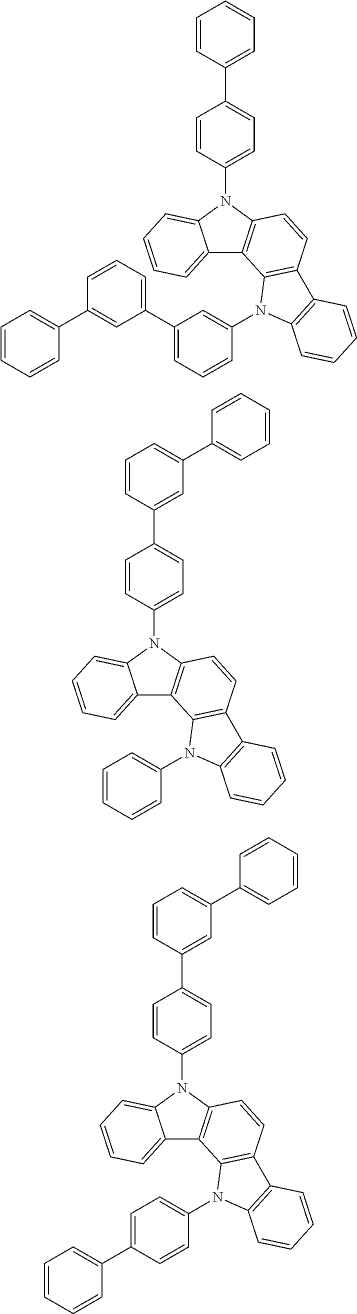

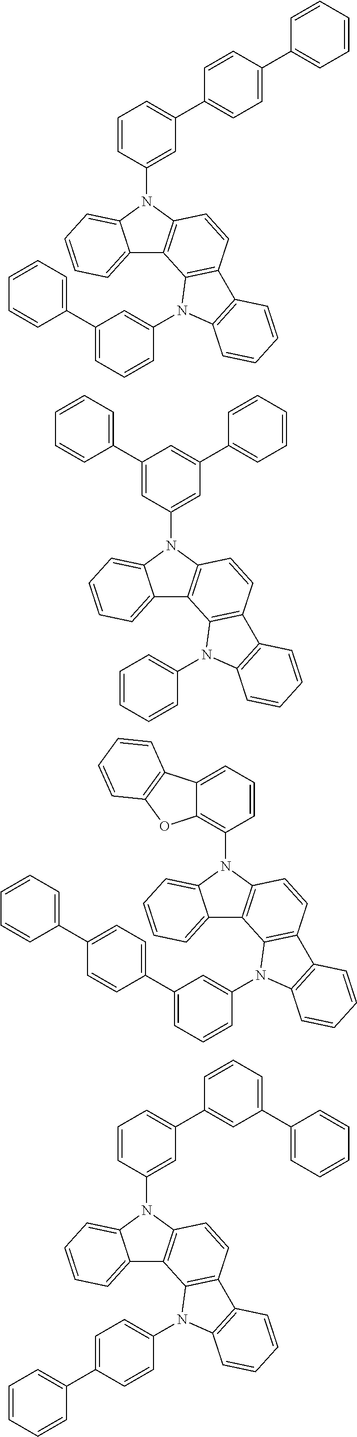

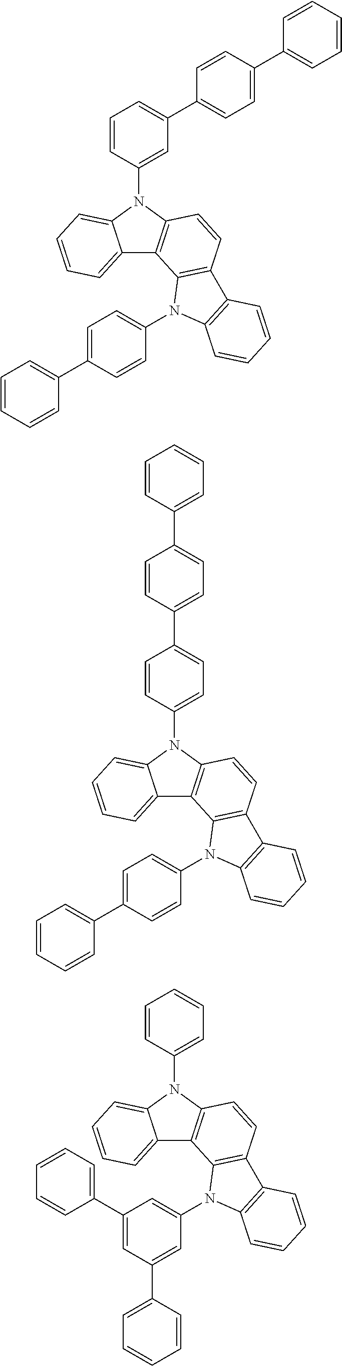 [SMILES: c1ccc(-c2ccc(-c3cccc(-n4c5ccccc5c5c4ccc4c6ccccc6n(-c6ccc(-c7ccccc7)cc6)c45)c3)cc2)cc1 ; c1ccc(-c2ccc(-c3ccc(-n4c5ccccc5c5c4ccc4c6ccccc6n(-c6ccc(-c7ccccc7)cc6)c45)cc3)cc2)cc1 ; c1ccc(-c2cc(-c3ccccc3)cc(-n3c4ccccc4c4ccc5c(c6ccccc6n5-c5ccccc5)c43)c2)cc1]

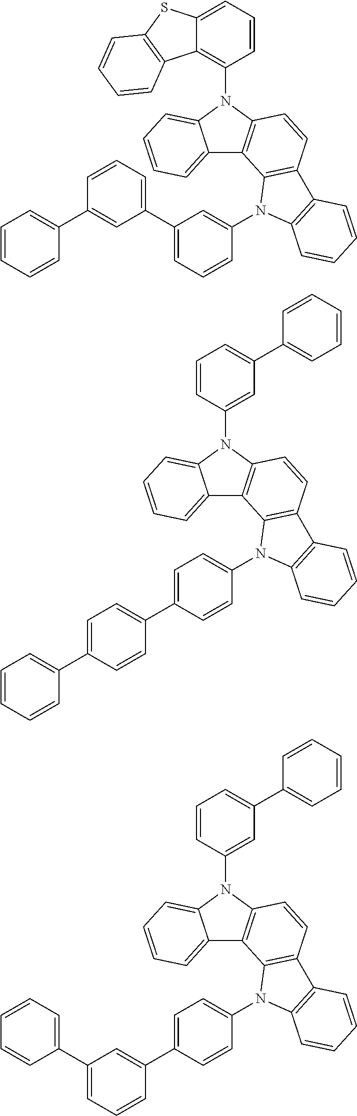

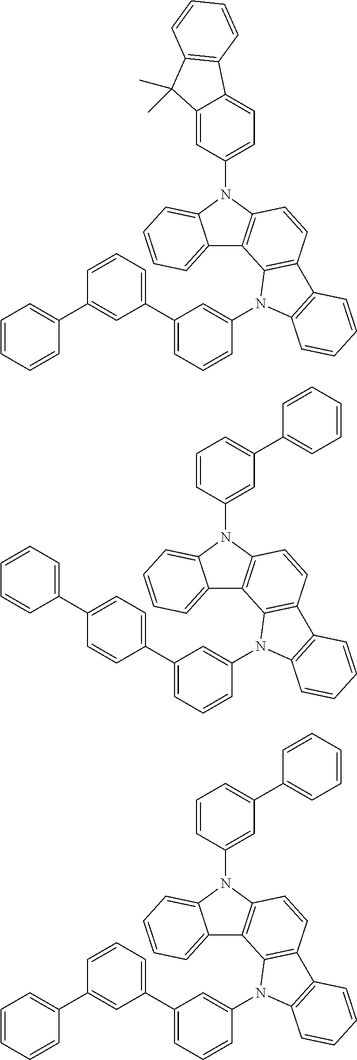

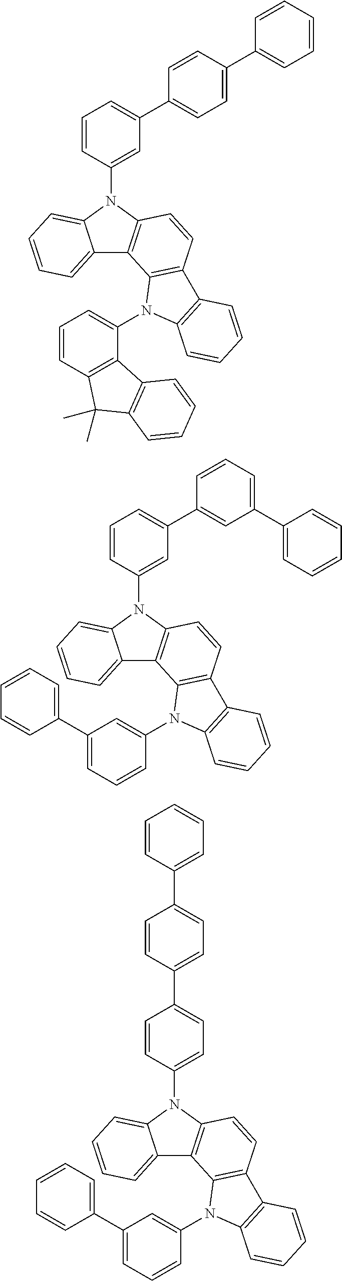

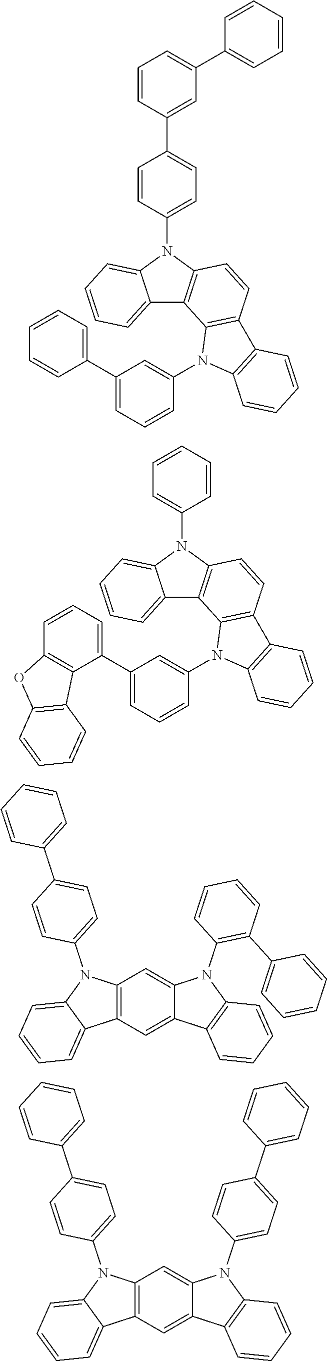

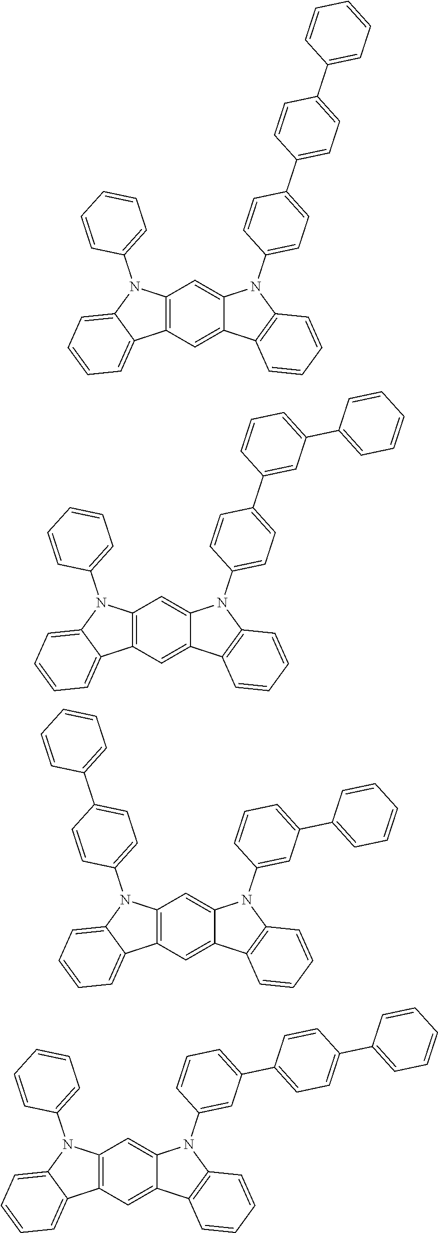

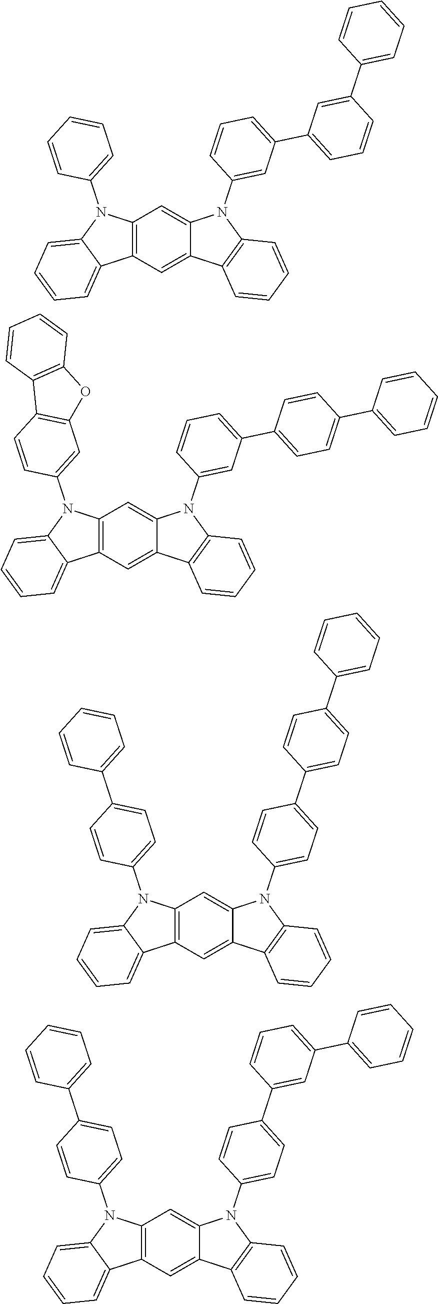

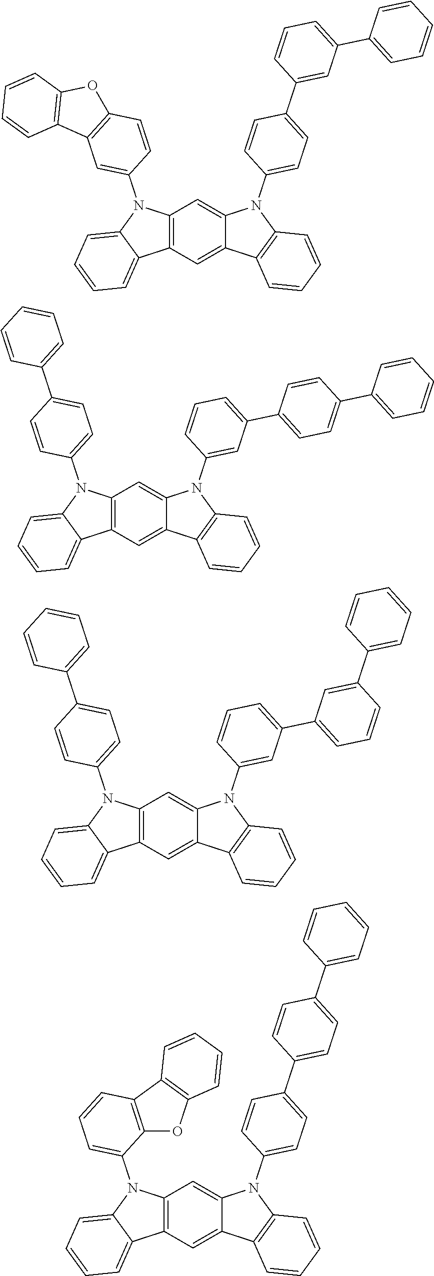

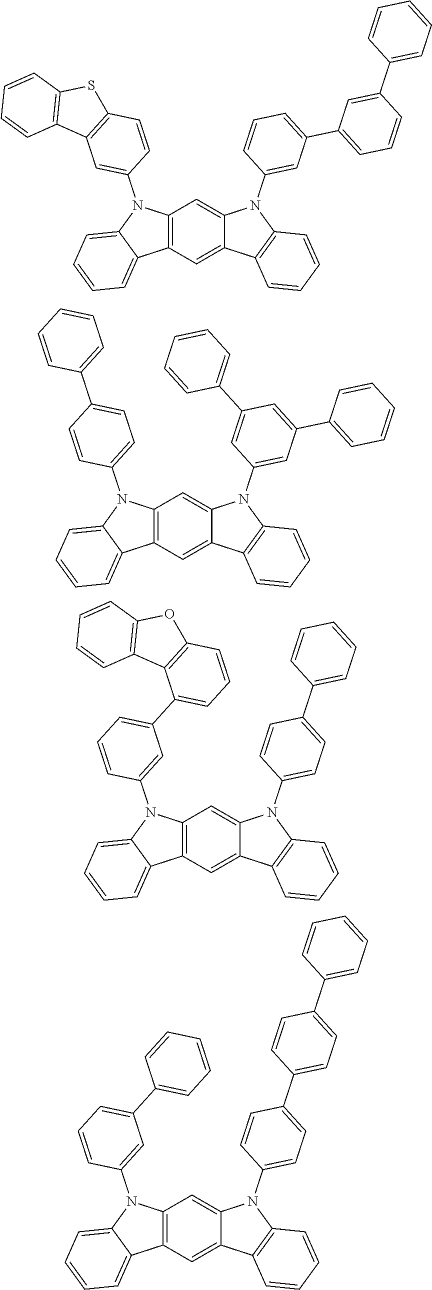

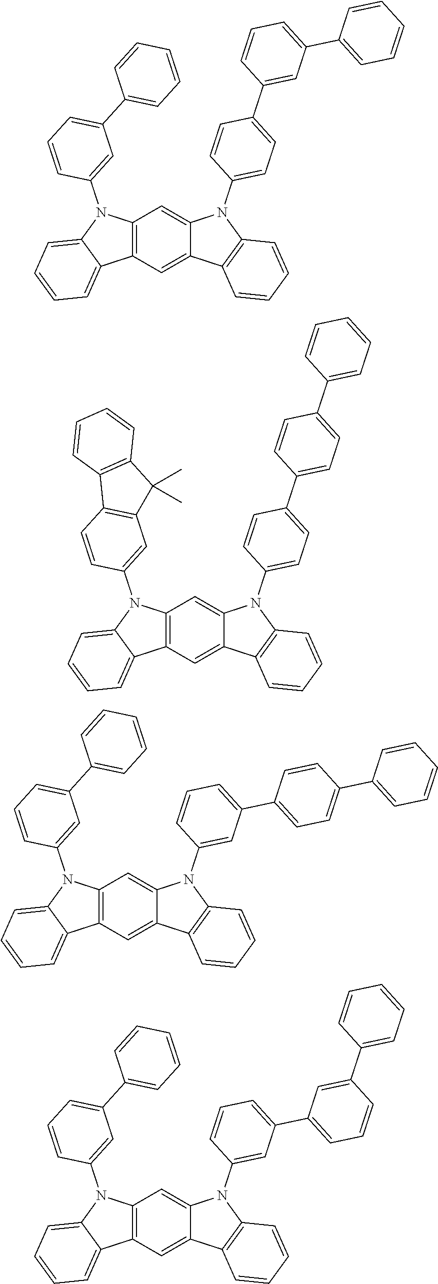

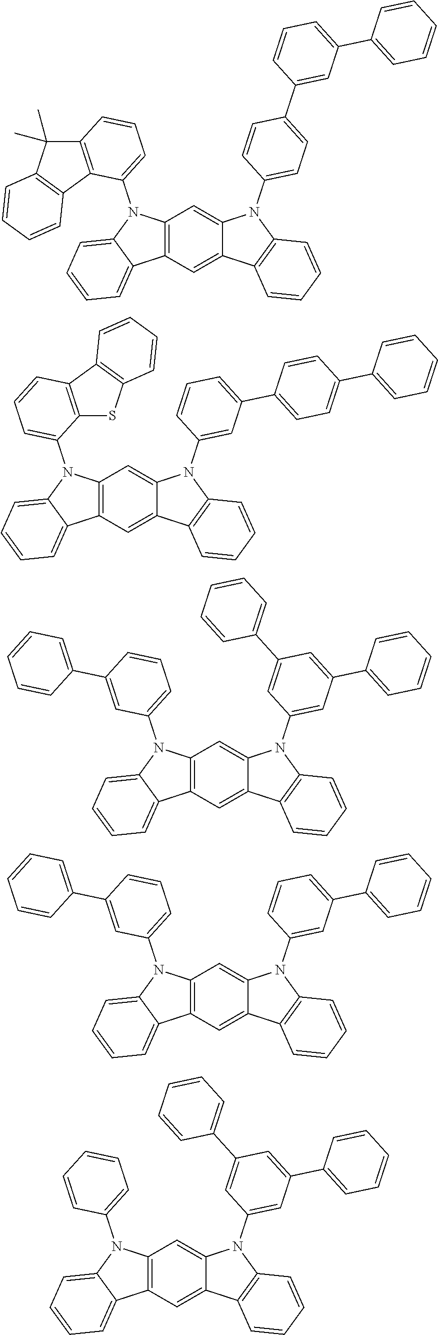

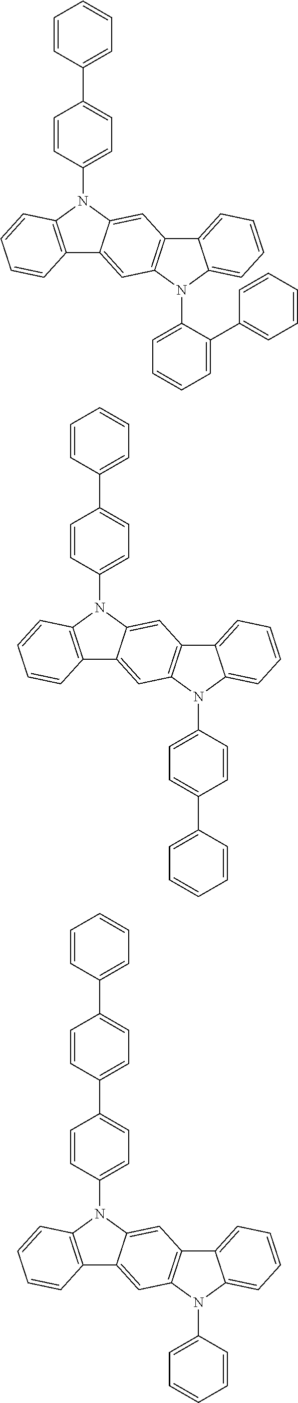 [SMILES: c1ccc(-c2ccc(-n3c4ccccc4c4cc5c(cc43)c3ccccc3n5-c3ccccc3-c3ccccc3)cc2)cc1 ; c1ccc(-c2ccc(-n3c4ccccc4c4cc5c(cc43)c3ccccc3n5-c3ccc(-c4ccccc4)cc3)cc2)cc1 ; c1ccc(-c2ccc(-c3ccc(-n4c5ccccc5c5cc6c(cc54)c4ccccc4n6-c4ccccc4)cc3)cc2)cc1]

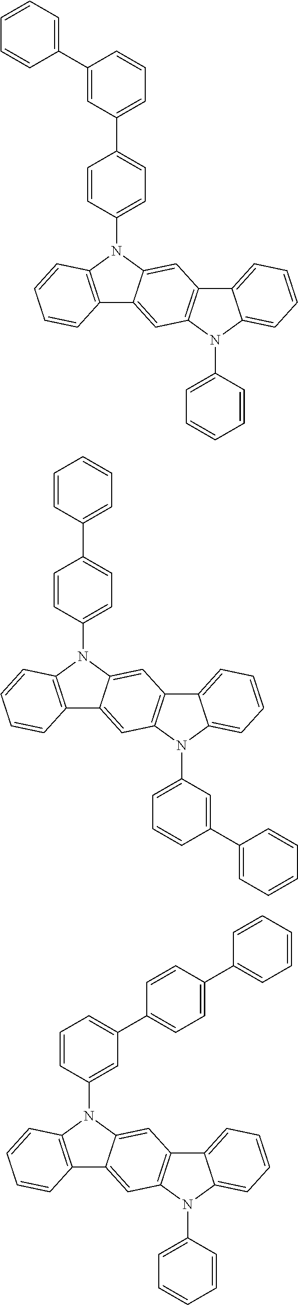

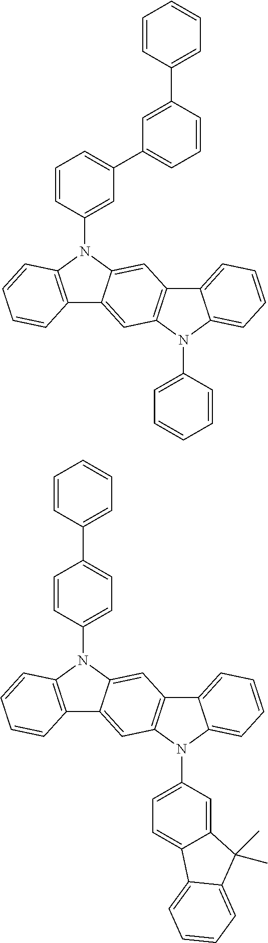 [SMILES: c1ccc(-c2cccc(-c3cccc(-n4c5ccccc5c5cc6c(cc54)c4ccccc4n6-c4ccccc4)c3)c2)cc1 ; CC1(C)c2ccccc2-c2ccc(-n3c4ccccc4c4cc5c(cc43)c3ccccc3n5-c3ccc(-c4ccccc4)cc3)cc21]

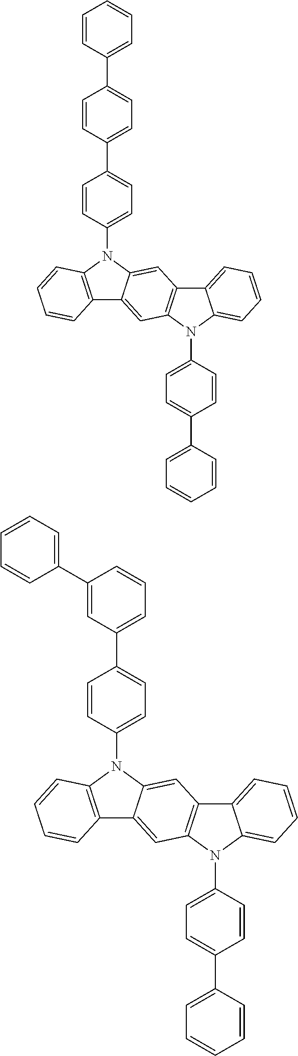 [SMILES: c1ccc(-c2ccc(-c3ccc(-n4c5ccccc5c5cc6c(cc54)c4ccccc4n6-c4ccc(-c5ccccc5)cc4)cc3)cc2)cc1 ; c1ccc(-c2ccc(-n3c4ccccc4c4cc5c(cc43)c3ccccc3n5-c3ccc(-c4cccc(-c5ccccc5)c4)cc3)cc2)cc1]

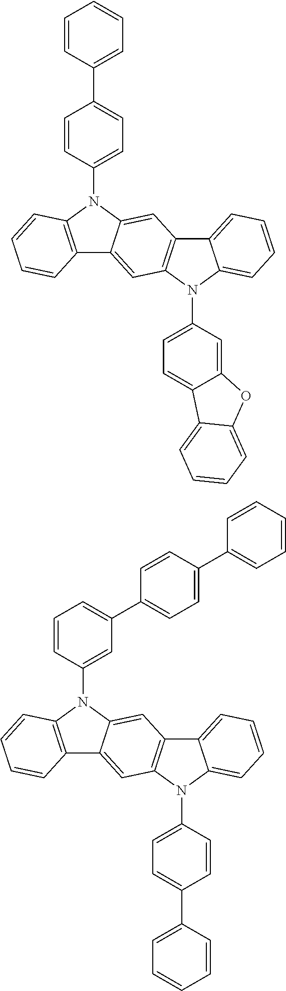

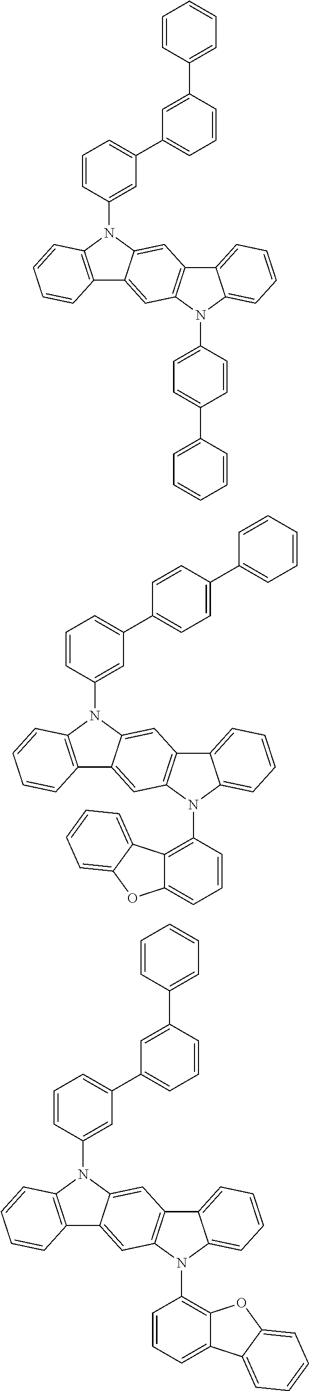

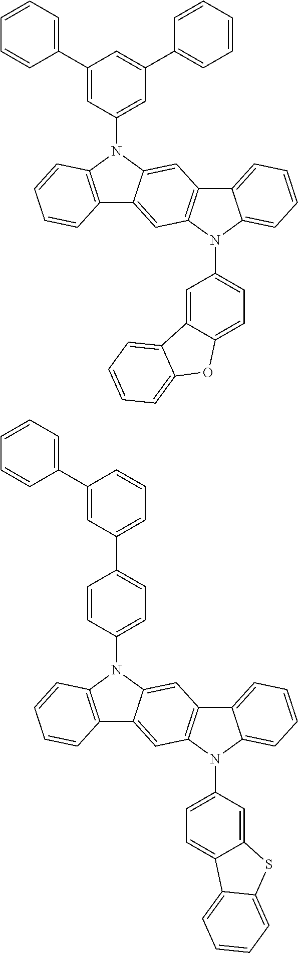 [SMILES: c1ccc(-c2cc(-c3ccccc3)cc(-n3c4ccccc4c4cc5c(cc43)c3ccccc3n5-c3ccc4oc5ccccc5c4c3)c2)cc1 ; c1ccc(-c2cccc(-c3ccc(-n4c5ccccc5c5cc6c(cc54)c4ccccc4n6-c4ccc5c(c4)sc4ccccc45)cc3)c2)cc1]

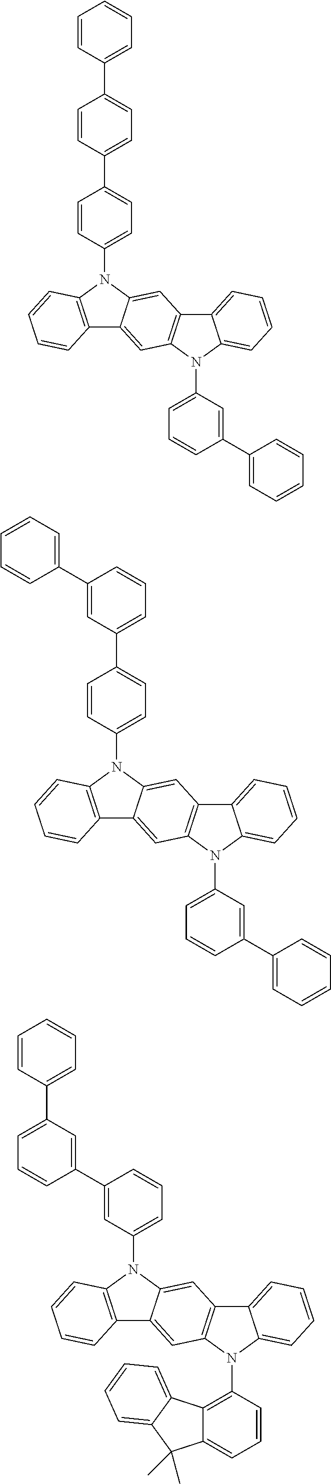 [SMILES: c1ccc(-c2ccc(-c3ccc(-n4c5ccccc5c5cc6c(cc54)c4ccccc4n6-c4cccc(-c5ccccc5)c4)cc3)cc2)cc1 ; c1ccc(-c2cccc(-c3ccc(-n4c5ccccc5c5cc6c(cc54)c4ccccc4n6-c4cccc(-c5ccccc5)c4)cc3)c2)cc1 ; CC1(C)c2ccccc2-c2c(-n3c4ccccc4c4cc5c(cc43)c3ccccc3n5-c3cccc(-c4cccc(-c5ccccc5)c4)c3)cccc21]

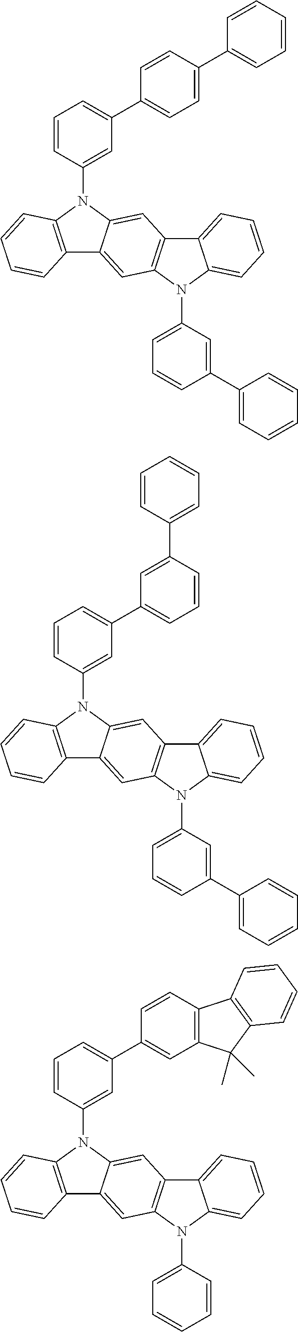

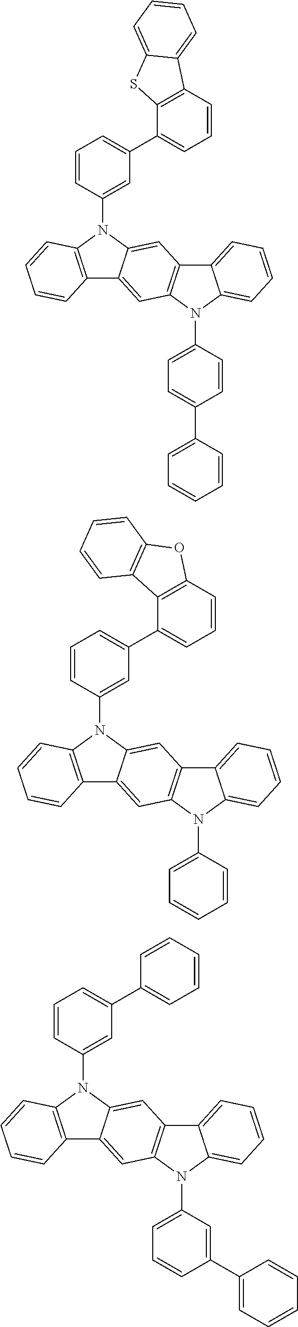

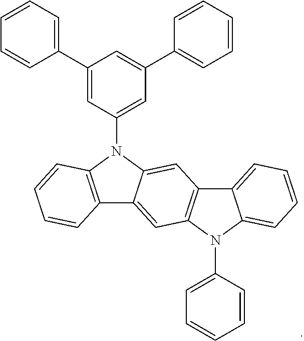 [SMILES: c1ccc(-c2cc(-c3ccccc3)cc(-n3c4ccccc4c4cc5c(cc43)c3ccccc3n5-c3ccccc3)c2)cc1]

7. The organic light emitting device of claim 1,
wherein Chemical Formula 2 is the following Chemical Formula 2-1: 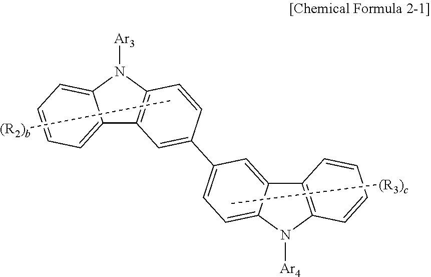
wherein in Chemical Formula 2-1, Ar₃, Ar₄, R₂, R₃, b and c have the same definitions as in claim 1.

8. The organic light emitting device of claim 1, wherein Arₙ and Ar₄ are each independently phenyl, biphenylyl, (phenyl)biphenylyl, naphthyl, dimethylfluorenyl, dibenzofuranyl, or dibenzothiophenyl.

9. The organic light emitting device of claim 1, wherein R₂ and R₃ are each independently hydrogen, deuterium, or phenyl.

10. The organic light emitting device of claim 9, wherein any one of R₂ and R₃ is phenyl, and the other is hydrogen or deuterium.

11. The organic light emitting device of claim 1, wherein the compound of Chemical Formula 2 is any one compound selected from the group consisting of the following compounds:

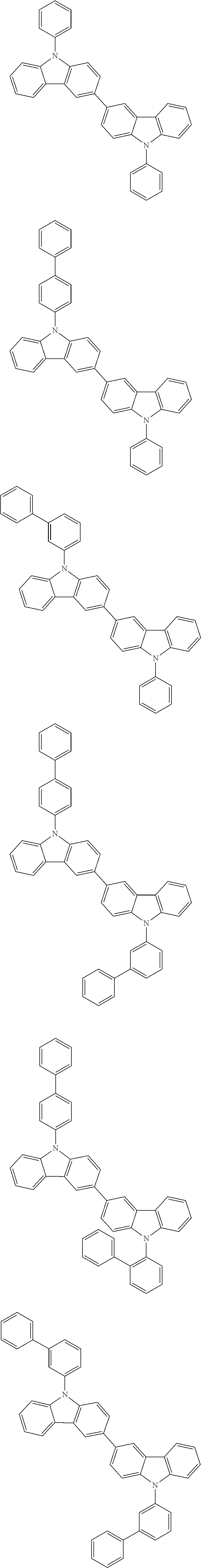 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

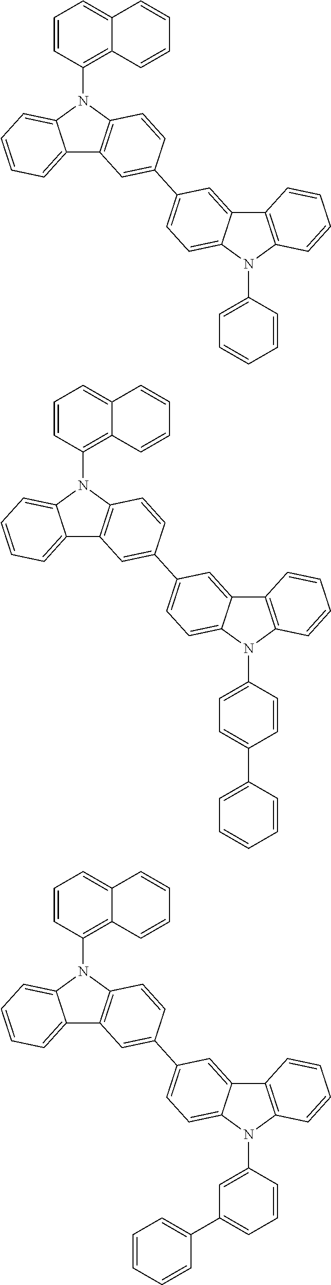 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2cccc(-n3c4ccccc4c4cc(-c5ccc6c(c5)c5ccccc5n6-c5cccc6ccccc56)ccc43)c2)cc1]

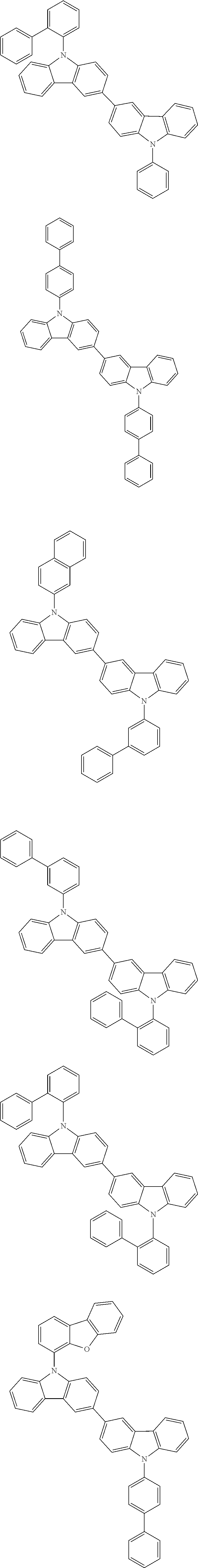

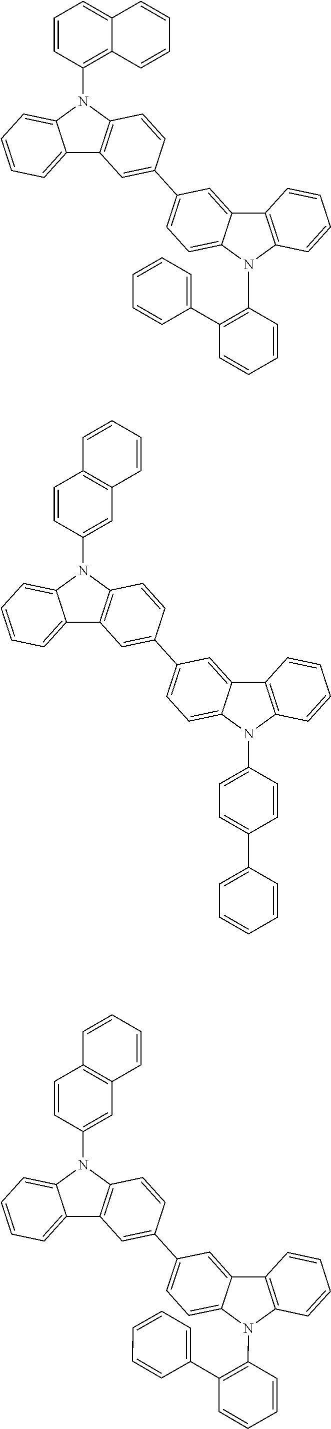 [SMILES: c1ccc(-c2ccccc2-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccccc2-n2c3ccccc3c3cc(-c4ccc5c(c4)c4ccccc4n5-c4ccc5ccccc5c4)ccc32)cc1]

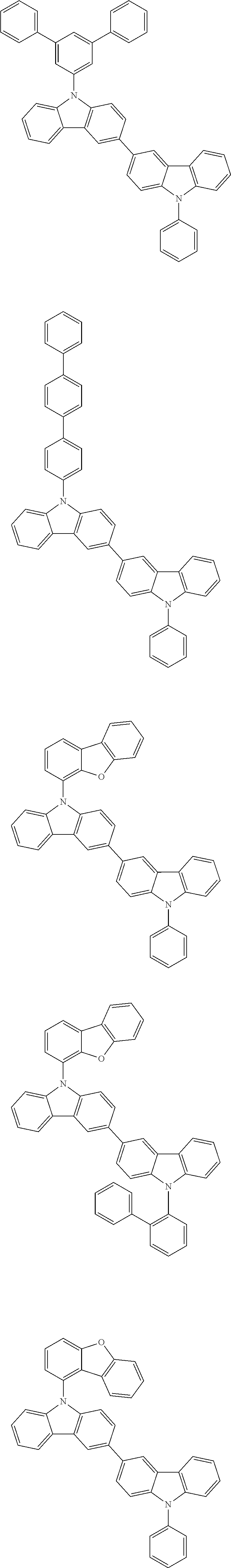 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-n2c3ccccc3c3cc(-c4ccc5c(c4)c4ccccc4n5-c4cccc5c4oc4ccccc45)ccc32)cc1 ; c1ccc(-c2ccccc2-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc5oc6ccccc6c45)ccc32)cc1]

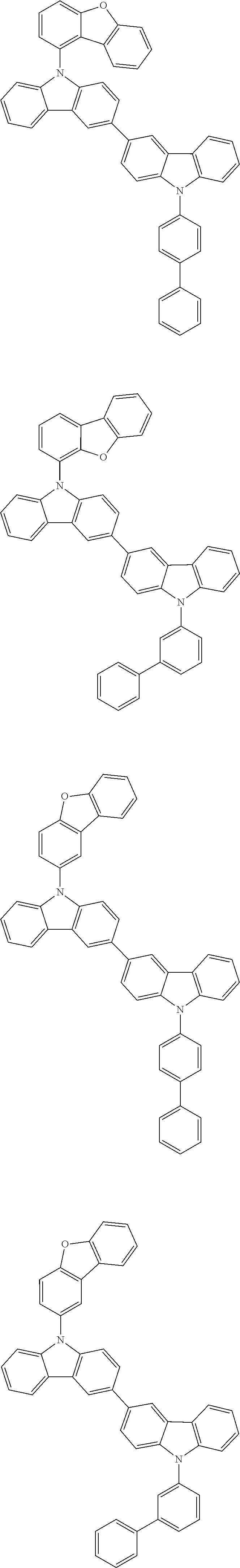 [SMILES: c1ccc(-c2ccc(-n3c4ccccc4c4cc(-c5ccc6c(c5)c5ccccc5n6-c5cccc6oc7ccccc7c56)ccc43)cc2)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

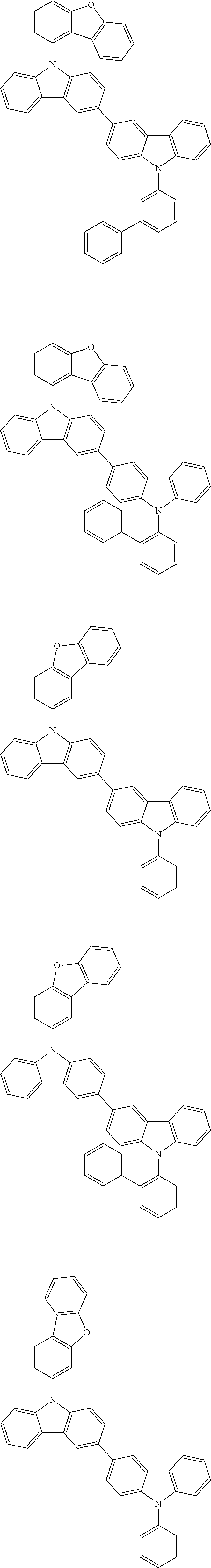 [SMILES: c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2ccccc2-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-c2ccccc2-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5c(c4)oc4ccccc45)ccc32)cc1]

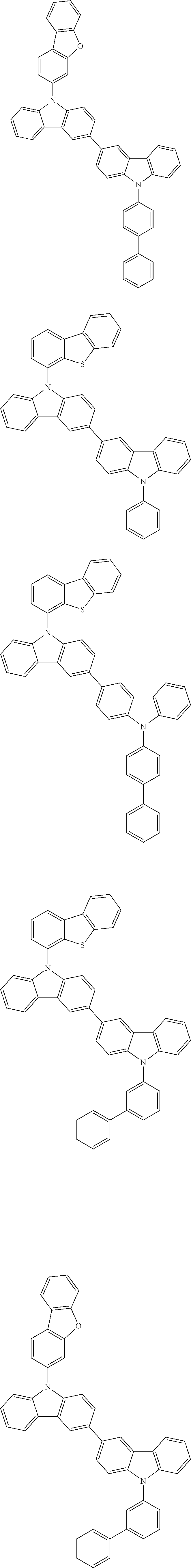

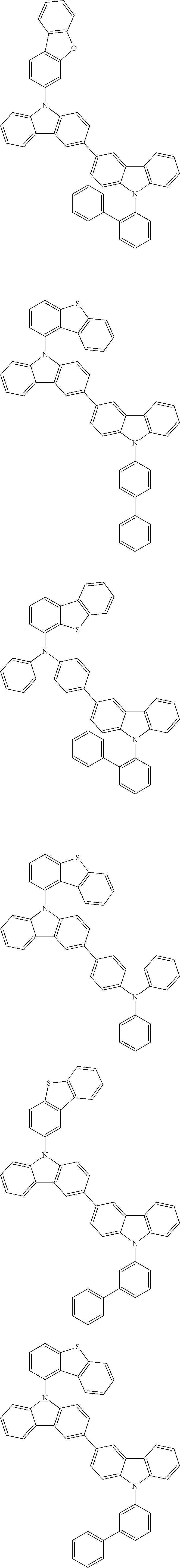

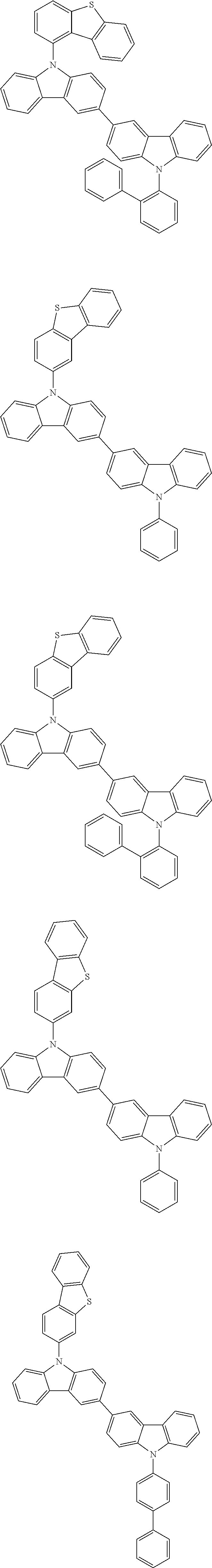 [SMILES: c1ccc(-c2ccccc2-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccccc2-n2c3ccccc3c3cc(-c4ccc5c(c4)c4ccccc4n5-c4ccc5sc6ccccc6c5c4)ccc32)cc1 ; c1ccc(-n2c3ccccc3c3cc(-c4ccc5c(c4)c4ccccc4n5-c4ccc5c(c4)sc4ccccc45)ccc32)cc1 ; c1ccc(-c2ccc(-n3c4ccccc4c4cc(-c5ccc6c(c5)c5ccccc5n6-c5ccc6c(c5)sc5ccccc56)ccc43)cc2)cc1]

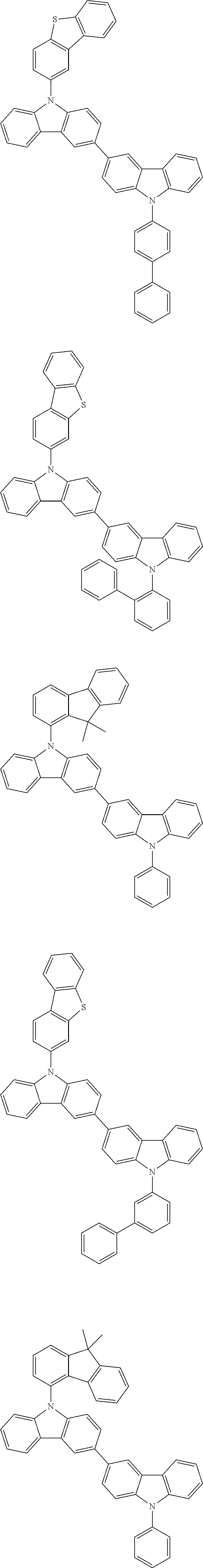 [SMILES: c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2ccccc2-n2c3ccccc3c3cc(-c4ccc5c(c4)c4ccccc4n5-c4ccc5c(c4)sc4ccccc45)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-c2cccc(-n3c4ccccc4c4cc(-c5ccc6c(c5)c5ccccc5n6-c5ccc6c(c5)sc5ccccc56)ccc43)c2)cc1 ; CC1(C)c2ccccc2-c2c(-n3c4ccccc4c4cc(-c5ccc6c(c5)c5ccccc5n6-c5ccccc5)ccc43)cccc21]

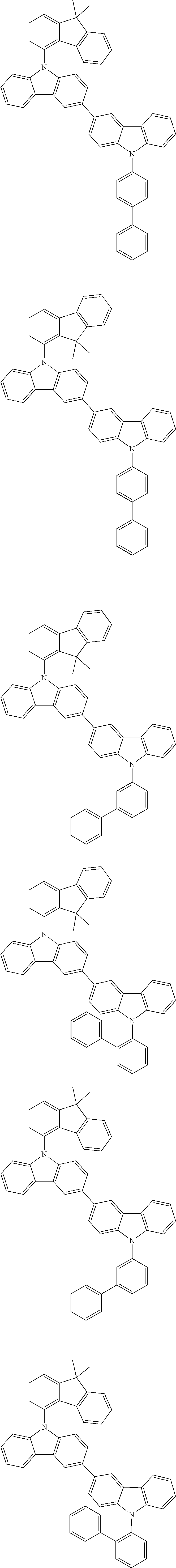

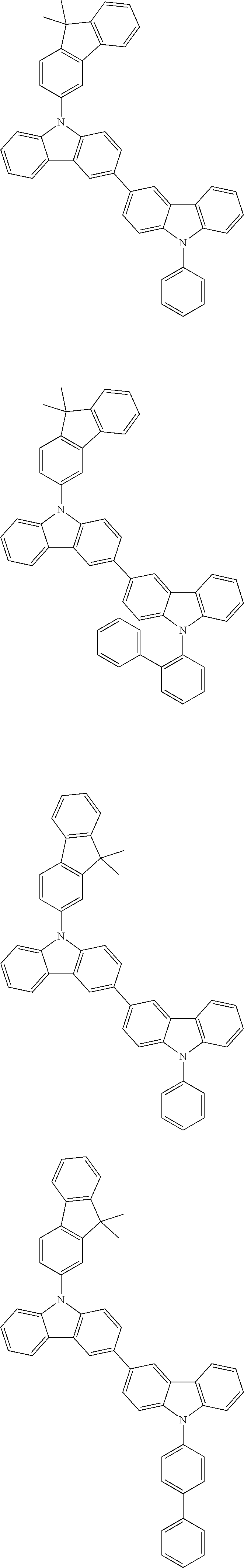 [SMILES: CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccccc5-c5ccccc5)ccc43)ccc21 ; CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21]

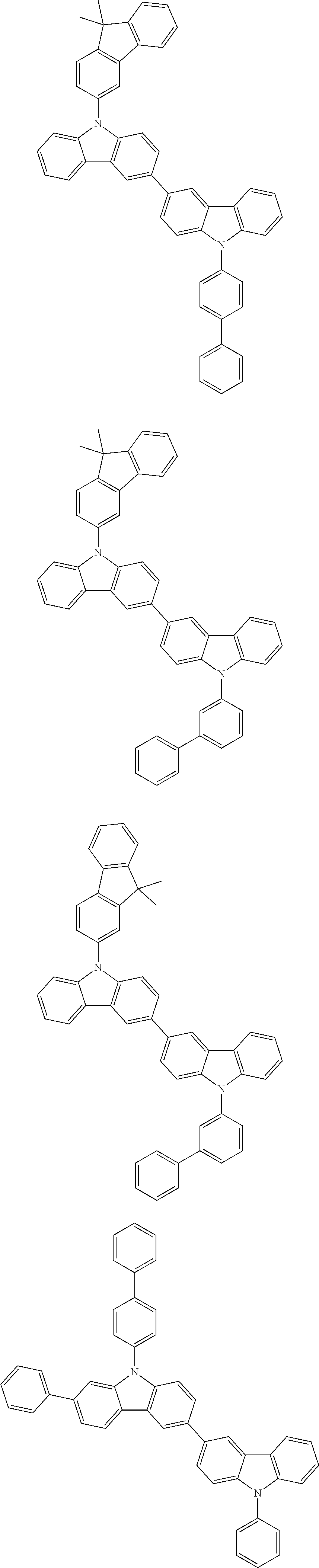

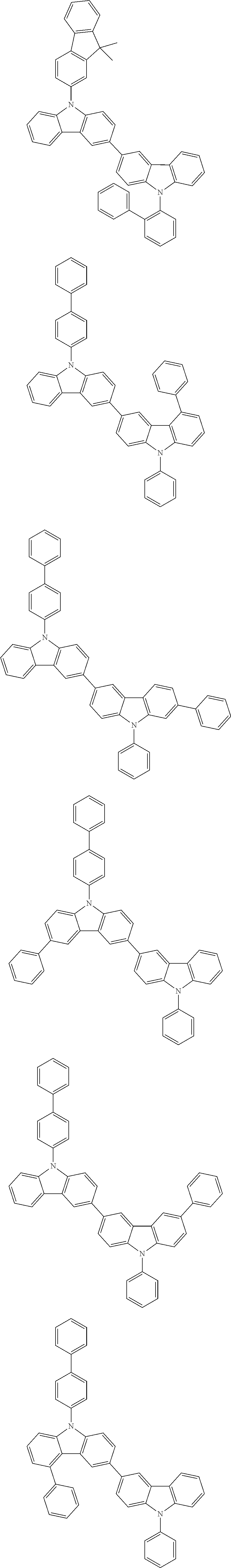

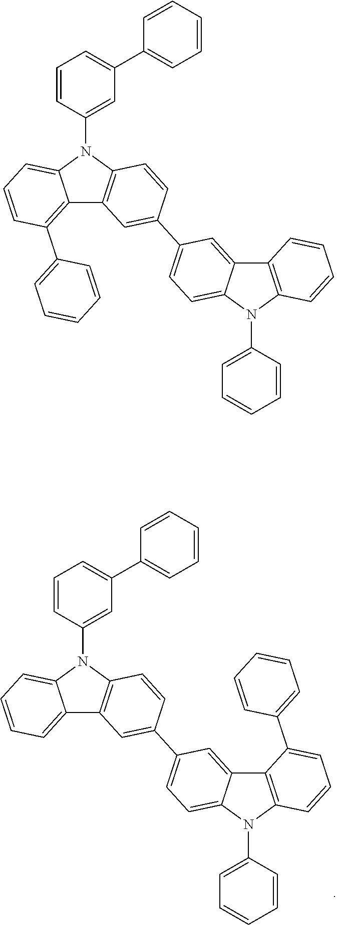 [SMILES: c1ccc(-c2cccc(-n3c4ccc(-c5ccc6c(c5)c5ccccc5n6-c5ccccc5)cc4c4c(-c5ccccc5)cccc43)c2)cc1 ; c1ccc(-c2cccc(-n3c4ccccc4c4cc(-c5ccc6c(c5)c5c(-c7ccccc7)cccc5n6-c5ccccc5)ccc43)c2)cc1]

12. The organic light emitting device of claim 1, wherein Chemical Formula 3 is any one of the following Chemical Formulae 3-1 to 3-6: 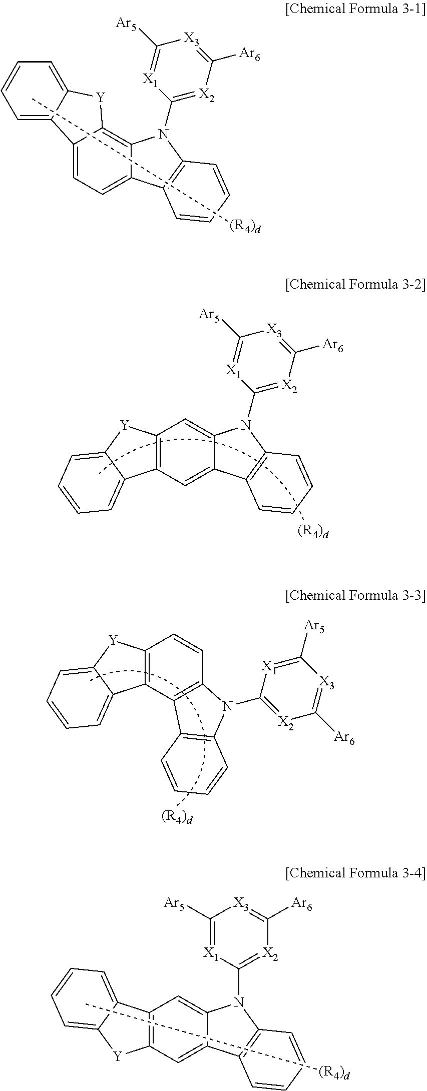 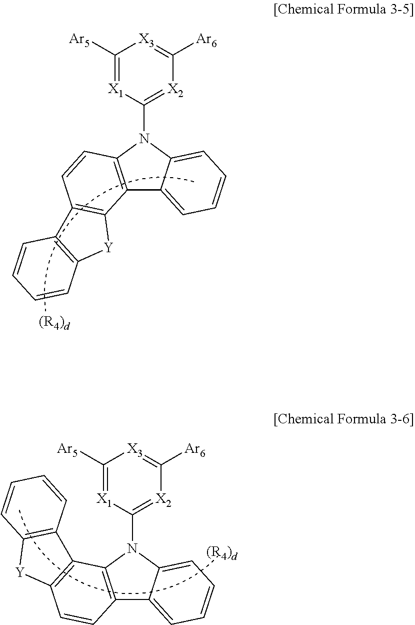
wherein in Chemical Formulae 3-1 to 3-6, X₁, X₂, X₃, Ar₅, Ar₆, R₄, Y, and d have the same definitions as in claim 1.

13. The organic light emitting device of claim 1, wherein:
Ar₅ and Ar₆ are each independently phenyl, biphenylyl, (phenyl)biphenylyl, dimethylfluorenyl, dibenzofuranyl, or dibenzothiophenyl; and
Ar₅ and Ar₆ are each independently unsubstituted or substituted with at least one deuterium.

14. The organic light emitting device of claim 1, wherein:
Ar₇ is phenyl, biphenylyl, or terphenylyl; and
Ar₇ is unsubstituted or substituted with at least one deuterium.

15. The organic light emitting device of claim 1, wherein R₄ is hydrogen or deuterium.

16. The organic light emitting device of claim 1,
wherein the compound of Chemical Formula 3 is any one compound selected from the group consisting of the following compounds: 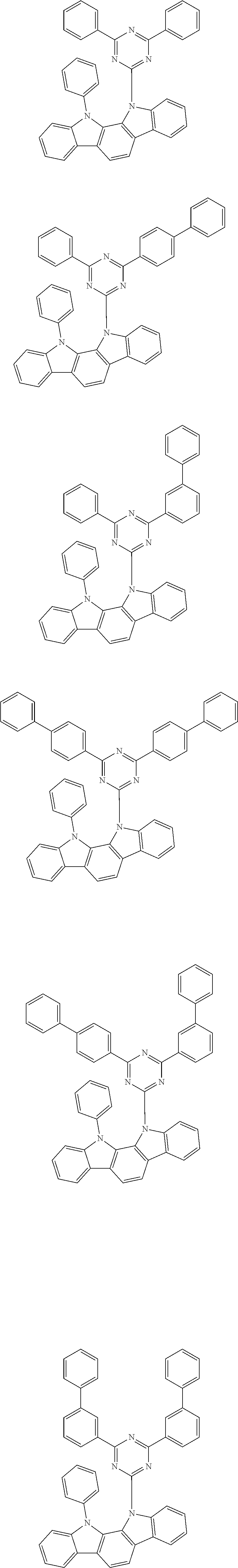 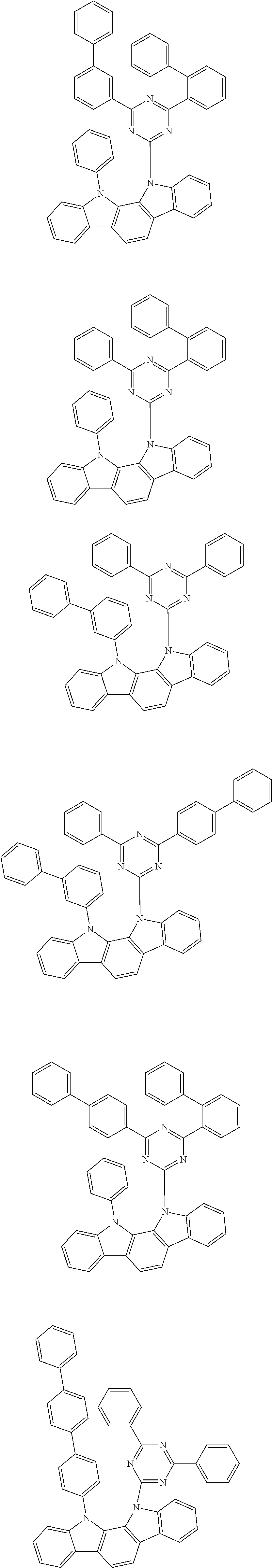 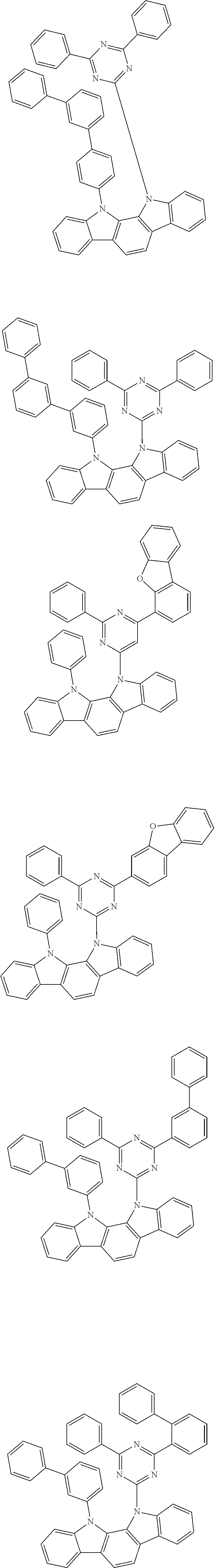 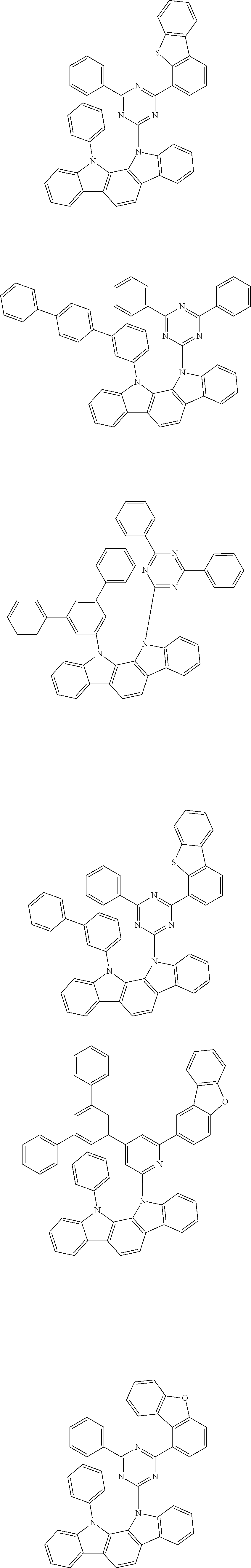 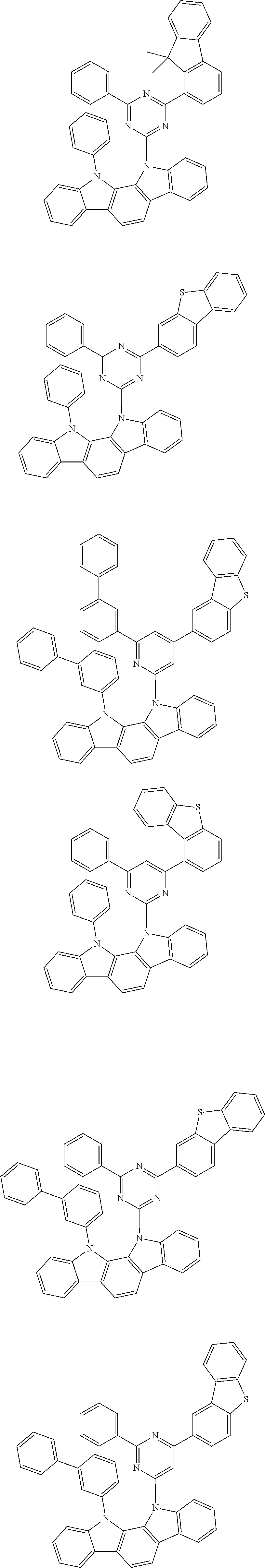 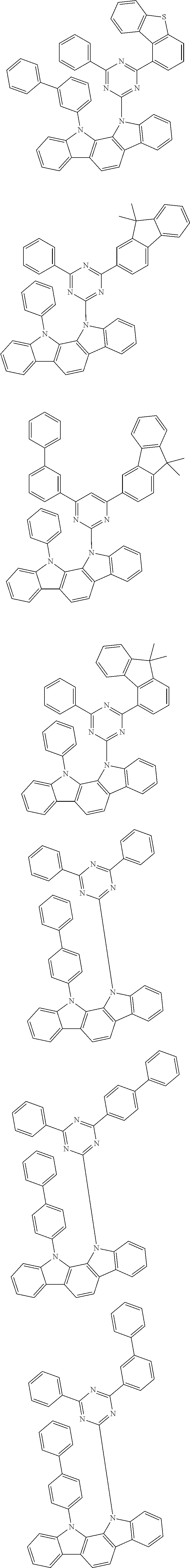 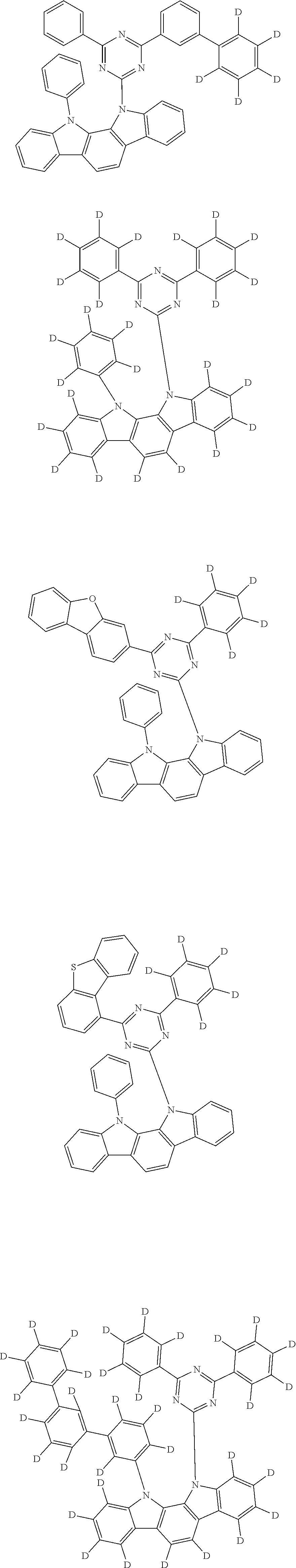 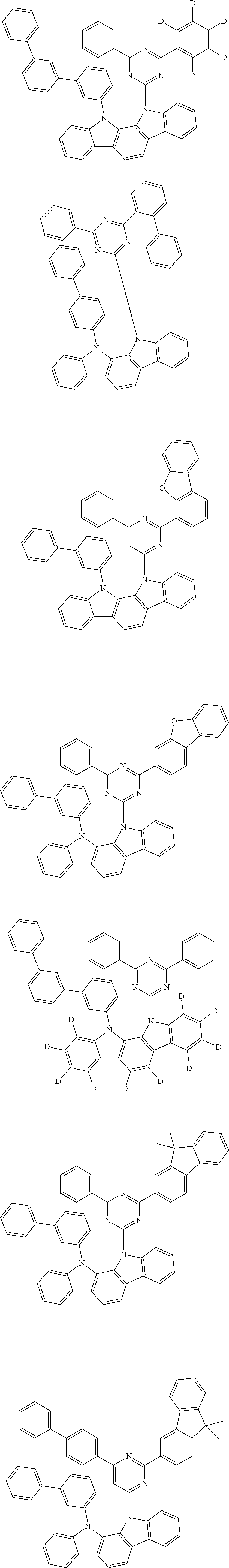 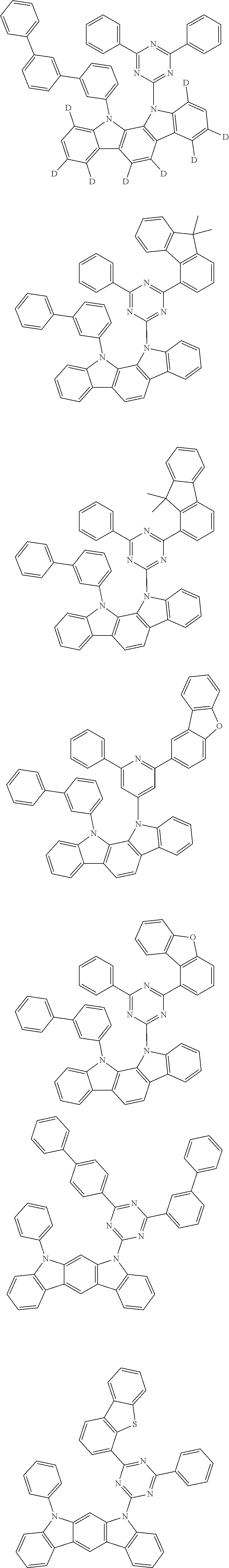 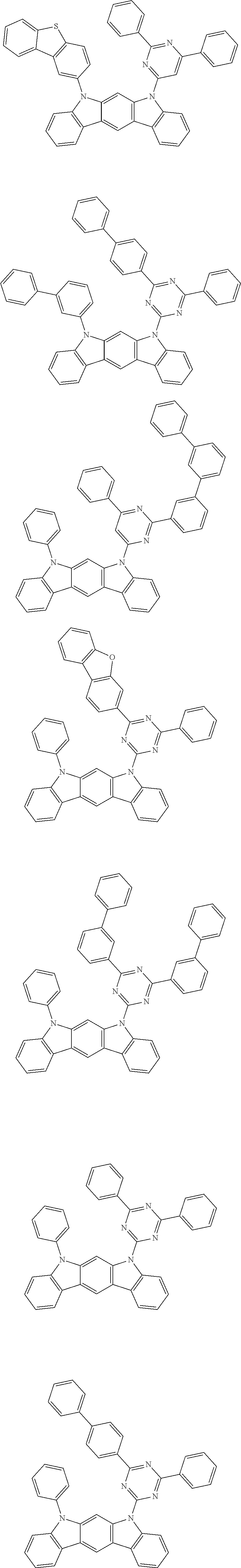 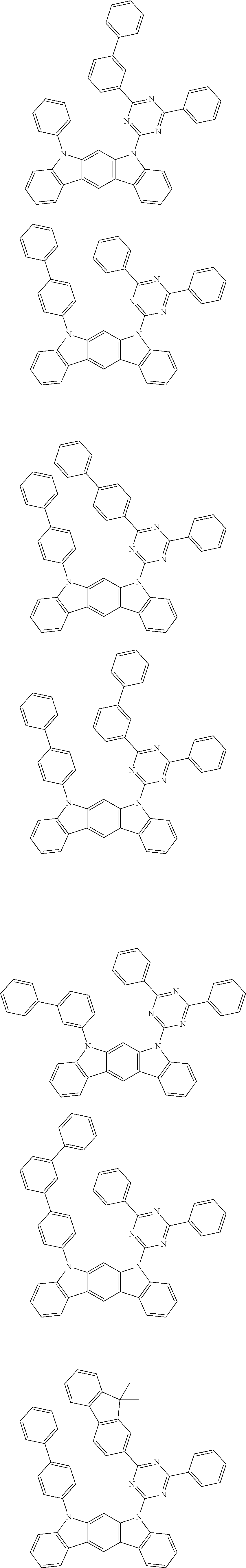 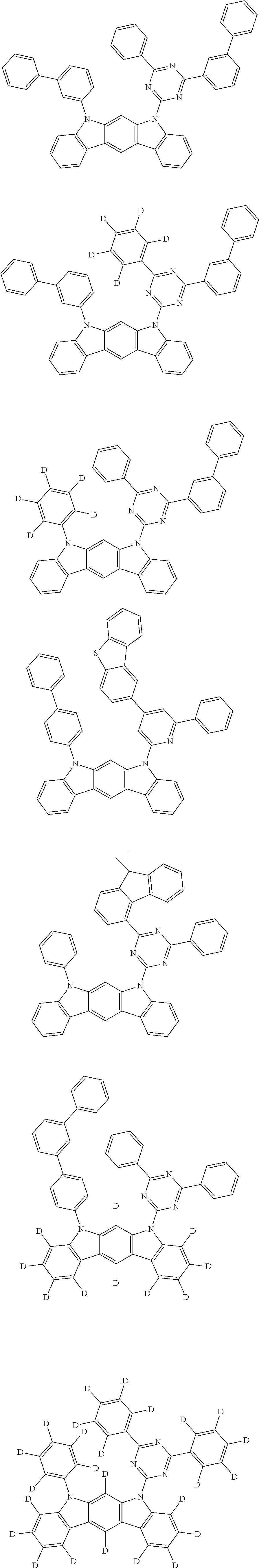 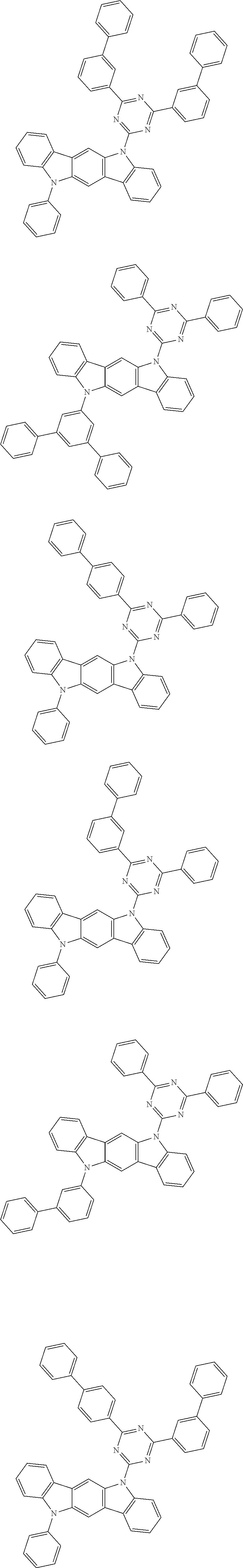 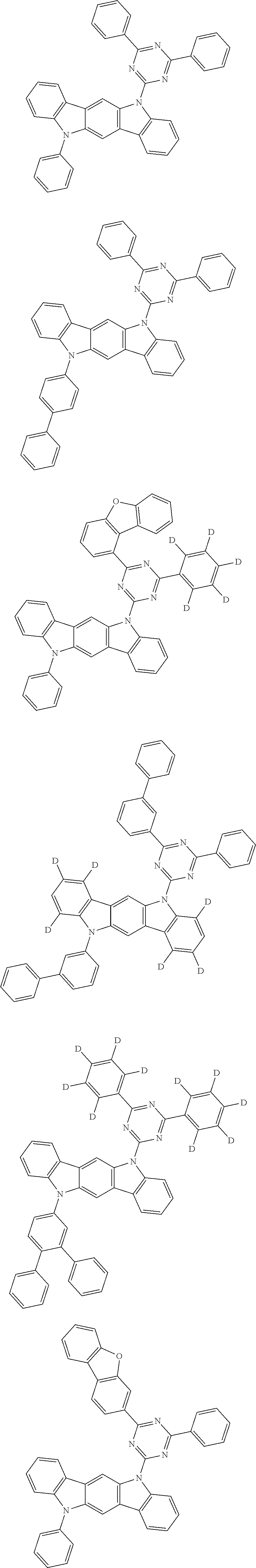 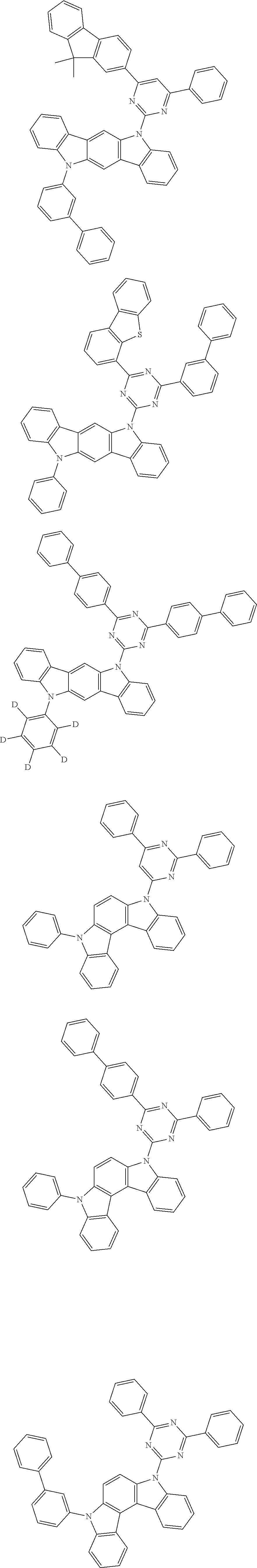 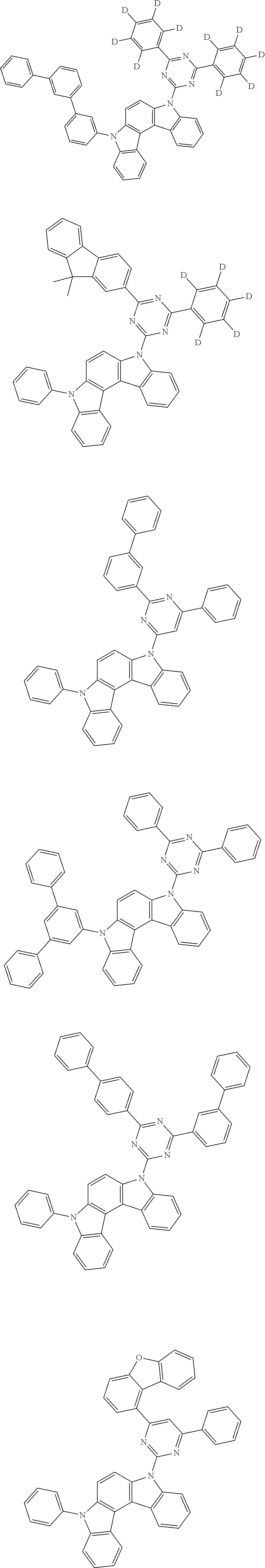 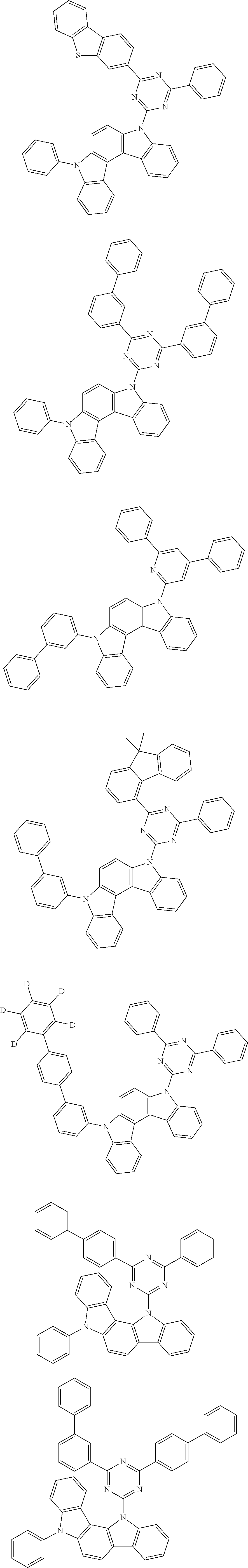 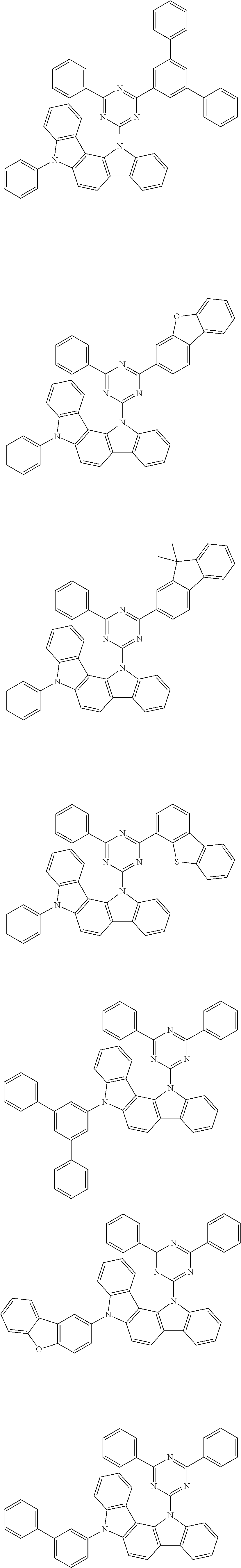 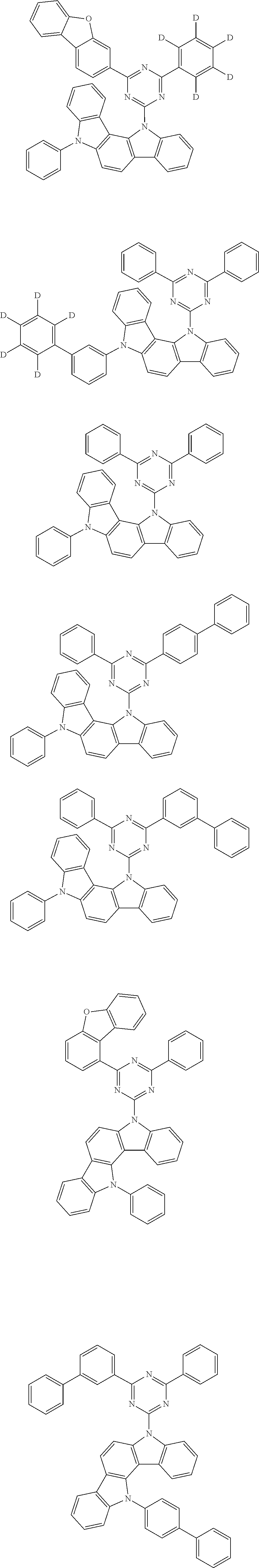 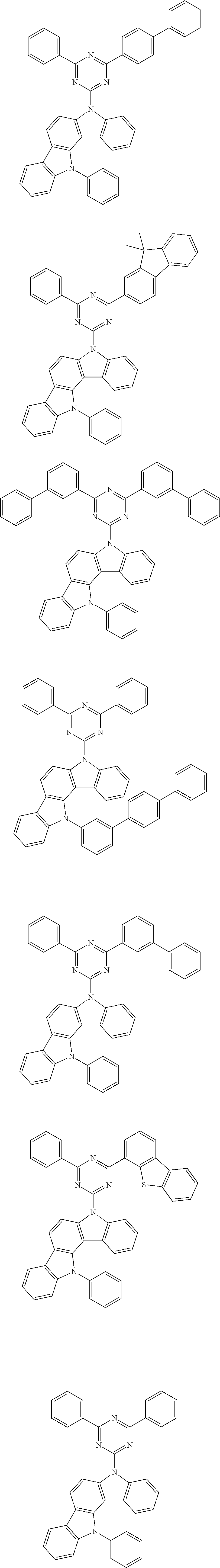 [chemical structure]
